(19) United States
(12) Patent Application Publication
(10) Pub. No.: US 2012/0171126 A1
(43) Pub. Date: Jul. 5, 2012

(54) NOVEL [3,2-c] HETEROARYL STEROIDS AS GLUCOCORTICOID RECEPTOR AGONISTS COMPOSITIONS AND USES THEREOF

(76) Inventors: Biju J. Purakkattle, Kenilworth, NJ (US); Michael Y. Berlin, Kenilworth, NJ (US); Yeon-Hee Lim, Kenilworth, NJ (US); Rema D. Bitar, Kenilworth, NJ (US); Kevin D. McCormick, Kenilworth, NJ (US); Robert G. Aslanian, Kenilworth, NJ (US); Yoon Joo Lee, Kenilworth, NJ (US); Junying Zheng, Kenilworth, NJ (US); Ying Huang, Kenilworth, NJ (US); Walter Won, Kenilworth, NJ (US)

(21) Appl. No.: 13/378,893

(22) PCT Filed: Jun. 15, 2010
(86) PCT No.: PCT/US10/38619

§ 371 Date: Mar. 9, 2012

Related U.S. Application Data
(60) Provisional application No. 61/187,521, filed on Jun. 16, 2009.

(51) Int. Cl. A61K 31/58; A61K 9/12; A61P 37/00; A61P 29/00; A61P 17/00; A61P 17/06; A61P 17/04; A61P 11/06; A61P 11/02; A61P 11/08; A61P 11/00; A61P 1/00; A61P 1/04; A61P 25/00; A61P 27/02; C07J 71/00

(52) U.S. Cl. 424/45; 540/53; 514/176; 514/171

(57) ABSTRACT

The present invention provides compounds of Formula (I), and pharmaceutically acceptable salts, solvates, esters, prodrugs, tautomers, or isomers of said compounds), having the general structure: Formula (I) wherein L, R¹, R², R³, R⁴, R⁵, and R⁶ are selected independently of each other and as defined herein. The present invention also provides compounds (and salts, solvates, esters, prodrugs, tautomers, and isomers) of Formulas (H-A), (II-A1), (II-A2), (II-A2.1), (ll-A-2.2), (ll-A-2.3), (II-A4), (H-B), (H-C), (III), (IV), (V), (VI), as described herein. Also provided are pharmaceutical compositions, methods of preparing, and methods of using such compounds in the treatment and prophylaxis of a wide range of immune, autoimmune, and inflammatory diseases and conditions.

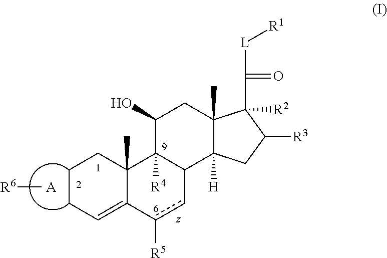

### FIELD OF THE INVENTION

This invention relates to novel A-ring modified derivatives that are agonists of the glucocorticoid receptor and methods for their preparation. The present invention also relates to pharmaceutical formulations comprising the compounds of the invention as well as to their use in the treatment of disease states involving inflammation and allergic conditions. In some embodiments, the compounds of the invention exhibit “dissociated” properties; i.e., the metabolic effects, which are associated with adverse side effects, are dissociated from the anti-inflammatory and anti-allergic effects, thereby providing glucocorticoid receptor agonists that exhibit desirable therapeutic profiles.

### BACKGROUND OF THE INVENTION

The glucocorticoid receptor is part of the family of nuclear receptors. The receptor is a nuclear transcription factor that when bound to a ligand promotes or suppresses the transcription of genes. Glucocorticoid receptor agonists occur naturally or may be prepared synthetically. Examples of synthetic glucocorticoid receptor agonists include prednisolone and dexamethasone. Glucocorticoid receptor agonists possess valuable anti-inflammatory properties and have found widespread use in the art in controlling wide range of allergic and inflammatory conditions, such as asthma, rheumatoid arthritis, eczema, psoriasis and others (see, for example, Barnes, P. “Corticosteroids: The drugs to beat” European Journal of Pharmacology 2006, 533, p. 2-14).

Steroid-based and nonsteroidal-based glucocorticoids analogues are well known in this art. For example, WO 1999/041256 describes glucocorticoids selective anti-inflammatory agents of nonsteroidal nature. GB 2,018,256, U.S. Pat. No. 3,989,686, U.S. Pat. No. 4,263,289, and EP 0 004 773 describe 17 thiocarboxylic acid steroid derivatives. WO 1997/23565 describes lactone derivatives of 17-β-carboxy, carboxythio, and amide andronstane derivative with anti-inflammatory or anti-allergic properties. WO 2006/043015 reports that the 6α,9α-difluoro-11β-hydroxy-16α-methyl-3-oxo-17α-pro-pionyloxy-androsta-1,4-diene-17β-carbothioic acid S-(2-oxo-tetrahydro-furan-3-yl)ester of the formula:

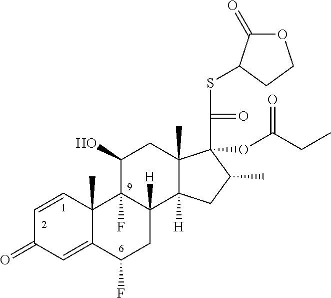

possesses useful anti-inflammatory activity, while having little or no systemic activity. Other derivatives are disclosed in WO 1997/24368, WO 2000/64882, WO 2003/035668, CN1414008, U.S. Pat. No. 3,598,816 and U.S. Pat. No. 5,645,404.

U.S. Pat. No. 4,861,765, discloses 21-substituted thioether glucocorticoid steroid derivatives of the formula:

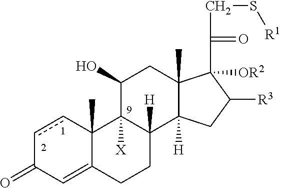

that are reported to have reduced systemic side effect and excellent anti-inflammatory properties. U.S. Pat. No. 5,420,120, also discloses 21-substituted thioether glucocorticoid steroid derivatives similar to those disclosed in U.S. Pat. No. 4,861,767; these compounds are said to be effective topical anti-inflammatory agents for the treatment of ophthalmic inflammatory disorders. Other C21-substituted thioether derivatives are disclosed in WO 1997/24367, U.S. Pat. No. 3,687,942 and S. Wu et al., Ann. Chim. Acta, vol 268, pp. 255-260 (1992).

DE20211718 discloses C21-substituted phenyl ether steroid derivatives. And WO95/18621 discloses steroids, including 6alpha,9alpha-fluoro-11beta,17-dihydroxy-16alpha-methyl-pregna-1,4-diene-3-one-17-carboxylic acid and related compounds. According to the description, the steroids disclosed in WO95/18621 have angiostatic activity and reduced glucocorticoid activity. One such compound exemplified (in example 23) in WO95/18621 has the following structure:

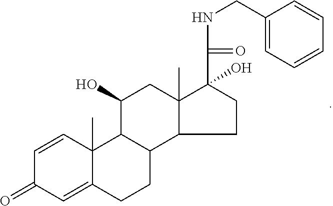

A-ring modified steroid derivatives are also known in the art. See, e.g., Ali, Amjad, et al., “Novel N-Aryl pyrazolo[3,2-c]-Based Ligands for the Glucocorticoid Receptor: Receptor Binding and In vivo Activity', J. Med. Chem., 47, 2441-2452 (Nov. 20, 2003). S. L. Steelman, “16-Methylated Steroids. IV. 6,16alpha-Dimethyl-delta-hydrocortsone and related compounds”, Merck Institute for Therapeutic Research, Nov. 30, 1962. Steelman, et al., “Synthesis and structure of steroidal 4-pregneno[3,2-c]pyrazoles. A novel class of potent anti-inflammatory steroids,”Nov. 30, 1062. Clinton, et al., “Steroidal [3,2-c]Pyrazoles”, Sterling-Winthrop Research Institute, Feb. 10, 1959. U.S. Pat. No. 3,223,701; BE633906; GB1044304(A); U.S. Pat. No. 3,067,193(A); U.S. Pat. No. 3,148,183(A); U.S. Pat. No. 3,148,183(A); and WO2009044200(A1). There remains a need in the art for glucocorticoid receptor agonists. The present invention addresses this need.

### SUMMARY OF THE INVENTION

The present invention provides novel steroid compounds, as described herein, which exhibit good pharmacological (e.g., glucocorticoid) activity. Such compounds may be referred to herein as “compound(s) of the invention.” In some embodiments, the compounds of the invention exhibit desirable pharmacological activity, such as anti-inflammatory activity and antiallergenic activity. In some embodiments, the compounds of the invention exhibit desirable pharmacological activity, such as anti-inflammatory activity and antiallergenic activity and reduced side effect activity typically associated with standard long-term steroidal treatments. Such side effect activity typically associated with standard long-term steroidal treatments include interference with carbohydrate metabolism, inappropriate calcium resorption, suppression of endogenous corticosteroids, and/or suppression of the pituitary, adrenal cortex and/or thymus gland function.

In each of the various embodiments of the compounds of the invention, all variables are selected independently of each other unless otherwise specifically noted.

In one embodiment, the compounds of the invention have the general structure shown in Formula (I):

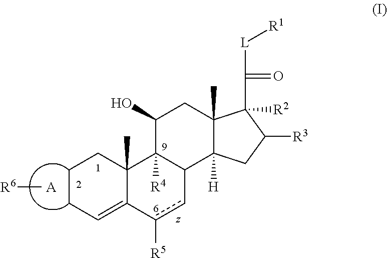

and include pharmaceutically acceptable salts, solvates, esters, prodrugs, tautomers, and/or isomers thereof, wherein:

ring A is a 5-membered heteroaryl ring containing from 1 to 2 ring heteroatoms, wherein each said ring heteroatom is independently selected from the group consisting of O, N, and S;

the dotted line at z represents an optional single or double bond;

L is a divalent moiety selected from the group consisting of

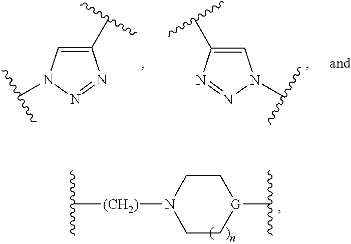

wherein G is N or CH and n is an integer from 0 to 2, with the proviso that when n is 0, G is CH,

or, alternatively, -L- is a divalent moiety selected from the group consisting of —CH₂S—, —S—, —CH₂—, —OCH₂—, —CH₂O—, —SCH₂—, —CH₂—S—CH₂—C(O)—NH—, —CH₂—OC(O)—NH—, —CH₂S(O)—, —CH₂S(O)₂—, —NR¹¹—, —N(R¹¹)—C(O)—, —N(R¹¹)—S(O)—, —N(R¹¹)—S(O)₂—, —NR¹¹O—, —CH₂N(R¹¹)—, —CH₂—N(R¹¹)—C(O)—, —CH₂—N(R¹¹)—C(O)—N(R¹¹)—, —CH₂—N(R¹¹)—C(O)O—, —CH₂N(R¹¹)C(═NH)NR¹¹—, —CH₂—N(R¹¹)—S(O)—, and —CH₂—N(R¹¹)—S(O)₂—,

R¹ is selected from the group consisting of —CN, alkyl, alkynyl, aryl, arylalkyl-, heteroarylfused aryl-, heteroarylfused arylalkyl-, cycloalkylfused aryl-, cycloalkylfused arylalkyl-, heteroaryl, heteroarylalkyl-, benzofused heteroaryl-, benzofused heteroarylalkyl-, heteroarylfused heteroaryl-, heteroarylfused heteroarylalkyl-, cycloalkyl, cycloalkenyl, cycloalkylalkyl-, cycloalkenylalkyl-, heterocycloalkyl, heterocycloalkenyl, heterocycloalkylalkyl-, heterocycloalkenylalkyl-, benzofused heterocycloalkyl-, benzofused heterocycloalkenyl-, benzofused heterocycloalkylalkyl-, benzofused heterocycloalkenylalkyl-, heteroarylfused heterocycloalkenyl-, and heteroarylfused heterocycloalkenylalkyl-,
wherein each said hetero ring-containing moiety of R¹ and each said heterofused containing moiety of R¹ independently contains 1, 2, or 3 ring heteroatoms independently selected from the group consisting of any combination of N, O, and S,
wherein each said R¹ group is unsubstituted or optionally substituted with from 1 to 5 substituents, which may be the same or different, each independently selected from the group consisting of halogen, hydroxy, —CN, oxo, oxide, alkyl, alkenyl, alkynyl, haloalkyl, haloalkoxy-, hydroxyalkyl-, heteroalkyl, cyanoalkyl-, alkoxy, optionally substituted aryl, optionally substituted —O-aryl, optionally substituted —O-alkyl-aryl, optionally substituted heteroaryl, optionally substituted arylalkyl-, optionally substituted arylalkoxy, optionally substituted heterocycloalkyl, optionally substituted heterocycloalkylalkyl-, optionally substituted —O-heterocycloalkyl, —N(R⁷)₂, -alkylN(R⁷)₂, —NC(O)R⁷, —C(O)R⁷, —CO₂R⁷, —SO₂R⁷, and —SO₂N(R⁷)₂, wherein said optional substituents are present from 1 to 4 times and may be the same or different, each independently selected from the group consisting of alkyl, halogen, haloalkyl, hydroxyl, —CN, and —N(R¹¹)₂;
and wherein the benzo portion of each said benzofused R¹ group is optionally further fused to another ring selected from the group consisting of heteroaryl, cycloalkyl, cycloalkenyl, heterocycloalkyl, and heterocycloalkenyl,
and wherein the alkyl- portion of said arylalkyl-, heteroarylfused arylalkyl-, cycloalkylfused arylalkyl-, heteroarylalkyl-, benzofused heteroarylalkyl-, heteroarylfused heteroarylalkyl-, cycloalkylalkyl-, cycloalkenylalkyl-, heterocycloalkylalkyl-, heterocycloalkenylalkyl-, benzofused heterocycloalkylalkyl-, benzofused heterocycloalkenylalkyl-, and heteroarylfused heterocycloalkenylalkyl- of R¹ is optionally substituted with one or more substituents independently selected from the group consisting of alkyl, haloalkyl, and spirocycloalkyl;

R² is selected from the group consisting of —OR⁸;

R³ is selected from the group consisting of H, —OH, and alkyl;

or R² and R³ are taken together to form a moiety of formula 2:

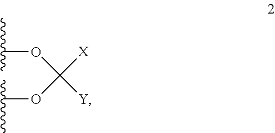

wherein X and Y are each independently selected from the group consisting of hydrogen, alkyl, haloalkyl, cycloalkyl, heterocycloalkyl, aryl, and heteroaryl,
wherein each of said alkyl, haloalkyl, cycloalkyl, heterocycloalkyl, aryl and heteroaryl of X and Y is optionally independently unsubstituted or substituted with from 1 to 4 substituents independently selected from the group consisting of alkyl, halogen, haloalkyl, hydroxy, —N(R⁷)₂, and —CN,
or X and Y of formula 2 are taken together with the carbon atom to which they are attached to form a 3 to 7-membered cycloalkyl or heterocycloalkyl ring, which ring is optionally substituted with from 1 to 4 substituents independently selected from the group consisting of alkyl, halogen, haloalkyl, hydroxy, —N(R⁷)₂ and —CN,
or R² and R³ taken together form a moiety of formula 3:

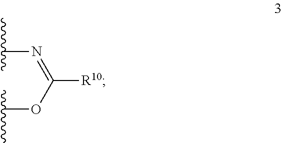

R⁴ is selected from the group consisting of H, halogen, and alkyl;

R⁵ is selected from the group consisting of H, halogen, and alkyl

R⁶ is selected from the group consisting of H, alkyl, -alkyl-CN, -alkyl-OH, alkoxy, heteroalkyl, —O-heteroalkyl, haloalkyl, aryl, arylalkyl-, naphthyl, naphthylalkyl-, heteroarylfused aryl, heteroarylfused arylalkyl-, cycloalkylfused aryl, cycloalkylfused arylalkyl-, heteroaryl, heteroarylalkyl-, benzofused heteroaryl, benzofused heteroarylalkyl-, heteroarylfused heteroaryl, heteroarylfused heteroarylalkyl-, cycloalkyl, cycloalkenyl, cycloalkylalkyl-, cycloalkenylalkyl-, heterocycloalkyl, heterocycloalkenyl, heterocycloalkylalkyl-, heterocycloalkenylalkyl-, benzofused heterocycloalkyl, benzofused heterocycloalkenyl, benzofused heterocycloalkylalkyl-, benzofused heterocycloalkenylalkyl-, heteroarylfused heterocycloalkenyl, and heteroarylfused heterocycloalkenylalkyl-,
wherein each said hetero ring-containing moiety of R⁶ contains 1, 2, or 3 ring heteroatoms independently selected from the group consisting of any combination of N, O, and S, and
wherein each said R⁶ (when other than H) is unsubstituted or substituted with from 1 to 4 groups independently selected from the group consisting of halogen, —CN, —OH, alkyl, haloalkyl, alkoxy, and —N(R⁷);

each R⁷ is independently selected from the group consisting of hydrogen, alkyl, haloalkyl, aryl, and heteroaryl,

or, two groups R⁷ attached to the same nitrogen atom form a 3- to 7-membered heterocycloalkyl group;

R⁸ selected from the group consisting of hydrogen, alkyl, haloalkyl, aryl, heteroaryl, arylalkyl, heteroarylalkyl, —C(O)R⁹, and —C(O)NHR⁹;

each R⁹ is independently selected from the group consisting of alkyl, haloalkyl, aryl, heteroaryl, cycloalkyl, and heterocycloalkyl, each optionally substituted with 1 to 4 substituents independently selected from the group consisting of alkyl, halogen, haloalkyl, hydroxy, —N(R⁷), and —CN;

each R¹⁰ is independently selected from the group consisting of hydrogen and alkyl; and

each R¹¹ is independently selected from the group consisting of hydrogen and alkyl.

In another embodiment, pharmaceutical formulations or compositions comprising a therapeutically effective amount of at least one of the compounds of the invention, and/or a pharmaceutically acceptable salt, solvate, ester, prodrug, or isomer thereof, and a pharmaceutically acceptable carrier also are provided. In another embodiment, pharmaceutical formulations or compositions comprising a therapeutically effective amount of at least one of the inventive compounds (and/or a pharmaceutically acceptable salt, solvate, ester, prodrug, or isomer thereof) and a pharmaceutically acceptable carrier together with one or more additional active ingredients are also contemplated.

In another embodiment, the present invention provides methods of treating inflammatory diseases and conditions, such methods comprising administering at least one compound or composition of the invention to a patient in need thereof.

In another embodiment, the present invention provides methods for the treatment of inflammatory diseases and conditions in a patient in need thereof, wherein the anti-inflammatory properties are dissociated from the systemic side-effects which comprises administering to said patient a dissociated steroid compound of the invention.

### DETAILED DESCRIPTION

The terms used herein have their ordinary meaning and the meaning of such terms is independent at each occurrence thereof. That notwithstanding and except where stated otherwise, the following definitions apply throughout the specification and claims. Chemical names, common names and chemical structures may be used interchangeably to describe that same structure. These definitions apply regardless of whether a term is used by itself or in combination with other terms, unless otherwise indicated. Hence the definition of “alkyl” applies to “alkyl” as well as the “alkyl” portion of “hydroxyalkyl”, “haloalkyl”, arylalkyl-, alkylaryl-, “alkoxy” etc.

As will be appreciated by those of ordinary skill in the art, conventions for depicting the stereoconfiguration of steroidal compounds have developed. The present disclosure conforms to such convention. Thus, for example, the C8, C14, 10-CH₃, and 18-CH₃ positions of the steroid core, when depicted herein as:

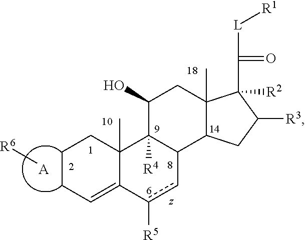

are for purposes of this disclosure and the appended claims considered equivalent to the stereoconfiguration shown as follows:

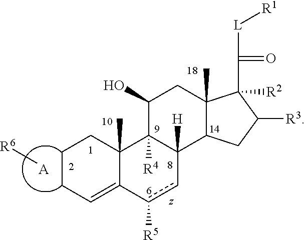

As described herein, the variable “-L-” (or “L”), when present in the various generic formulas depicting compounds of the invention, is shown as a divalent moiety. It shall be understood that the various moieties within the definitions of L, throughout the description and claims, are to be read from left to right as written, such that the point of attachment of the left-most bond of L is to the rest of the compound, and the point of attachment of the right-most bond of L as written is understood to be R¹. Thus, as a non-limiting example, when -L- is written as —CH₂—S—, the points of attachment of -L- are understood to be as follows: “rest of molecule” —CH₂—S—R¹.

“Patient” includes both human and animals.

“Mammal” means humans and other mammalian animals.

“Halogen” means fluorine, chlorine, bromine, or iodine. Preferred are fluorine, chlorine and bromine.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being as described herein or independently selected from the group consisting of halo, alkyl, haloalkyl, spirocycloalkyl, aryl, cycloalkyl, cyano, hydroxy, alkoxy, alkylthio, amino, —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Haloalkyl” means an alkyl as defined above wherein one or more hydrogen atoms on the alkyl is replaced by a halo group defined above.

“Heteroalkyl” means an alkyl moiety as defined above, having one or more carbon atoms, for example one, two or three carbon atoms, replaced with one or more heteroatoms, which may be the same or different, where the point of attachment to the remainder of the molecule is through a carbon atom of the heteroalkyl radical. Suitable such heteroatoms include O, S, and N. Non-limiting examples include ethers, thioethers, amines, hydroxymethyl, 3-hydroxypropyl, 1,2-dihydroxyethyl, 2-methoxyethyl, 2-aminoethyl, 2-dimethylaminoethyl, and the like.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene. More generally, the suffix “ene” on alkyl, aryl, heterocycloalkyl, etc. indicates a divalent moiety, e.g., —CH₂CH₂— is ethylene, and

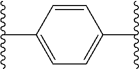

is para-phenylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Alkenylene” means a difunctional group obtained by removal of a hydrogen from an alkenyl group that is defined above. Non-limiting examples of alkenylene include —CH═CH—, —C(CH₃)═CH—, and —CH═CHCH₂—.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined herein. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like. Further non-limiting examples of cycloalkyl include the following:

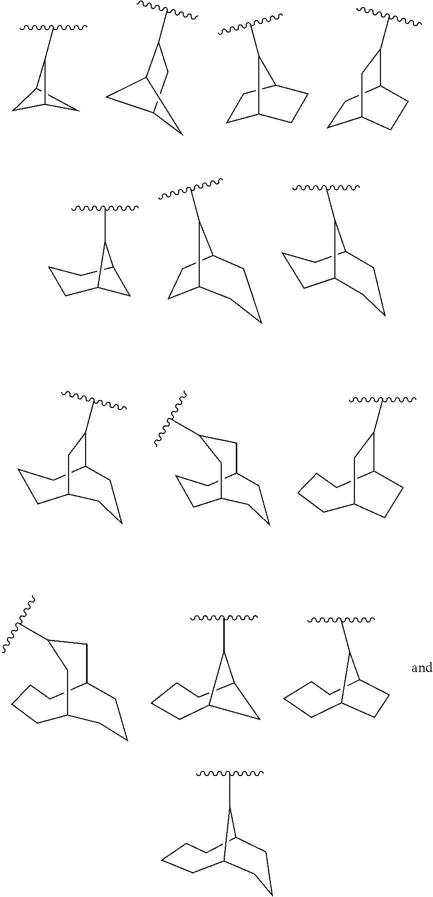

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl, as well as unsaturated moieties of the examples shown above for cycloalkyl.

“Heterocycloalkyl” (or “heterocyclyl”) means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Thus, the term “oxide,” when it appears in a definition of a variable in a general structure described herein, refers to the corresponding N-oxide, S-oxide, or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes rings wherein =0 replaces two available hydrogens on the same carbon atom (i.e., heterocyclyl includes rings having a carbonyl group in the ring). Such ═O groups may be referred to herein as “oxo.” Example of such moiety is pyrrolidone:

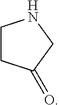

“Heterocycloalkenyl” (or “heterocyclenyl”) means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes rings wherein =0 replaces two available hydrogens on the same carbon atom (i.e., heterocyclyl includes rings having a carbonyl group in the ring). Example of such moiety is pyrrolidinone:

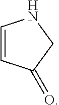

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

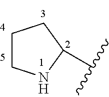

there is no —OH attached directly to carbons marked 2 and 5.

It should also be noted that tautomeric forms such as, for example, the moieties:

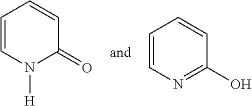

are considered equivalent in certain embodiments of this invention.

“Arylcycloalkyl” (or “arylfused cycloalkyl”) means a group derived from a fused aryl and cycloalkyl as defined herein. Preferred arylcycloalkyls are those wherein aryl is phenyl (which may be referred to as “benzofused”) and cycloalkyl consists of about 5 to about 6 ring atoms. The arylcycloalkyl can be optionally substituted as described herein. Non-limiting examples of suitable arylcycloalkyls include indanyl (a benzofused cycloalkyl) and 1,2,3,4-tetrahydronaphthyl and the like. The bond to the parent moiety is through a non-aromatic carbon atom.

“Arylheterocycloalkyl” (or “arylfused heterocycloalkyl”) means a group derived from a fused aryl and heterocycloalkyl as defined herein. Preferred arylcycloalkyls are those wherein aryl is phenyl (which may be referred to as “benzofused”) and heterocycloalkyl consists of about 5 to about 6 ring atoms. The aryiheterocycloalkyl can be optionally substituted, and/or contain the oxide or oxo, as described herein. Non-limiting examples of suitable arylfused heterocycloalkyls include:

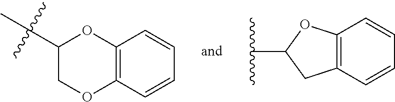

The bond to the parent moiety is through a non-aromatic carbon atom.

It is also understood that the terms “arylfused aryl-”, “arylfused cycloalkyl-”, “arylfused cycloalkenyl-”, “arylfused heterocycloalkyl-”, “arylfused heterocycloalkenyl-”, “arylfused heteroaryl-”, “cycloalkylfused aryl-”, “cycloalkylfused cycloalkyl-”, “cycloalkylfused cycloalkenyl-”, “cycloalkylfused heterocycloalkyl-”, “cycloalkylfused heterocycloalkenyl-”, “cycloalkylfused heteroaryl-, “cycloalkenylfused aryl-”, “cycloalkenylfused cycloalkyl-”, “cycloalkenylfused cycloalkenyl-”, “cycloalkenylfused heterocycloalkyl-”, “cycloalkenylfused heterocycloalkenyl-”, “cycloalkenylfused heteroaryl-”, “heterocycloalkylfused aryl-”, “heterocycloalkylfused cycloalkyl-”, “heterocycloalkylfused cycloalkenyl-”, “heterocycloalkylfused heterocycloalkyl-”, “heterocycloalkylfused heterocycloalkenyl-”, “heterocycloalkylfused heteroaryl-”, “heterocycloalkenylfused aryl-”, “heterocycloalkenylfused cycloalkyl-”, “heterocycloalkenylfused cycloalkenyl-”, “heterocycloalkenylfused heterocycloalkyl-”, “heterocycloalkenylfused heterocycloalkenyl-”, “heterocycloalkenylfused heteroaryl-”, “heteroarylfused aryl-”, “heteroarylfused cycloalkyl-”, “heteroarylfused cycloalkenyl-”, “heteroarylfused heterocycloalkyl-”, “heteroarylfused heterocycloalkenyl-”, and “heteroarylfused heteroaryl-” are similarly represented by the combination of the groups aryl, cycloalkyl, cycloalkenyl, heterocycloalkyl, heterocycloalkenyl, and heteroaryl, as previously described. Any such groups may be unsubstituted or substituted with one or more ring system substituents at any available position as described herein. The point of attachment to the parent moiety, which may be indicated by a “-”, is to the non-fused moiety.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl. The term (and similar terms) may be written as “arylalkyl-” to indicate the point of attachment to the parent moiety.

Similarly, “heteroarylalkyl”, “cycloalkylalkyl”, “cycloalkenylalkyl”, “heterocycloalkylalkyl”, “heterocycloalkenylalkyl”, etc., mean a heteroaryl, cycloalkyl, cycloalkenyl, heterocycloalkyl, heterocycloalkenyl, etc. as described herein bound to a parent moiety through an alkyl group. Preferred groups contain a lower alkyl group. Such alkyl groups may be straight or branched, unsubstituted and/or substituted as described herein.

Similarly, “arylfused arylalkyl-”, arylfused cycloalkylalkyl-, etc., means an arylfused aryl group, arylfused cycloalkyl group, etc. linked to a parent moiety through an alkyl group. Preferred groups contain a lower alkyl group. Such alkyl groups may be straight or branched, unsubstituted and/or substituted as described herein.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkylether” means a non-aromatic ring of 3 to 7 members comprising an oxygen atom and 2 to 7 carbon atoms. Ring carbon atoms can be substituted, provided that substituents adjacent to the ring oxygen do not include halo or substituents joined to the ring through an oxygen, nitrogen or sulfur atom.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl, adamantylpropyl, and the like.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclylalkyl” (or “heterocycloalkylalkyl”) means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenylalkyl” means a heterocyclenyl moiety as defined above linked via an alkyl moiety (defined above) to a parent core.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Cyanoalkyl” means a CN-alkyl- group in which alkyl is as previously defined. Preferred cyanalkyls contain lower alkyl. Non-limiting examples of suitable cyanoalkyl groups include cyanomethyl and 2-cyanoethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Alkyoxyalkyl” means a group derived from an alkoxy and alkyl as defined herein. The bond to the parent moiety is through the alkyl.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” (or “arylalkyloxy”) means an aralkyl-O— group (an arylaklyl-O-group) in which the aralkyl group is as previously described. Non-limiting examples of suitable aralkyloxy groups include benzyloxy and 1- or 2-naphthalenemethoxy. The bond to the parent moiety is through the ether oxygen.

“Arylalkenyl” means a group derived from an aryl and alkenyl as defined herein. Preferred arylalkenyls are those wherein aryl is phenyl and the alkenyl consists of about 3 to about 6 atoms. The arylalkenyl can be optionally substituted by one or more R²⁷ substituents. The bond to the parent moiety is through a non-aromatic carbon atom.

“Arylalkynyl” means a group derived from a aryl and alkenyl as defined herein. Preferred arylalkynyls are those wherein aryl is phenyl and the alkynyl consists of about 3 to about 6 atoms. The arylalkynyl can be optionally substituted by one or more R²⁷ substituents. The bond to the parent moiety is through a non-aromatic carbon atom.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

“Spirocycloalkyl” means a cycloalkyl group attached to a parent moiety at a single carbon atom. Non-limiting examples of spirocycloalkyl wherein the parent moiety is a cycloalkyl include spiro[2.5] octane, spiro[2.4] heptane, etc.

Non-limiting examples of spirocycloalkyl wherein the parent moiety is an alkyl moiety linking fused ring systems (such as the alkyl moiety in heteroarylfused heteroarylalkyl-) may optionally be substituted with spirocycloalkyl or other groups as described herein. Non-limiting spirocycloalkyl groups include spirocyclopropyl, spriorcyclobutyl, spirocycloheptyl, and spirocyclohexyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” it is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

Substitution on a cycloalkylalkyl, heterocycloalkylalkyl, arylalkyl, heteroarylalkyl, arylfused cycloalkylalkyl- moiety or the like includes substitution on any ring portion and/or on the alkyl portion of the group.

When a variable appears more than once in a group, e.g., R⁷ in —N(R⁷)₂, or a variable appears more than once in a structure presented herein such as Formula (I), the variables can be the same or different.

“Compound(s) of the invention” (or “inventive compound(s)”) refers, individually and/or collectively, to the inventive compounds encompassed by the general Formulas (I)-(VI) and (VIII), and the various embodiments described therein or the individual compounds encompassed thereby.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art. With respect to the compositions and methods comprising the use of “at least one compound of the invention, e.g., of Formula (I),” one to three compounds of the invention, e.g., of Formula (I), can be administered at the same time.

Compounds of the invention may contain one or more rings having one or more ring system substituents. “Ring system substituent” means a substituent attached to an aromatic or non-aromatic ring system which, for example, replaces an available hydrogen on the ring system. Ring system substituents may be the same or different, each being as described herein or independently selected from the group consisting of alkyl, alkenyl, alkynyl, haloalkyl, heteroalkyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)— NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moieties are rings such as heteroaryl, cycloalkyl, cycloalkenyl, heterocycloalkyl, and heterocycloalkenyl rings. Additional non-limiting examples include methylene dioxy, ethylenedioxy, —C(CH₃)₂— and the like which form moieties such as, for example:

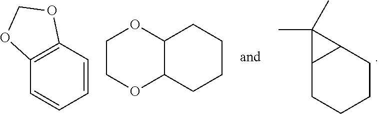

As used herein, the term “composition” is intended to encompass a product comprising the specified ingredients in the specified amounts, as well as any product which results, directly or indirectly, from combination of the specified ingredients in the specified amounts.

The line [symbol] as a bond generally indicates a mixture of, or either of, the possible isomers, e.g., containing (R)- and (S)-stereochemistry. For example:

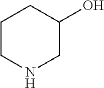

means containing both

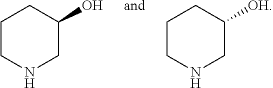

The wavy line [symbol] , as used herein, indicates a point of attachment to the rest of the compound. For example, each wavy line in the following structure:

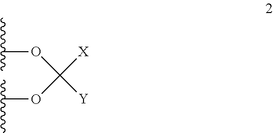

indicates a point of attachment to the core structure, as described herein.

Lines drawn into the ring systems, such as, for example:

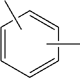

indicate that the indicated line (bond) may be attached to any of the substitutable ring carbon atoms.

“Oxo” is defined as a oxygen atom that is double bonded to a ring carbon in a cycloalkyl, cycloalkenyl, heterocyclyl, heterocyclenyl, or other ring described herein, e.g.,

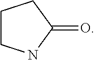

In this specification, where there are multiple oxygen and/or sulfur atoms in a ring system, there cannot be any adjacent oxygen and/or sulfur present in said ring system.

It is noted that the carbon atoms for compounds of the invention may be replaced with 1 to 3 silicon atoms so long as all valency requirements are satisfied.

As well known in the art, a bond drawn from a particular atom wherein no moiety is depicted at the terminal end of the bond indicates a methyl group bound through that bond to the atom, unless stated otherwise. For example:

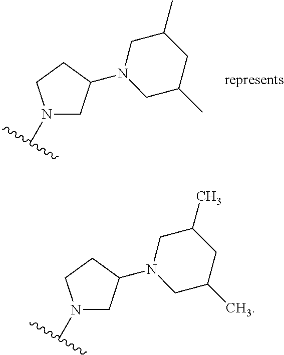

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like) in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of the invention or a pharmaceutically acceptable salt, hydrate or solvate of the compound. The transformation may occur by various mechanisms (e.g., by metabolic or chemical processes), such as, for example, through hydrolysis in blood, in the gastrointestinal tract, or in the lungs. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of the invention or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as 6-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of the invention contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₈)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

Compounds of the invention contain a hydroxyl group at the C-11 position. 11-keto prodrugs of any of the compounds of the invention may be obtained by conversion of the starting core moiety from the C-11 hydroxy to the corresponding C-11 keto compound, then following the procedures described herein. Examples of prodrugs of the compounds of the invention are shown in Table 5 below.

If a compound of the invention incorporates an amine functional group, a prodrug can be formed by the replacement of a hydrogen atom in the amine group with a group such as, for example, R-carbonyl, RO-carbonyl, NRR'-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of the invention can form salts which are also within the scope of this invention. Reference to a compound of the invention herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of the invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the invention may be formed, for example, by reacting a compound of the invention with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of the invention, and salts, solvates, esters and prodrugs thereof, may exist in their tautomeric form (for example, as an amide or imino ether). All such tautomeric forms are contemplated herein as part of the present invention.

The compounds of the invention may contain asymmetric or chiral centers, and, therefore, exist in different stereoisomeric forms. It is intended that all stereoisomeric forms of the compounds of the invention as well as mixtures thereof, including racemic mixtures, form part of the present invention. In addition, the present invention embraces all geometric and positional isomers. For example, if a compound of the invention incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the invention may be atropisomers (e.g., substituted biaryls) and are considered as part of this invention. Enantiomers can also be separated by use of chiral HPLC column.

It is also possible that the compounds of the invention may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of the invention incorporates a double bond or a fused ring, both the cis- and trans-forms, as well as mixtures, are embraced within the scope of the invention. Also, for example, all keto-enol and imine-enamine forms of the compounds are included in the invention.).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of the invention (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the invention can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of the invention, and of the salts, solvates, esters and prodrugs of the compounds of the invention, are intended to be included in the present invention.

The term “pharmaceutical composition” is also intended to encompass both the bulk composition and individual dosage units comprised of more than one (e.g., two or more) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills, aerosols and other forms suitable for inhalation, and the like. Similarly, the herein-described method of treating a patient by administering a pharmaceutical composition of the present invention is also intended to encompass the administration of the afore-said bulk composition and individual dosage units.

The following embodiments (stated as “In one embodiment” or as “In another embodiment” or “In other embodiments” and the like) are independent of each other; different such embodiments can be independently selected and combined in various combinations. Such combinations should be considered as part of the invention.

In all the embodiments shown below, where moieties for more than one variable are listed for the same embodiment, each variable should be considered as being selected independently of one another.

In the various embodiments described herein, unless otherwise stated, variables of each of the general formulas not explicitly defined in the context of the respective formula are as defined in the formula to which they refer.

In one embodiment, the compounds of the invention have the general structure shown in Formula (I) as described above and include pharmaceutically acceptable salts, solvates, esters, prodrugs, and isomers of said compounds.

In one embodiment, in Formula (I), ring A is a 5-membered heteroaryl ring containing 1 ring heteroatom, wherein said ring heteroatom is selected from the group consisting of O, N, and S.

In one embodiment, in Formula (I), ring A is a 5-membered heteroaryl ring containing 1 ring heteroatom, wherein said ring heteroatom is N.

In one embodiment, in Formula (I), ring A is a 5-membered heteroaryl ring containing 1 ring heteroatom, wherein said ring heteroatom is O.

In one embodiment, in Formula (I), ring A is a 5-membered heteroaryl ring containing 1 ring heteroatom, wherein said ring heteroatom is S.

In one embodiment, in Formula (I), ring A is a 5-membered heteroaryl ring containing 2 ring heteroatoms, wherein each said ring heteroatom is independently selected from the group consisting of O, N, and S.

In one embodiment, in Formula (I), ring A is a 5-membered heteroaryl ring containing 2 ring nitrogen atoms.

In one embodiment, in Formula (I), ring A is a 5-membered heteroaryl ring containing 2 ring heteroatoms, wherein one said ring heteroatom is N and the other said ring heteroatom is O.

In one embodiment, in Formula (I), ring A is a 5-membered heteroaryl ring containing 2 ring heteroatoms, wherein one said ring heteroatom is N and the other said ring heteroatom is S.

In one embodiment, the compounds of the invention have the general structure shown in Formula (II-A):

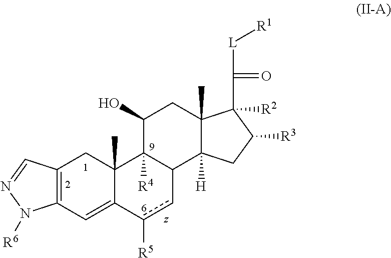

and include pharmaceutically acceptable salts, solvates, esters, prodrugs, tautomers, and/or isomers thereof, wherein:

z represents an optional single or double bond, and wherein L, R¹, R², R³, R⁴, R⁵, and R⁶ are each as defined in Formula (I), or alternatively, are as described in each of the other various embodiments described herein.

In one embodiment, in Formula (II-A), z is a single bond.

In one embodiment, in Formula (II-A), z is a double bond.

In one embodiment, the compounds of the invention have the general structure shown in Formula (II-A1):

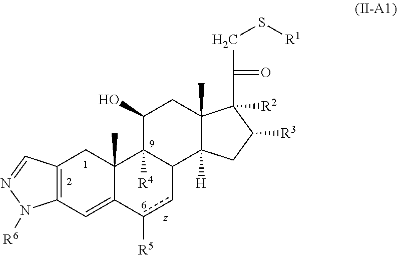

and include pharmaceutically acceptable salts, solvates, esters, prodrugs, tautomers, and/or isomers thereof, wherein:

z represents an optional single or double bond, and wherein R¹, R², R³, R⁴, R⁵, and R⁶ are each as defined in Formula (I), or alternatively, are as described in each of the other various embodiments described herein.

In one embodiment, in Formula (II-A1), z is a single bond.

In one embodiment, in Formula (II-A1), z is a double bond.

In one embodiment, the compounds of the invention have the general structure shown in Formula (II-A2):

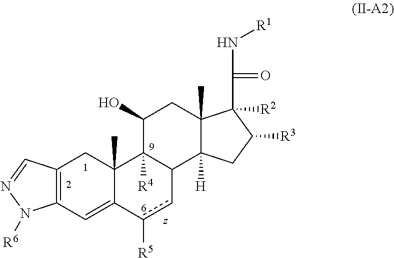

and include pharmaceutically acceptable salts, solvates, esters, prodrugs, tautomers, and/or isomers thereof, wherein:

z represents an optional single or double bond, and wherein R¹, R², R³, R⁴, R⁵, and R⁶ are each as defined in Formula (I), or alternatively, are as described in each of the other various embodiments described herein.

In one embodiment, in Formula (II-A2), z is a single bond.

In one embodiment, in Formula (II-A2), z is a double bond.

In one embodiment, the compounds of the invention have the general structure shown in Formula (II-A2.1):

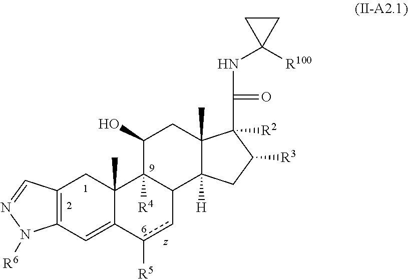

and include pharmaceutically acceptable salts, solvates, esters, prodrugs, tautomers, and/or isomers thereof, wherein:

z represents an optional single or double bond;

R¹⁰⁰ is selected from the group consisting of aryl, heteroarylfused aryl, heteroaryl, benzofused heteroaryl-, and heteroarylfused heteroaryl-,
wherein each said R¹³³ group is unsubstituted or optionally substituted with 1 to 2 substituents, which may be the same or different, each independently selected from halogen, hydroxy, —CN, alkyl, haloalkyl, alkoxy, aryl, —O-aryl and heteroaryl; and

R², R³, R⁴, R⁵, and R⁶ are each as defined in Formula (I), or, alternatively, are as described in each of the other various embodiments described herein.

In one embodiment, in Formula (II-A2.1), z is a single bond.

In one embodiment, in Formula (II-A2.1), z is a double bond.

In one embodiment, the compounds of the invention have the general structure shown in Formula (II-A2.2):

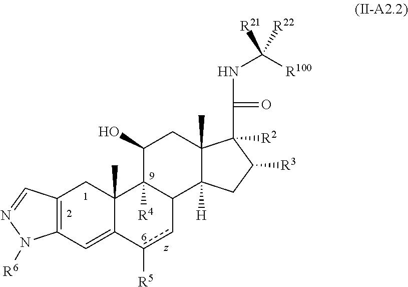

and include pharmaceutically acceptable salts, solvates, esters, prodrugs, tautomers, and/or isomers thereof, wherein:

z represents an optional single or double bond;

R¹⁰⁰ is selected from the group consisting of aryl, heteroarylfused heteroaryl, benzofused heteroaryl-, and heteroarylfused heteroaryl-,
wherein each said R¹⁰⁰ group is unsubstituted or optionally substituted with 1 to 2 substituents, which may be the same or different, each independently selected from the group consisting of halogen, hydroxy, —CN, alkyl, haloalkyl, alkoxy, aryl, —O-aryl and heteroaryl;
one of R²¹ and R²² is hydrogen and the other is selected from the group consisting of C₁-C₂ alkyl, C₁-C₂ haloalkyl, fluorine, and hydroxyl; and

R², R³, R⁴, R⁵, and R⁶ are each as defined in Formula (I), or, alternatively, are as described in each of the other various embodiments described herein.

In one embodiment, in Formula (II-A2.2), z is a single bond.

In one embodiment, in Formula (II-A2.2), z is a double bond.

In one embodiment, in Formula (II-A2.2), one of R²¹ and R²² is hydrogen and the other is selected from the group consisting of methyl and —CF₃.

In one embodiment, the compounds of the invention have the general structure shown in Formula (II-A2.3):

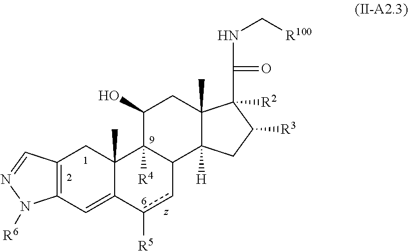

and include pharmaceutically acceptable salts, solvates, esters, prodrugs, tautomers, and/or isomers thereof, wherein:

z represents an optional single or double bond;

R¹⁰⁰ is selected from the group consisting of aryl, heteroarylfused aryl, heteroaryl, benzofused heteroaryl-, and heteroarylfused heteroaryl-,
wherein each said R¹⁰⁰ group is unsubstituted or optionally substituted with 1 to 2 substituents, which may be the same or different, each independently selected from the group consisting of halogen, hydroxy, —CN, alkyl, haloalkyl, alkoxy, aryl, —O-aryl and heteroaryl; and

R², R³, R⁴, R⁵, and R⁶ are each as defined in Formula (I), or alternatively, are as described in each of the other various embodiments described herein.

In one embodiment, in Formula (II-A2.3), z is a single bond.

In one embodiment, in Formula (II-A2.3), z is a double bond.

In one embodiment, the compounds of the invention have the general structure shown in Formula (II-A3):

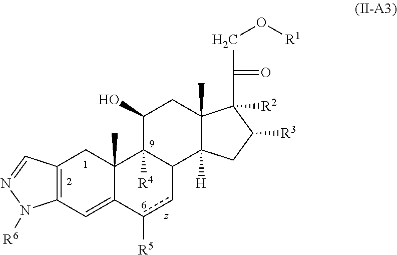

and include pharmaceutically acceptable salts, solvates, esters, prodrugs, tautomers, and/or isomers thereof, wherein:

z represents an optional single or double bond;

R¹, R², R³, R⁴, R⁵, and R⁶ are each as defined in Formula (I), or alternatively, are as described in each of the other various embodiments described herein.

In one embodiment, in Formula (II-A3), z is a single bond.

In one embodiment, in Formula (II-A3), z is a double bond.

In one embodiment, the compounds of the invention have the general structure shown in Formula (II-A4):

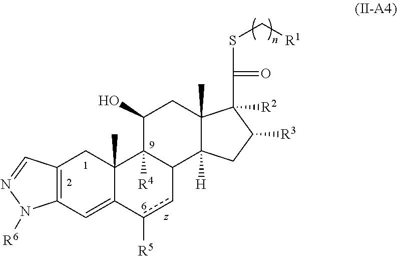

and include pharmaceutically acceptable salts, solvates, esters, prodrugs, tautomers, and/or isomers thereof, wherein:

n is 0 or 1; and

z represents an optional single or double bond;

R¹, R², R³, R⁴, R⁵, and R⁶ are each as defined in Formula (I), or alternatively, are as described in each of the other various embodiments described herein.

In one embodiment, in Formula (II-A4), z is a single bond.

In one embodiment, in Formula (II-A4), z is a double bond.

In one embodiment, the compounds of the invention have the general structure shown in Formula (II-B):

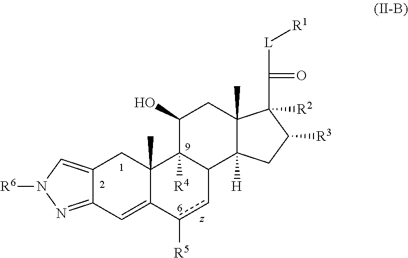

and include pharmaceutically acceptable salts, solvates, esters, prodrugs, tautomers, and/or isomers thereof, wherein:

z represents an optional single or double bond;

L, R¹, R², R³, R⁴, R⁵, and R⁶ are each as defined in Formula (I), or alternatively, are as described in each of the other various embodiments described herein.

In one embodiment, in Formula (II-B), z is a single bond.

In one embodiment, in Formula (II-B), z is a double bond.

In one embodiment, the compounds of the invention have the general structure shown in Formula (II-C):

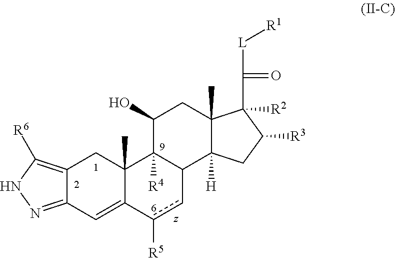

and include pharmaceutically acceptable salts, solvates, esters, prodrugs, tautomers, and/or isomers thereof, wherein:

z represents an optional single or double bond;

L, R¹, R², R³, R⁴, R⁵, and R⁶ are each as defined in Formula (I), or alternatively, are as described in each of the other various embodiments described herein.

In one embodiment, in Formula (II-C), z is a single bond.

In one embodiment, in Formula (II-C), z is a double bond.

In one embodiment, the compounds of the invention have the general structure shown in Formula (III):

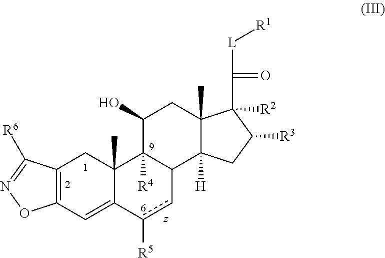

and include pharmaceutically acceptable salts, solvates, esters, prodrugs, tautomers, and/or isomers thereof, wherein:

z represents an optional single or double bond;

L, R¹, R², R³, R⁴, R⁶, and R⁶ are each as defined in Formula (I), or alternatively, are as described in each of the other various embodiments described herein.

In one embodiment, in Formula (III), z is a single bond.

In one embodiment, in Formula (III), z is a double bond.

In one embodiment, the compounds of the invention have the general structure shown in Formula (IV):

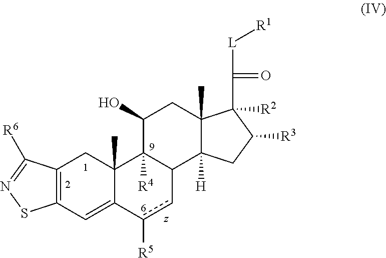

and include pharmaceutically acceptable salts, solvates, esters, prodrugs, tautomers, and/or isomers thereof, wherein:

z represents an optional single or double bond;

L, R¹, R², R³, R⁴, R⁵, and R⁶ are each as defined in Formula (I), or alternatively, are as described in each of the other various embodiments described herein.

In one embodiment, in Formula (IV), z is a single bond.

In one embodiment, in Formula (IV), z is a double bond.

In one embodiment, the compounds of the invention have the general structure shown in Formula (V):

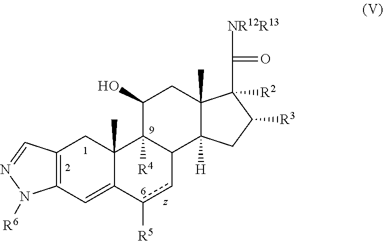

and include pharmaceutically acceptable salts, solvates, esters, prodrugs, tautomers, and/or isomers thereof, wherein:

z represents an optional single or double bond;

R¹² and R¹³ are taken together with the nitrogen to which they are shown attached to form a 3- to 7-membered heterocycloalkyl ring, a 3- to 7-membered heterocycloalkenyl ring, a 3- to 7-membered benzofused heterocycloalkyl- ring, or a 3- to 7-membered benzofused heterocycloalkenyl-ring,
wherein each said 3- to 7-membered heterocycloalkyl ring, 3- to 7-membered heterocycloalkenyl ring, 3- to 7-membered benzofused heterocycloalkyl ring, and said 3- to 7-membered benzofused heterocycloalkenyl ring is unsubstituted or substituted with from 1 to 4 substituents, which may be the same or different, independently selected from the group consisting of halogen, hydroxy, —CN, oxo, oxide, alkyl, haloalkyl, -alkyl-CN, alkoxy, aryl, halo-substituted aryl, —O-aryl, —O-alkyl-aryl, heteroaryl, arylalkyl-, arylalkoxy, haloalkoxy, —N(R⁷)₂, -alkylN(R⁷)₂, —NC(O)R⁷, —CO₂R⁷, —SO₂R⁷, and —SO₂N(R⁷)₂; and

R², R³, R⁴, R⁵, and R⁶ are each as defined in Formula (I), or alternatively, are as described in each of the other various embodiments described herein.

In one embodiment, in Formula (V), rings represented by —NR¹¹R¹² are selected from the group consisting of:

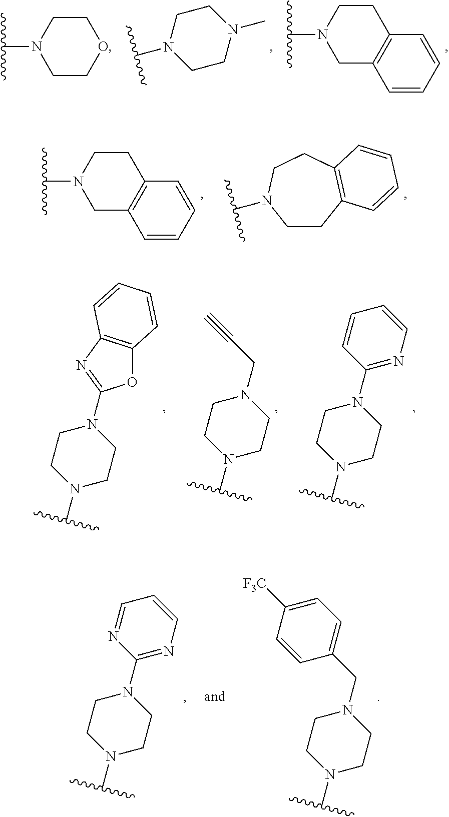

In one embodiment, in Formula (V), z is a single bond.

In one embodiment, in Formula (V), z is a double bond.

In one embodiment, the compounds of the invention have the general structure shown in Formula (VI):

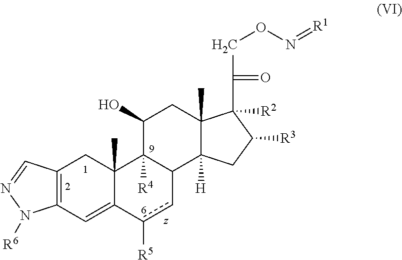

and include pharmaceutically acceptable salts, solvates, esters, prodrugs, tautomers, and/or isomers thereof, wherein:

R¹ is cycloalkyl which is unsubstituted or optionally substituted with from 1 to 5 groups, which may be the same or different, each independently selected from the group consisting of halogen, hydroxy, —CN, oxo, oxide, alkyl, haloalkyl, -alkyl-CN, alkoxy, spirocycloalkyl, aryl, halo-substituted aryl, —O-aryl, —O-alkyl-aryl, heteroaryl, arylalkyl-, arylalkoxy, haloalkoxy, —N(R⁷)₂, -alkylN(R⁷)₂, —NC(O)R⁷, —CO₂R⁷, —SO₂R⁷, and —SO₂N(R⁷)₂;

Z represents an optional single or double bond;

R², R³, R⁴, R⁵, and R⁶ are each as defined in Formula (I), or alternatively, are as described in each of the other various embodiments described herein.

In one embodiment, in Formula (VI), -L-R¹ is selected from the group

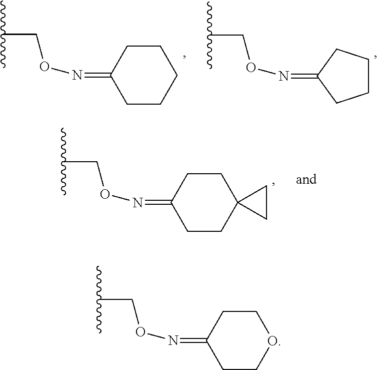

consisting of:

In one embodiment, in Formula (VI), z is a single bond.

In one embodiment, in Formula (VI), z is a double bond.

In other embodiments, in each of Formulas (I), (II-A), (II-B), (II-C), (III), and (IV):

L is a divalent moiety selected from the group consisting of

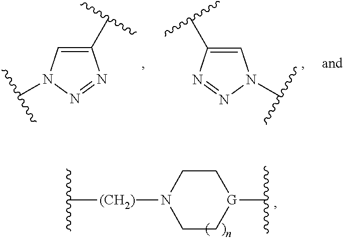

wherein G

is N or CH and n is an integer from 0 to 2, with the proviso that when n is 0, G is CH.

In other embodiments, in each of Formulas (I), (II-A), (II-B), (II-C), (III), and (IV): L is selected from the group consisting of —S—, —CH₂S—, —SCH₂—, —CH₂O—, —CH₂—S—CH₂—C(O)—NH—, —CH₂O—, —CH₂—OC(O)—NH—, —CH₂S(O)—, —CH₂S(O)₂—, —NR¹¹—, —N(R¹¹)—C(O)—, —N(R¹¹)—S(O)—, —N(R¹¹)—S(O)₂—, —NR¹¹O—, —CH₂N(R¹¹)—, —CH₂—N(R¹¹)—C(O)—, —CH₂—N(R¹¹)—C(O)—N(R¹¹)—, —CH₂—N(R¹¹)—C(O)O—, —CH₂—N(R¹¹)—OC(O)—, —CH₂N(R¹¹)C(═NH)NR¹¹—, —CH₂—N(R¹¹)—S(O)—, and —CH₂—N(R¹¹)—S(O)₂—.

In other embodiments, in each of Formulas (I), (II-A), (II-B), (II-C), (III), and (IV):

-L- is a divalent moiety selected from the group consisting of —CH₂S—, —S—, —CH₂—, —OCH₂—, —CH₂O—, —SCH₂—, and —NR¹¹—.

In other embodiments, in each of Formulas (I), (II-A), (II-B), (II-C), (III), and (IV):

-L- is —CH₂S—.

In other embodiments, in each of Formulas (I), (II-A), (II-B), (II-C), (III), and (IV):

-L- is —S—.

In other embodiments, in each of Formulas (I), (II-A), (II-B), (II-C), (III), and (IV):

-L- is —CH₂—.

In other embodiments, in each of Formulas (I), (II-A), (II-B), (II-C), (III), and (IV):

-L- is —OCH₂—.

In other embodiments, in each of Formulas (I), (II-A), (II-B), (II-C), (III), and (IV):

-L- is —CH₂O—.

In other embodiments, in each of Formulas (I), (II-A), (II-B), (II-C), (III), and (IV):

-L- is, —SCH₂—.

In other embodiments, in each of Formulas (I), (II-A), (II-B), (II-C), (III), and (IV):

-L- is, and —NR¹¹—, wherein R¹¹ is H.

In other embodiments, in each of Formulas (I), (II-A), (II-B), (II-C), (III), and (IV):

-L- is, and —NR¹¹—, wherein R¹¹ is alkyl.

In other embodiments, in each of Formulas (I), (II-A), (II-B), (II-C), (III), and (IV):

-L- is, and —NR¹¹—, wherein R¹¹ is selected from the group consisting of methyl and ethyl.

In other embodiments, in each of Formulas (I), (II-A), (II-A1), (II-A2), (II-A2.1), (II-A-2.2), (II-A-2.3), (II-A4), (II-B), (II-C), (III), (IV): R¹ is selected from the group consisting of —CN, (C₁-C₆) alkyl, and (C₁-C₆) alkynyl.

In other embodiments, in each of Formulas (I), (II-A), (II-A1), (II-A2), (II-A2.1), (II-A-2.2), (II-A-2.3), (II-A4), (II-B), (II-C), (III), (IV):

R¹ is selected from the group consisting of:

phenyl, phenylalkyl-, naphthyl, naphthylalkyl-, 4- to 6-membered heteroarylfused phenyl, 4- to 6-membered heteroarylfused phenylalkyl-, 3- to 7-membered cycloalkylfused phenyl-, 3- to 7-membered cycloalkylfused phenylalkyl-, 3- to 7-membered cycloalkenylfused phenyl-, 3- to 7-membered cycloalkenylfused phenylalkyl-, 4- to 6-membered heteroaryl-, 4- to 6-membered heteroarylalkyl-, benzofused 4- to 6-membered heteroaryl-, benzofused 4- to 6-membered heteroarylalkyl-, 4- to 6-membered heteroarylfused 4- to 6-membered heteroaryl-, 4- to 6-membered heteroarylfused 4- to 6-membered heteroarylalkyl-, 3- to 7-membered cycloalkyl-, 3- to 7-membered cycloalkylalkyl-, 3- to 7-membered cycloalkenyl-, 3- to 7-membered cycloalkenylalkyl-, 4- to 6-membered heterocycloalkyl-, 4- to 6-membered heterocycloalkylalkyl-, 4- to 6-membered heterocycloalkenyl-, 4- to 6-membered heterocycloalkenylalkyl-, benzofused 4- to 6-membered heterocycloalkyl-, benzofused 4- to 6-membered heterocycloalkylalkyl-, benzofused 4- to 6-membered heterocycloalkenyl-, benzofused 4- to 6-membered heterocycloalkenylalkyl-, 4- to 6-membered heteroarylfused 4- to 6-membered heterocycloalkenyl-, and 4- to 6-membered heteroarylfused 4- to 6-membered heterocycloalkenylalkyl-,
wherein each said hetero ring-containing moiety of R¹ and each said heterofused containing moiety of R¹ independently contains 1, 2, or 3 ring heteroatoms independently selected from the group consisting of N, O, and S,
wherein each said R¹ group is unsubstituted or optionally substituted with from 1 to 5 substituents, which may be the same or different, each independently selected from the group consisting of halogen, hydroxy, —CN, oxo, oxide, alkyl, alkenyl, alkynyl, haloalkyl, haloalkoxy-, hydroxyalkyl-, heteroalkyl, cyanoalkyl-, alkoxy, optionally substituted aryl, optionally substituted —O-aryl, optionally substituted —O-alkyl-aryl, optionally substituted heteroaryl, optionally substituted arylalkyl-, optionally substituted arylalkoxy, optionally substituted heterocycloalkyl, optionally substituted heterocycloalkylalkyl-, optionally substituted —O-heterocycloalkyl, —N(R⁷)₂, -alkylN(R⁷)₂, —NC(O)R⁷, —C(O)R⁷, —CO₂R⁷, —SO₂R⁷, and —SO₂N(R⁷)₂, wherein said optional substituents are present from 1 to 4 times and may be the same or different, each independently selected from the group consisting of alkyl, halogen, haloalkyl, hydroxyl, —CN, and —N(R¹¹)₂;
and wherein the benzo portion of each said benzofused R¹ group is optionally further fused to another ring selected from the group consisting of heteroaryl, cycloalkyl, cycloalkenyl, heterocycloalkyl, and heterocycloalkenyl,
and wherein the alkyl- portion of said phenylalkyl-, naphthylalkyl-, 4-to 6-membered heteroarylfused phenylalkyl-, 3- to 7-membered cycloalkylfused phenylalkyl-, 3- to 7-membered cycloalkenylfused phenylalkyl-, 4- to 6-membered heteroarylalkyl-, benzofused 4- to 6-membered heteroarylalkyl-, 4- to 6-membered heteroarylfused O-5 to 6-membered heteroarylalkyl-, 3- to 7-membered cycloalkylalkyl-, 3- to 7-membered cycloalkenylalkyl-, 4- to 6-membered heterocycloalkylalkyl-, 4- to 6-membered heterocycloalkenylalkyl-, benzofused 4- to 6-membered heterocycloalkylalkyl-, benzofused 4- to 6-membered heterocycloalkenylalkyl-, and 4- to 6-membered heteroarylfused 4- to 6-membered heterocycloalkenylalkyl-, of R¹ is optionally substituted with one or more substituents independently selected from the group consisting of alkyl, haloalkyl, and spirocycloalkyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is alkyl. Non-limiting examples of R¹, when R¹ is alkyl (which may be unsubstituted or further substituted as described herein), include: lower alkyl. Non-limiting examples of lower alkyl include methyl, ethyl, propyl (n-propyl and i-propyl), butyl (n-butyl, i-butyl, and t-butyl), pentyl (straight or branched), hexyl (straight or branched), octyl (straight or branched), and the like.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is alkynyl. Non-limiting examples of R¹, when R¹ is alkynyl (which may be unsubstituted or further substituted as described herein), include: lower alkynyl. Non-limiting examples of lower alkyl include ethynyl, propynyl (straight or branched), butynyl (straight or branched), pentynyl (straight or branched), hexynyl (straight or branched), octynyl (straight or branched), and the like. In one such non-limiting embodiment, R¹ is

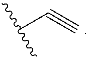

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is aryl. Non-limiting examples of R¹, when R¹ is aryl (which may be unsubstituted or further substituted as described herein), include phenyl and naphthyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is arylalkyl-. Non-limiting examples of R¹, when R′ is arylalkyl- (which may be unsubstituted or further substituted as described herein), include those moieties wherein the aryl portion of arylalkyl- is selected from the group consisting o phenyl and naphthyl, and wherein the alkyl portion of said arylalkyl- (which may be unsubstituted or further substituted as described herein), is selected from the group consisting of divalent lower alkyl. Non-limiting examples of divalent lower alkyl include—methyl-, -ethyl-, -propyl- (n-propyl and i-propyl), -butyl- (n-butyl, i-butyl, and t-butyl), -pentyl- (straight or branched), -hexyl- (straight or branched), -octyl-(straight or branched), and the like.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is heteroaryl. Non-limiting examples of R¹, when R′ is heteroaryl (which may be unsubstituted or further substituted as described herein), include: pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, and benzothiazolyl. The point of attachment of said R¹ group to -L- is by replacement of any available hydrogen atom on a ring carbon or ring heteroatom.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is heteroarylalkyl-. Non-limiting examples of R¹, when R¹ is heteroarylalkyl- (which may be unsubstituted or further substituted as described herein), include: those moieties wherein the heteroaryl portion of said heteroarylalkyl- is selected from heteroaryl as described herein, and wherein said alkyl- portion of said heteroarylalkyl- is selected from divalent -alkyl-, as described herein. The point of attachment of said R¹ to -L- is through the alkyl- group.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is cycloalkyl. Non-limiting examples of R¹, when R¹ is cycloalkyl- (which may be unsubstituted or further substituted as described herein), include: cyclopropyl, cyclopentyl, cyclohexyl, cycloheptyl and the like. Non-limiting examples of suitable multicyclic cycloalkyls include 1-decalinyl, norbornyl, adamantyl and the like. Further non-limiting examples of cycloalkyl are also described herein.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is cycloalkylalkyl-. Non-limiting examples of R¹, when R¹ is cycloalkylalkyl- (which may be unsubstituted or further substituted as described herein), include those moieties wherein the cycloalkyl portion of said cycloalkylalkyl- is selected a cycloalkyl group as described herein, and wherein said alkyl- portion of said cycloalkylalkyl- is selected from divalent -alkyl-, as described herein. The point of attachment of said R¹ to -L- is through the alkyl- group.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is cycloalkenyl. Non-limiting examples of R¹, when R¹ is cycloalkenyl- (which may be unsubstituted or further substituted as described herein), include unsaturated versions of any of the following: cyclopropyl, cyclopentyl, cyclohexyl, cycloheptyl and the like. Non-limiting examples of suitable multicyclic cycloalkyls include unsaturated versions of any of the following: 1-decalinyl, norbornyl, adamantyl and the like. Further non-limiting examples of cycloalkenyl are also described herein.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is cycloalkenylalkyl-. Non-limiting examples of R¹, when R¹ is cycloalkenylalkyl-(which may be unsubstituted or further substituted as described herein), include those moieties wherein the cycloalkenyl portion of said cycloalkenylalkyl- is selected a cycloalkenyl group as described herein, and wherein said alkyl-portion of said cycloalkenylalkyl- is selected from divalent -alkyl-, as described herein. The point of attachment of said R¹ to -L- is through the alkyl- group.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is heterocycloalkyl. Non-limiting examples of R¹, when R¹ is heterocycloalkyl-(which may be unsubstituted or further substituted as described herein), include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and oxides and oo-substituted versions thereof.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is heterocycloalkylalkyl-. Non-limiting examples of R¹, when R¹ is heterocycloalkylalkyl- (which may be unsubstituted or further substituted as described herein), include), include those moieties wherein the heterocycloalkyl portion of said heterocycloalkylalkyl- is selected a heterocycloalkyl group as described herein, and wherein said alkyl- portion of said heterocycloalkylalkyl- is selected from divalent -alkyl-, as described herein. The point of attachment of said R¹ to -L- is through the alkyl- group.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is heterocycloalkenyl. Non-limiting examples of R¹, when R¹ is heterocycloalkenyl-(which may be unsubstituted or further substituted as described herein), include: 1,2,3,4- tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like, and oxides thereof or oxo-substituted versions thereof.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is heterocycloalkenylalkyl-. Non-limiting examples of R¹, when R¹ is heterocycloalkenylalkyl- (which may be unsubstituted or further substituted as described herein), include those moieties wherein the heterocycloalkenyl portion of said heterocycloalkenylalkyl- is selected a heterocycloalkenyl group as described herein, and wherein said alkyl- portion of said heterocycloalkylalkyl- is selected from divalent -alkyl-, as described herein. The point of attachment of said R¹ to -L- is through the alkyl- group.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ comprises a multicyclic moiety wherein an aryl, heteroaryl, cycloalkyl, cycloalkenyl, heterocycloalkyl or heterocycloalkenyl moiety (non-limiting examples of which are as described above) is fused to another moiety selected from the group consisting of aryl, arylalkyl-, heteroaryl, heteroarylalkyl-, cycloalkyl, cycloalkylalkyl-, cycloalkenyl, cycloalkenylalkyl-, heterocycloalkyl, heterocycloalkylalkyl-, heterocycloalkenyl, and heterocycloalkenylalkyl- (non-limiting examples of which moieties are as described above). In such moieties, the point of attachment of R¹ is indicated by “-”.

Non-limiting examples of R¹, when R¹ is benzofused 5- to 6-membered heteroaryl (which may be unsubstituted or further substituted as described herein), include:

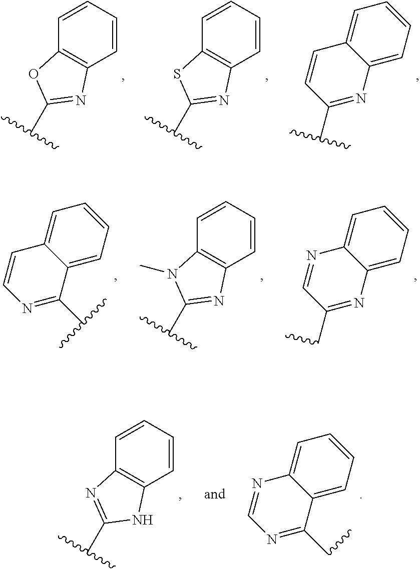

Non-limiting examples of R¹, when R¹ is heteroarylfused 5- to 6-membered heteroaryl (which may be unsubstituted or further substituted with one or more groups selected from the group consisting of halogen, —CN, —OH, alkyl, haloalkyl, alkoxy, and —N(R⁷) as described herein), include:

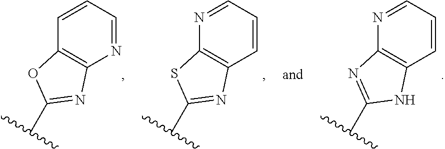

Non-limiting examples of R¹, when R¹ is heteroarylfused aryl (which may be unsubstituted or further substituted with one or more groups selected from the group consisting of halogen, —CN, —OH, alkyl, haloalkyl, alkoxy, and —N(R⁷) as described herein), include:

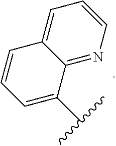

In other embodiments, in each of Formulas (I), (II-A), (II-A1), (II-A2), (II-A2.1), (II-A-2.2), (II-A-2.3), (II-A4), (II-B), (II-C), (III), (IV):

R¹ is selected from the group consisting of:

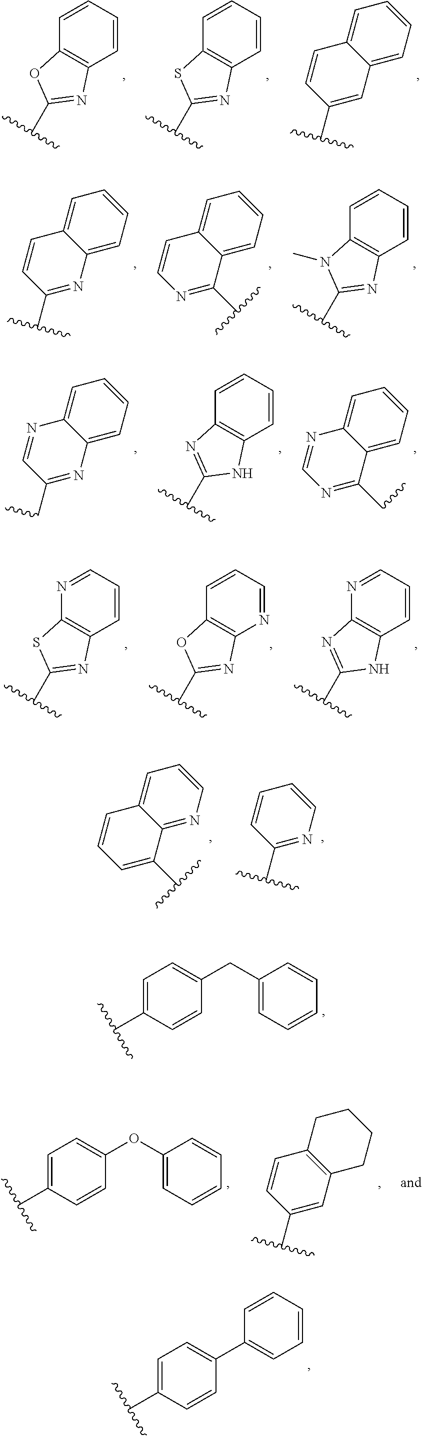

wherein each said group R¹ is unsubstituted or substituted with from 1 to 3 groups independently selected from the group consisting of halogen, hydroxyl, —CN, —N(R¹¹)₂, alkyl, haloalkyl, alkoxy, aryl, —O-aryl, heterocycloalkyl, and heteroaryl.

In other embodiments, in each of Formulas (I), (II-A), (II-A1), (II-A2), (II-A2.1), (II-A-2.2), (II-A-2.3), (II-A4), (II-B), (II-C), (III), (IV):

R¹ is selected from the group consisting of: —CN and alkynyl.

In other embodiments, in each of Formulas (I), (II-A), (II-A1), (II-A2), (II-A2.1), (II-A-2.2), (II-A-2.3), (II-A4), (II-B), (II-C), (III), (IV):

R¹ is selected from the group consisting of:

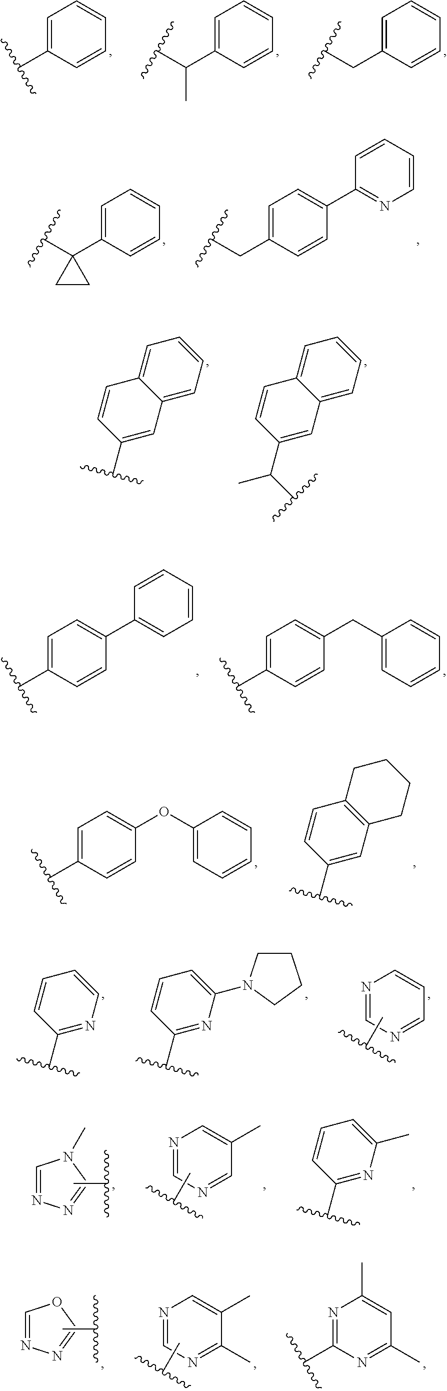

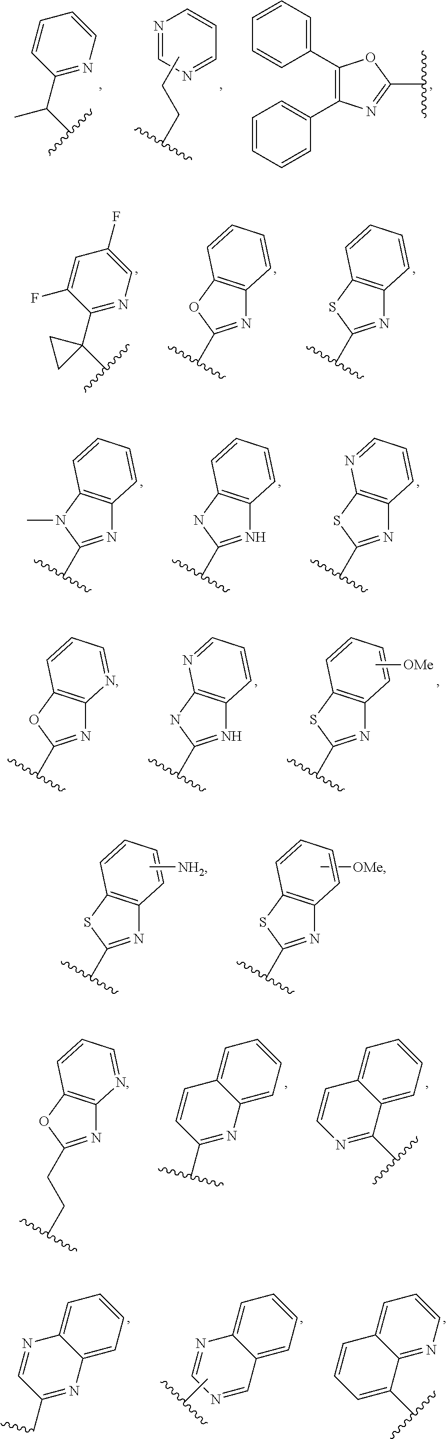

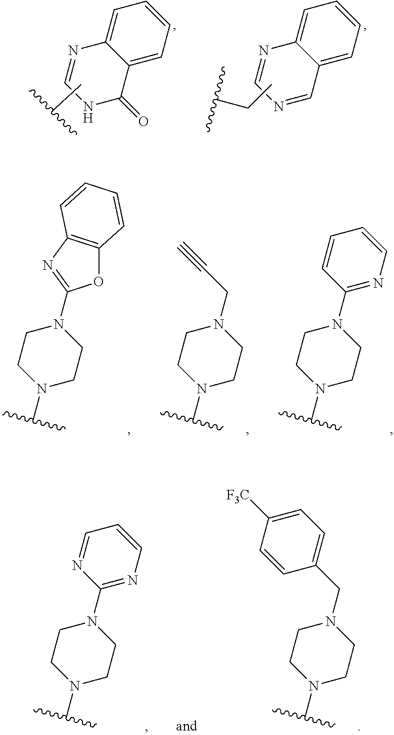

In other embodiments, in each of Formulas (I), (II-A), (II-A1), (II-A2), (II-A2.1), (II-A-2.2), (II-A-2.3), (II-A4), (II-B), (II-C), (III), (IV):

R¹ is selected from the group consisting of:

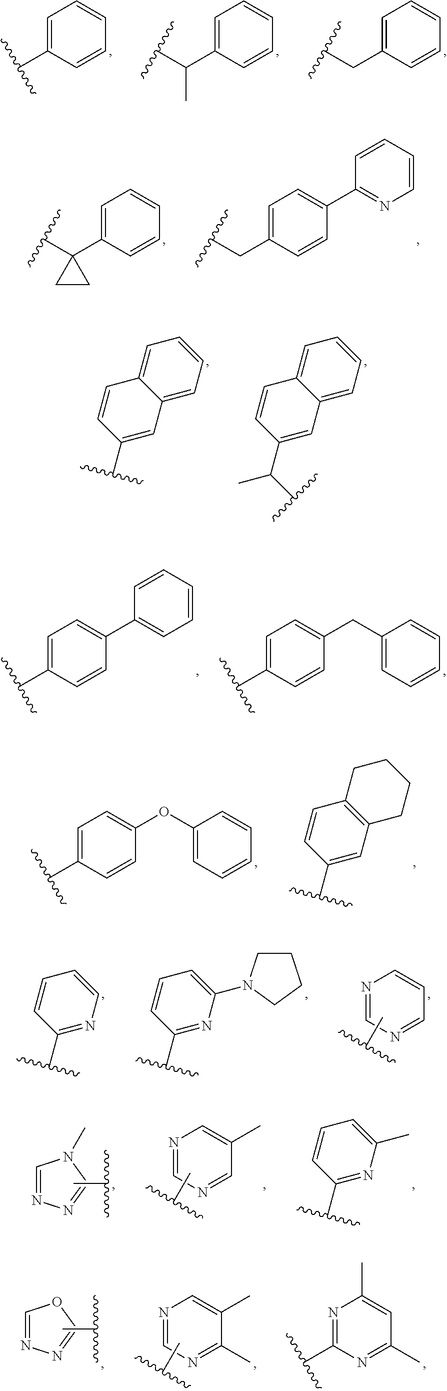

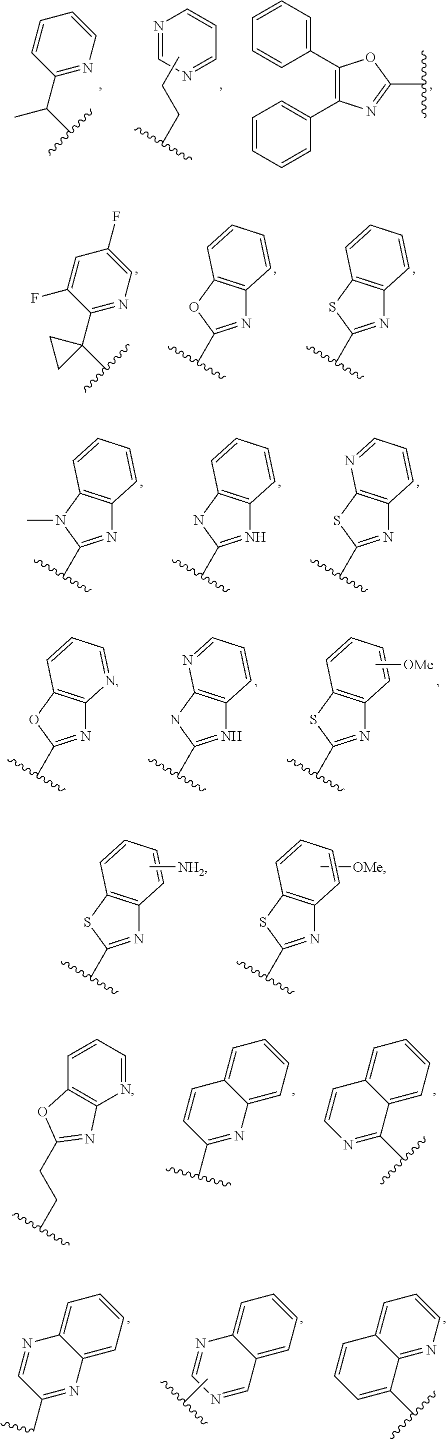

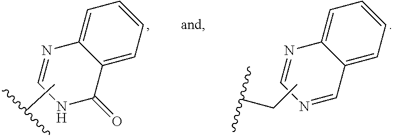

In other embodiments, in each of Formulas (I), (II-A), (II-A1), (II-A2), (II-A2.1), (II-A-2.2), (II-A-2.3), (II-A4), (II-B), (II-C), (III), (IV):
the alkyl- portion of said phenylalkyl-, naphthylalkyl-, 4- to 6-membered heteroarylfused phenylalkyl-, 3- to 7-membered cycloalkylfused phenylalkyl-, 3- to 7-membered cycloalkenylfused phenylalkyl-, 4- to 6-membered heteroarylalkyl-, benzofused 4- to 6-membered heteroarylalkyl-, 4- to 6-membered heteroarylfused O-5 to 6-membered heteroarylalkyl-, 3- to 7-membered cycloalkylalkyl-, 3- to 7-membered cycloalkenylalkyl-, 4- to 6-membered heterocycloalkylalkyl-, 4- to 6-membered heterocycloalkenylalkyl-, benzofused 4- to 6-membered heterocycloalkylalkyl-, benzofused 4- to 6-membered heterocycloalkenylalkyl-, and 4- to 6-membered heteroarylfused 4- to 6-membered heterocycloalkenylalkyl-, of R¹ is optionally substituted with from 1 to 2 substituents independently selected from the group consisting of alkyl, haloalkyl, and spirocycloalkyl.

In other embodiments, in each of Formulas (I), (II-A), (II-A1), (II-A2), (II-A2.1), (II-A-2.2), (II-A-2.3), (II-A4), (II-B), (II-C), (III), (IV):
the alkyl- portion of said phenylalkyl-, naphthylalkyl-, 4- to 6-membered heteroarylfused phenylalkyl-, 3- to 7-membered cycloalkylfused phenylalkyl-, 3- to 7-membered cycloalkenylfused phenylalkyl-, 4- to 6-membered heteroarylalkyl-, benzofused 4- to 6-membered heteroarylalkyl-, 4- to 6-membered heteroarylfused 4-to 6-membered heteroarylalkyl-, 3- to 7-membered cycloalkylalkyl-, 3- to 7-membered cycloalkenylalkyl-, 4- to 6-membered heterocycloalkylalkyl-, 4- to 6-membered heterocycloalkenylalkyl-, benzofused 4- to 6-membered heterocycloalkylalkyl-, benzofused 4- to 6-membered heterocycloalkenylalkyl-, and 4- to 6-membered heteroarylfused 4- to 6-membered heterocycloalkenylalkyl-, of R¹ is optionally substituted with from 1 to 2 substituents independently selected from the group consisting of (C₁-C₃)alkyl, and (C₁-C₃)haloalkyl.

In other embodiments, in each of Formulas (I), (II-A), (II-A1), (II-A2), (II-A2.1), (II-A-2.2), (II-A-2.3), (II-A4), (II-B), (II-C), (III), (IV):
the alkyl- portion of said phenylalkyl-, naphthylalkyl-, 4- to 6-membered heteroarylfused phenylalkyl-, 3- to 7-membered cycloalkylfused phenylalkyl-, 3- to 7-membered cycloalkenylfused phenylalkyl-, 4- to 6-membered heteroarylalkyl-, benzofused 4- to 6-membered heteroarylalkyl-, 4- to 6-membered heteroarylfused 4-to 6-membered heteroarylalkyl-, 3- to 7-membered cycloalkylalkyl-, 3- to 7-membered cycloalkenylalkyl-, 4- to 6-membered heterocycloalkylalkyl-, 4- to 6-membered heterocycloalkenylalkyl-, benzofused 4- to 6-membered heterocycloalkylalkyl-, benzofused 4- to 6-membered heterocycloalkenylalkyl-, and 4- to 6-membered heteroarylfused 4- to 6-membered heterocycloalkenylalkyl-, of R¹ is optionally substituted with 1 substituent selected from the group consisting of spirocycloalkyl.

In other embodiments, in each of Formulas (I), (II-A), (II-A1), (II-A2), (II-A2.1), (II-A-2.2), (II-A-2.3), (II-A4), (II-B), (II-C), (III), (IV):
the alkyl- portion of said phenylalkyl-, naphthylalkyl-, 4- to 6-membered heteroarylfused phenylalkyl-, 3- to 7-membered cycloalkylfused phenylalkyl-, 3- to 7-membered cycloalkenylfused phenylalkyl-, 4- to 6-membered heteroarylalkyl-, benzofused 4- to 6-membered heteroarylalkyl-, 4- to 6-membered heteroarylfused O-5 to 6-membered heteroarylalkyl-, 3- to 7-membered cycloalkylalkyl-, 3- to 7-membered cycloalkenylalkyl-, 4- to 6-membered heterocycloalkylalkyl-, 4- to 6-membered heterocycloalkenylalkyl-, benzofused 4- to 6-membered heterocycloalkylalkyl-, benzofused 4- to 6-membered heterocycloalkenylalkyl-, and 4- to 6-membered heteroarylfused 4- to 6-membered heterocycloalkenylalkyl-, of R¹ is optionally substituted with 1 substituent selected from the group consisting of spirocyclopropyl.

In other embodiments, in each of Formulas (I), (II-A), (II-A1), (II-A2), (II-A2.1), (II-A-2.2), (II-A-2.3), (II-A4), (II-B), (II-C), (III), (IV):

the alkyl- portion of said phenylalkyl-, naphthylalkyl-, 4- to 6-membered heteroarylfused phenylalkyl-, 3- to 7-membered cycloalkylfused phenylalkyl-, 3-to 7-membered cycloalkenylfused phenylalkyl-, 4- to 6-membered heteroarylalkyl-, benzofused 4- to 6-membered heteroarylalkyl-, 4- to 6-membered heteroarylfused 4- to 6-membered heteroarylalkyl-, 3- to 7-membered cycloalkylalkyl-, 3- to 7-membered cycloalkenylalkyl-, 4- to 6-membered heterocycloalkylalkyl-, 4- to 6-membered heterocycloalkenylalkyl-, benzofused 4-to 6-membered heterocycloalkylalkyl-, benzofused 4- to 6-membered heterocycloalkenylalkyl-, and 4- to 6-membered heteroarylfused 4- to 6-membered heterocycloalkenylalkyl- of R¹ is a moiety of the formula:

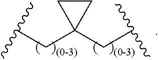

In other embodiments, in each of Formulas (I), (II-A), (II-A1), (II-A2), (II-A2.1), (II-A-2.2), (II-A-2.3), (II-A4), (II-B), (II-C), (III), (IV):

the alkyl- portion of said phenylalkyl-, naphthylalkyl-, 4- to 6-membered heteroarylfused phenylalkyl-, 3- to 7-membered cycloalkylfused phenylalkyl-, 3-to 7-membered cycloalkenylfused phenylalkyl-, 4- to 6-membered heteroarylalkyl-, benzofused 4- to 6-membered heteroarylalkyl-, 4- to 6-membered heteroarylfused 4- to 6-membered heteroarylalkyl-, 3- to 7-membered cycloalkylalkyl-, 3- to 7-membered cycloalkenylalkyl-, 4- to 6-membered heterocycloalkylalkyl-, 4- to 6-membered heterocycloalkenylalkyl-, benzofused 4-to 6-membered heterocycloalkylalkyl-, benzofused 4- to 6-membered heterocycloalkenylalkyl-, and 4- to 6-membered heteroarylfused 4- to 6-membered heterocycloalkenylalkyl- of R¹ is a moiety of the formula:

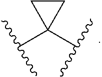

In other embodiments, in each of Formulas (I), (II-A), (II-A1), (II-A2), (II-A2.1), (II-A-2.2), (II-A-2.3), (II-A4), (II-B), (II-C), (III), (IV):

the alkyl- portion of said phenylalkyl-, naphthylalkyl-, 4- to 6-membered heteroarylfused phenylalkyl-, 3- to 7-membered cycloalkylfused phenylalkyl-, 3-to 7-membered cycloalkenylfused phenylalkyl-, 4- to 6-membered heteroarylalkyl-, benzofused 4- to 6-membered heteroarylalkyl-, 4- to 6-membered heteroarylfused 4- to 6-membered heteroarylalkyl-, 3- to 7-membered cycloalkylalkyl-, 3- to 7-membered cycloalkenylalkyl-, 4- to 6-membered heterocycloalkylalkyl-, 4- to 6-membered heterocycloalkenylalkyl-, benzofused 4-to 6-membered heterocycloalkylalkyl-, benzofused 4- to 6-membered heterocycloalkenylalkyl-, and 4- to 6-membered heteroarylfused 4- to 6-membered heterocycloalkenylalkyl- of R¹ is a moiety of the formula:

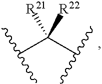

wherein one of R²¹ and R²² is hydrogen and the other is selected from the group consisting of C₁-C₂ alkyl, C₁-C₂ haloalkyl, fluorine, and hydroxyl. In other such embodiments, one of R²¹ and R²² is hydrogen and the other is selected from the group consisting of methyl and —CF₃.

In other embodiments, in each of Formulas (I), (II-A), (II-A1), (II-A2), (II-A2.1), (II-A-2.2), (II-A-2.3), (II-A4), (II-B), (II-C), (III), (IV): R¹ is unsubstituted.

In other embodiments, in each of Formulas (I), (II-A), (II-A1), (II-A2), (II-A2.1), (II-A-2.2), (II-A-2.3), (II-A4), (II-B), (II-C), (III), (IV): R¹ is substituted with from 1 to 4 substituents.

In other embodiments, in each of Formulas (I), (II-A), (II-A1), (II-A2), (II-A2.1), (II-A-2.2), (II-A-2.3), (II-A4), (II-B), (II-C), (III), (IV): R¹ is substituted with from 1 to 3 substituents.

In other embodiments, in each of Formulas (I), (II-A), (II-A1), (II-A2), (II-A2.1), (II-A-2.2), (II-A-2.3), (II-A4), (II-B), (II-C), (III), (IV): R¹ is substituted with from 1 to 2 substituents.

In other embodiments, in each of Formulas (I), (II-A), (II-A1), (II-A2), (II-A2.1), (II-A-2.2), (II-A-2.3), (II-A4), (II-B), (II-C), (III), (IV): R¹ is substituted with 1 substituent.

In other embodiments, in each of Formulas (I), (II-A), (II-A1), (II-A2), (II-A2.1), (II-A-2.2), (II-A-2.3), (II-A4), (II-B), (1′-C), (III), (IV): R¹ is substituted with from 1 to 2 substituents, which may be the same or different, each independently selected from the group consisting of halogen, hydroxy, —CN, —N(R¹¹)₂, alkyl, haloalkyl, optionally substituted aryl, optionally substituted heteroaryl, optionally substituted heterocycloalkyl, and optionally substituted arylalkoxy.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is —OH.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is —OH;

R³ is selected from the group consisting of H and methyl; R⁴ is H; and R⁵ is H. In other such embodiments, R³ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is —OH; R³ is selected from the group consisting of H and methyl; R⁴ is halo; and R⁵ is halo. In other such embodiments, R³ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is —OH;

R³ is selected from the group consisting of H and methyl; R⁴ is alkyl; and R⁵ is alkyl. In other such embodiments, R³ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is —OH; R³ is selected from the group consisting of H and methyl; R⁴ is halo; and R⁵ is alkyl. In other such embodiments, R³ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is —OH; R³ is selected from the group consisting of H and methyl; R⁴ is alkyl; and R⁵ is halo. In other such embodiments, R³ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is selected from the group consisting of —OH and —OC(O)R⁹.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁹ is unsubstituted.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁹ is substituted with from 1 to 3 substituents.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁹ is substituted with from 1 to 2 substituents.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁹ is substituted with 1 substituent.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁹ is substituted with from 1 to 2 substituents, which may be the same or different, each independently selected from the group consisting of alkyl, halogen, and haloalkyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁹ is unsubstituted or substituted heterocycloalkyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁹ is unsubstituted or substituted heterocycloalkenyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁹ is unsubstituted or substituted heteroaryl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is

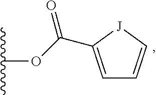

wherein J is selected from the group consisting of O, S, and N, or the oxides thereof.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is

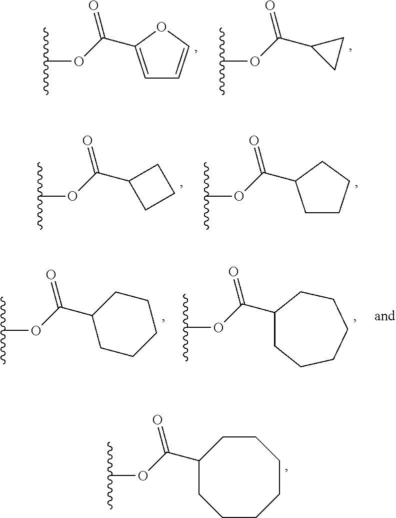

wherein the cycloalkyl portion of said moiety is unsubstituted or substituted with from 1 to 4 substituents independently selected from the group consisting of alkyl, halogen, haloalkyl, hydroxyl, —N(R⁷)₂, and CN. In other embodiments, R² is:

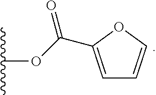

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is selected from the group consisting of:

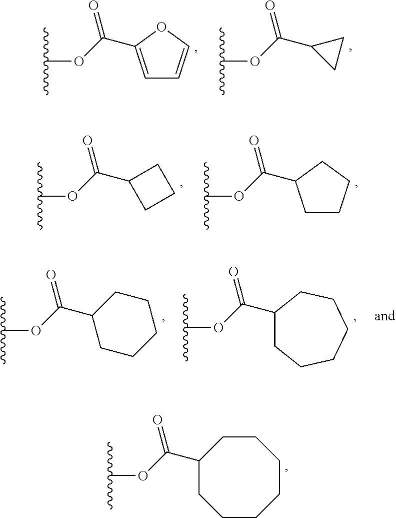

wherein the cycloalkyl portion of said moiety is unsubstituted or substituted with from 1 to 4 substituents independently selected from the group consisting of alkyl, halogen, haloalkyl, hydroxyl, —N(R⁷)₂, and CN; R³ is selected from the group consisting of H and methyl; R⁴ is H; and R⁵ is H. In other embodiments, R² is

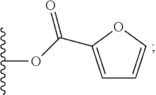

### and R³ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is selected from the group consisting of:

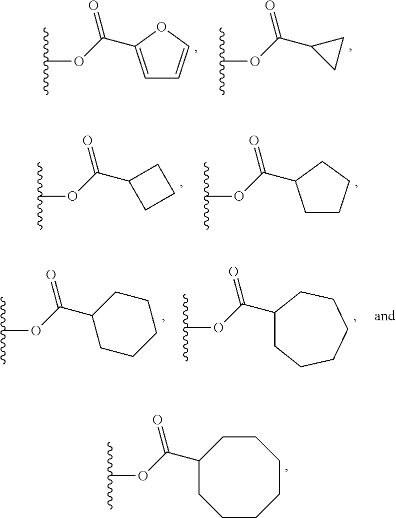

wherein the cycloalkyl portion of said moiety is unsubstituted or substituted with from 1 to 4 substituents independently selected from the group consisting of alkyl, halogen, haloalkyl, hydroxyl, —N(R⁷)₂, and CN; R³ is selected from the group consisting of H and methyl; R⁴ is halo; and R⁵ is halo. In other such embodiments, R² is

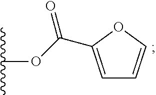

### and R³ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is selected from the group consisting of:

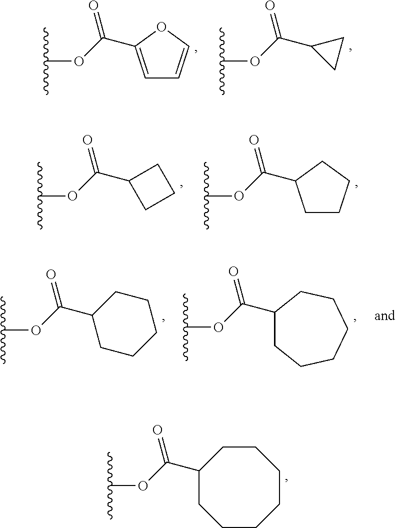

wherein the cycloalkyl portion of said moiety is unsubstituted or substituted with from 1 to 4 substituents independently selected from the group consisting of alkyl, halogen, haloalkyl, hydroxyl, —N(R⁷)₂, and CN; R³ is selected from the group consisting of H and methyl; R⁴ is alkyl; and R⁵ is alkyl. In other such embodiments, R² is

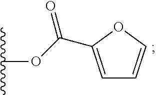

### and R³ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is selected from the group consisting of:

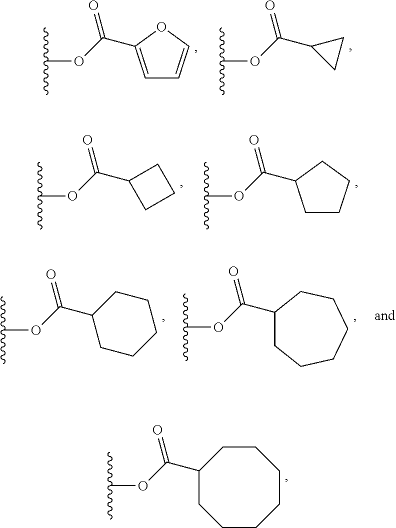

wherein the cycloalkyl portion of said moiety is unsubstituted or substituted with from 1 to 4 substituents independently selected from the group consisting of alkyl, halogen, haloalkyl, hydroxyl, —N(R⁷)₂, and CN; R³ is selected from the group consisting of H and methyl; R⁴ is halo; and R⁵ is alkyl. In other such embodiments, R² is

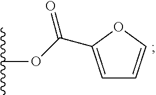

### and R³ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is selected from the group consisting of:

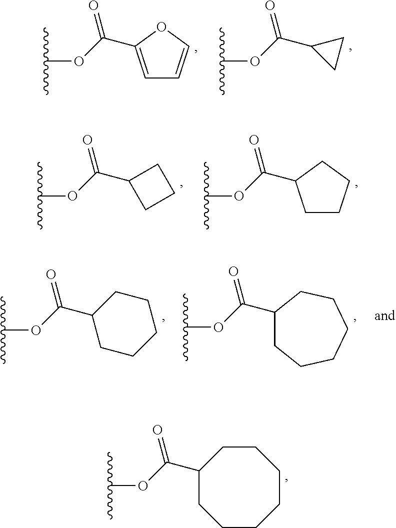

wherein the cycloalkyl portion of said moiety is unsubstituted or substituted with from 1 to 4 substituents independently selected from the group consisting of alkyl, halogen, haloalkyl, hydroxyl, —N(R⁷)₂, and CN; R³ is selected from the group consisting of H and methyl; R⁴ is alkyl; and R⁵ is halo. In other such embodiments, R² is

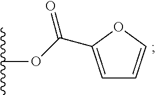

### and R³ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is a moiety selected from the group consisting of

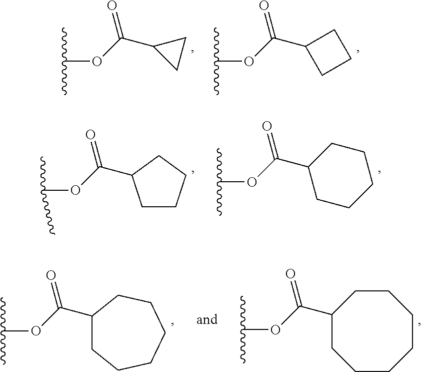

wherein the cycloalkyl portion of said moiety is unsubstituted or substituted with from 1 to 4 substituents independently selected from the group consisting of alkyl, halogen, haloalkyl, hydroxyl, —N(R⁷)₂, and CN.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is

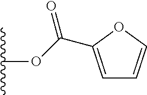

### and R³ is H.

In other embodiments, in each of Formulas (I), (II-A), (II-A2), (II-A2.1), (II-A-2.2), (II-A-2.3), (II-A4), (II-B), (II-C), (III), (IV), (V), (VI): R² is

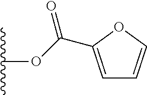

and R³ is methyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R³ is selected from the group consisting of hydrogen, hydroxyl, and methyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R³ is selected from the group consisting of hydrogen and methyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R³ is hydrogen.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R³ is hydroxy.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R³ is alkyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R³ is methyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R³ is ethyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R³ is straight or branched propyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is —OR⁸, wherein R⁸ is hydrogen, and R³ is methyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² is —OR⁸, wherein R⁸ is hydrogen, and R³ is hydrogen.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² and R³ are taken together form a moiety of formula 2:

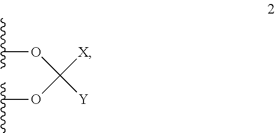

wherein X and Y are each alkyl. In other such embodiments, X and Y are each methyl. In another such embodiment, X and Y are each ethyl. In another such embodiment, X is methyl and Y is ethyl. In another such embodiment, X is hydrogen and Y is selected from the group consisting of alkyl, haloalkyl, and cycloalkyl. In other such embodiments, X is hydrogen and Y is selected from the group consisting of methyl. In other such embodiments, X is hydrogen and Y is selected from the group consisting of ethyl. In other such embodiments, X is hydrogen and Y is selected from the group consisting of straight or branched propyl. In other such embodiments, X is hydrogen and Y is selected from the group consisting of straight or branched butyl. In other such embodiments, X is hydrogen and Y is selected from the group consisting of haloalkyl. In other such embodiments, X is hydrogen and Y is selected from the group consisting of cyclopropyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² and R³ are taken together form a moiety of formula:

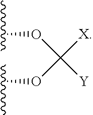

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² and R³ are taken together form a moiety of formula:

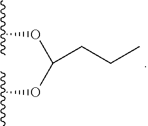

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² and R³ are taken together form a moiety of formula:

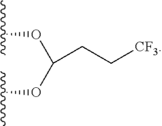

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² and R³ are taken together form a moiety selected from the group consisting of:

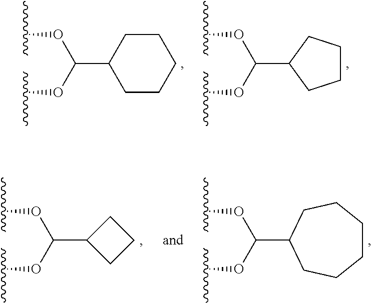

wherein said cycloalkyl ring is unsubstituted or substituted with from 1 to 4 substituents independently selected from the group consisting of alkyl, halogen, haloalkyl, hydroxyl, —N(R⁷)₂, and CN.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² and R³ are taken together form a moiety of the formula:

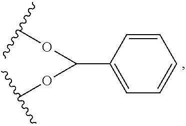

wherein the phenyl group of said moiety is unsubstituted or substituted with from 1 to 4 substituents independently selected from the group consisting of alkyl, halogen, haloalkyl, hydroxyl, —N(R⁷)₂, and CN.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R² and R³ are taken together form a moiety of formula 3:

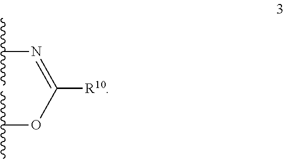

In other such embodiments, R¹⁰ is H. In other such embodiments, R¹⁰ is alkyl. In another such embodiment, R¹⁰ is methyl. In other such embodiments, R¹⁰ is ethyl. In other such embodiments, R¹⁰ is straight or branched propyl.

In other embodiments, in each of Formulas (I), (II-A), (II-A1), (II-A2), (II-A2.1), (II-A-2.2), (II-A-2.3), (II-A4), (II-B), (1′-C), (III), (IV), (V), (VI): R⁴ is hydrogen.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is halogen.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is fluoro.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is chloro.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁵ is selected from the group consisting of hydrogen and alkyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁵ is methyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁵ is ethyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁵ is straight or branched propyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁵ is halogen.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁵ is fluoro.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁵ is chloro.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is hydrogen and R⁵ is hydrogen.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is hydrogen and R⁵ is halogen.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is hydrogen and R⁵ is fluoro.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is hydrogen and R⁵ is chloro.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is hydrogen and R⁵ is alkyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is hydrogen and R⁵ is methyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is hydrogen and R⁵ is ethyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is hydrogen and R⁵ is straight or branched propyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is halogen and R⁵ is hydrogen.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is halogen and R⁵ is halogen.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is fluoro and R⁵ is fluoro.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is chloro and R⁵ is chloro.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is halogen and R⁵ is alkyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is halogen and R⁵ is methyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is halogen and R⁵ is ethyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is halogen and R⁵ is straight or branched propyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI): R⁴ is fluoro or chloro and R⁵ is methyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is selected from the group consisting of H, alkyl, -alkyl-CN, -alkyl-OH, alkoxy, heteroalkyl, —O-heteroalkyl, haloalkyl, phenyl, phenylalkyl-, naphthyl, naphthylalkyl-, heteroarylfused aryl, heteroarylfused arylalkyl-, cycloalkylfused aryl, cycloalkylfused arylalkyl-, heteroaryl, heteroarylalkyl-, benzofused heteroaryl, benzofused heteroarylalkyl-, heteroarylfused heteroaryl, heteroarylfused heteroarylalkyl-, cycloalkyl, cycloalkenyl, cycloalkylalkyl-, cycloalkenylalkyl-, heterocycloalkyl, heterocycloalkenyl, heterocycloalkylalkyl-, heterocycloalkenylalkyl-, benzofused heterocycloalkyl, benzofused heterocycloalkenyl, benzofused heterocycloalkylalkyl-, benzofused heterocycloalkenylalkyl-, heteroarylfused heterocycloalkenyl, and heteroarylfused heterocycloalkenylalkyl-,
wherein each said hetero ring-containing moiety of R⁶ contains 1, 2, or 3 ring heteroatoms independently selected from the group consisting of any combination of N, O, and S, and
wherein each said R⁶ (when other than H) is unsubstituted or substituted with from 1 to 3 groups independently selected from the group consisting of halogen, —CN, —OH, alkyl, haloalkyl, alkoxy, and —N(R⁷).

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is selected from the group consisting of H, alkyl, -alkyl-CN, -alkyl-OH, alkoxy, heteroalkyl, —O-heteroalkyl, haloalkyl, aryl, arylalkyl-, naphthyl, naphthylalkyl-, heteroarylfused aryl, heteroarylfused arylalkyl-, cycloalkylfused aryl, cycloalkylfused arylalkyl-, heteroaryl, heteroarylalkyl-, benzofused heteroaryl, benzofused heteroarylalkyl-, heteroarylfused heteroaryl, heteroarylfused heteroarylalkyl-, cycloalkyl, cycloalkenyl, cycloalkylalkyl-, cycloalkenylalkyl-, heterocycloalkyl, heterocycloalkenyl, heterocycloalkylalkyl-, heterocycloalkenylalkyl-, benzofused heterocycloalkyl, benzofused heterocycloalkenyl, benzofused heterocycloalkylalkyl-, benzofused heterocycloalkenylalkyl-, heteroarylfused heterocycloalkenyl, and heteroarylfused heterocycloalkenylalkyl-,
wherein each said hetero ring-containing moiety of R⁶ contains 1, 2, or 3 ring heteroatoms independently selected from the group consisting of any combination of N, O, and S, and
wherein each said R⁶ (when other than H) is unsubstituted or substituted with from 1 to 2 groups independently selected from the group consisting of halogen, —CN, —OH, alkyl, haloalkyl, alkoxy, and —N(R⁷).

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is selected from the group consisting of H, alkyl, -alkyl-CN, -alkyl-OH, alkoxy, heteroalkyl, —O-heteroalkyl, haloalkyl, aryl, arylalkyl-, naphthyl, naphthylalkyl-, heteroarylfused aryl, heteroarylfused arylalkyl-, cycloalkylfused aryl, cycloalkylfused arylalkyl-, heteroaryl, heteroarylalkyl-, benzofused heteroaryl, benzofused heteroarylalkyl-, heteroarylfused heteroaryl, heteroarylfused heteroarylalkyl-, cycloalkyl, cycloalkenyl, cycloalkylalkyl-, cycloalkenylalkyl-, heterocycloalkyl, heterocycloalkenyl, heterocycloalkylalkyl-, heterocycloalkenylalkyl-, benzofused heterocycloalkyl, benzofused heterocycloalkenyl, benzofused heterocycloalkylalkyl-, benzofused heterocycloalkenylalkyl-, heteroarylfused heterocycloalkenyl, and heteroarylfused heterocycloalkenylalkyl-,
wherein each said hetero ring-containing moiety of R⁶ contains 1, 2, or 3 ring heteroatoms independently selected from the group consisting of any combination of N, O, and S, and
wherein each said R⁶ (when other than H) is unsubstituted or substituted with 1 group selected from the group consisting of halogen, —CN, —OH, alkyl, haloalkyl, alkoxy, and —N(R⁷).

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is selected from the group consisting of H, alkyl, -alkyl-CN, -alkyl-OH, alkoxy, haloalkyl, aryl, arylalkyl-, naphthyl, benzofused heteroaryl, heteroarylfused aryl, heteroarylfused arylalkyl-, benzofused heterocycloalkenyl, heteroaryl, heteroarylalkyl-, benzofused heteroarylalkyl-, cycloalkyl, and heterocycloalkyl,
wherein each said hetero ring-containing moiety of R⁶ contains 1, 2, or 3 ring heteroatoms independently selected from the group consisting of any combination of N, O, and S, and
wherein each said R⁶ (when other than H) is unsubstituted or substituted with 1 group selected from the group consisting of halogen, —CN, —OH, alkyl, haloalkyl, alkoxy, and —N(R⁷).

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is selected from the group consisting of H, alkyl, -alkyl-CN, -alkyl-OH, alkoxy, haloalkyl, aryl, arylalkyl-, benzofused heteroaryl, benzofused heterocycloalkenyl, heteroaryl, benzofused heteroarylalkyl-, cycloalkyl, and heterocycloalkyl,
wherein each said hetero ring-containing moiety of R⁶ contains 1, 2, or 3 ring heteroatoms independently selected from the group consisting of any combination of N, O, and S, and
wherein each said R⁶ (when other than H) is unsubstituted or substituted with 1 group selected from the group consisting of halogen, —CN, —OH, alkyl, haloalkyl, alkoxy, and —N(R⁷).

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is selected from the group consisting of H, alkyl, -alkyl-CN, -alkyl-OH, alkoxy, haloalkyl, phenyl, benzyl, 5- to 6-membered heteroaryl, benzofused 5- to 6-membered heteroaryl, benzofused 5- to 6-membered heterocycloalkenyl, benzofused 5- to 6-membered heteroarylalkyl-, cyclopropyl, cyclobutyl, cyclopentyl, cyclohexyl, cycloheptyl, tetrahydropyran, and tetrahydrofuran,
wherein each said hetero ring-containing moiety of R⁶ contains 1, 2, or 3 ring heteroatoms independently selected from the group consisting of any combination of N, O, and S, and
wherein each said R⁶ (when other than H) is unsubstituted or substituted with 1 group selected from the group consisting of halogen, —CN, —OH, alkyl, haloalkyl, alkoxy, and —N(R⁷).

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is alkyl (which is unsubstituted or substituted as described herein). Non-limiting examples of R⁶, when R⁶ is alkyl (which may be unsubstituted or further substituted with one or more groups selected from the group consisting of halogen, —CN, —OH, alkyl, haloalkyl, alkoxy, and —N(R⁷) as described herein), include: lower alkyl. Non-limiting examples of lower alkyl (which may be unsubstituted or substituted as described herein) include methyl, ethyl, propyl (n-propyl and i-propyl), butyl (n-butyl, i-butyl, and t-butyl), pentyl (straight or branched), hexyl (straight or branched), octyl (straight or branched), etc.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is selected from the group consisting of -alkyl-CN and -alkyl-OH (which is unsubstituted or substituted as described herein). Non-limiting examples of the alkyl portion of said -alkyl-CN and -alkyl-OH (which may be unsubstituted or substituted as described herein) include Non-limiting examples of lower alkyl include methyl, ethyl, propyl (n-propyl and i-propyl), butyl (n-butyl, i-butyl, and t-butyl), pentyl (straight or branched), hexyl (straight or branched), octyl (straight or branched), etc., as described above.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is alkoxy (which is unsubstituted or substituted as described herein). Non-limiting examples of the alkyl portion of said alkoxy (which may be unsubstituted or substituted as described herein) include methyl, ethyl, propyl (n-propyl and i-propyl), butyl (n-butyl, i-butyl, and t-butyl), pentyl (straight or branched), hexyl (straight or branched), octyl (straight or branched), etc., as described above. In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is heteroalkyl (which is unsubstituted or substituted as described herein). Non-limiting examples of R⁶, when R⁶ is heteroalkyl (which may be unsubstituted or further substituted with one or more groups selected from the group consisting of halogen, —CN, —OH, alkyl, haloalkyl, alkoxy, and —N(R⁷) as described herein), include: ethers and thioethers and other heteroalkyl groups as described herein.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is —O-heteroalkyl (which is unsubstituted or substituted as described herein). Non-limiting examples of the heteroalkyl portion of said —O-heteroalkyl include the heteroalkyl groups described above.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is haloalkyl (which is unsubstituted or substituted as described herein). Non-limiting examples of said haloalkyl groups (which may be unsubstituted or substituted as described herein) include those alkyl groups described above in which one or more available hydrogen atoms of said alkyl group is replaced with one or more halogen groups, respectively. Additional non-limiting examples of R⁶ when R⁶ is haloalkyl include —CF₃, —CHF₂, —CH₂F, —CH₂CF₃, —CHFCF₃, —CF₂CF₃, —CH₂CHF₂, —CHFCH₂F, —CF₂CF₃, etc.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is aryl (which is unsubstituted or substituted as described herein). Non-limiting examples of R⁶ when R⁶ is aryl (which may be unsubstituted or substituted as described herein) include phenyl and naphthyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is arylalkyl-(which is unsubstituted or substituted as described herein). Non-limiting examples of R⁶, when R⁶ is arylalkyl- (which may be unsubstituted or substituted as described herein), include those moieties wherein the aryl portion of said arylalkyl- is selected from the group consisting of phenyl and naphthyl, and wherein the alkyl portion of said arylalkyl- (which may be unsubstituted or substituted as described herein) is selected from the group consisting of divalent lower alkyl. Non-limiting examples of divalent lower alkyl include -methylene-, -ethylene-, -propylene- (straight or branched), -butylene- (straight or branched), -pentylene- (straight or branched), -hexylene- (straight or branched), -octylene-(straight or branched), etc., as described above.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is heteroaryl. Non-limiting examples of R⁶, when R⁶ is heteroaryl (which may be unsubstituted or further substituted as described herein), include: pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, and benzothiazolyl. The point of attachment of said R¹ group to -L- is by replacement of any available hydrogen atom on a ring carbon or ring heteroatom.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is heteroarylalkyl-. Non-limiting examples of R⁶, when R⁶ is heteroarylalkyl- (which may be unsubstituted or further substituted as described herein), include: those moieties wherein the heteroaryl portion of said heteroarylalkyl- is selected from heteroaryl as described herein, and wherein said alkyl- portion of said heteroarylalkyl- is selected from divalent -alkyl-, as described herein. The point of attachment of said R⁶ to ring A is through the alkyl- group.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is cycloalkyl. Non-limiting examples of R⁶, when R⁶ is cycloalkyl- (which may be unsubstituted or further substituted as described herein), include: cyclopropyl, cyclopentyl, cyclohexyl, cycloheptyl and the like. Non-limiting examples of suitable multicyclic cycloalkyls include 1-decalinyl, norbornyl, adamantyl and the like. Further non-limiting examples of cycloalkyl are also described herein.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is cycloalkylalkyl-. Non-limiting examples of R⁶, when R⁶ is cycloalkylalkyl- (which may be unsubstituted or further substituted as described herein), include those moieties wherein the cycloalkyl portion of said cycloalkylalkyl- is selected a cycloalkyl group as described herein, and wherein said alkyl- portion of said cycloalkylalkyl- is selected from divalent -alkyl-, as described herein. The point of attachment of said R⁶ to ring A is through the alkyl- group.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is cycloalkenyl. Non-limiting examples of R⁶, when R⁶ is cycloalkenyl- (which may be unsubstituted or further substituted as described herein), include unsaturated versions of any of the following: cyclopropyl, cyclopentyl, cyclohexyl, cycloheptyl and the like. Non-limiting examples of suitable multicyclic cycloalkyls include unsaturated versions of any of the following: 1-decalinyl, norbornyl, adamantyl and the like. Further non-limiting examples of cycloalkenyl are also described herein.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is cycloalkenylalkyl-. Non-limiting examples of R⁶ when R⁶ is cycloalkenylalkyl-(which may be unsubstituted or further substituted as described herein), include those moieties wherein the cycloalkenyl portion of said cycloalkenylalkyl- is selected a cycloalkenyl group as described herein, and wherein said alkyl-portion of said cycloalkenylalkyl- is selected from divalent -alkyl-, as described herein. The point of attachment of said R⁶ to ring A is through the alkyl- group.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R¹ is heterocycloalkyl. Non-limiting examples of R⁶, when R⁶ is heterocycloalkyl-(which may be unsubstituted or further substituted as described herein), include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and oxides and oo-substituted versions thereof.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is heterocycloalkylalkyl-. Non-limiting examples of R⁶, when R⁶ is heterocycloalkylalkyl- (which may be unsubstituted or further substituted as described herein), include), include those moieties wherein the heterocycloalkyl portion of said heterocycloalkylalkyl- is selected a heterocycloalkyl group as described herein, and wherein said alkyl- portion of said heterocycloalkylalkyl- is selected from divalent -alkyl-, as described herein. The point of attachment of said R⁶ to ring A is through the alkyl- group.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is heterocycloalkenyl. Non-limiting examples of R⁶, when R⁶ is heterocycloalkenyl-(which may be unsubstituted or further substituted as described herein), include: 1,2,3,4- tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like, and oxides thereof or oxo-substituted versions thereof.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ is heterocycloalkenylalkyl-. Non-limiting examples of R⁶, when R⁶ is heterocycloalkenylalkyl- (which may be unsubstituted or further substituted as described herein), include those moieties wherein the heterocycloalkenyl portion of said heterocycloalkenylalkyl- is selected a heterocycloalkenyl group as described herein, and wherein said alkyl- portion of said heterocycloalkylalkyl- is selected from divalent -alkyl-, as described herein. The point of attachment of said R⁶ to ring A is through the alkyl- group.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI), R⁶ comprises a multicyclic moiety wherein an aryl (e.g., benzo), heteroaryl, cycloalkyl, cycloalkenyl, heterocycloalkyl or heterocycloalkenyl moiety (each of which moieties may be unsubstituted or substituted as described herein) (non-limiting examples of which multicyclic moieties are as described above) is fused to another moiety selected from the group consisting of aryl, arylalkyl-, heteroaryl, heteroarylalkyl-, cycloalkyl, cycloalkylalkyl-, cycloalkenyl, cycloalkenylalkyl-, heterocycloalkyl, heterocycloalkylalkyl-, heterocycloalkenyl, and heterocycloalkenylalkyl- (non-limiting examples of which moieties are as described above). In such moieties, the point of attachment of R⁶ to ring A is indicated by “-”.

Non-limiting examples of R⁶, when R⁶ is benzofused 5- to 6-membered heteroaryl (which may be unsubstituted or further substituted with one or more groups selected from the group consisting of halogen, —CN, —OH, alkyl, haloalkyl, alkoxy, and —N(R⁷) as described herein), include:

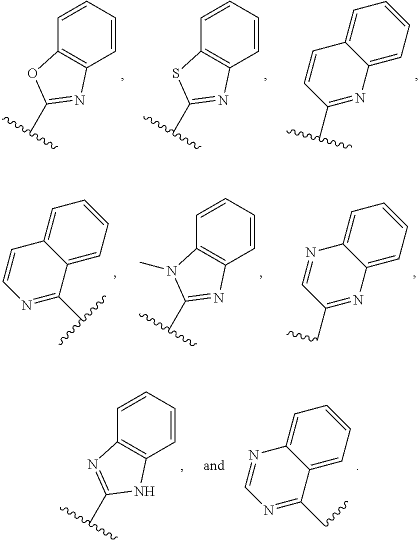

Non-limiting examples of R⁶, when R⁶ is heteroarylfused 5- to 6-membered heteroaryl (which may be unsubstituted or further substituted with one or more groups selected from the group consisting of halogen, —CN, —OH, alkyl, haloalkyl, alkoxy, and —N(R⁷) as described herein), include:

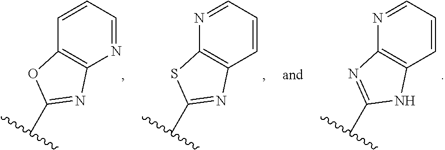

Non-limiting examples of R⁶, when R⁶ is heteroarylfused aryl (which may be unsubstituted or further substituted with one or more groups selected from the group consisting of halogen, —CN, —OH, alkyl, haloalkyl, alkoxy, and —N(R⁷) as described herein), include:

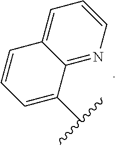

Further non-limiting examples of R⁶, when R⁶ is heteroaryl (which may be unsubstituted or further substituted with one or more groups selected from the group consisting of halogen, —CN, —OH, alkyl, haloalkyl, alkoxy, and —N(R⁷) as described herein), include: pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, and benzothiazolyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is selected from the group consisting of H, alkyl, -alkyl-CN, -alkyl-OH, alkoxy, haloalkyl, unsubstituted phenyl; phenyl substituted with 1 group selected from the group consisting of halo, alkyl, haloalkyl, and alkoxy; unsubstituted pyridyl; pyridyl substituted with 1 group selected from the group consisting of halo, alkyl, haloalkyl, and alkoxy; oxanyl; oxanyl substituted with 1 group selected from the group consisting of halo, alkyl, haloalkyl, and alkoxy; and unsubstituted and substituted benzofused heteroaryl selected from the group consisting of

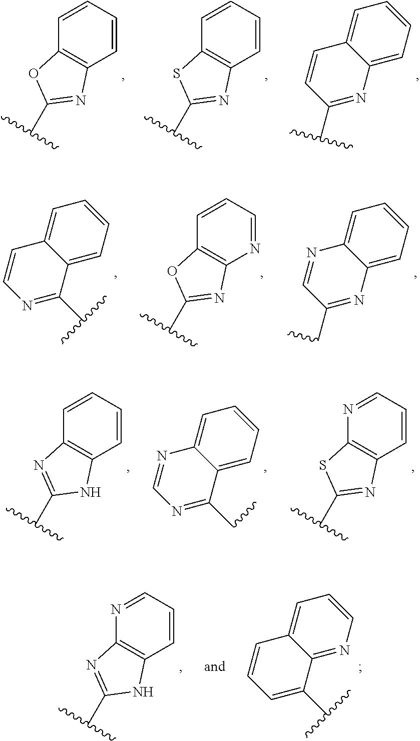

wherein said substitutents (when present) are selected from the group consisting of halo, alkyl, haloalkyl, and alkoxy.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is selected from the group consisting of H, alkyl, -((C₁-C₄) straight or branched alkyl)-CN, —((C₁-C₄) straight or branched alkyl)—OH,

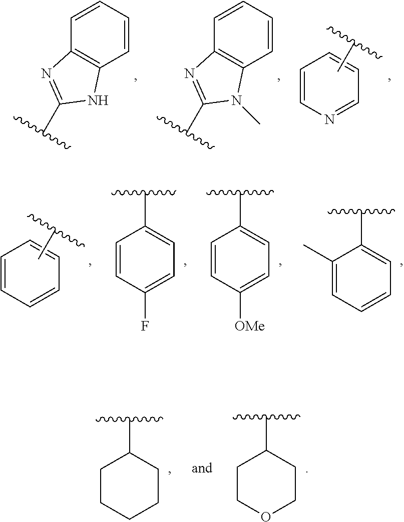

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is H.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is —((C₁-C₆) straight or branched alkyl).

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is methyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is t-butyl.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is haloalkyl (straight or branched).

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is —((C₁-C₄) straight or branched alkyl)-CN.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is —((C₁-C₄)straight or branched alkyl)-OH.

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is

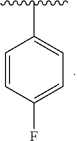

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is

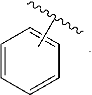

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is

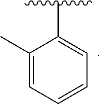

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is

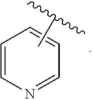

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is

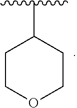

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is

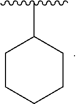

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is

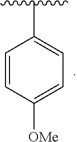

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is

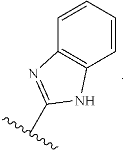

In other embodiments, in each of Formulas (I), (II-A), (II-A1), (II-A2), (II-A2.1), (II-A-2.2), (II-A-2.3), (II-A4), (II-B), (II-C), (III), (IV), (V), (VI):

R⁶ is

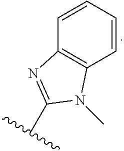

In one embodiment, the compounds of the invention have the general structure shown in Formula (II-A):

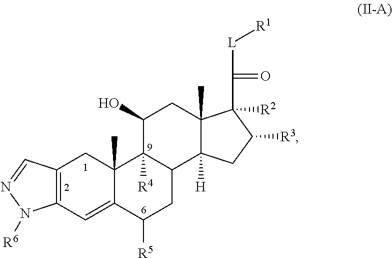

and include pharmaceutically acceptable salts, solvates, esters, prodrugs, or isomers thereof, wherein:

-L- is a divalent moiety selected from the group consisting of —CH₂S—, —S—, —CH₂—, —OCH₂—, —CH₂O—, —SCH₂—, and —NR¹¹—,

R¹ is selected from the group consisting of:

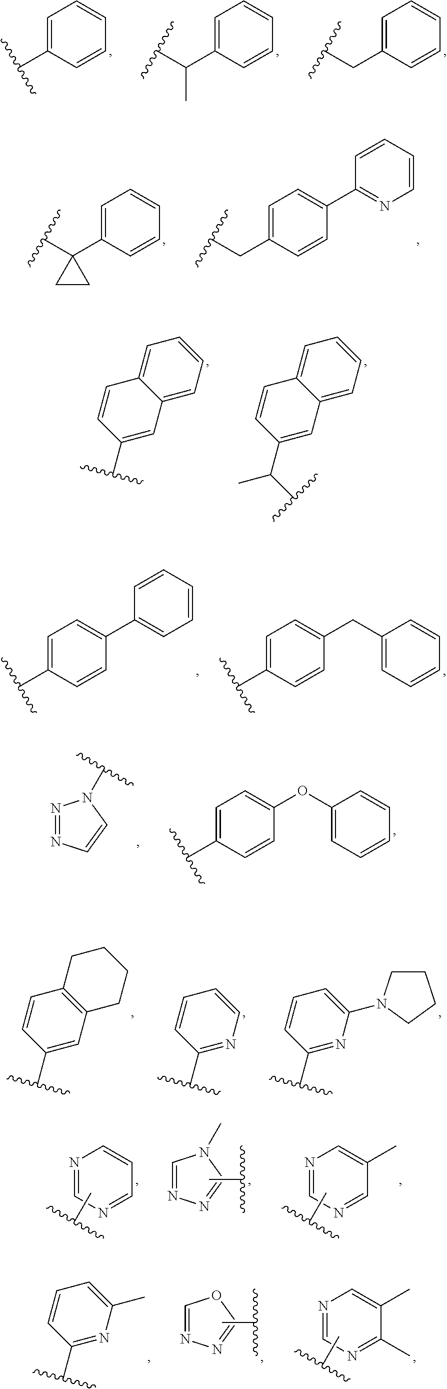

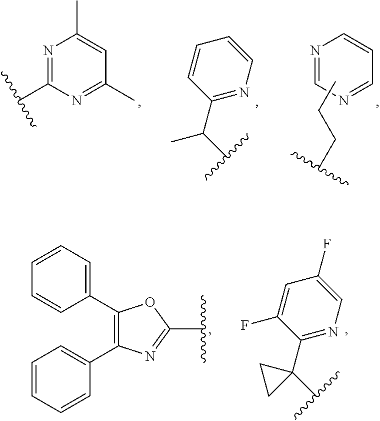

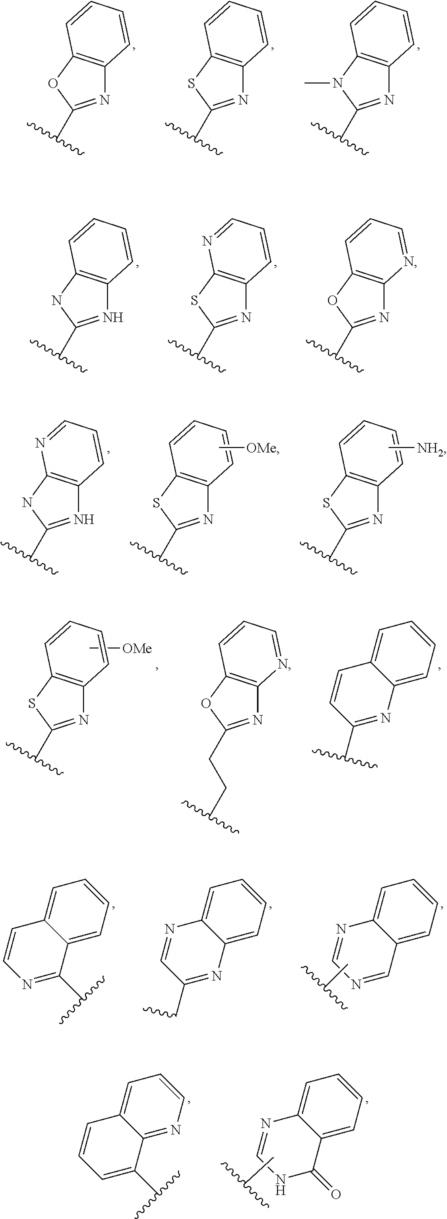

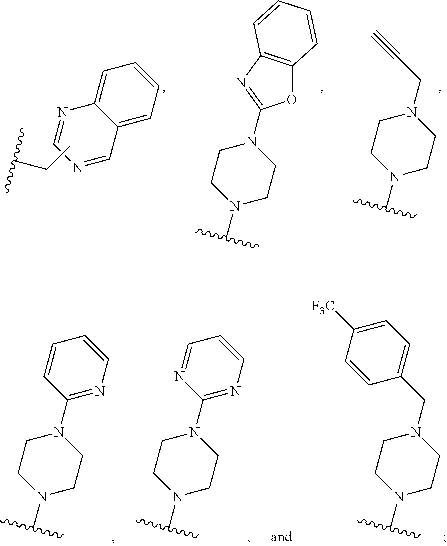

R² is selected from the group consisting of —OH and

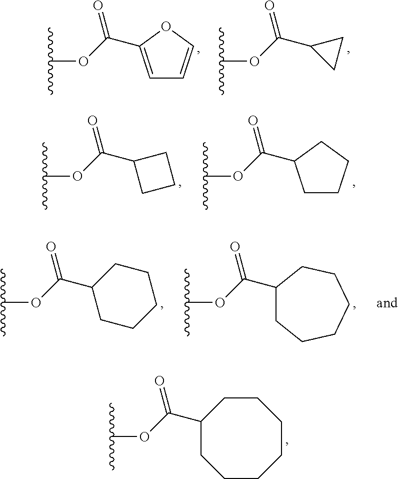

wherein the cycloalkyl portion of said moiety is unsubstituted or substituted with from 1 to 4 substituents independently selected from the group consisting of alkyl, halogen, haloalkyl, hydroxyl, —N(R⁷)₂, and CN;

R³ is H;

R⁴ is H;

R⁵ is H; and

R⁶ is selected from the group consisting of H, alkyl, —((C₁-C₄) straight or branched alkyl)-CN, —((C₁-C₄) straight or branched alkyl)-OH,

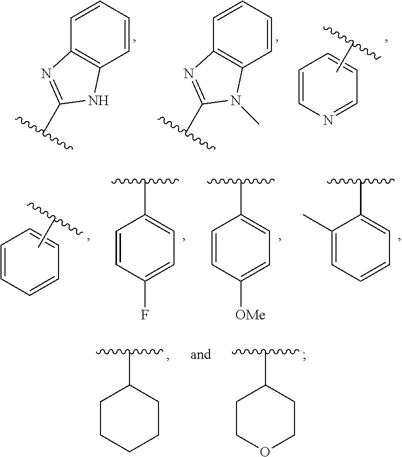

and each R⁷ and each R¹¹ is as defined in Formula (I).

Non-limiting examples of compounds of the invention include the compounds of Tables 1-5 shown in the preparative examples below and include pharmaceutically acceptable salts, solvates, esters, prodrugs, and isomers thereof.

### PREPARATIVE EXAMPLES

Generally, the compounds of the invention can be prepared by a variety of methods well known to those skilled in the art, for example, by the methods as outlined below. The examples should not be construed to limit the scope of the disclosure. Alternative mechanistic pathways and analogous structures will be apparent to those skilled in the art.

Generally, the compounds having the general structure shown in Formula A can be prepared by the following procedure:

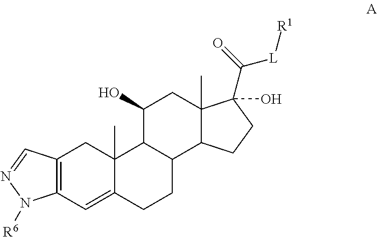

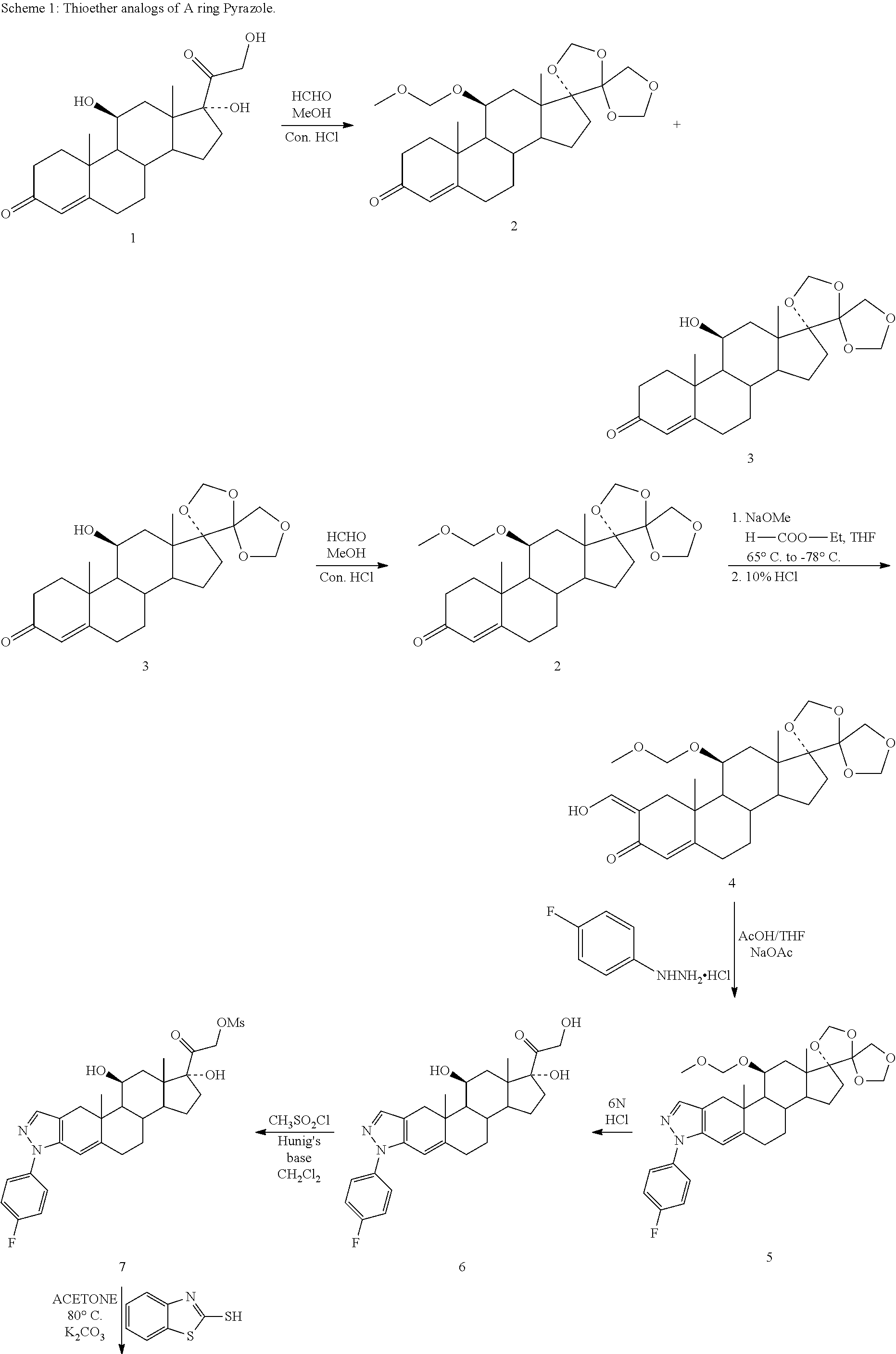

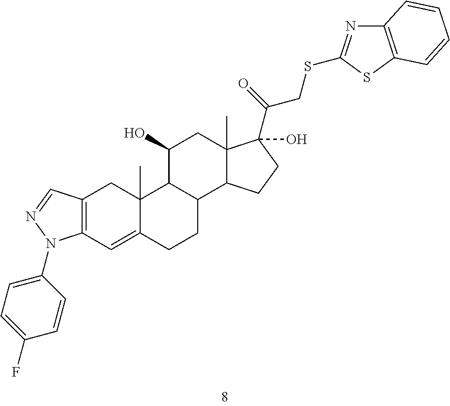

### Step 1

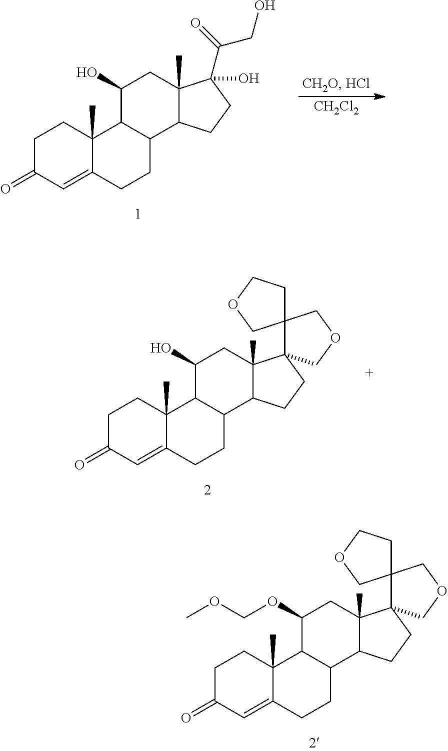

To a suspension of hydrocortisone 1 (1.5 g, 4.15 mmol) in CH₂Cl₂ (60 mL) was added formaldehyde (23.7 mL, 37 Wt % solution in water), conc. HCl (23.7 mL). The bilayer system was vigorously stirred at room temperature for 3 hr. The organic layer was separated, washed with saturated NaHCO₃ solution, water, brine, dried over anhydrous MgSO₄, filtered and concentrated. The resulting crude product was flushed through a short pad of silica gel plug to give 2.05 g of the mixture of 2 (major) and 2′ (minor). Without further purification, the mixture was used for the next step.

### Step 2

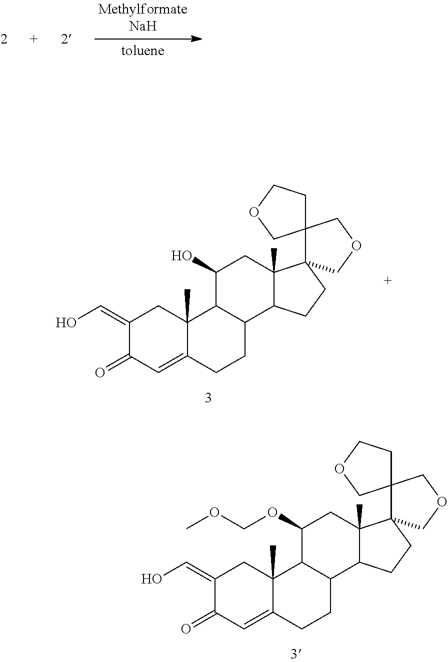

To a stirred solution of 2 and 2′ mixture (2.05 g, 5.08 mmol) and methyl formate (1.5 mL, 25.40 mmol) in toluene (11 mL) was added 60% NaH (409 mg, 10.68 mmol) at 0° C. After 10 min at 0° C., the reaction mixture was warmed to room temperature and stirred for 3.5 hr. 1N HCl (aq) was added and the mixture was extracted with CH₂Cl₂ (×3). The combined organic layer was extracted with 1N NaOH (×3). The aqueous solution was reacidified with 6 N HCl and subsequently re-extracted with CH₂Cl₂. The solvent was dried over MgSO₄, filtered, and concentrated to give 1.7 g of the mixture of 3 (major) and 3′ (minor). Without further purification, the mixture was used for the next step.

### Step 3

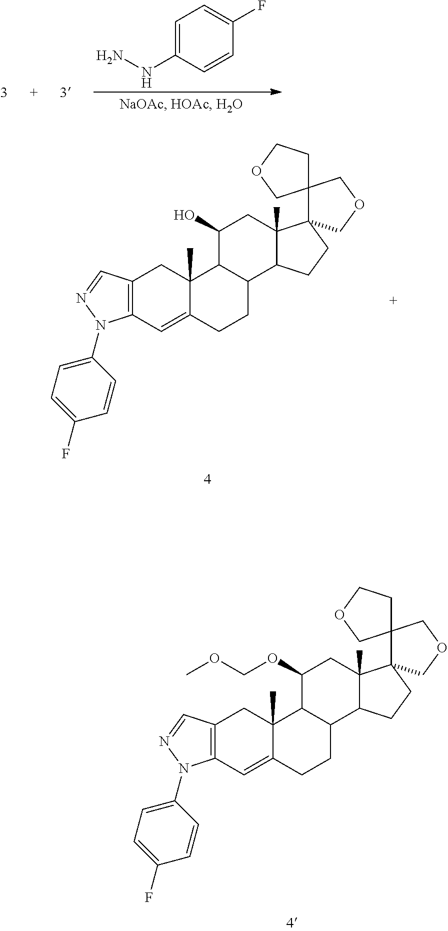

To a stirred solution of the mixture 3 and 3′ (1.7 g, 3.93 mmol) in HOAc (33 mL) was added solution of NaOAc (322.3 mg, 3.93 mmol) and 4-fluorophenyldydrazine-HCl (639 mg, 3.93 mmol) in HOAc (16 mL) and H₂O (8 mL). The reaction mixture was stirred at room temperature for 7 hrs. 1N HCl was added followed by extraction with CH₂Cl₂. The organic layer was washed with H₂O (×2), saturated NaHCO₃ (aq), H₂O and brine solution, dried over MgSO₄, filtered, and concentrated to give 2.05 g of the crude mixture product of 4 (major) and 4′ (minor). Without further purification, the mixture was used for the next step.

### Step 4

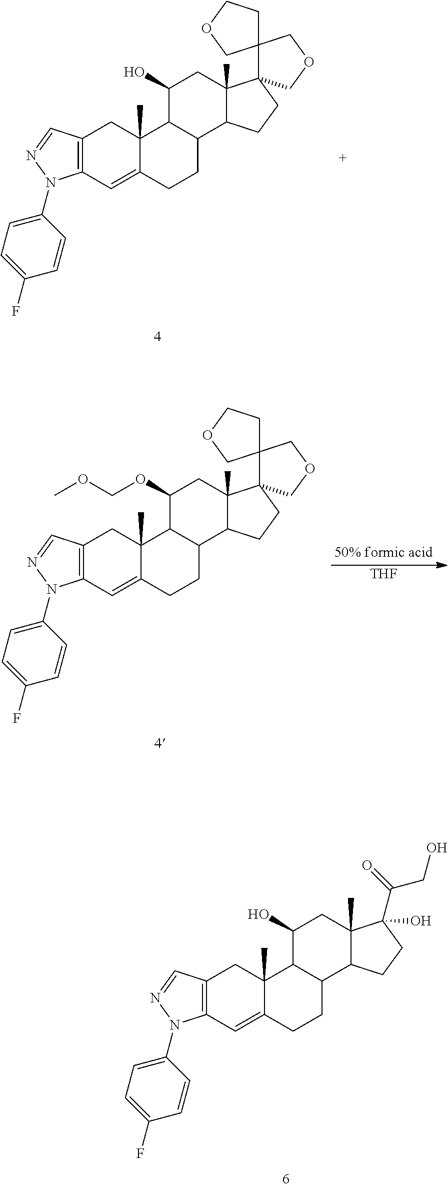

To a stirred solution of the mixture of 4 and 4′ (16 g, 30.6 mmol) in THF (90 mL) was added 50% formic acid (500 mL). The reaction mixture was heated to 95-98° C. for 5 hrs. After cooling down, formic acid was evaporated in vacuo. Cold water was added to the crude product, and yellow solid was precipitated and washed with cold H₂O (×3). The solid was purified by column chromatography to give 4.5 g of 5.

### Step 5

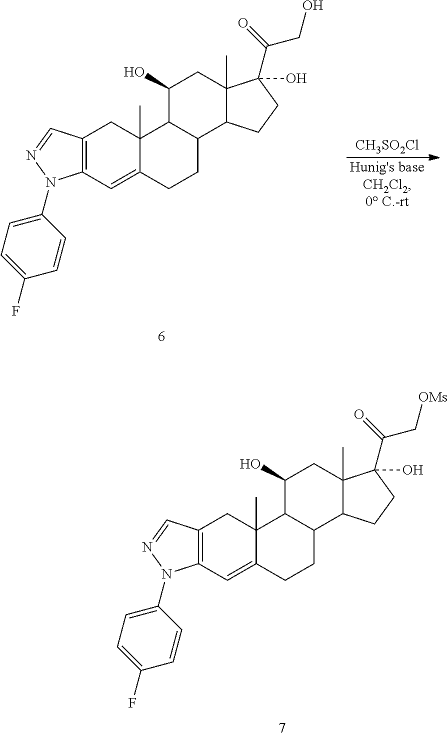

To a solution of the pyrazole 7 (0.76 g, 1.58 mmol) in dichloromethane was treated with Hunig's base and followed by dropwise addition of methanesulfonyl chloride at 0° C. The resulting reaction mixture was stirred at room temperature for 4-6 hours. The reaction mixture was taken in to a separatory funnel, diluted with dichloromethane and washed with 10% HCl, water, brine and dried over anhydrous sodium sulfate. Filtration and removal of solvent gave the mesylate 7 in good yield (0.8 g, 91%, M+1=559.3).

### Step 6

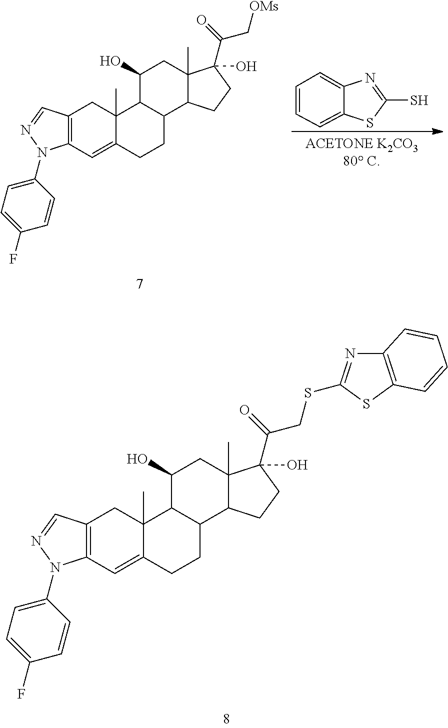

A solution of the mesylate 7 (50 mg, 0.0896 mmol), thiol (24 mg, 0.143 mmol) and potassium carbonate (62 mg, 0.449 mmol) in acetone was heated at 80° C. for 6-8 hours. The reaction mixture was allowed to cool to room temperature and filtered. The filtrate was concentrated under vaccuo and purified by preparative thin layer chromatography using dichloromethane and methanol to afford compound 8 (0.008 g, 13%, M+1=630.3)

### General Procedure for the Furoate Synthesis:

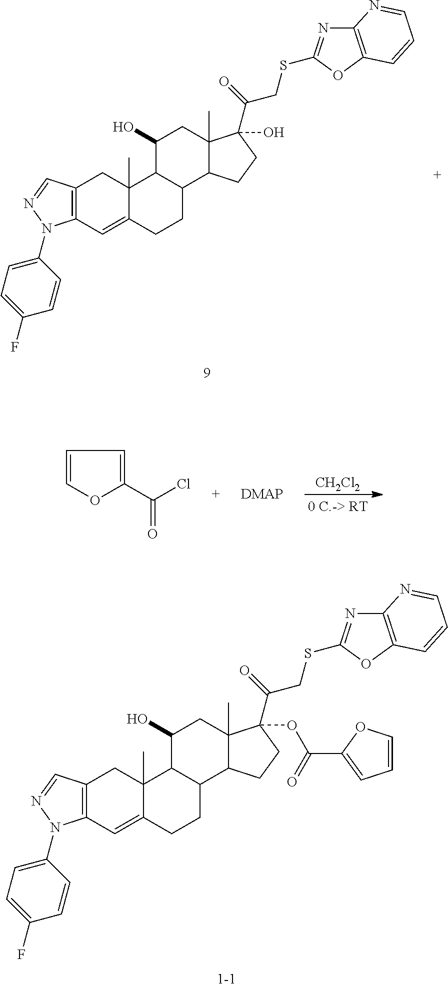

DMAP (0.071 g, 0.584 mmol) was dissolved in methylene chloride (3 mL) under Nitrogen. The solution was cooled to 0° C. and 2-furoyl chloride (0.007 mL, 0.076 mmol) was added dropwise. Starting material 9 (0.045 g, 0.073 mmol) was then taken up in methylene chloride (3 mL) and added to the solution, dropwise. The solution went from 0° C. to room temperature for 24 hours. The solution was concentrated in vacuo and the material was purified on the Gilson HPLC using a gradient of 45% to 90% acetonitrile and water with 0.1% formic acid to afford the final compound 10 [1.3 mg, 2%] (M+1: 709.82).

Using procedures described above for compound 1-1, compounds 1 through 133 were prepared as disclosed in Table 1.

TABLE 1

| | | | IL-8 inhibition |
|---|---|---|---|
| Compound | | | (TR) IC₅₀/Eₘₐₓ |
| # | Structure | M + 1 | (nM, % inhibition) |
| 1 | 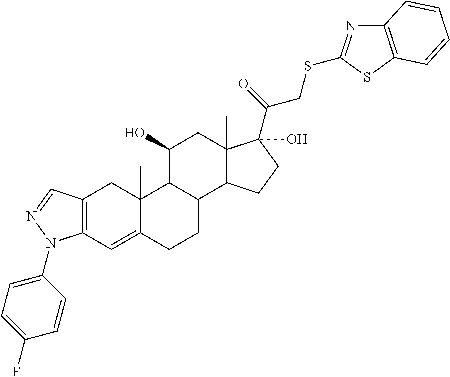 | 630.3 | A/A |
| 2 | 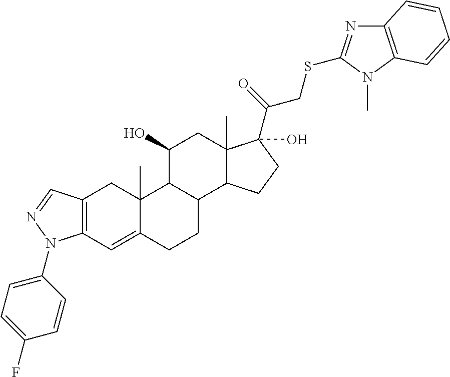 | 627.3 | A/A |
| 3 | 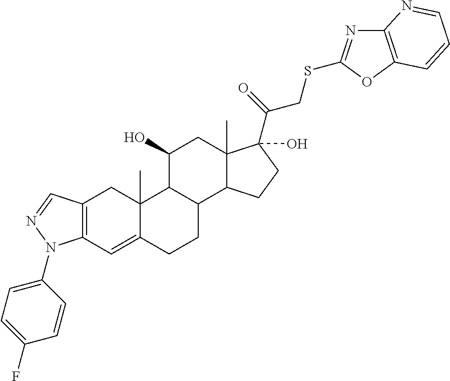 | 615.322 | A/A |
| 4 | 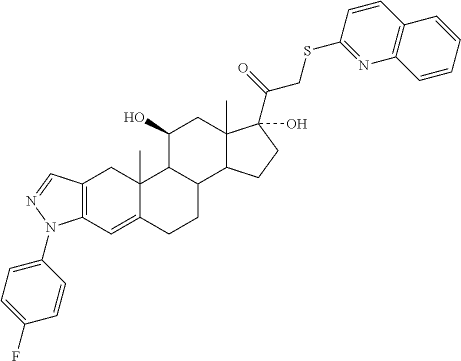 | 624.3 | A/A |
| 5 | 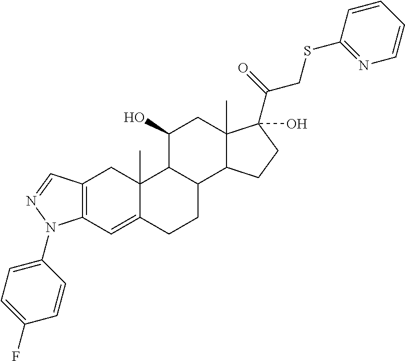 | 574.3 | A/A |
| 6 | 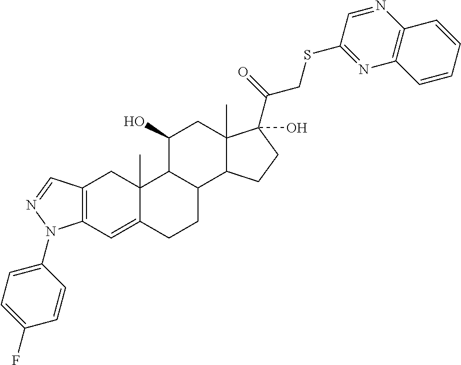 | 625.3 | A/A |
| 7 | 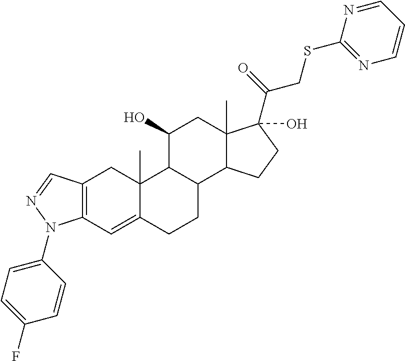 | 575.3 | A/A |
| 9 | 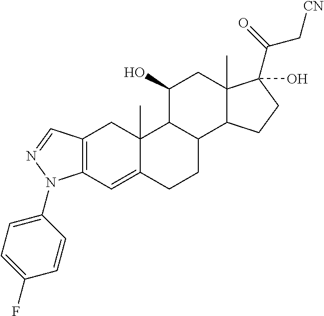 | 490.3 | A/A |
| 10 | 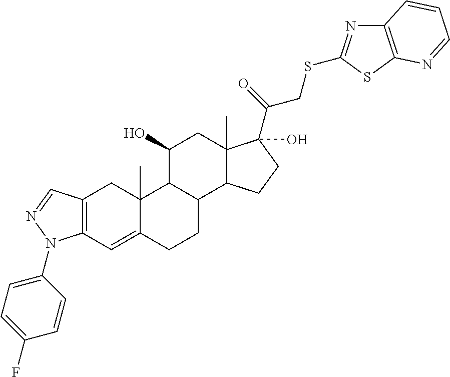 | 631.3 | A/A |
| 11 | 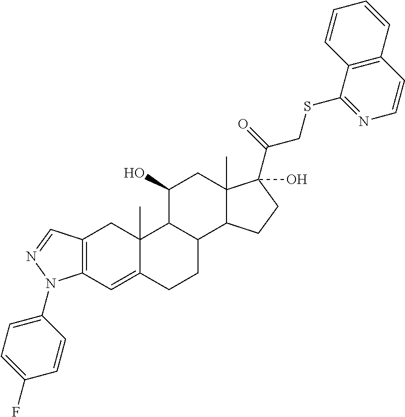 | 624.3 | A/A |
| 12 | 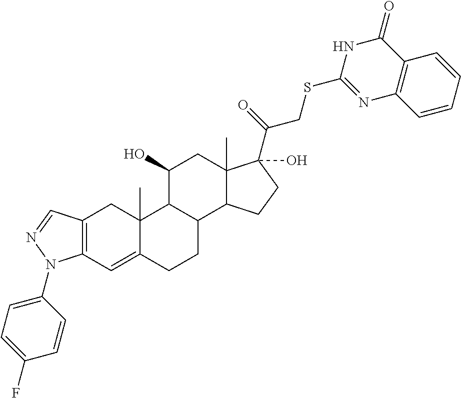 | 641.4 | A/A |
| 13 | 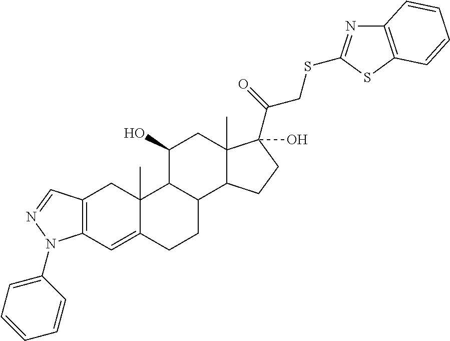 | 612.3 | A/A |
| 14 | 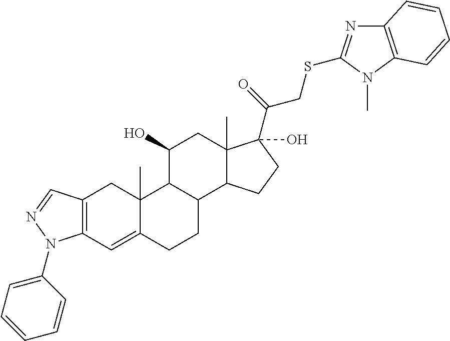 | 609.3 | A/A |
| 15 | 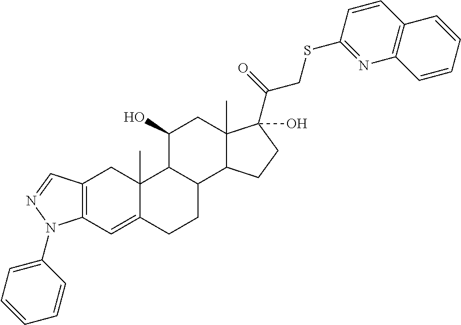 | 606.3 | A/A |
| 16 | 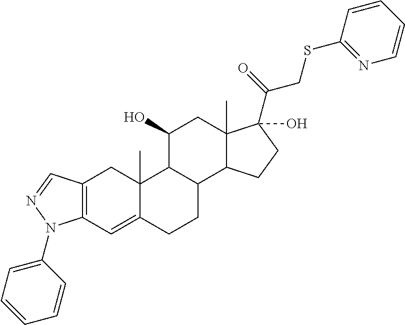 | 556.3 | A/A |
| 17 | 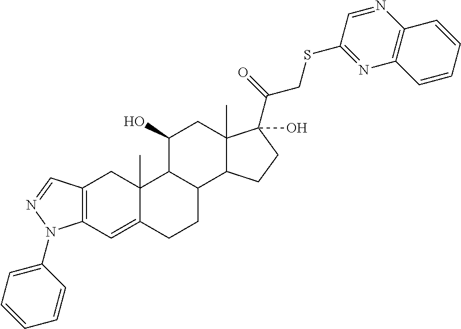 | 607.3 | A/A |
| 18 | 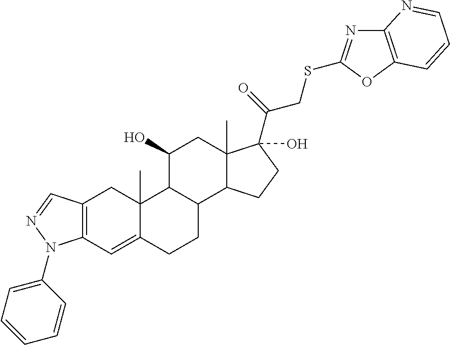 | 597.3 | A/A |
| 19 | 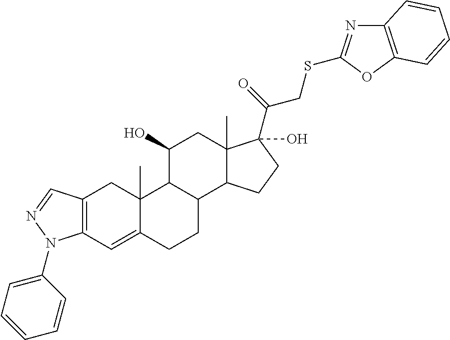 | 596.3 | D/D |
| 20 | 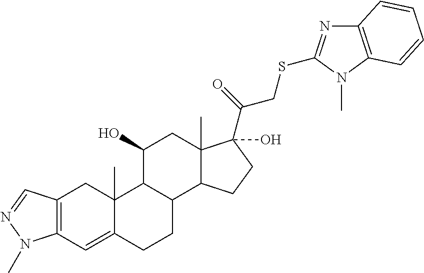 | 547.3 | C/C |
| 21 | 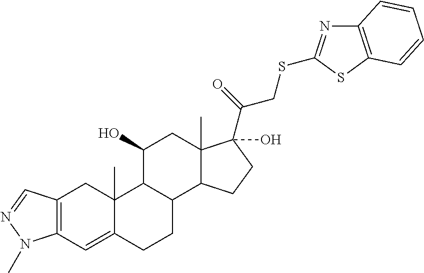 | 550.3 | **/D |
| 22 | 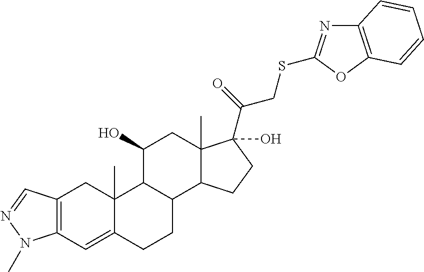 | 534.3 | C/D |
| 23 | 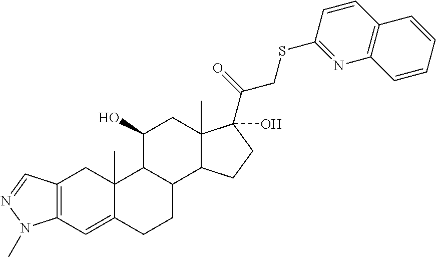 | 544.3 | C/C |
| 24 | 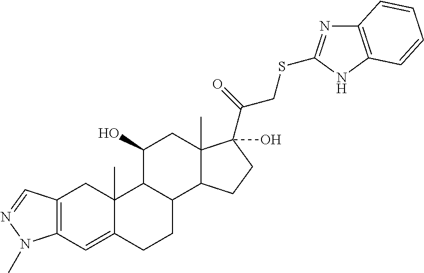 | 533.3 | C/B |
| 25 | 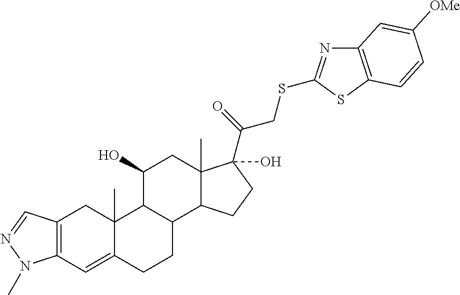 | 580.3 | **/D |
| 26 | 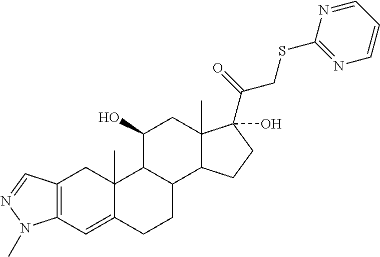 | 495.3 | **/D |
| 27 | 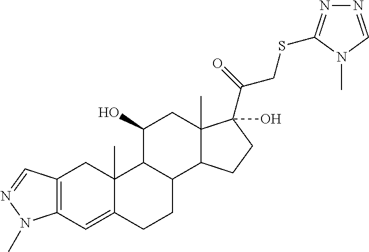 | 480.3 | **/D |
| 28 | 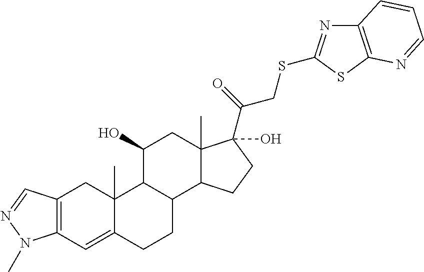 | 551.3 | **/D |
| 29 | 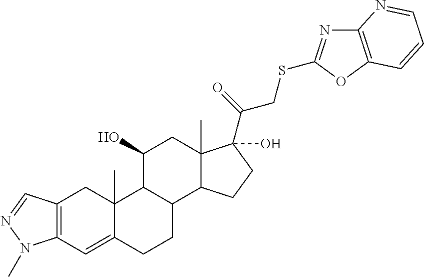 | 535.3 | D/D |
| 30 | 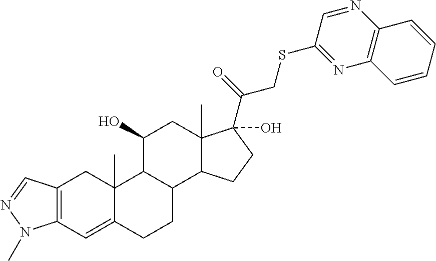 | 545.3 | C/C |
| 31 | 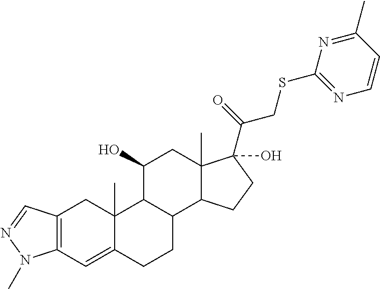 | 509.3 | **/D |
| 32 | 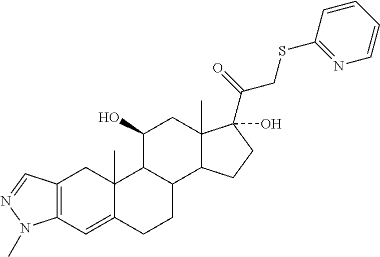 | 494.3 | **/D |
| 33 | 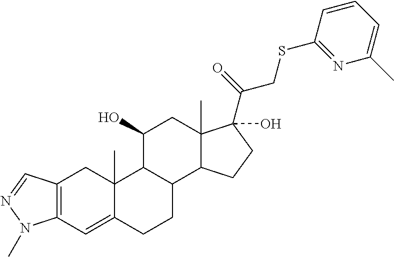 | 508.3 | C/D |
| 34 | 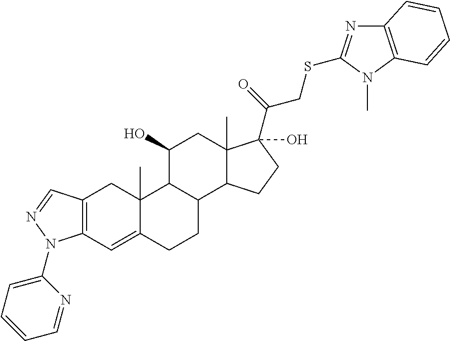 | 610.3 | C/C |
| 35 | 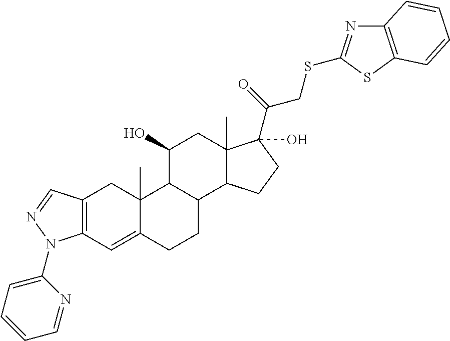 | 613.3 | C/C |
| 36 | 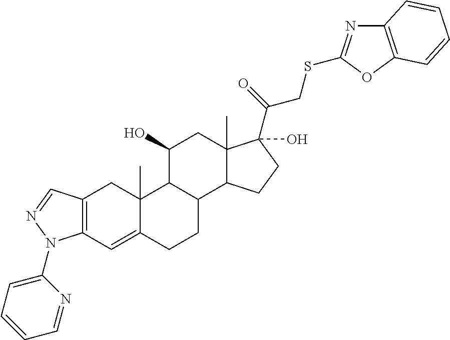 | 597.3 | A/A |
| 37 | 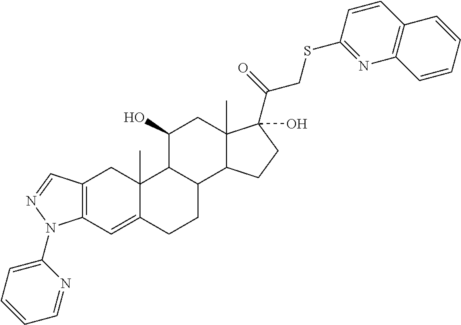 | 607.3 | **/D |
| 38 | 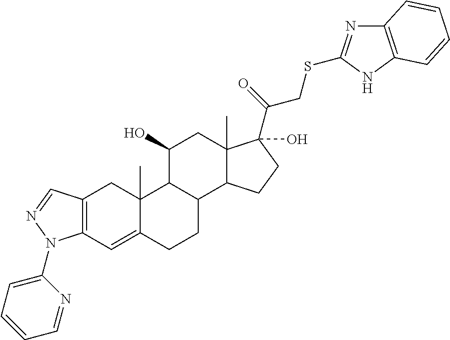 | 596.3 | D/C |
| 39 | 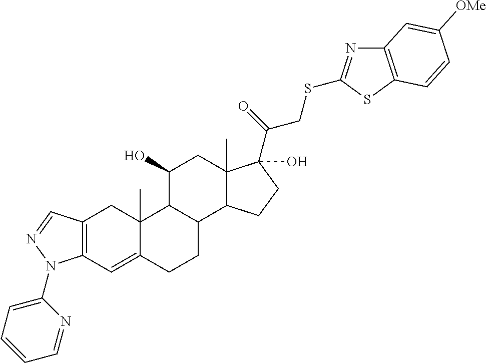 | 643.4 | **/D |
| 40 | 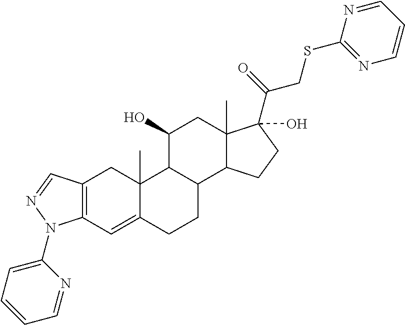 | 558.3 | C/C |
| 41 | 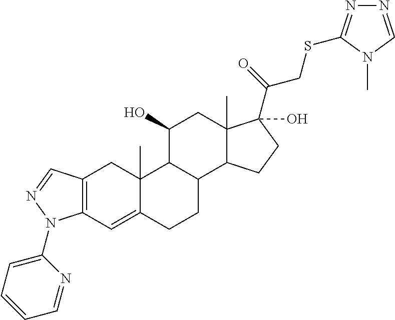 | 561.3 | **/D |
| 42 | 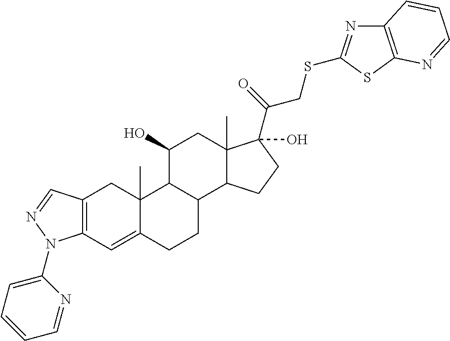 | 614.3 | C/B |
| 43 | 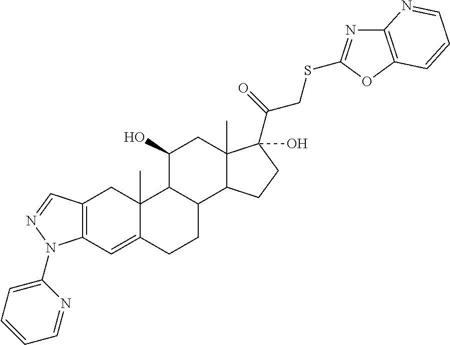 | 598.3 | B/B |
| 44 | 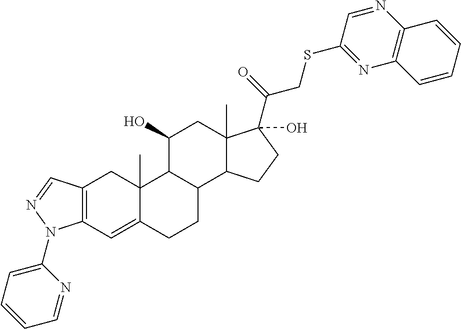 | 608.3 | D/D |
| 45 | 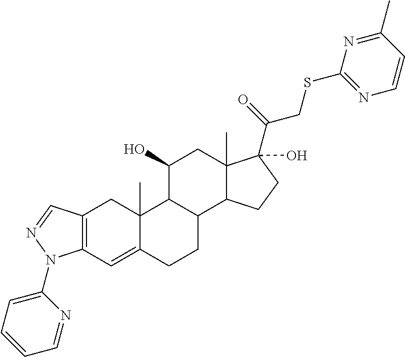 | 572.3 | B/A |
| 46 | 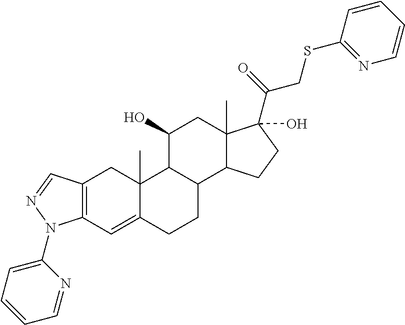 | 557.3 | C/B |
| 47 | 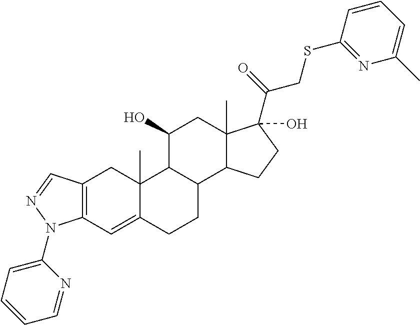 | 571.3 | C/A |
| 48 | 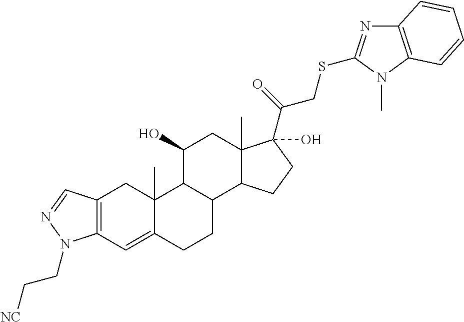 | 586.3 | C/D |
| 49 | 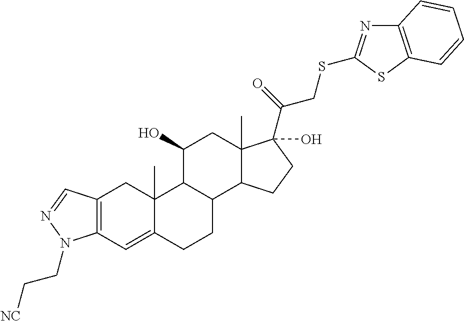 | 589.3 | **/D |
| 50 | 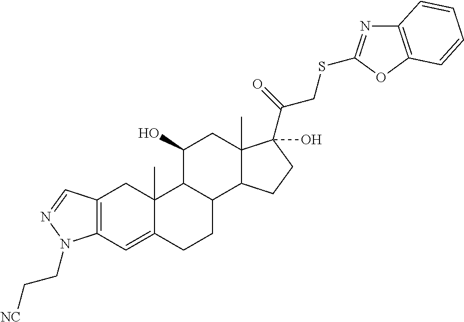 | 573.3 | C/B |
| 51 | 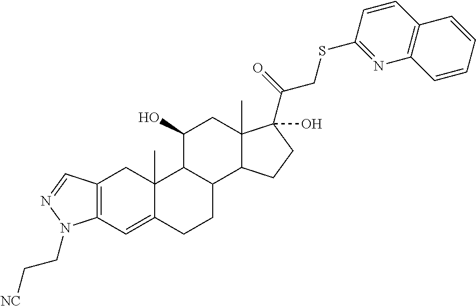 | 583.3 | **/D |
| 52 | 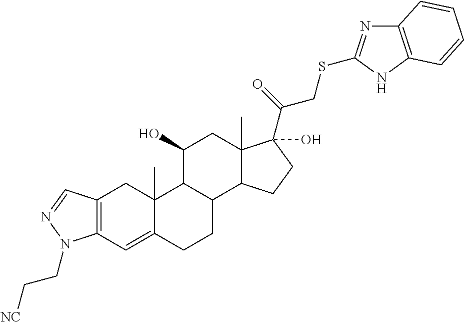 | 572.3 | D/D |
| 53 | 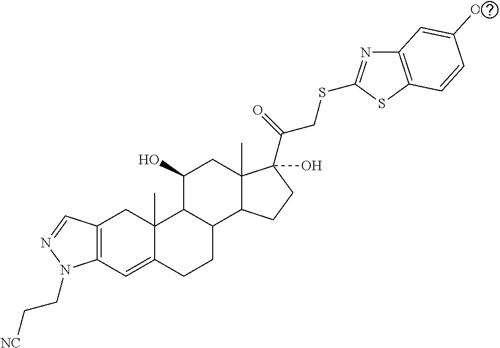 | 619.3 | **/D |
| 54 | 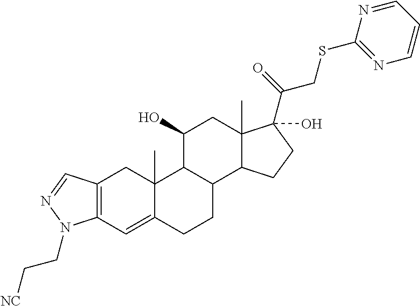 | 534.3 | D/D |
| 55 | 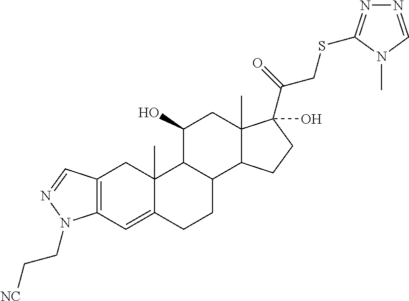 | 537.3 | **/D |
| 56 | 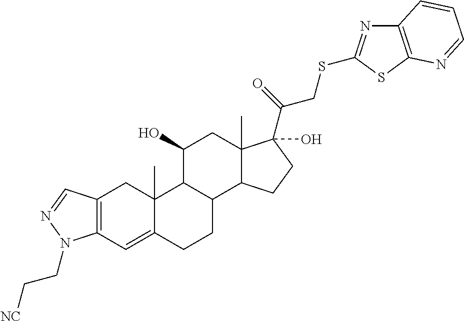 | 590.3 | **/D |
| 57 | 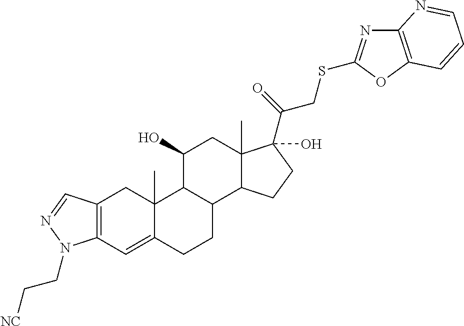 | 556.3 | C/D |
| 58 | 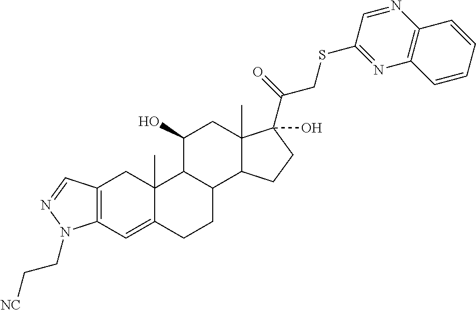 | 584.3 | **/D |
| 59 | 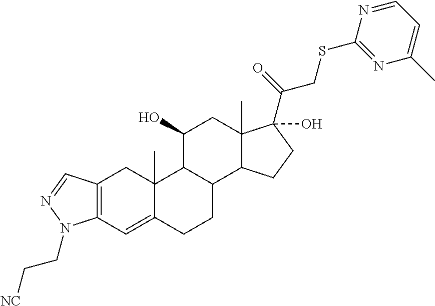 | 548.3 | C/C |
| 60 | 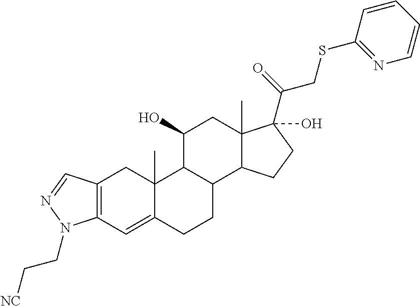 | 533.3 | C/D |
| 61 | 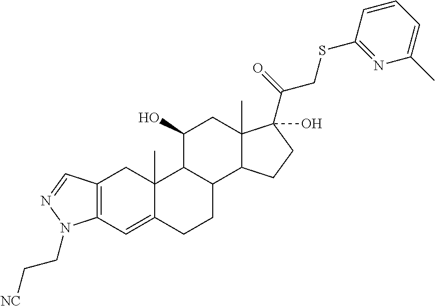 | 547.3 | C/B |
| 62 | 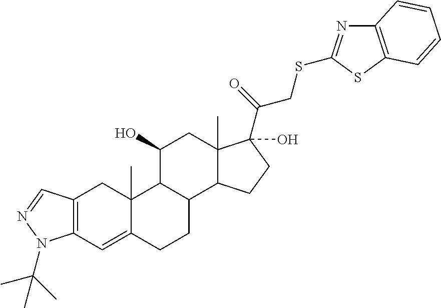 | 592.3 | **/D |
| 63 | 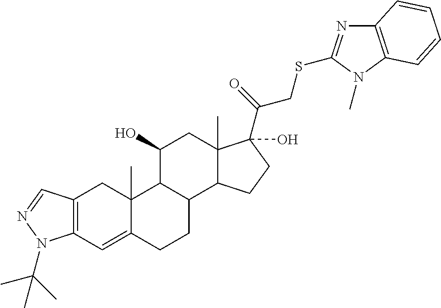 | 589.3 | **/D |
| 64 | 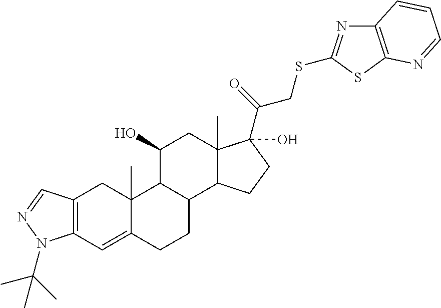 | 593.3 | **/D |
| 65 | 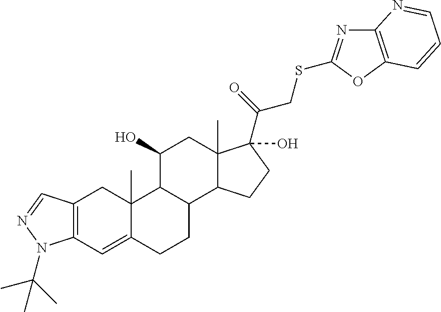 | 577.3 | **/D |
| 66 | 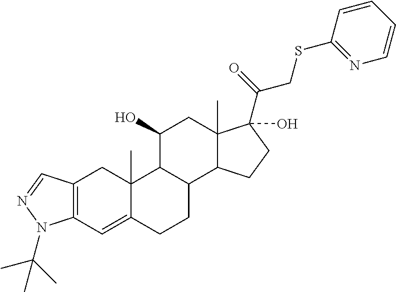 | 536.3 | **/D |
| 67 | 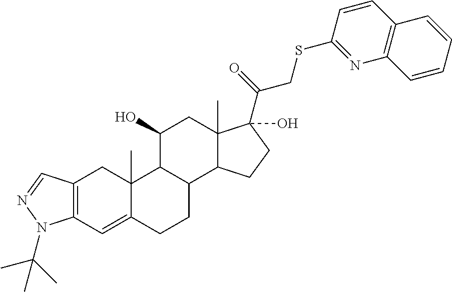 | 586.3 | **/D |
| 68 | 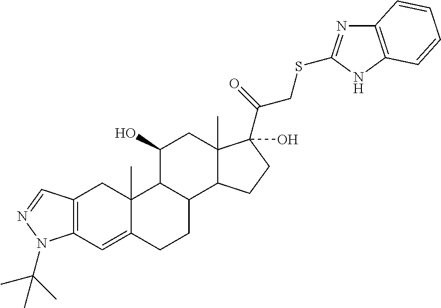 | 575.3 | **/D |
| 69 | 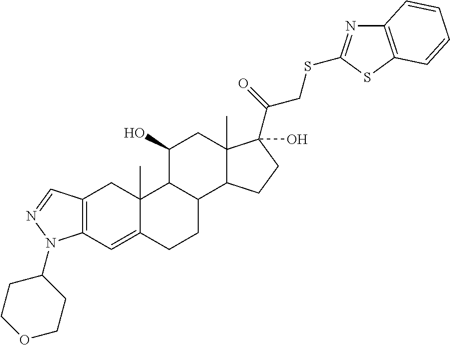 | 620.3 | C/B |
| 70 | 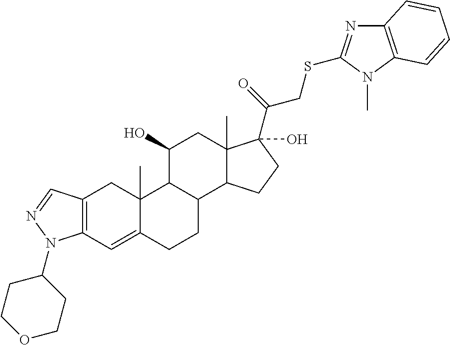 | 617.3 | B/A |
| 71 | 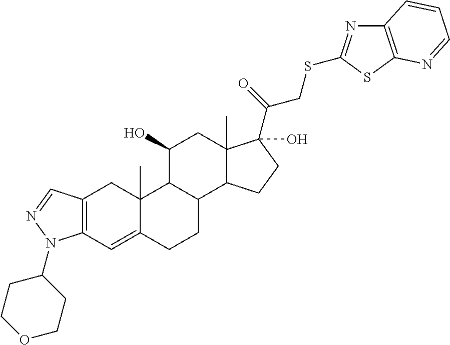 | 621.3 | C/A |
| 72 | 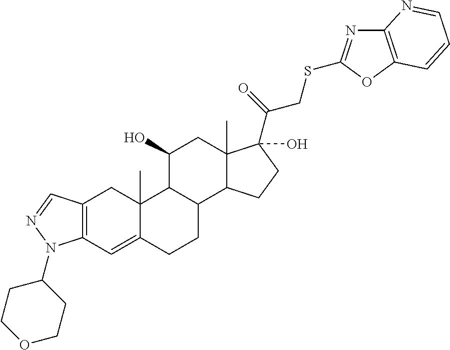 | 605.3 | B/A |
| 73 | 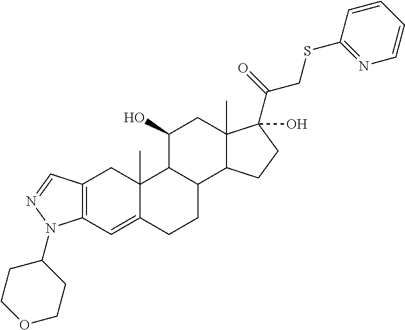 | 564.3 | A/A |
| 74 | 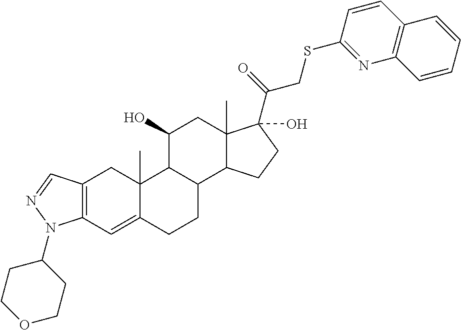 | 614.3 | C/B |
| 75 | 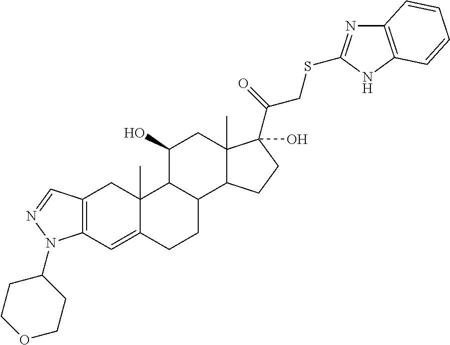 | 603.3 | **/D |
| 76 | 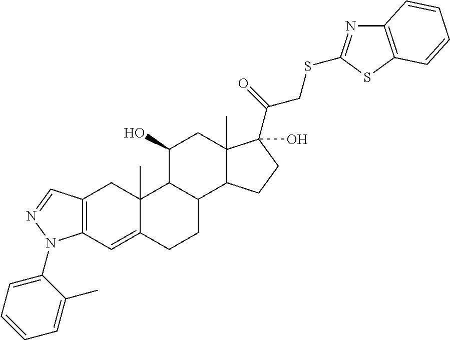 | 626.3 | **/D |
| 77 | 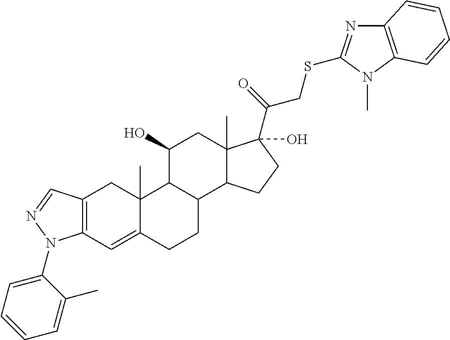 | 623.3 | **/D |
| 78 | 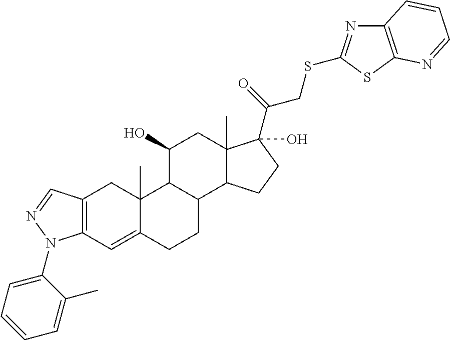 | 627.3 | **/D |
| 79 | 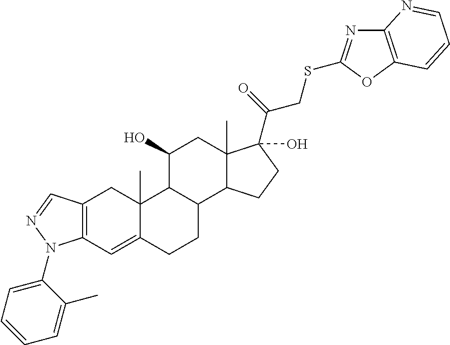 | 611.3 | **/D |
| 80 | 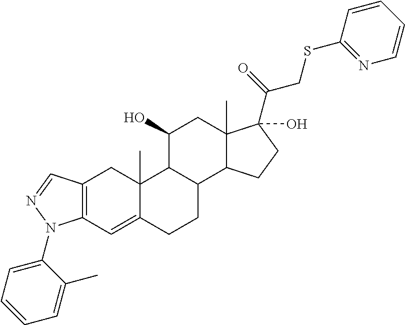 | 570.3 | **/D |
| 81 | 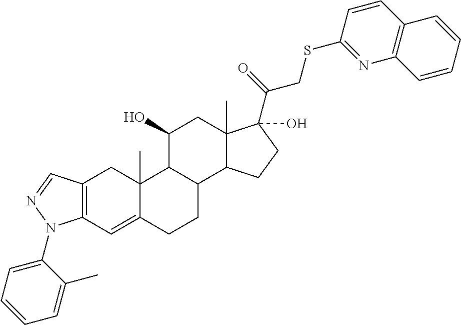 | 620.3 | **/D |
| 82 | 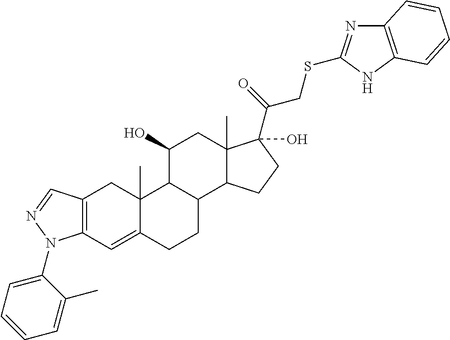 | 609.3 | **/D |
| 83 | 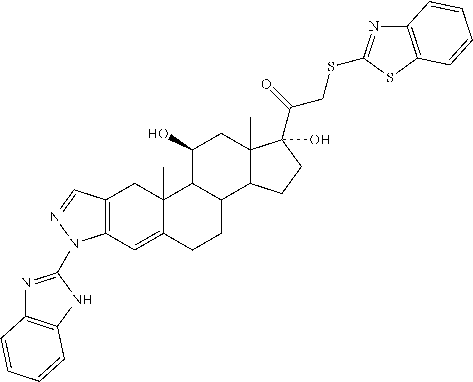 | 652.4 | **/D |
| 84 | 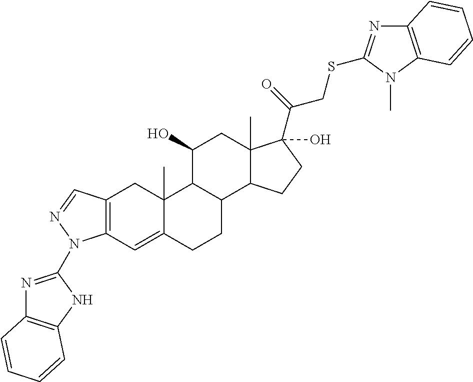 | 649.4 | **/D |
| 85 | 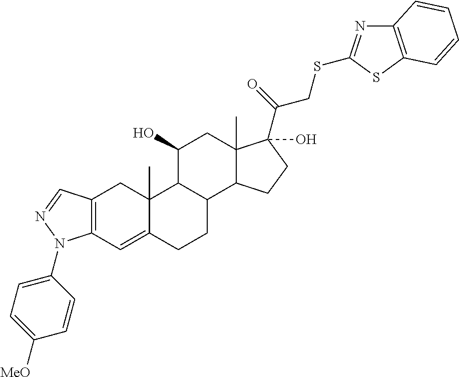 | 642.4 | C/B |
| 86 | 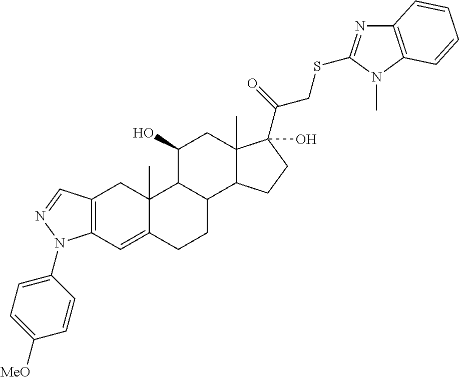 | 639.4 | C/B |
| 87 | 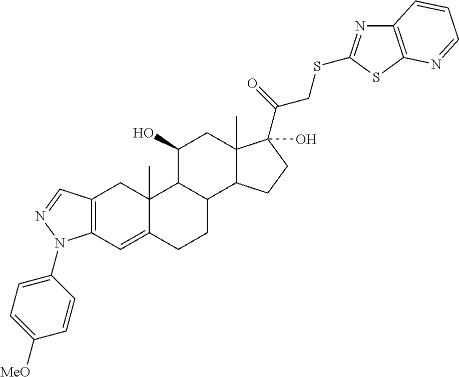 | 643.4 | C/C |
| 88 | 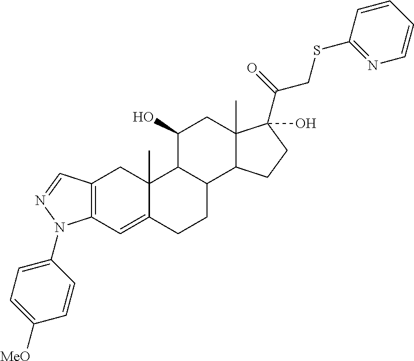 | 586.3 | C/C |
| 89 | 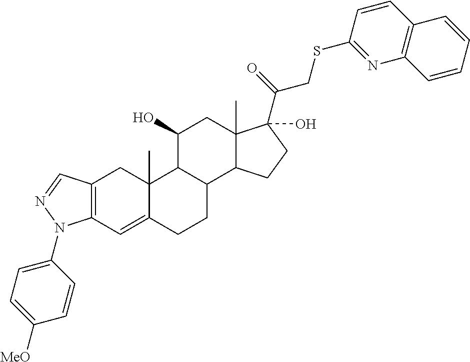 | 636.3 | **/D |
| 90 | 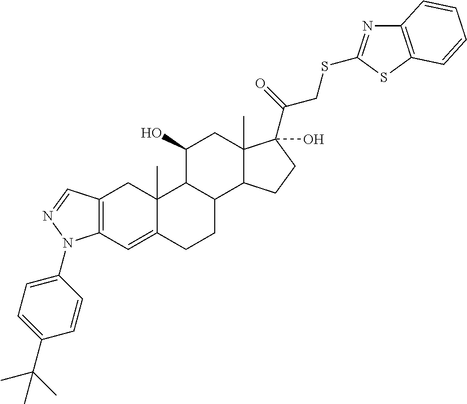 | 668.4 | **/D |
| 91 | 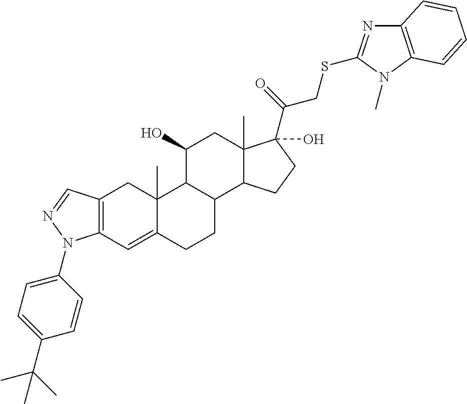 | 665.4 | **/D |
| 92 | 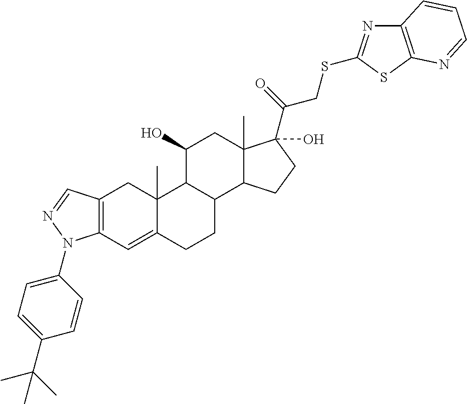 | 669.4 | **/D |
| 93 | 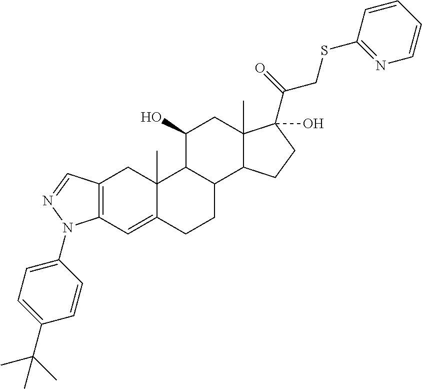 | 612.3 | **/D |
| 94 | 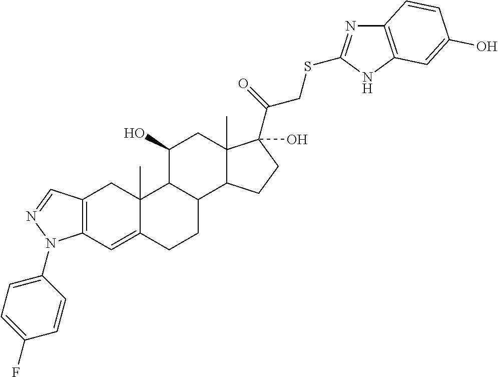 | 629.3 | A/A |
| 95 | 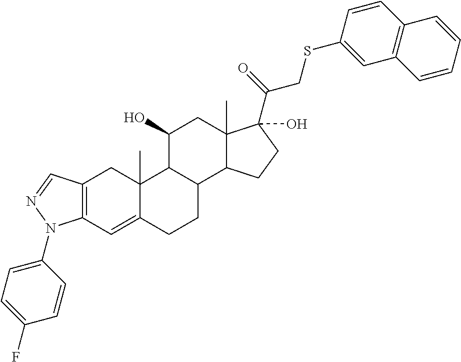 | 623.3 | A/A |
| 96 | 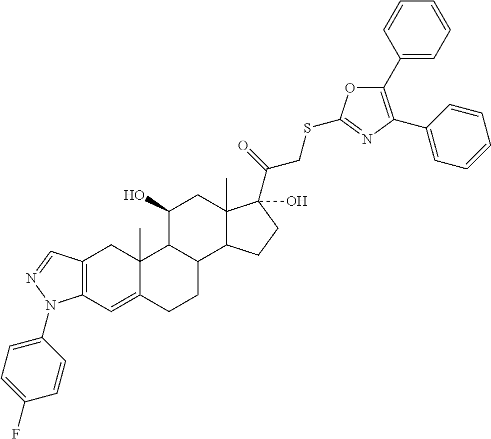 | 716.4 | C/A |
| 97 | 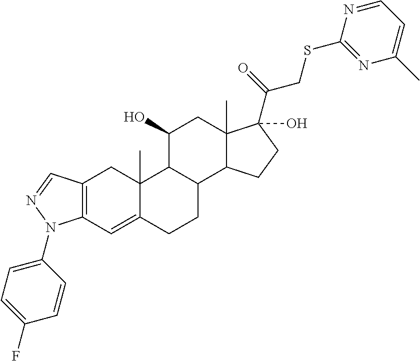 | 589.3 | A/A |
| 98 | 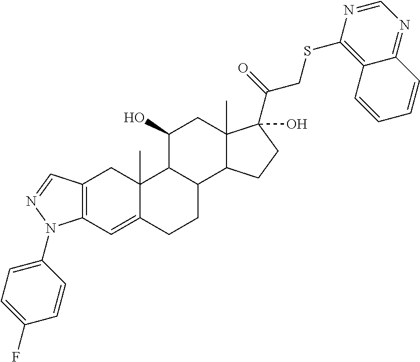 | 625.3 | A/A |
| 99 | 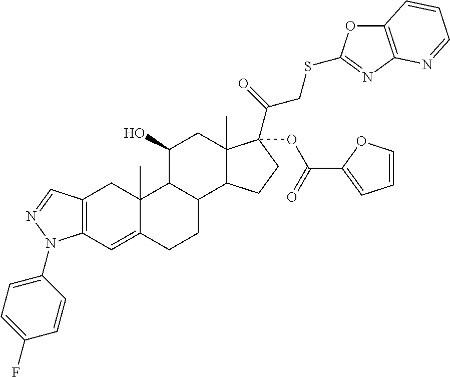 | 709.8 | A/A |
| 100 | 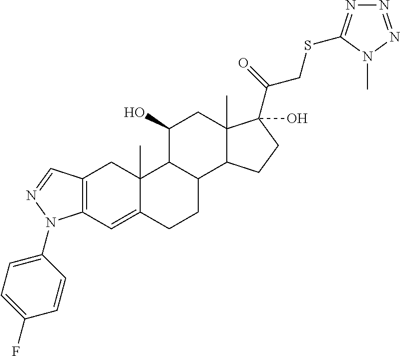 | 579.3 | A/A |
| 101 | 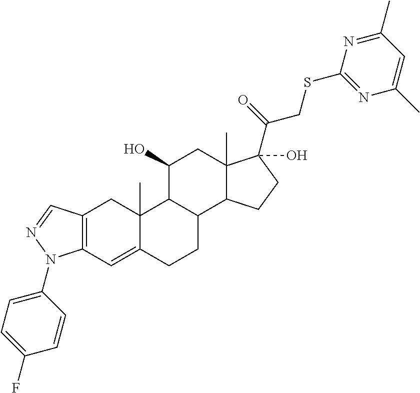 | 603.3 | A/A |
| 102 | 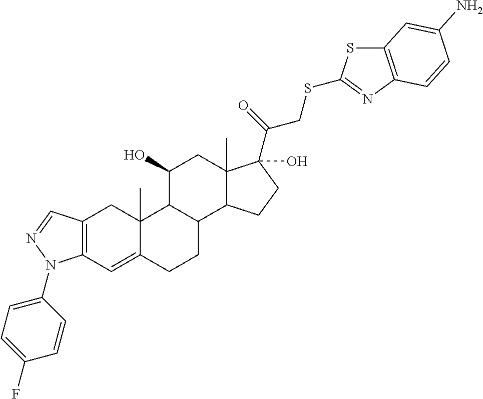 | 645.4 | A/A |
| 103 | 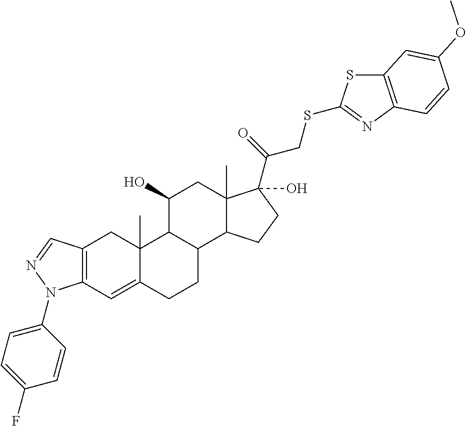 | 660.4 | A/A |
| 104 | 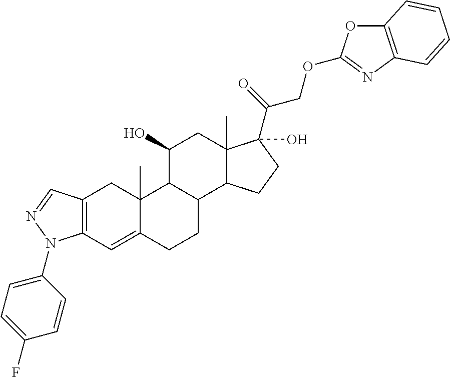 | 598.3 | A/A |
| 110 | 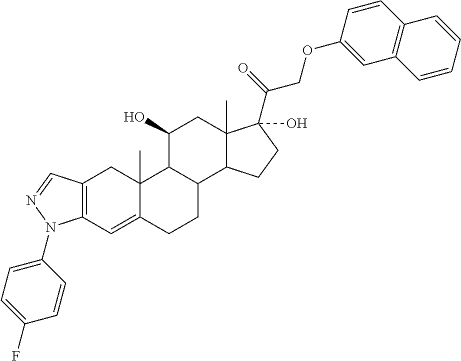 | 607.3 | A/A |
| 111 | 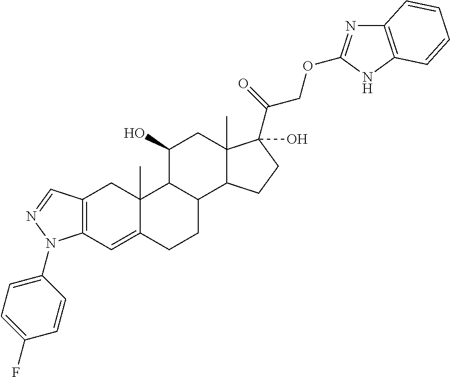 | 597.3 | A/A |
| 112 | 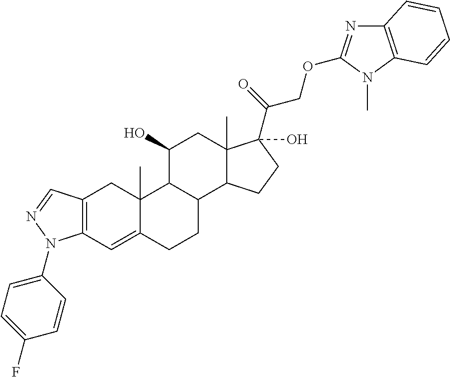 | 611.3 | A/A |
| 113 | 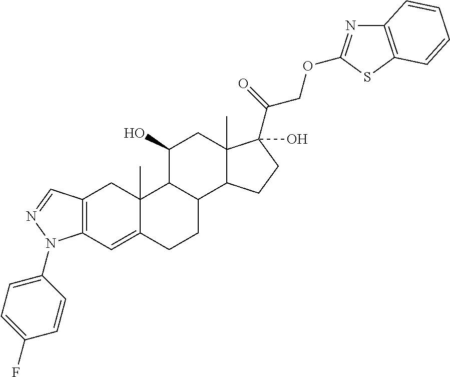 | 614.3 | A/A |
| 114 | 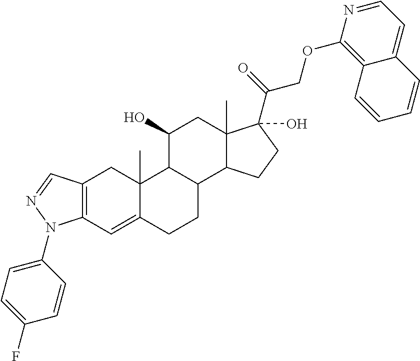 | 608.3 | A/A |
| 115 | 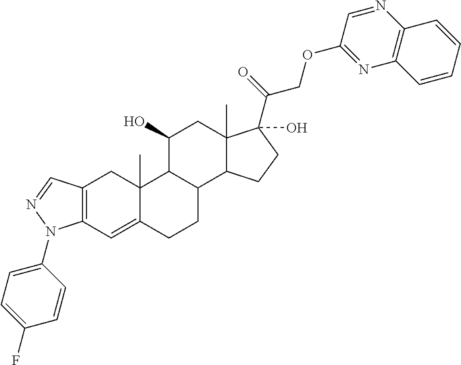 | 591.3 | A/A |
| 116 | 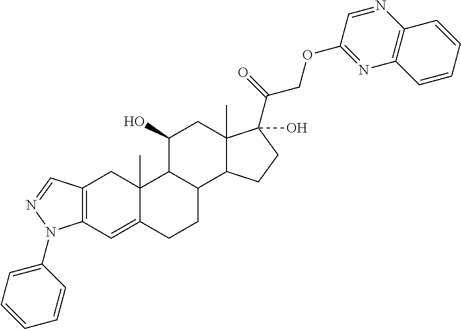 | 607.3 | A/A |
| 117 | 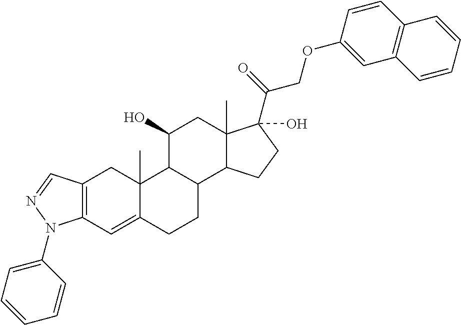 | 589.3 | A/A |
| 118 | 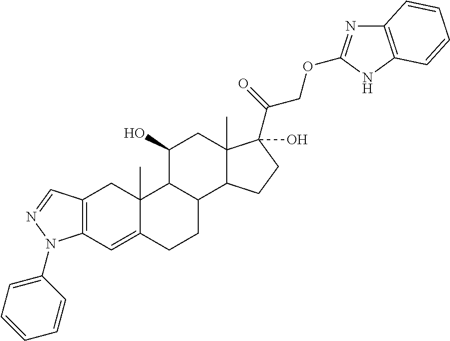 | 579.3 | A/A |
| 119 | 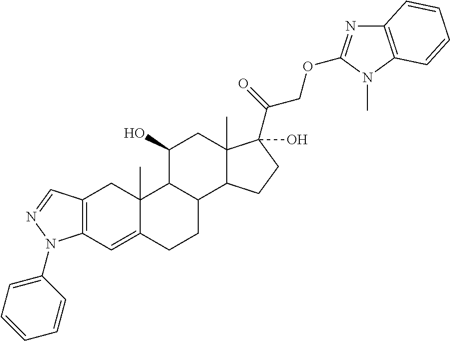 | 593.3 | A/A |
| 120 | 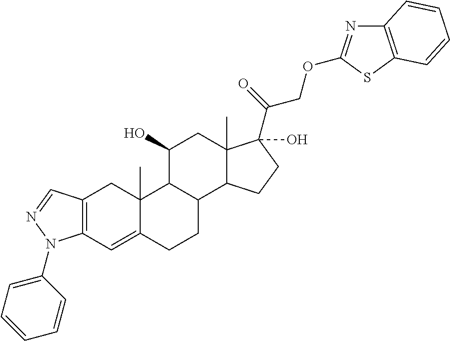 | 596.3 | A/A |
| 121 | 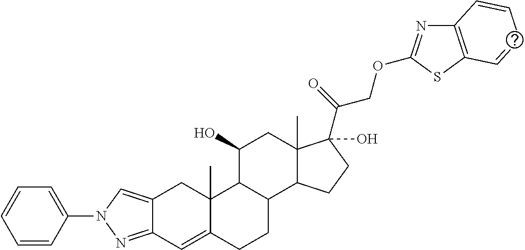 | 596.3 | **/D |
| 122 | 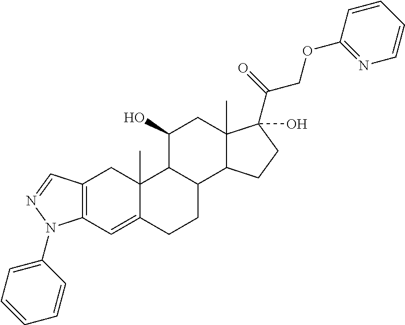 | 540.3 | A/A |
| 123 | 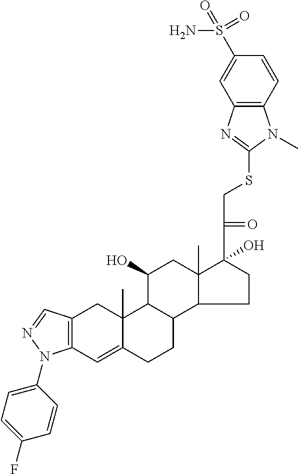 | 706 | A/A |
| 124 | 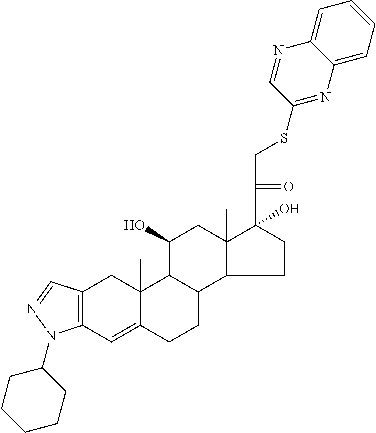 | 613 | A/A |
| 125 | 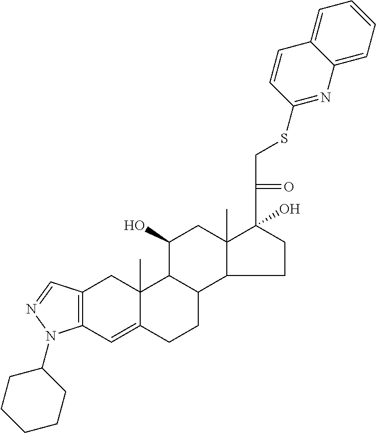 | 612 | B/A |
| 126 | 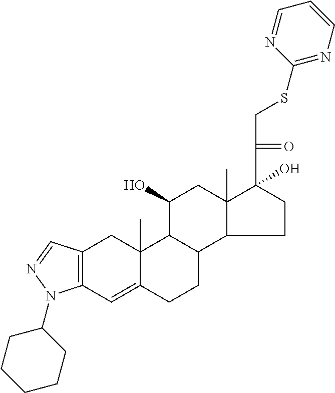 | 563 | A/A |
| 127 | 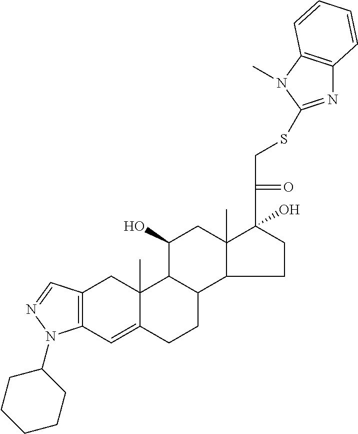 | 615 | A/A |
| 128 | 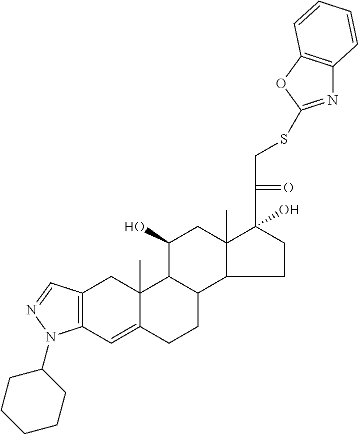 | 602 | A/A |
| 129 | 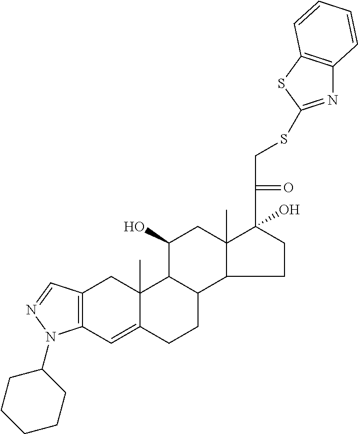 | 618 | A/A |
| 130 | 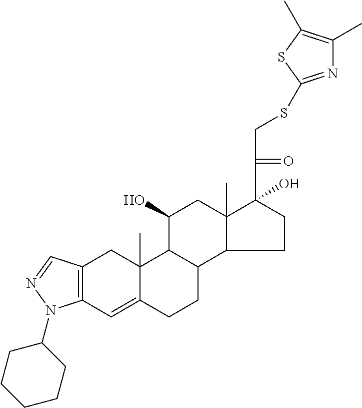 | 596 | A/A |
| 131 | 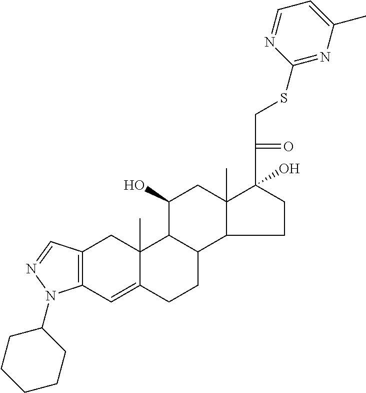 | 577 | A/A |
| 132 | 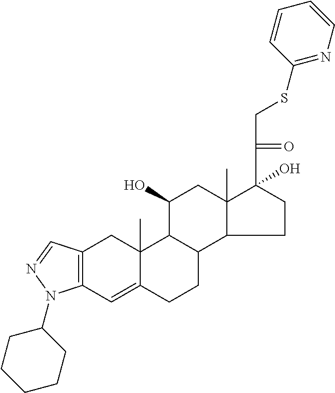 | 562 | A/A |
| 133 | 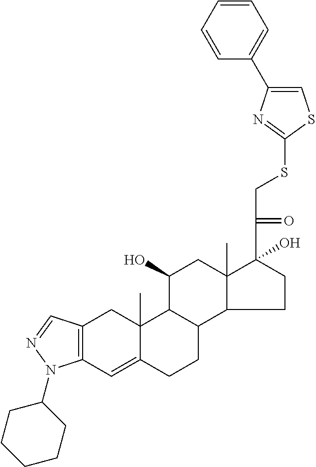 | 644 | B/A |

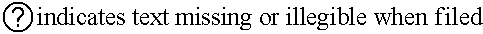

Generally, the compounds having the general structure shown in Formula B can be prepared by the following procedure:

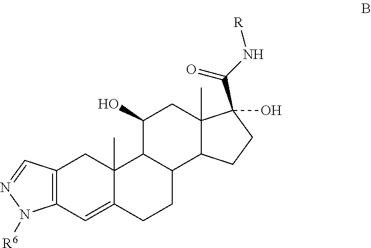

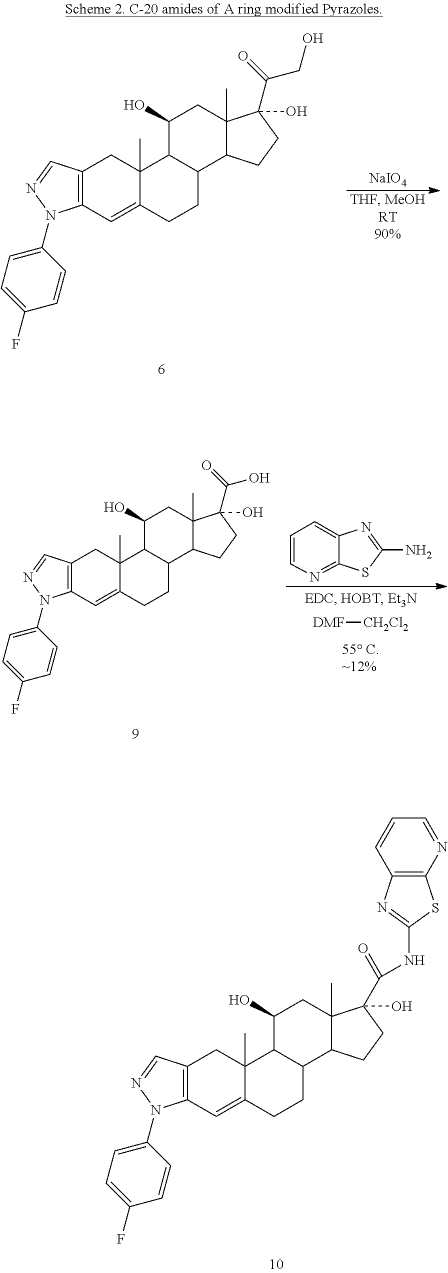

### Step 1)

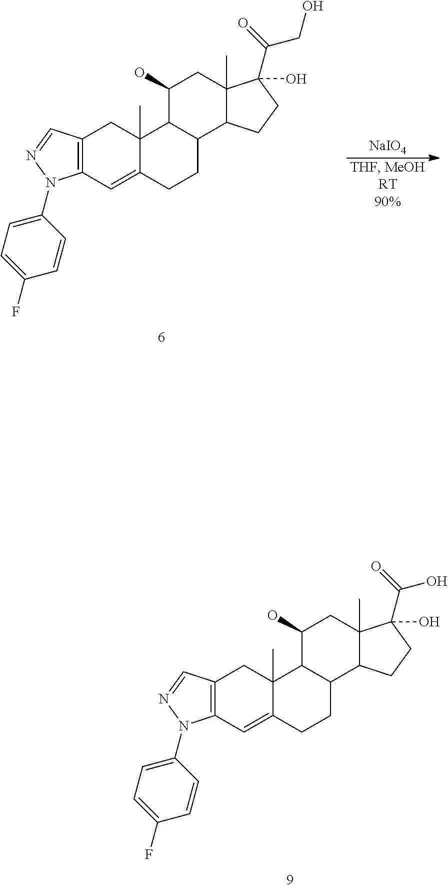

To a solution of the compound 6 (0.28 g, 0.58 mmol) in THF (3 mL) and methanol (1 mL), was added, drop-wise, a warmed solution (−50° C.) of NalO₄ in water (2 mL). Reaction mixture stirred at room temperature for 2 hours. Residue was filtered, washed with water and dried under vacuum to afford the product 7 (0.24 g, 90%)

### Step 2)

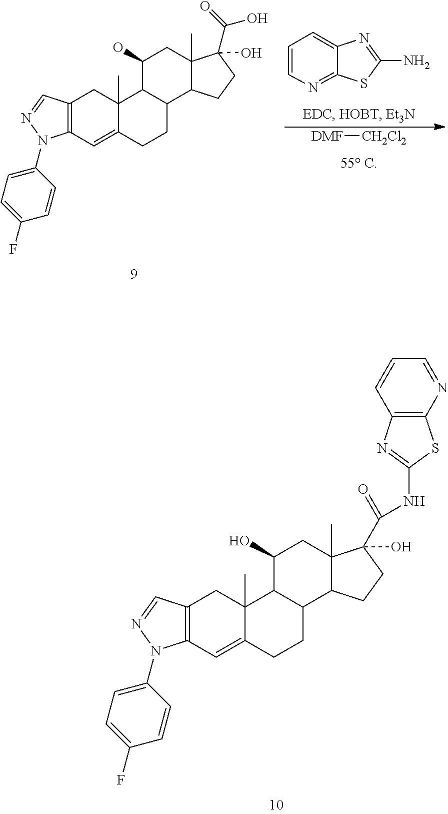

To a solution of pyrazole 7 (0.050 g, 0.107 mmol) in dichloromethane (5 mL) and DMF (1 mL) was added amine (0.017 g, 0.117 mmol), EDC (0.030 g, 0.160 mmol), HOBT (0.021 g, 0.160 mmol), and triethylamine (0.037 mL, 0.267 mmol), respectively. Solution heated to 55° C. for 22 hours. The reaction mixture was concentrated in vacuo and the residue was purified by flash chromatography on silica gel eluting with 0 to 20% of 20% methanol in dichloromethane solution in dichloromethane to afford the product 8 a white solid (8 mg, 12%).

### Alternate Coupling Procedure:

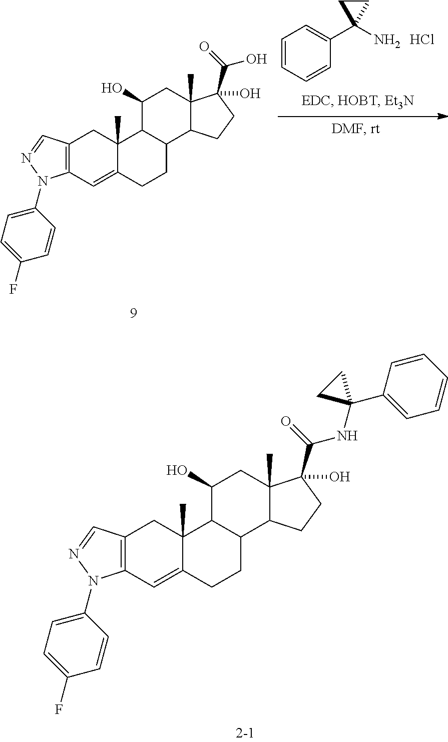

To a stirred solution of 9 (122 mg, 0.261 mmol) in DMF (3.70 mL) was added amine (57.6 mg, 0.339 mmol), EDC (75 mg, 0.391 mmol), HOBT (52.8 mg, 0.391 mmol), and triethylamine (109 0.783 mmol). The reaction mixture was stirred at room temperature for 20 hr. The reaction mixture was poured into sat. NaHCO₃ (aq) solution and extracted with EtOAc. The organic layer was washed with H₂O and brine, dried over MgSO4, filtered and concentrated. The resulting crude product was purified by flash column chromatography on silica gel eluting with EtOAc/Hexanes (1/1) to afford the desired product, 2-1 (75.6 mg, 50%) as a white foam.

Using procedures described above for compound 2-1, compounds 1 through 25 were prepared as disclosed in Table 2.

TABLE 2

| | | | IL-8 inhibition |
|---|---|---|---|
| Compound | | | (TR) IC₅₀/Eₘₐₓ |
| # | Structure | M + 1 | (nM, % inhibition) |
| 1 | 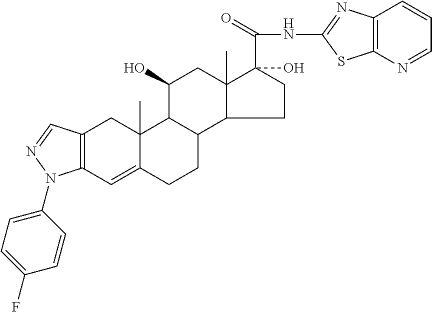 | 600.3 | A/A |
| 2 | 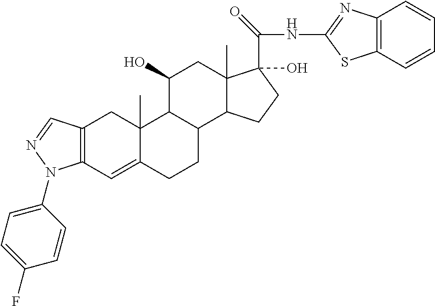 | 599.3 | A/A |
| 3 | 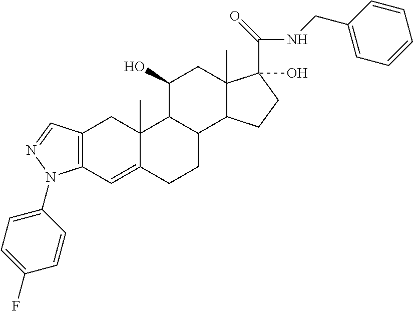 | 556.3 | A/A |
| 4 | 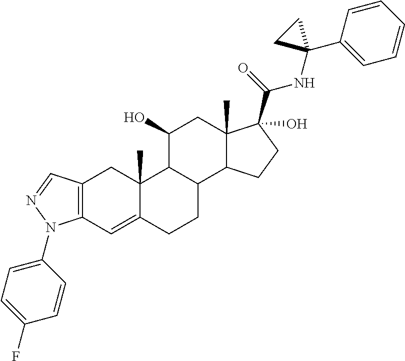 | 582.3 | A/A |
| 5 | 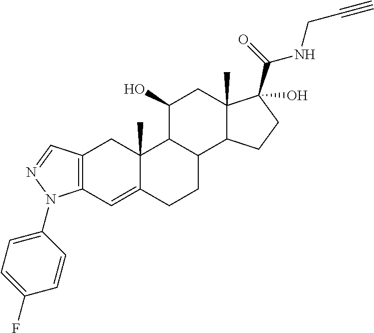 | 504.3 | A/A |
| 6 | 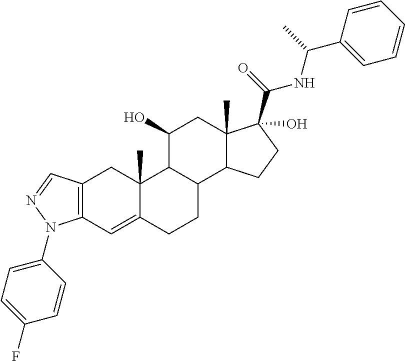 | 570.3 | A/A |
| 7 | 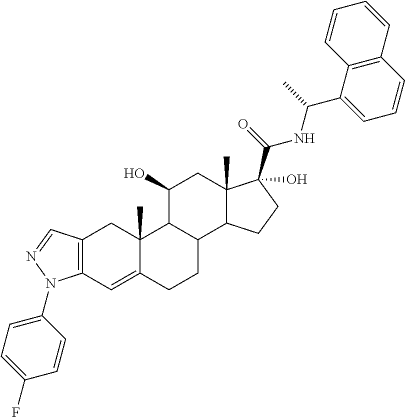 | 620.3 | A/A |
| 8 | 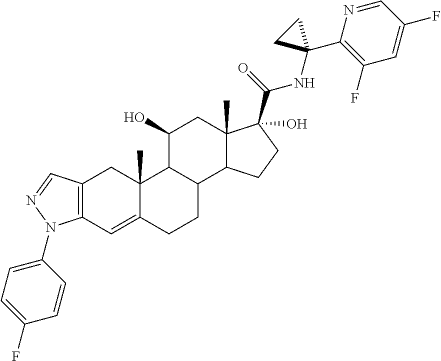 | 619.3 | A/A |
| 9 | 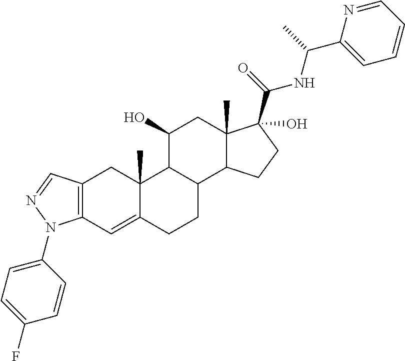 | 571.3 | A/A |
| 10 | 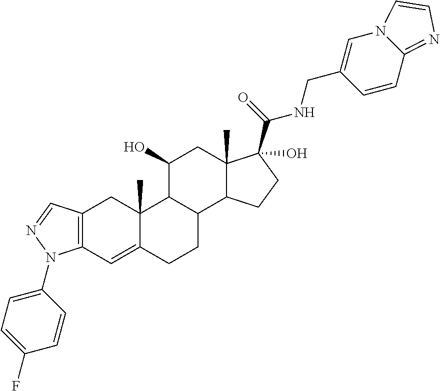 | 596.3 | A/A |
| 11 | 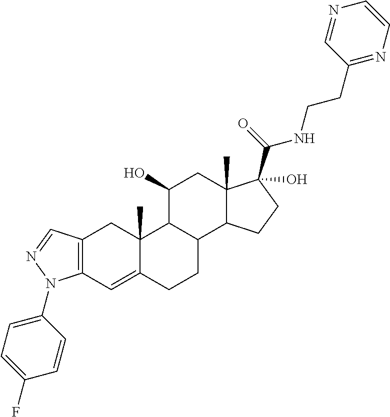 | 572.3 | A/A |
| 12 | 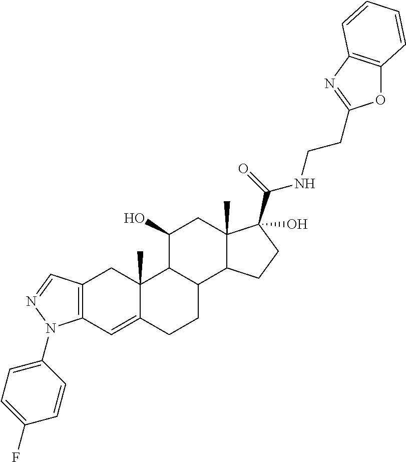 | 611.3 | A/A |
| 13 | 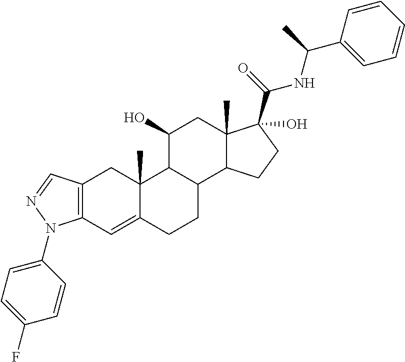 | 570.3 | A/A |
| 14 | 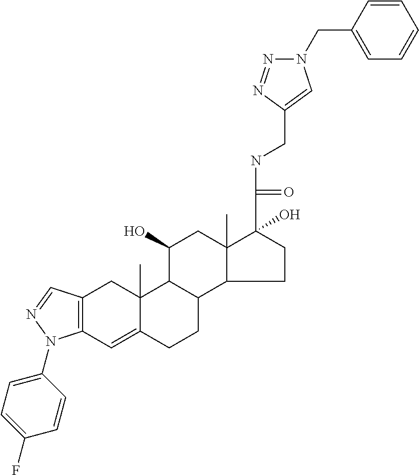 | 637 | A/A |
| 15 | 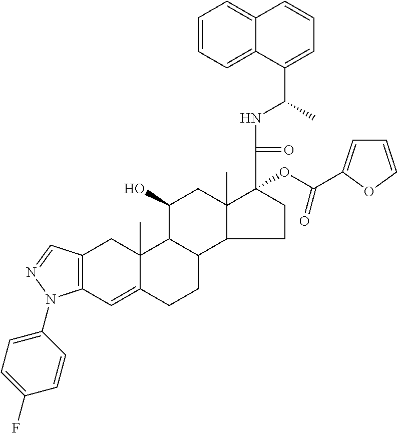 | 714 | A/A |
| 16 | 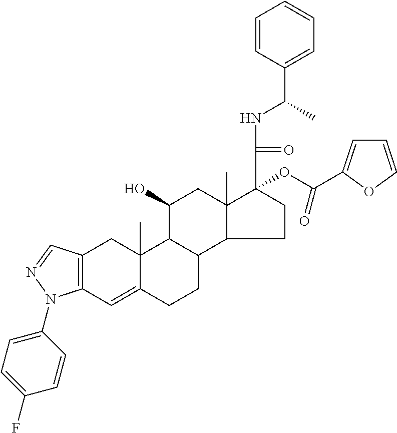 | 664 | A/A |
| 17 | 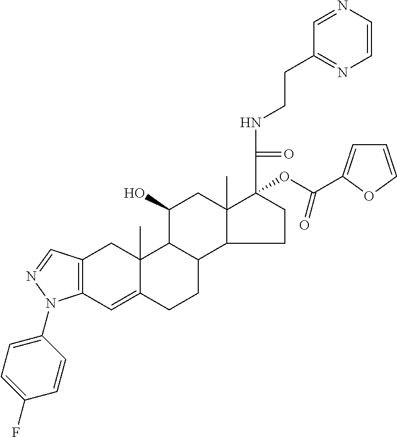 | 666 | A/A |
| 18 | 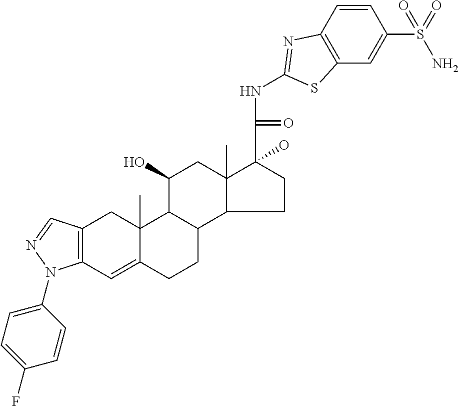 | 678 | A/A |
| 19 | 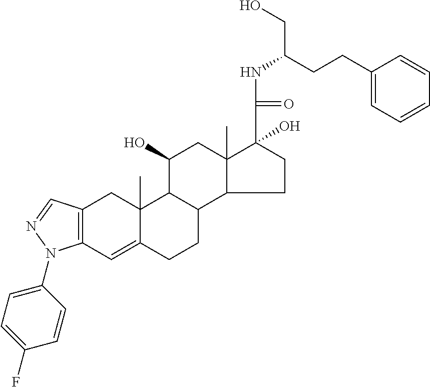 | 614 | A/A |
| 20 | 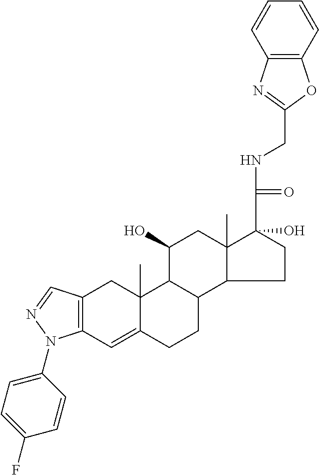 | 597 | A/A |
| 21 | 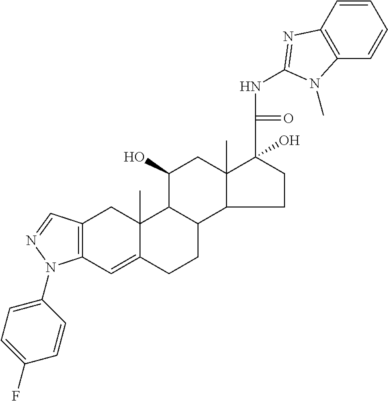 | 596 | A/A |
| 22 | 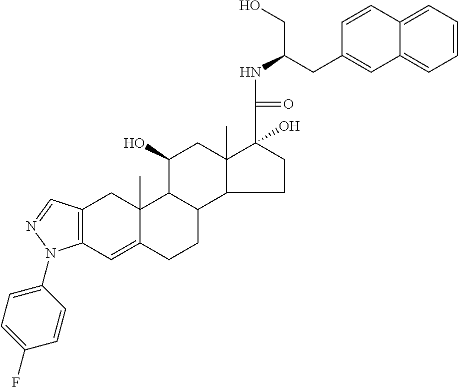 | 650 | A/A |
| 23 | 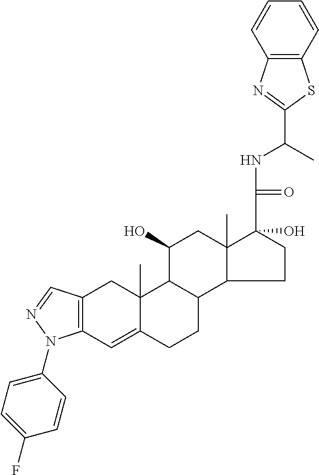 | 627 | A/A |
| 24 | 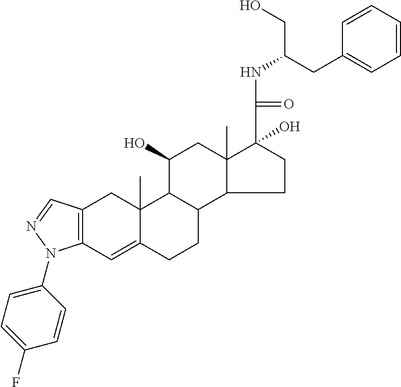 | 600 | A/A |
| 25 | 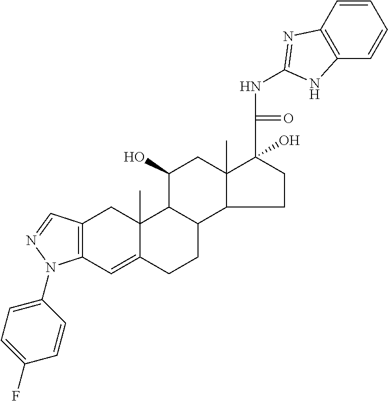 | 582 | |

Generally, compounds of Formula C can be prepared by the following the procedure.

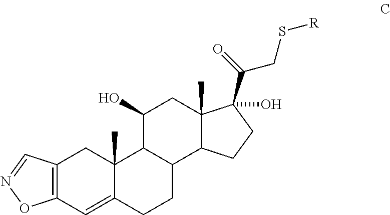

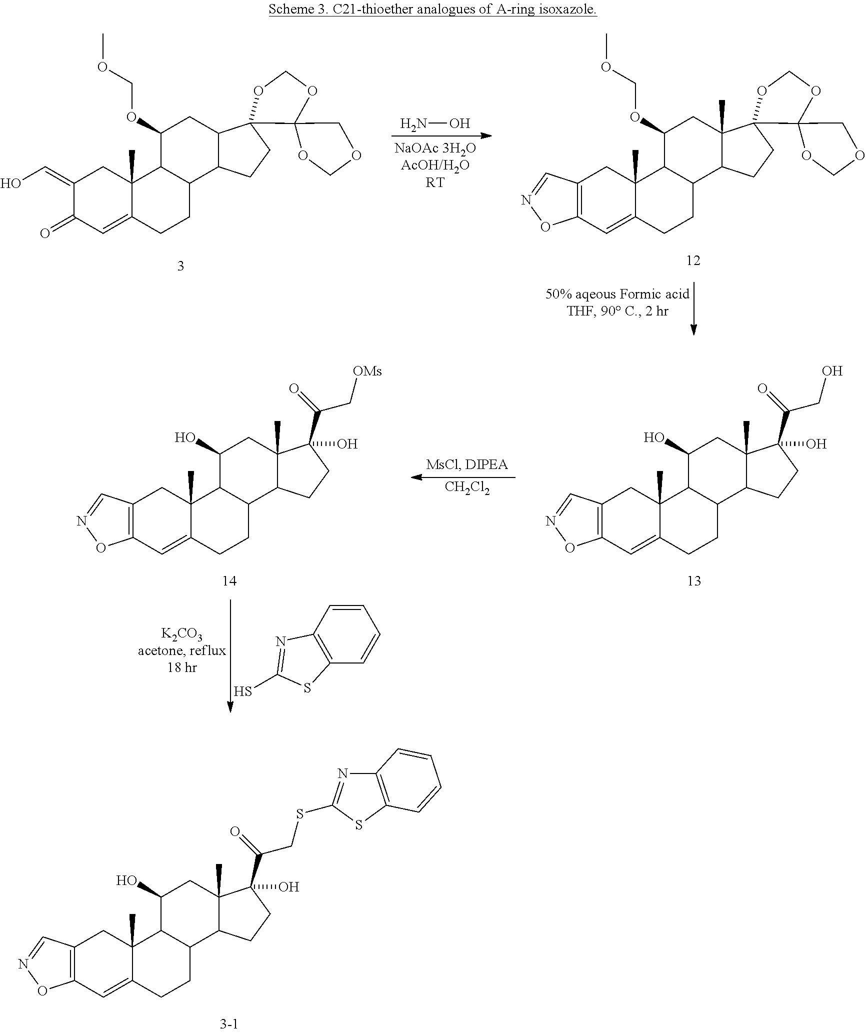

### Step 1)

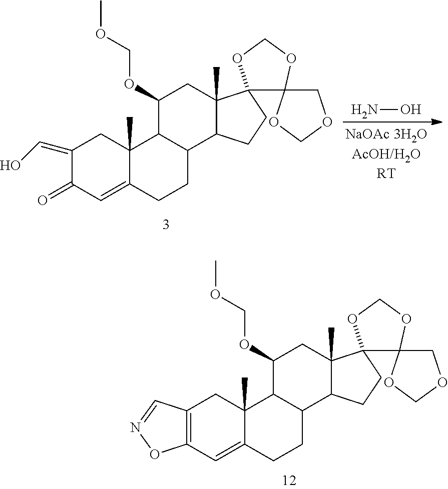

To a stirred solution of [086152-105-29] (5 g, 10.5 mmol) in acetic acid (33 mL) was added a solution of hydroxylamine-HCl (802 mg, 11.5 mol) and sodium acetate-3H₂O (1.44 g, 10.6 mmol) in H₂O (5 mL). The reaction mixture was stirred at room temperature for overnight. Distilled water (150 mL) was added and the aqueous layer was extracted with ethyl acetate (100 mL×3). The combined organic layer was washed with H₂O and brine solution, dried over MgSO₄, filtered and concentrated by rotary evaporator. The resulting light yellow foam 12 was used for the next step without further purification.

### Step 2)

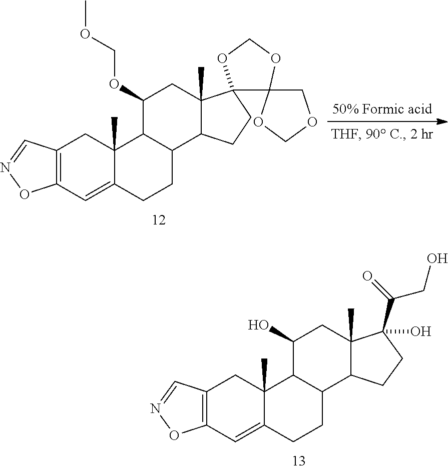

To a stirred solution of 12 (4.5 g, 9.50 mmol) in THF (47 mL) was added 50% formic acid (475 mL) at room temperature. The reaction mixture was heated to 95° C. for 2 hrs and then cooled to room temperature. The formic acid was evaporated by rotary evaporator and the residue was redissolved in MeOH (47 mL) and 1N NaOH (−10 mL) was added. After stirring 3 min at room temperature, the solution was acidified with 1N HCl (−20 mL) and diluted with dichloromethane (200 mL). The aqueous solution was extracted with dichloromethane (100 mL×3). The combined organic layer was washed with H₂O and brine solution, dried over MgSO₄, filtered and concentrated by rotary evaporator. The crude product was purified by column chromatography eluting with EtOAc/Hexanes (1/1) to give compound 13 (1.5 g, 41%)

### Step 3)

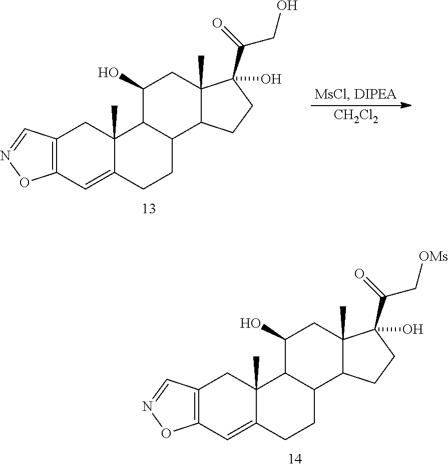

To a stirred solution of 13 (920 mg, 2.37 mmol) in dichloromethane (34 mL) was added diisopropylamine (DIPEA, 1.24 mL, 7.11 mmol), 4-dimethylaminopyridine (DMAP, 28.9 mg, 0.24 mmol) and methanesulfonyl chloride (0.276 mL, 3.55 mmol) at 0° C. The reaction mixture was stirred at 0° C. for 2 hr and then was poured into ice-cold 1N HCl (aq) solution. The aqueous layer was extracted with dichloromethane (100 mL×2) and the combined organic layer was washed with 1N HCl, NaHCO₃, H₂O and brine solution, dried over MgSO₄, filtered and concentrated by rotary evaporator. The resulting light yellow foam 14 was used for the next step without further purification.

### Step 4)

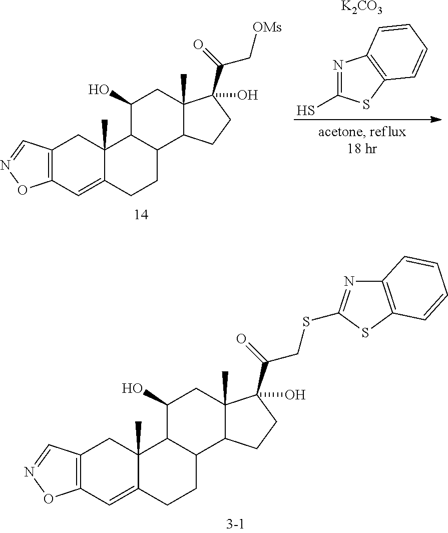

To a stirred solution of 14 (107 mg, 0.23 mmol) in anhydrous acetone (4 mL) was added 2-mercaptobenzothiazole (57.3 mg, 0.35 mmol) and anhydrous potassium carbonate (158 mg, 1.15 mmol) at room temperature. The reaction mixture was heated to 70° C. for 18 hrs and then cooled to room temperature.

The reaction mixture was filtered. The filtrate was concentrated by rotary evaporator and the resulting crude product was purified by column chromatography eluting with EtOAc/Hexanes (1/5) to give compound 3-1 (98 mg, 79%).

Using procedures described above for compound 3-1, compounds 1 through 6 were prepared as disclosed in Table 3.

TABLE 3

| | | | IL-8 inhibition |
|---|---|---|---|
| Compound | | | (TR) IC₅₀/Eₘₐₓ |
| # | Structure | M + 1 | (nM, % inhibition) |
| 1 | 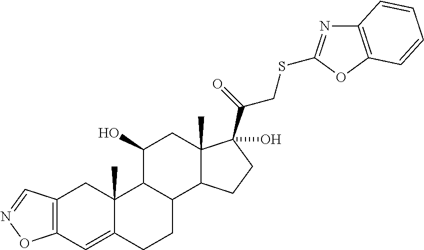 | 521.2 | **/D |
| 2 | 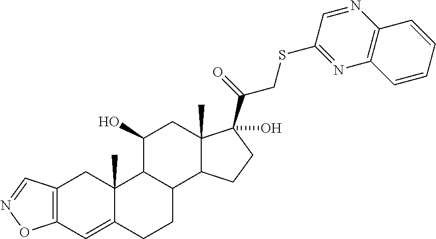 | 532.2 | **/D |
| 3 | 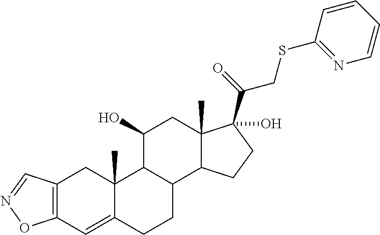 | 481.2 | **/D |
| 4 | 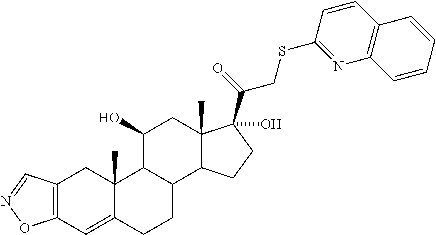 | 531.2 | **/D |
| 5 | 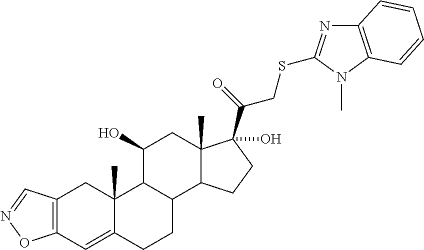 | 534.2 | D/D |
| 6 | 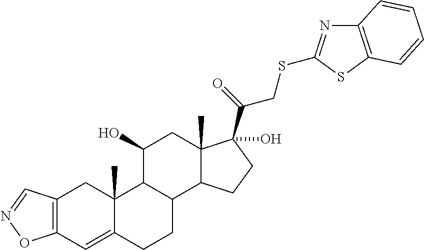 | 537.2 | **/D |

Generally, the compounds of Formula D can be prepared by the following procedure:

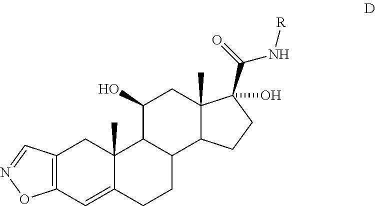

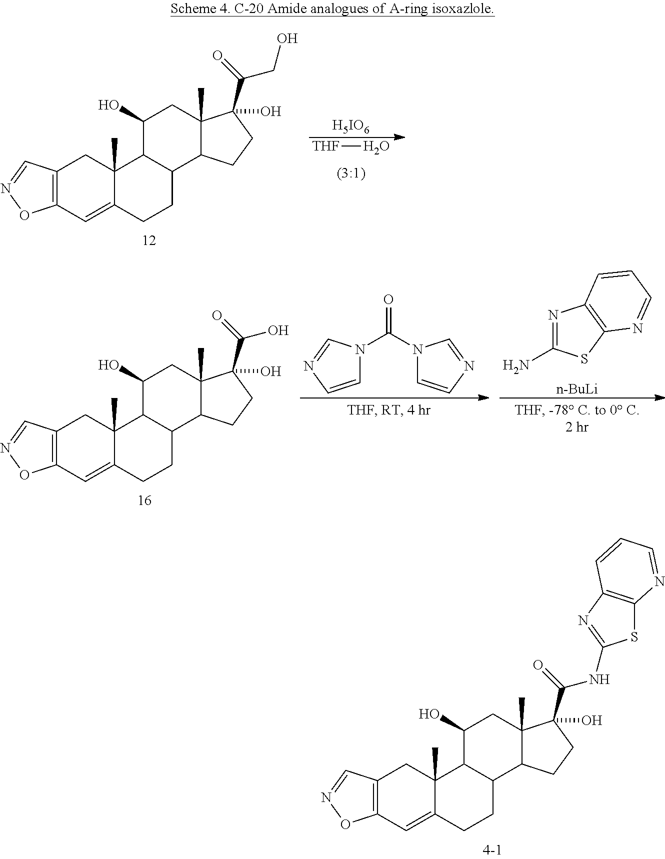

### Step 1)

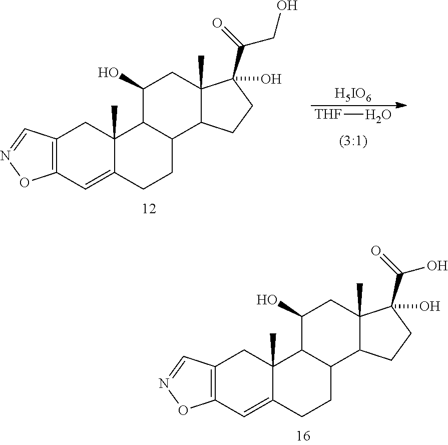

To a stirred solution of 12 (1.14 g, 2.94 mmol) in THF (15 mL) was added a solution of periodic acid (891 mg, 3.91 mmol) in H₂O (5 mL) at room temperature. The reaction mixture was stirred at room temperature for overnight. The solvent (THF) was evaporated and the crude product was redissolved in dichloromethane (100 mL). The aqueous solution was extracted with dichloromethane (100 mL×3). The combined organic layer was washed with H₂O and brine solution, dried over MgSO₄, filtered and concentrated by rotary evaporator. The crude product was purified by column chromatography eluting with 10% MeOH in dichloromethane to give compound 16 (0.91 g, 83%)

### Step 2)

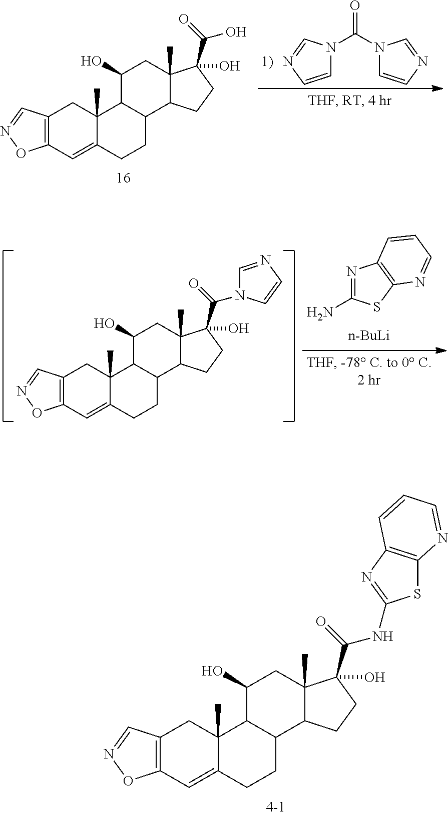

To a stirred solution of 16 (240 mg, 0.64 mmol) in anhydrous THF (5 mL) was added 1,1′-carbonyldiimidazole (CDI, 125 mg, 0.77 mmol) at room temperature. The reaction mixture was stirred at room temperature for 4 hrs. The solution was used in situ for the next step. To a stirred suspension of 2-aminothiazolo[5,4-b]pyridine (388 mg, 2.56 mmol) in anhydrous THF (5 mL) was added dropwise n-BuLi (1.6 M in hexanes, 1.6 mL, 2.56 mmol) at −78° C. and the reaction mixture was stirred at to −78° C. for 1.5 hrs. A solution in THF which is freshly generated above was added slowly into the reaction mixture at −78° C. and the reaction mixture was warmed to 0° C. for 2 hrs. The reaction mixture was acidified with 1N HCl. The aqueous layer was extracted with dichloromethane (50 mL×2) and the combined organic layer was washed with 1N HCl, NaHCO₃, H₂O and brine solution, dried over MgSO₄, filtered and concentrated by rotary evaporator. The crude product was purified by column chromatography eluting with ethyl acetate/hexanes (1/1) to give 4-1 (0.167 g, 52%, [M+1]=507.3)

IL-8 inhibition(TR) IC₅₀/Eₘₐₓ(nM, % inhibition): 23%

H Hela-GRE luciferase(TA) IC₅₀/Eₘₐₓ(nM, % inhibition): 0%

Generally, the compounds of Formula E can be prepared by the following procedure:

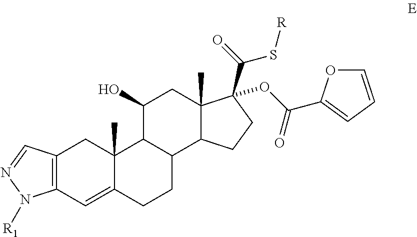

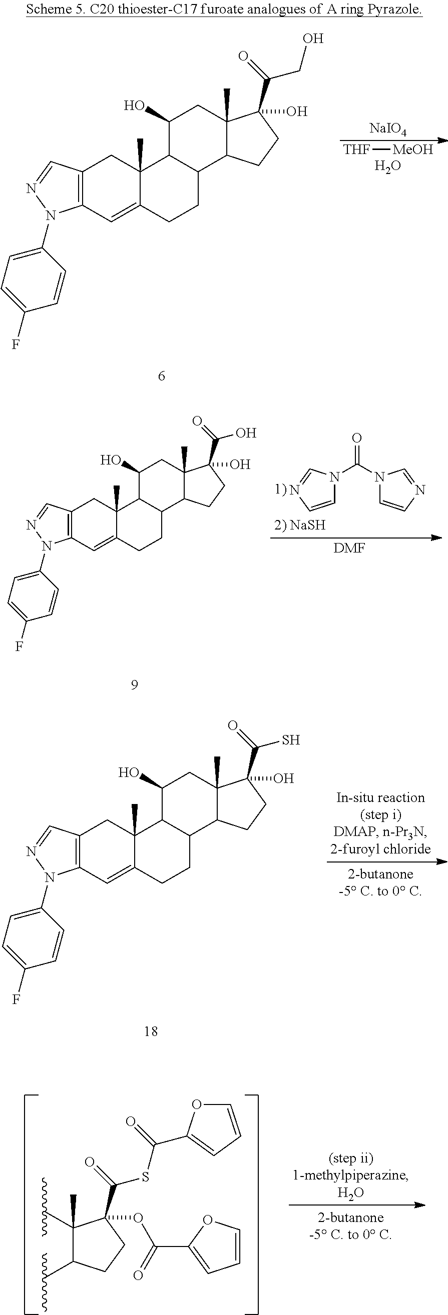

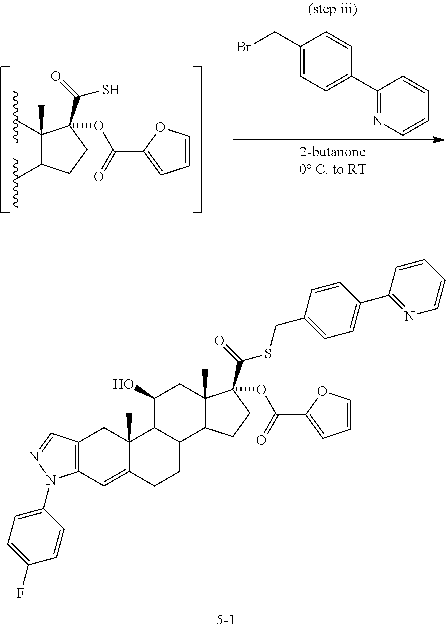

### Step 1)

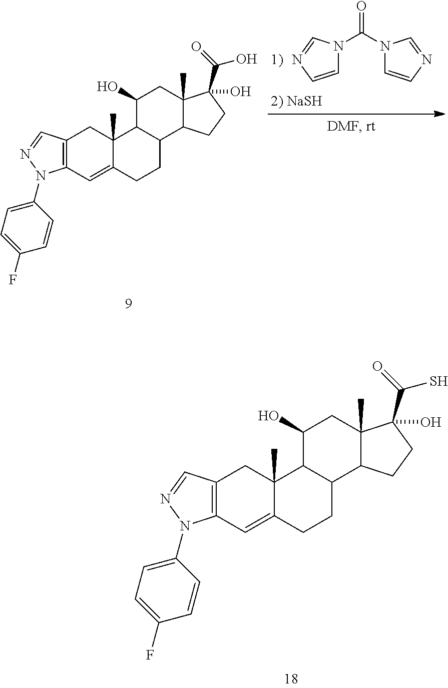

To a stirred solution of 9 (472 mg, 1.01 mmol) in DMF (1.6 mL) was added carbodiimidazole (CDI, 328 mg, 2.02 mmol) at room temperature and the reaction mixture was stirred at room temperature for 4 hr. Anhydrous NaSH (226 mg, 4.04 mmol) was then added and the reaction mixture was stirred at room temperature for 16 hr. The reaction mixture was poured into a mixture of 2M HCl (aq) and ice. The resulting precipitate was filtered, washed with cold water, dried in vacuum oven to give 476 mg (98%) of 18.

### Step 2)

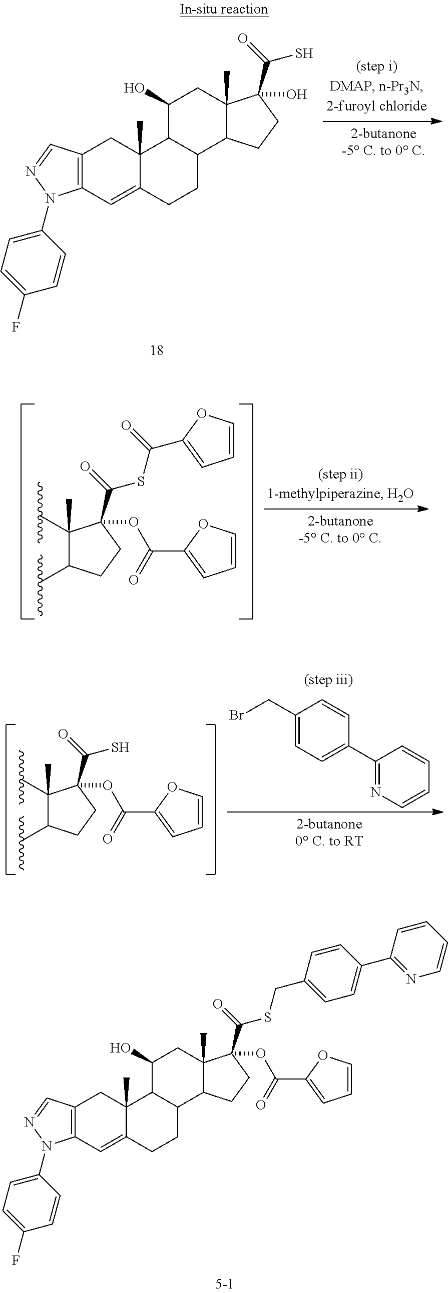

Step 1: To a stirred solution of 18 (48.8 mg, 0.10 mmol) in 2-butanone (505 μL) was added 4-dimethylaminopyridine (DMAP, 1.2 mg, 0.01 mmol) at room temperature. After 10 min, tripropylamine (59.2 μL, 0.30 mmol) was added and the resulting solution cooled to −5° C. Neat 2-furoylchloride (14.7 μL, 0.15 mmol) was added dropwise and the reaction mixture was stirred for 15 min at −5° C.-0° C.

Step 2: A solution of N-methylpiperazine (6.6 μL, 0.06 mmol) in H₂O (500 μL) was added dripwise to the reaction mixture at −5° C.-0° C. The reaction mixture was stirred for 10 min at −5° C.-0° C.

Step 3: A solution of 2-(4-(bromomethyl)phenyl)pyridine (29.7 mg, 0.12 mmol) in 2-butanone (500 μL) was added at 0° C. The solution mixture was warmed to room temperature and stirred for 5 hr at room temperature. The reaction mixture was diluted with ethylacetate and the organic layer was washed with aq. 1N HCl, H₂O, sat. NaHCO₃, H₂O and brine solution, dried over MgSO₄, filtered and concentrated by rotary evaporator. The crude product was purified by column chromatography (1/1 EtOAc/Hexanes) to give 38 mg (51% for in-situ 3 step) of 5-1.

Using procedures described above for compound 5-1, compounds 1 through 4 were prepared as disclosed in Table 4.

TABLE 4

| | | | IL-6 inhibition |
|---|---|---|---|
| Compound | | | (TR) IC₅₀/Emax |
| # | Structure | M + 1 | (nM, % inhibition) |
| 1 | 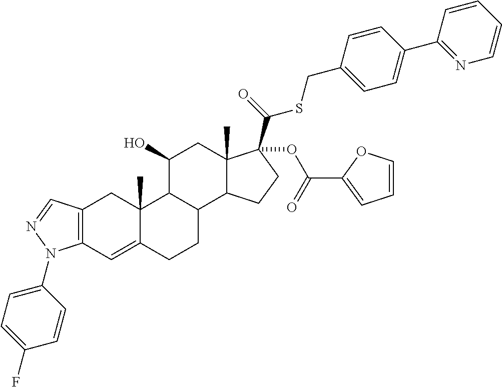 | 744.4 | A/A |
| 2 | 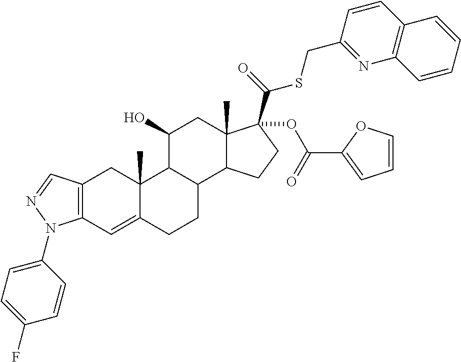 | 718.4 | A/A |
| 3 | 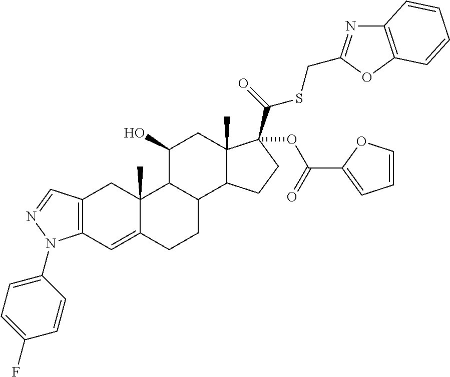 | 708.4 | A/A |
| 4 | 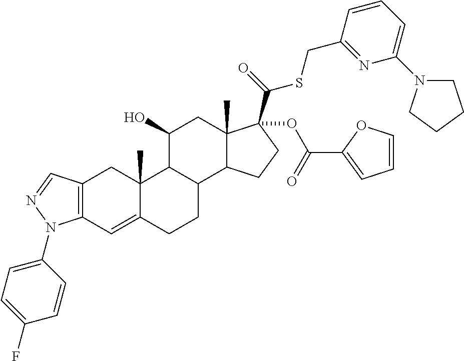 | 737.4 | A/A |

Generally, the compounds of Formula F can be prepared by the following procedure:

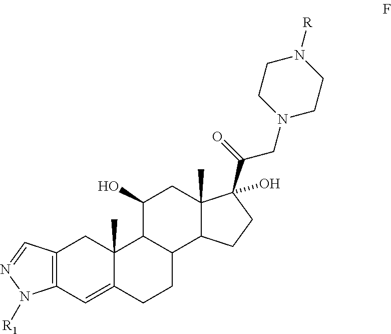

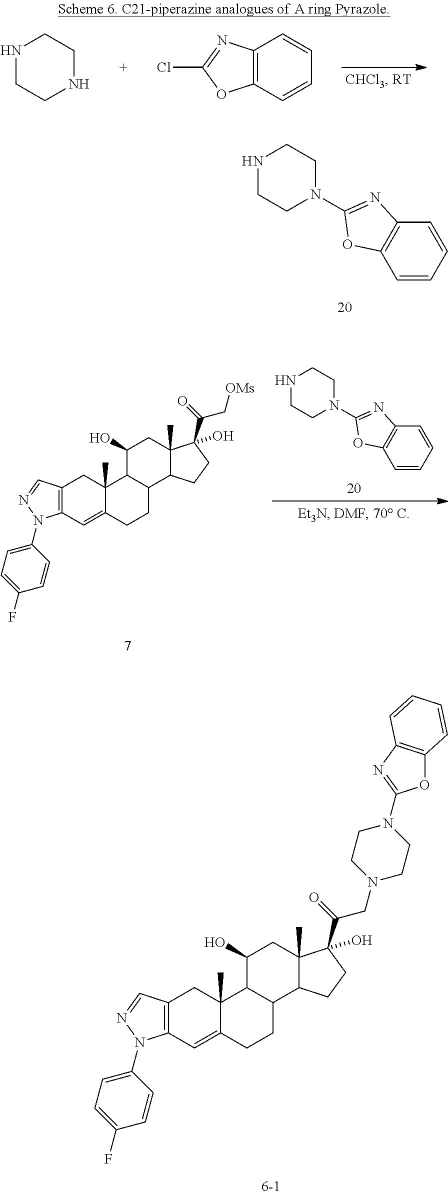

### Step 1)

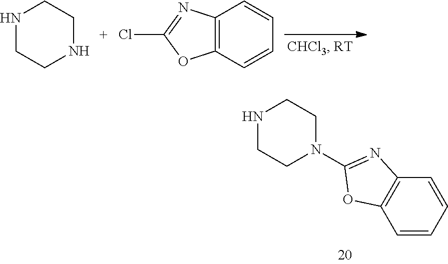

To a stirred solution of piperazine (560.9 mg, 6.50 mmol) in CHCl₃ (16 mL) was added 2-chlorobenzoxazole (500 mg, 3.25 mmol) at room temperature. The reaction mixture was stirred at room temperature for 16 hr. CHCl₃ was removed and the resulting white solid was dissolved in water. After stirring in water for 30 min, the aqueous layer was extracted with CH₂Cl₂. The combined organic layer was dried over MgSO₄, filtered, and concentrated. The white solid (20, 439 mg, 67%) was used for the next step without purification.

### Step 2)

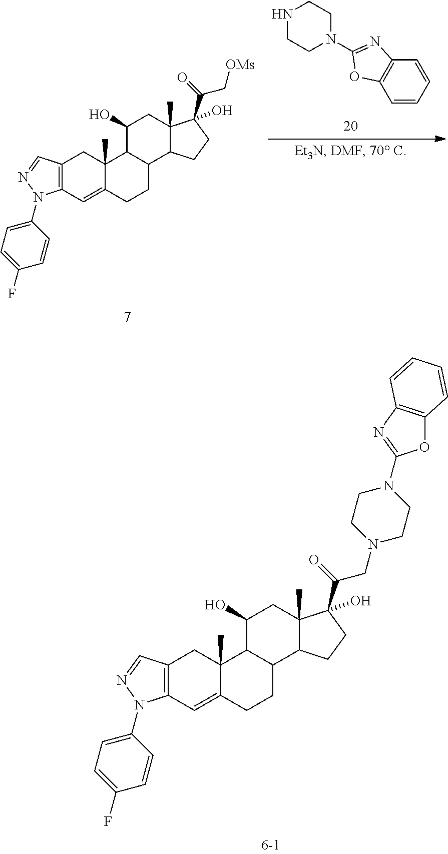

To as stirred solution of 7 (200 mg, 0.358 mmol) in DMF (3.60 mL) was added piperazine 20 (182 mg, 0.511 mmol) and Et₃N (160. 7 μL, 1.15 mmol) at room temperature. The reaction mixture was heated to 70-80° C. for 16 hr. After cooling down to room temperature, the reaction mixture was poured into sat. NaHCO₃ (aq) solution and extracted with EtOAc. The organic layer was washed with H₂O and brine, dried over MgSO₄, filtered and concentrated. The resulting crude product was purified by flash column chromatography on silica gel eluting with 10% MeOH in CH₂Cl₂ to afford the desired product, 6-1 (98 mg, 41%).

Using procedures described above for compound 6-1, compounds 1 through 5 were prepared as disclosed in Table 5.

TABLE 5

| | | | IL-8 inhibition |
|---|---|---|---|
| Compound | | | (TR) IC50/Emax |
| # | Structure | M + 1 | (nM, % inhibition) |
| 1 | 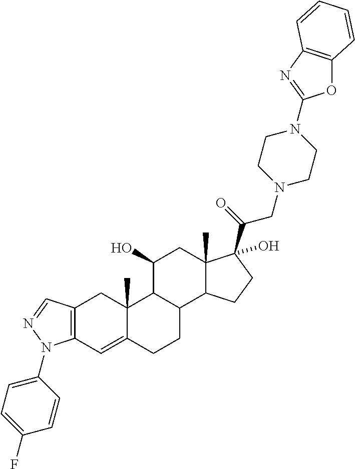 | 666.4 | A/A |
| 2 | 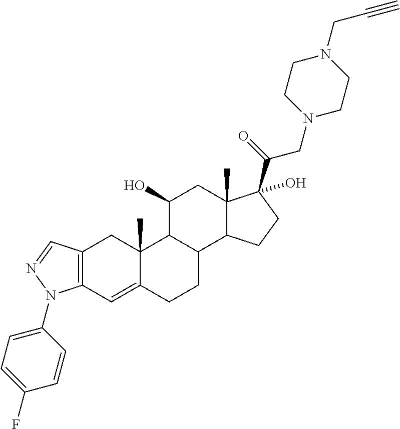 | 587.3 | **/D |
| 3 | 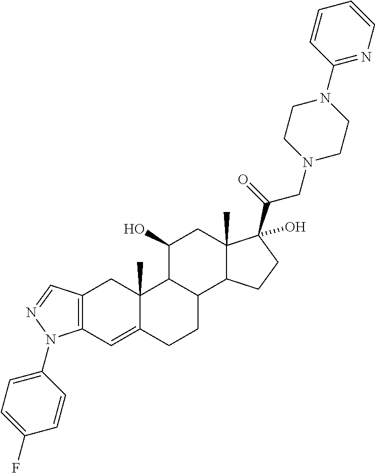 | 626.3 | A/A |
| 4 | 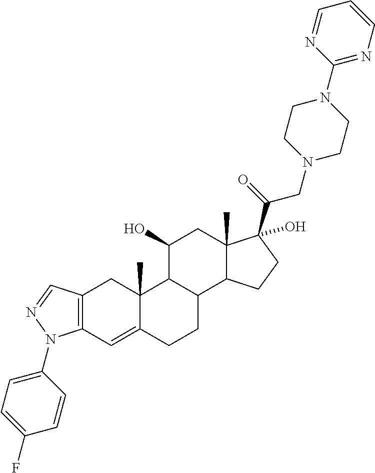 | 627.3 | A/A |
| 5 | 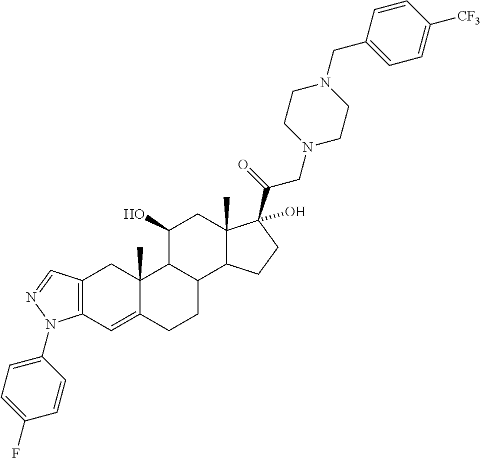 | 707.3 | A/A |

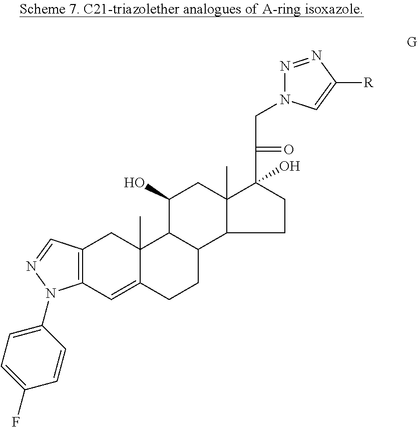

### Step 1

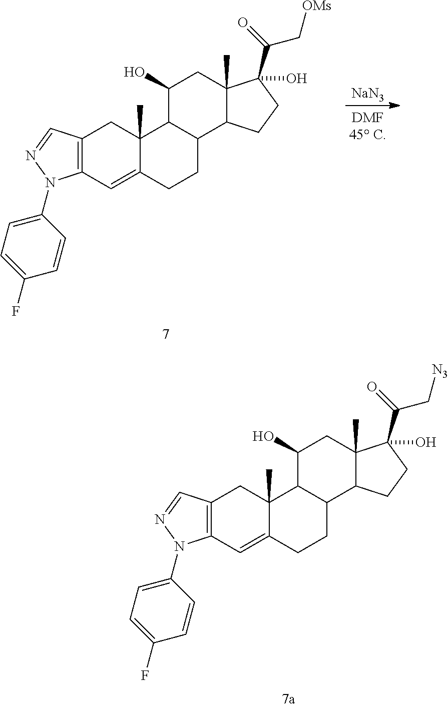

Mesylate 7 was prepared as described above for Scheme 1. To a stirred solution of 7 (1.25 g, 2.23 mmol) in DMF (27 mL) was added sodium azide (290 mg, 4.46 mmol) at room temperature. The reaction mixture was heated to 45° C. After 3 hr, TLC indicated the full consumption of the reactants. The reaction was cooled to RT and poured into cold NaHCO₃ (aq) solution, and the aqueous layer was extracted with CH₂Cl₂ (100 mL×3). The combined organic layer was washed with H₂O, dried over MgSO₄, filtered, and concentrated. The resulting crude residue was diluted with cold water. The pale yellowish solid precipitated, was filtered and washed with water, and dried in vacuum oven to give 998 mg of azide 7a.

### Step 2

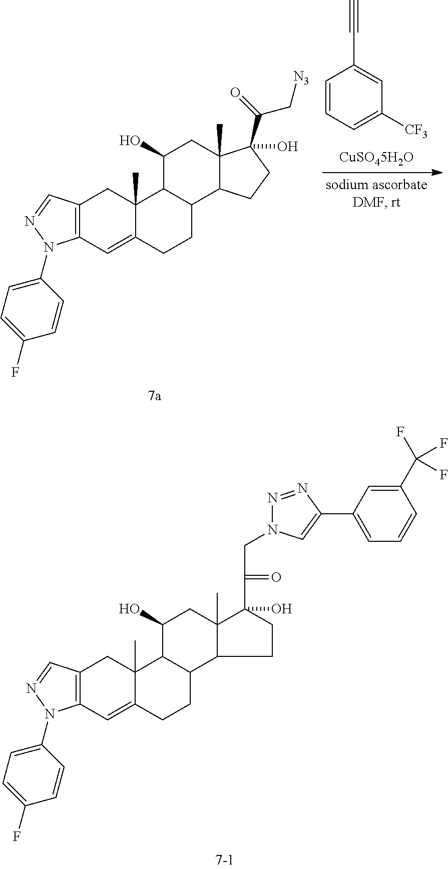

To a stirred solution of 7a (104.5 mg, 0.206 mmol) in DMF (1 mL) was added alkyne (19.6 μL, 0.206 mmol) at room temperature. 1M Sodium ascorbate solution in water (41.2 μL, 0.0412 mmol) was added, followed by the addition of 1M CuSO₄-5H₂O solution (20.6 4 μL, 0.0206 mmol). The heterogeneous mixture was stirred vigorously at room temperature overnight, at which point it cleared, and TLC analysis indicated reaction completion. The resulting reaction mixture was diluted with H₂O, treated with two drops of 30% H₂O₂ solution (aq) and 1 mL of saturated EDTA (aq). The aqueous layer was extracted with CH₂Cl₂ (50 mL×2). The combined organic layer was washed with H₂O, dried over MgSO₄, filtered, and concentrated. The resulting crude product was purified by column chromatography to afford 67 mg (48%) of the title compound 7-1. MH⁺ 676

Using procedures described above for 7-1 the compounds 1 through 3 were prepared as shown in Table 6:

TABLE 6

| | | | IL-8 inhibition |
|---|---|---|---|
| Compound | | | (TR) IC50/Emax |
| # | Structure | M + 1 | (nM, % inhibition) |
| 1 | 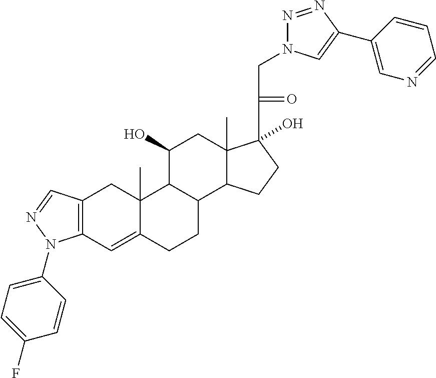 | 609 | A/A |
| 2 | 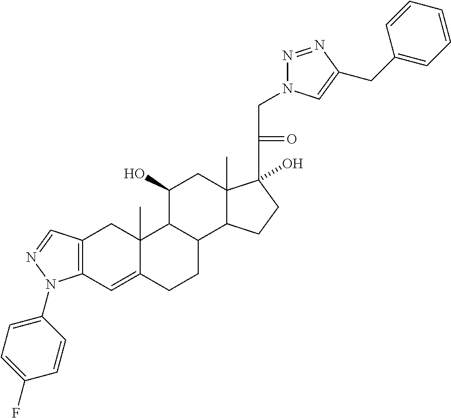 | 622 | A/A |
| 3 | 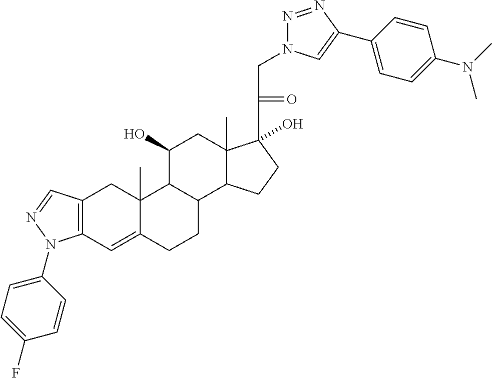 | 651 | |

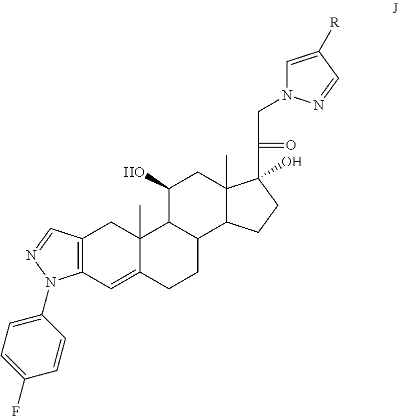

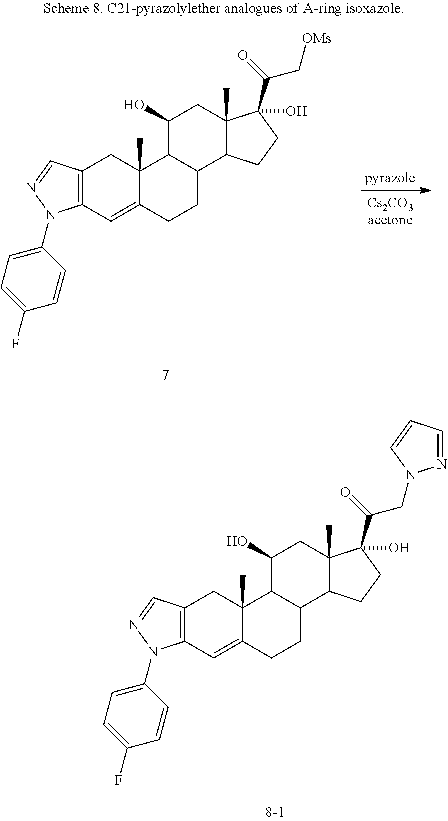

To a solution of starting mesylate 7 (50 mg, 0.089 mmol) in acetone (2 mL) was added pyrazole (11 mg, 0.134 mmol) and cesium carbonate (52 mg, 0.134 mmol). The reaction mixture was stirred at 50° C. for 12 h. The reaction mixture was cooled to room temperature and filtered through a pad of celite, washing with MeOH and CH₂Cl₂. The resulting filtrate was concentrated in vacuo. The residue was purified by prep TLC (50% EtOAc/Hexanes) to give the title compound, 8-1 (13 mg, 28%). MH⁺ 531

Using procedures described above for 8-1 the compounds 1 through 4 were prepared as disclosed in Table 7.

TABLE 7

| | | | IL-8 inhibition |
|---|---|---|---|
| Compound | | | (TR) IC50/Emax |
| # | Structure | M + 1 | (nM, % inhibition) |
| 1 | 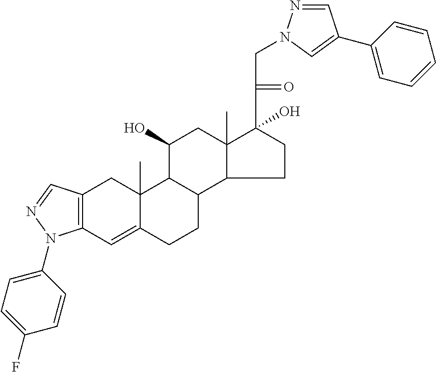 | 607 | A/A |
| 2 | 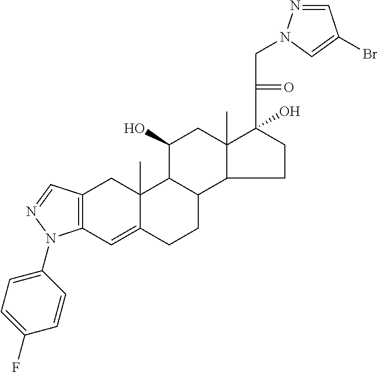 | 609 | A/A |
| 3 | 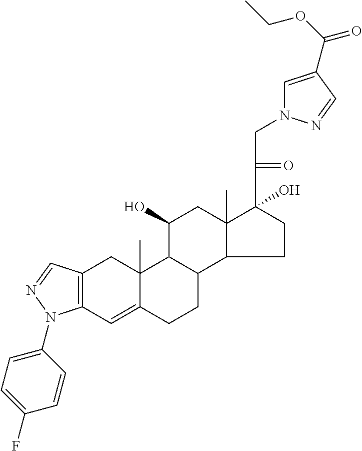 | 603 | A/A |
| 4 | 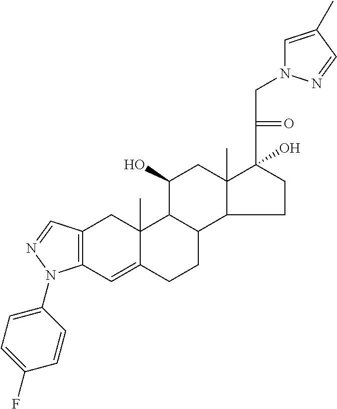 | 545 | A/A |

### Assays

### Glucocorticoid Receptor Binding Assay

Glucocorticoid receptor competitor assay kits can be obtained under license from Invitrogen (product #P2893) and the protocol followed. The assay is a competition binding assay, used to measure the affinity of test compound for the human glucocorticoid receptor. Affinity is measured based on the ability of test compounds to displace a fluorescent glucocorticoid. The presence of effective competitors prevents the formation of a fluorescent-labeled glucocorticoid to bind to the glucocorticoid receptor complex, resulting in a decrease of the polarization value. The shift in polarization value in the presence of test compounds is used to determine the relative affinity of test compounds for the glucocorticoid receptor.

### Glucocorticoid Transrepression Assay

Human Lung epithelial cell line NCl-H292 cells were dissociated from stock flask using 0.05% trypsin/0.53 mM EDTA. Cells were suspended in complete medium and counted. Cells were plated in 96-well flat-bottom plates at 20K cells /well in 0.2 ml/well. Plates were incubated for 24-48 hours until cells were between 75-90% confluent. Medium was aspirated and replaced with medium containing various concentrations of steroids or antagonists. After 1 hour incubation at 37°, TNFα (10 ng/ml final concentration in 0.2 ml) was added and the cells incubated overnight. Control wells with and without TNF were included on each plate, as well as wells with TNF in addition to a maximum (10 μM) concentration of dexamethasone. The cell culture medium was sampled and IL-6 and IL-8 cytokine production was measured using the MSD Multi-Spot immunoassay.

Exemplary compounds of the invention that were tested in the assay exhibited IC₅₀ values according to the following ranges:

| | |
|---|---|
| A | about 0.06 nM to about 20 nM |
| B | about 21 nM to about 65 nM |
| C | about 66 nM to about 400 nM |
| D | about 401 nM to about 1000 nM |
| E | > about 1000 nM |

Compounds of the invention that were tested in this assay exhibited a E_{MAX}% inhibition according to the following ranges:

| | |
|---|---|
| A | > about 80% |
| B | about 70% to about 80% |
| C | about 60% to about 70% |
| D | < about 60% |

The above values are reported in Tables 1-5 above as IC₅₀ values/E_{MAX}% inhibition. ** indicates value not measured.

### GRE-Transactivation Assay

HeLa cells were stably transfected with a human glucocorticoid response element coupled with a luciferase reporter gene.

Cells were plated in 96 well Packard View Plates (black sides/ clear bottom) at 20 K cells/0.2 ml complete medium. Plates were incubated overnight at 37°/5% CO₂. Medium was aspirated and replaced with 150 μl medium containing 5% charcoal-treated FBS and cells incubated overnight again. Test compounds were prepared in 5% charcoal-treated FBS medium. Medium was aspirated from plates and replaced with 100 μl of test compounds or controls. Plates were returned to incubator for exactly 24 hours. To measure induced luciferase, 100 μl of Steady-Glo luciferase assay substrate (Promega) was added to each well. Plates were sealed and mixed on a plate shaker for 5 minutes. Plate bottom opaque seals were added and the plates were allowed to stand for 60 minutes. Luminescence was measured on a Top-Count instrument (Perkin-Elmer).

All of the compounds tested in this assay exhibited E_{MAX} values of from 0% to 100% or above. Some of the compounds that were tested exhibited E_{MAX} values of from 0% to about 30%. Some of the compounds that were tested exhibited E_{MAX} values of from 30% to about 70%. Some of the compounds that were tested exhibited E_{MAX} values of greater than about 70%. For example, compound 60 (Table 1) exhibited E_{MAX} value of 15%, compound 74 (Table 1) exhibited E_{MAX} value of 56%, compound 1 (Table 7) exhibited E_{MAX} value of 69%, compound 1 (Table 1) exhibited E_{MAX} value of 71%, compound 71 (Table 1) exhibited E_{MAX} value of 85%, and compound 131 (Table 1) exhibited E_{MAX} value of 83%. Nondissociated glucocorticoids dexamethasone and fluticasone propionate exhibited E_{MAX} values in this assay of 100% and 99% respectively.

### Compositions and Methods

The compounds of the invention are beneficial, inter alia, their ability to bind glucocorticoid receptor and to illicit a response via that receptor. Hence, the compounds of the invention are useful wherever glucocorticoid agonists are useful. Such uses include, but are not limited to, the treatment of any diseases, conditions, or disorders for which steroids (or other glucocorticoid agonists) are believed useful, including a wide range of immune, autoimmune, and/or inflammatory diseases and conditions. Ex vivo use, e.g., as test instruments, is also contemplated. In some embodiments, the compounds of the invention possess the advantage of having little or no systemic activity. Therefore, in some embodiments, the compounds of the invention may be safer than those known glucocorticoids which have poor side effect profiles.

Non-limiting examples of inflammatory, immune, autoimmune and other diseases or conditions in which the compounds of the invention are useful include skin diseases such as eczema, posriasis, allergic dermatitis, atopic dermatitis, neurodermatitis, pruritis, and hypersensitivity reactions; inflammatory conditions of the nose, throat, or lungs such as asthma (including allergen-induced asthmatic reactions), rhinitis (including hayfever), allergic rhinitis, rhinosinusitis, sinusitis, nasal polyps, chronic bronchitis, chronic obstructive pulmonary disease, interstitial lung disease, and fibrosis; inflammatory bowel conditions such as ulcerative colitis and Chron's disease; and autoimmune diseases such as rheumatoid arthritis. Treatment of inflammation associated with CNS or peripheral nervous system disorders is also contemplated. Non-limiting examples include CNS trauma (e.g., brain trauma). Treatment of multiple sclerosis is also contemplated. Compounds of the invention may also be useful in treatment or prophylaxis of diseases and conditions of the eye, non-limiting examples of which include treatment of conjunctiva and allergic and nonallergic conjunctivitis.

Those skilled in the art will appreciate that, in some embodiments, the compounds and compositions of the invention are useful for both treatment and prophylaxis conditions and/or symptoms thereof described herein.

In another embodiment, the present invention provides for the use (and/or preparation) of a compound of the invention, or a pharmaceutically acceptable salt, solvate, ester, prodrug, tautomer, or isomer thereof, or the manufacture of a medicament for the treatment or prophylaxis of patients for the various diseases, conditions, and/or disorders described herein, including immune, autoimmune, and/or inflammatory diseases and/or conditions.

In another embodiment, the compounds of the invention may be used in acute treatment a wide range of immune, autoimmune, and inflammatory diseases and conditions, such as those listed above. In some embodiments, the compounds of the invention exhibit diminished side effect profiles in respect of one or more side effects associated with standard long-term steroidal treatments. Side effects associated with standard steroidal treatments include, for example, interference with carbohydrate metabolism, calcium resorption, suppression of endogenous corticosteroids, and suppression of the pituitary gland, adrenal cortex, and thymus. In such embodiments, compounds of the invention are useful for long-term treatment (as well as short- and medium-term treatment) of a wide range of chronic immune, autoimmune, and inflammatory diseases and conditions.

In another embodiment, the present invention provides a method for the treatment of neonatal sepsis, ALS, multiple sclerosis, type I diabetes, viral induced infections of the upper and lower airways, viral meningitis, and life-threatening diseases such as chronic meningeoencephalitis, neonatal enteroviral disease, polio, and myocarditis. The compounds and compositions of the present invention may also be used prophylactically to prevent exacerbations of symptoms associated with such diseases.

In another embodiment, the present invention provides a method for the treatment of viral related disorders. In one embodiment, the viral disorder is associated with the common cold. Compounds and compositions of the present invention may be utilized also in preventing exacerbation of disorders of the upper and lower airways. With respect to upper airway disorders, for example, the congestion and nasal blockage associated with allergic rhinitis, sinusitis, fungal induced sinusitis, bacterial based sinusitis, polyposis and the like. Examples with regard to disorders of the lower airways include administration of compositions of the present invention to prevent the need for the use of rescue medications for disorders of the lower airways, for example, asthma, chronic obstructive pulmonary disorder, allergic asthma, and emphysema. The compounds and compositions of the present invention may be useful also for the treatment and prevention of the nasal (stuffiness/congestion, rhinorrhea, nasal itching, sneezing) and non-nasal (itchy/burning eyes, tearing/watery eyes, redness of the eyes, itching of the ears/palate) symptoms of seasonal and perennial

In another embodiment, the present invention provides a method for the treatment of a patient with an immune, autoimmune, or an inflammatory disease or condition, which method comprises administering to a patient in need thereof an effective amount of a compound of the invention or a pharmaceutically acceptable salt, solvate, ester, prodrug, tautomers, or isomers thereof. The present invention also provides the use of a compound of the invention, (or a pharmaceutically acceptable salt, solvate, ester, prodrug, tautomers, or isomers thereof), for the treatment of patients with immune, autoimmune, and/or inflammatory diseases and conditions.

In another embodiment, the present invention provides a method for the treatment of corticosteroid-responsive diseases of the airway passage ways and lungs. Such diseases include those allergic, non-allergic and/or inflammatory diseases of the upper or lower airway passages or of the lungs which are treatable by administering corticosteroids. Typical corticosteroid-responsive diseases include allergic and non-allergic rhinitis, nasal polyps, chronic obstructive pulmonary disease (COPD), and non-malignant proliferative and inflammatory diseases of the airways passages and lungs.

In another embodiment, the present invention provides a method for the treatment of allergic and non-allergic rhinitis as well as non-malignant proliferative and/or inflammatory disease of the airway passages and lungs. Exemplary allergic or inflammatory conditions of the upper and lower airway passages which can be treated or relieved according to various embodiments of the present invention include nasal symptoms associated with allergic rhinitis, such as seasonal allergic rhinitis, intermittent allergic rhinitis, persistent allergic rhinitis and/or perennial allergic rhinitis as well as congestion in moderate to severe seasonal allergic rhinitis patients. Other conditions that may be treated or prevented include corticosteroid responsive diseases, nasal polyps, asthma, chronic obstructive pulmonary disease (COPD), rhinovirus, rhinosinusitis including acute rhinosinusitis and chronic rhinosinusitis, congestion, total nasal symptoms (stuffiness/congestion, rhinorrhea, nasal itching, sneezing) and non-nasal symptoms (itchy/burning eyes, tearing/watery eyes, redness of the eyes, itching of the ears/palate) and nasal blockage associated with sinusitis, fungal induced sinusitis, bacterial based sinusitis.

The term “allergic rhinitis” as used herein means any allergic reaction of the nasal mucosa and includes hay fever (seasonal allergic rhinitis) and perennial rhinitis (non-seasonal allergic rhinitis) which are characterized by seasonal or perennial sneezing, rhinorrhea, nasal congestion, pruritis and eye itching, redness and tearing.

The term “non-allergic rhinitis” as used herein means eosinophilic nonallergic rhinitis which is found in patients with negative skin tests and those who have numerous eosinophils in their nasal secretions.

The term “asthma” as used herein includes any asthmatic condition marked by recurrent attacks of paroxysmal dyspnea (i.e., “reversible obstructive airway passage disease”) with wheezing due to spasmodic contraction of the bronchi (so called “bronchospasm”). Asthmatic conditions which may be treated or even prevented in accordance with this invention include allergic asthma and bronchial allergy characterized by manifestations in sensitized persons provoked by a variety of factors including exercise, especially vigorous exercise (“exercise-induced bronchospasm”), irritant particles (pollen, dust, cotton, cat dander) as well as mild to moderate asthma, chronic asthma, severe chronic asthma, severe and unstable asthma, nocturnal asthma, and psychologic stresses. The invention is particularly useful in preventing the onset of asthma in mammals e.g., humans afflicted with reversible obstructive disease of the lower airway passages and lungs as well as exercise-induced bronchospasm.

The term “non-malignant prolifertive and/or inflammatory disease” as used herein in reference to the pulmonary system means one or more of (1) alveolitis, such as extrinsic allergic alveolitis, and drug toxicity such as caused by, e.g. cytotoxic and/or alkylating agents; (2) vasculitis such as Wegener's granulomatosis, allergic granulomatosis, pulmonary hemangiomatosis and idiopathic pulmonary fibrosis, chronic eosinophilic pneumonia, eosinophilic granuloma and sarcoidoses.

The compounds of the invention may be formulated for administration in any way known to those of skill in the art, and the invention therefore also provides within its scope pharmaceutical compositions comprising a compound of the invention (or a pharmaceutically acceptable salt, solvate, ester, prodrug, tautomers, or isomers thereof) together, if desirable, in admixture with one or more pharmaceutically acceptable diluents, excipients, and/or carriers. Further, in one embodiment, the present invention provides a process for the preparation of such pharmaceutical compositions comprising mixing the ingredients.

The compounds of the invention may, for example, be formulated for oral, buccal, sublingual, parenteral, local, or rectal administration. Local administration includes, but is not limited to, insufflation, inhalation, and dermal. Examples of various types of preparation for local administration include ointments, lotions, creams, gels, foams, preparations for delivery by transdermal patches, powders, sprays, aerosols, capsules, or cartridges for use in an inhaler or insufflator or drops (e.g., eye or nose drops), solutions or suspensions for nebulization, suppositories, pessaries, retention enemas, and chewable or suckable or fast dissolving tablets or pellets (e.g., for the treatment of aphthous ulcers) or liposome or microencapsulation preparations. Compositions for topical administration, e.g., to the lung, include dry powder compositions and spray compositions.

Dry powder compositions for topical delivery to the lung may, for example, be presented in capsules and cartridges for use in an inhaler or insufflator of, for example, gelatine. Formulations generally contain a powder mix for inhalation of a compound (or compounds) of the invention and a suitable powder base such as lactose or starch. Each capsule or cartridge may generally contain between 20 micrograms to 10 milligrams of a compound (or compounds) of the invention. Other amounts of such compounds are also included within the scope of the invention and may be readily determined by those of ordinary skill in the art, such as a pharmacist or attending physician. Alternatively, compounds of the invention may be administered without exicipients. Packaging of the formulation may be suitable for unit dose or multi-dose delivery. In the case of multi-dose delivery, the formulation can be pre-metered (e.g., as in Diskus, see GB 2242134 or Diskhaler, see GB2178965, 2129691, and 2169265) or metered in use (e.g., as in Turbuhaler, see EP69715). An example of a unit-dose device is Rotahaler (see GB2064336).

Spray compositions may, for example, be formulated as aqueous solutions or as suspensions or as aerosols delivered from pressurized packs, such as a metered dose inhaler, with the use of a suitable liquefied propellant. Aerosol compositions suitable for inhalation can be either a suspension or a solution and generally contain a compound of the invention and a suitable propellant such as a fluorocarbon or a hydrogen-containing chlorofluorocarbon or other suitable propellants or mixtures of any of the foregoing. The aerosol composition may optionally contain additional formulation excipients well known in the art such as surfactants, e.g., oleic acid or lecithin and cosolvents, e.g., ethanol. One example formulation is excipient free and consists essentially of (e.g., consists of) a compound of the invention (optionally together with another active ingredient) and a propellant selected from 1,1,1,2-tetrafuloroethane, 1,1,1,2,3,3,3-heptafuloro-n-propand and mixtures thereof. Another example formulation comprises particulate compound of the invention, a propellant selected from 1,1,1,2-tetrafluoroethane, 1,1,1,2,3,3,3-heptafluoro-n-propane, and mixtures thereof and a suspending agent which is soluble in the propellant, e.g., an oligolactic acid or derivative thereof, as described, for example, in WO94/21229. A preferred propellant is 1,1,1,2-tetrafluoroethane. Pressurized formulations will generally be retained in a canister (e.g., an aluminium canister) closed with a valve (e.g., a metering valve) and fitted into an actuator provided with a mouthpiece.

Medicaments for administration by inhalation are also contemplated. As will be appreciated by those of ordinary skill in the art, such medicaments desirably have controlled particle size. The optimum particle sizes for inhalation into the bronchial system are well known to those skilled in the art and typically range from 1-10 micrometers, preferably 2-5 micrometers. Particles having a size above 20 micrometers are generally not preferred for reaching small airways. To achieve these or other desired particle sizes the particles of a compound of the invention as produced may be reduced in size by conventional means, e.g., by microencapsulation. The desired fraction may be separated by any suitable means such as by air classification or by sieving. Preferably, the particles will be crystalline. Crystalline particles may be prepared for example by a process which comprises mixing in a continuous flow cell, in the presence of ultrasonic radiation, a flowing solution of a compound of the invention in a liquid solvent with a flowing liquid antisolvent for said compound (e.g., as described in PCT/GB99/04368). Alternatively, crystalline particles may be prepared by a process comprising admitting a stream of solution of the substance in a liquid solvent and a stream of liquid antisolvent for the substance tangentially into a cylindrical mixing chamber having an axial outlet port such that the streams are thereby intimately mixed through formulation of a vortex which causes precipitation of crystalline particles of the substance (e.g., as described in International Patent Application PCT/GB00/04327). When an excipient such as lactose is employed, generally, the particle size of the excipient will be much greater than the inhaled compound of the invention. When the excipient is lactose it will typically be present as milled lactose, wherein not more than about 85% of lactose particles will have a MMD of 60-90 micrometers and not less than about 15% will have a MMD of less than 15 micrometers.

Formulations for administration topically to the nose are also contemplated. Such formulations include pressurized arosol formulations and aqueous formulations administered to the nose by pressurized pump.

Aqueous formulations for administration to the lung or nose may be provided with conventional excipients such as buffering agents, tonicity modifying agents and the like. Aqueous formulations may also be administered to the nose by nebulisation or other means known in the art.

Other non-limiting examples of modes of administration include which are contemplated include: ointments, creams and gels, which may, for example, be formulated with an aqueous or oily base with the addition of suitable thickening and/or gelling agent and/or solvents. Such bases may, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions are also contemplated. Lotions may be formulated with an aqueous or oily base and will in general also contain one or more emulsifying agents, stabilizing agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilizing agents, suspending agents or preservatives.

If appropriate, the formulations of the invention may be buffered by the addition of suitable buffering agents.

The proportion of the active compound of the invention in compositions according to the invention depends on the precise type of formulation to be prepared but will generally be within the range of from 0.001 to 50% by weight. Generally, however for most types of preparations the proportion used will be within the range of from 0.005 to 1% and preferably 0.01 to 0.5%. However, in powders for inhalation or insufflation, the proportion used will usually be within the range of from 0.1 to 50%.

Aerosol formulations are contemplated. In some embodiments, aerosol formulations are preferably arranged so that each metered dose or “puff” of aerosol contains 1 micrograms to 2000 micrograms, eg 20 micrograms to 2000 micrograms, alternatively about 20 micrograms to about 1500 micrograms of a compound of the invention. Administration may be once daily or several times daily, for example 2, 3, 4 or 8 times, giving for example 1, 2 or 3 doses each time. Preferably the compound of the invention is delivered once or twice daily. The overall daily dose with an aerosol will typically be within the range 10 micrograms to 10 milligrams, eg 100 micrograms to 10 milligrams, alternatively, 200 micrograms to 2000 micrograms, alternatively about 1500 micrograms.

Topical preparations may be administered by one or more applications per day to the affected area; over skin areas occlusive dressings may advantageously be used. Continuous or prolonged delivery may be achieved, e.g., by an adhesive reservoir system.

For internal administration the compounds according to the invention may, for example, be formulated in conventional manner for oral, parenteral or rectal administration. Formulations for oral administration include syrups, elixirs, powders, granules, tablets and capsules which typically contain conventional excipients such as binding agents, fillers, lubricants, disintegrants, wetting agents, suspending agents, emulsifying agents, preservatives, buffer salts, flavouring, colouring and/or sweetening agents as appropriate. Dosage unit forms are, however, preferred as described herein.

Preferred forms of preparation for internal administration are dosage unit forms, i.e., tablets and capsules. Such dosage unit forms contain from 0.1 mg to 20 mg preferably from 2.5 to 10 mg of the compounds of the invention.

The compounds according to the invention may, in general, may be given by internal administration in cases where systemic adreno-cortical therapy is indicated.

In general terms, preparations for internal administration may contain from 0.05 to 10% of the active ingredient, depending upon the type of preparation involved. The daily dose may vary from 0.1 mg to 60 mg, e.g. 5-30 mg, dependent on the condition being treated, and the duration of treatment desired.

Slow release or enteric coated formulations may be advantageous, particularly for the treatment of inflammatory bowel disorders.

In some embodiments, administration may be accomplished utilizing inhalation devices. Non-limiting examples of such devices include, but are not limited to, nebulizers, metered pump-spray devices, soft mist inhalers, and pressurized metered dosing inhalers. A single pressurized metered dose inhaler may be adapted for oral or nasal inhalation routes simply by switching between an actuator that is designed for nasal delivery and an actuator designed for oral delivery.

Solutions may be administered intranasally by inserting an appropriate device (such as a nasal spray bottle and actuator used to deliver NASONEX® Nasal Spray) into each nostril. Active drug, which would include at least one compound of the invention, is then expelled from the nasal spray device. Efficacy can be generally assessed in a double blind fashion by a reduction in nasal and non-nasal symptoms (e.g., sneezing, itching, congestion, and discharge). Other objective measurements (e.g., nasal peak flow and resistance) can be used as supportive indices of efficacy. Any suitable pump spray may be used, such as pump sprays used for NASONEX® as sold by Schering-Plough or AFRIN® as sold by Schering-Plough.

Pressurized metered-dose inhalers (“MDI”) contain propellants, for example, chlorofluorocarbon propellants, for example, CFC-11, CFC-12, hydrofluorocarbon propellants, for example, HFC-134A, HFC-227 or combinations thereof, to produce a precise quantity of an aerosol of the medicament contained with the device, which is administered by inhaling the aerosol nasally, treating the nasal mucosa and/or the sinus cavities.

A suitable MDI formulation will include a propellant such as 1,1,1,2,3,3,3 heptafluoropropane; an excipient, including but not limited to alcohols, MIGLYOL® 812, MIGLYOL® 840, PEG-400, menthol, lauroglycol, VERTREL®_{—}245, TRANSCUTOL®, LABRAFAC® Hydro WL 1219, perfluorocyclobutane, eucalyptus oil, short chain fatty adds, and combinations thereof; a steroid and optionally a surfactant. MDI's may be prepared by conventional processes such as cold filling or pressure filling.

A “soft-mist” inhaler is a multi-dose, metered aerosol delivery device typically used to deliver aqueous based solution medicaments to the lungs via oral inhalation. The aerosol plume that they create is both slow in velocity and lasts for approximately 6×that of a typical pMDI (e.g. typically 1-2 sec. vs. milliseconds). An example of such a device would be Boehringer Ingelheim's (BI) RESPIMAT® which is currently used to deliver ipatropium bromide to the lungs.

In some embodiments, medicament formulations of the present invention may also be administered utilizing a nebulizer device. Typical commercial nebulizer devices produce dispersions of droplets in gas streams by one of two methods. Jet nebulizers use a compressed air supply to draw liquid up a tube and through an orifice by venturi action and introduce it into a flowing gas stream as droplets suspended therein, after which the fluid is caused to impact one or more stationary baffles to remove excessively large droplets. Ultrasonic nebulizers use an electrically driven transducer to subject a fluid to high-frequency oscillations, producing a cloud of droplets which can be entrained in a moving gas stream; these devices are less preferred for delivering suspensions. For instance, from about 2 to about 4 mL of the mometasone furoate solution may be placed in a plastic nebulizer container and the patient would inhale for 1-30 minutes. The total dosage placed in such a container may be determined by those skilled in the art. A non-limiting example would be in the range of 5 to about 100 mcg.

Also contemplated are hand-held nebulizers which atomize a liquid with a squeeze bulb air supply, but the more widely used equipment incorporates an electrically powered compressor or connects to a cylinder of compressed gas. Although the various devices which are commercially available vary considerably in their delivery efficiency for a given medicament since their respective outputs of respirable droplets are far from identical, any may be used for delivery of the medicaments of the present invention when a prescriber specifies an exact amount of medicament formulation which is to be charged to each particular device.

As noted herein, in some embodiments, the present invention provides compositions comprising at least one compound of the invention (optionally together with one or more additional active ingredients), formulated for nasal spray administration. Suitable nasal spray formulations can include, inter alia, water, auxiliaries and/or one or more of the excipients, such as: suspending agents, e.g., microcrystalline cellulose, sodium carboxymethylcellulose, hydroxpropyl-methyl cellulose; humectants, e.g. glycerin and propylene glycol; acids, bases or buffer substances for adjusting the pH, e.g., citric acid, sodium citrate, phosphoric acid, sodium phosphate as well as mixtures of citrate and phosphate buffers; surfactants, e.g. polysorbate 80; and antimicrobial preservatives, e.g., benzalkonium chloride, phenylethyl alcohol and potassium sorbate.

Depending on the intended application, it may be desirable to incorporate up to about 5 percent by weight, more typically about 0.5 to about 5 weight percent, of an additional rheology-modifying agent, such as a polymer or other material. Useful materials include, without limitation thereto, sodium carboxymethyl cellulose, algin, carageenans, carbomers, galactomannans, hydroxypropyl methylcellulose, hydroxypropyl cellulose, polyethylene glycols, polyvinyl alcohol, polyvinylpyrrolidone, sodium carboxymethyl chitin, sodium carboxymethyl dextran, sodium carboxymethyl starch and xanthan gum. Combinations of any two or more of the foregoing are also useful.

Mixtures of microcrystalline cellulose and an alkali metal carboxyalkylcellulose are commercially available, a non-limiting example of which includes one being sold by FMC Corporation, Philadelphia, Pa. U.S.A. as AVICEL® RC-591. This material contains approximately 89 weight percent microcrystalline cellulose and approximately 11 weight percent sodium carboxymethylcellulose, and is known for use as a suspending agent in preparing various pharmaceutical suspensions and emulsions. The compositions of the present invention may contain at least about 1.0 to about 10 weight percent, or from about 1 to about 4 weight percent of the mixture of the cellulose/carboxyalkylcellulose compound mixture.

A closely related mixture is available from the same source as AVICEL® RC-581, having the same bulk chemical composition as the RC-591, and this material is also useful in the invention. Microcrystalline cellulose and alkali metal carboxyalkylcellulose are commercially available separately, and can be mixed in desired proportions for use in the invention, with the amount of microcrystalline cellulose may be between about 85 and about 95 weight percent of the mixture for both separately mixed and co-processed mixtures.

When the compositions of the invention are intended for application to sensitive mucosal membranes, it may be desirable to adjust the pH to a relatively neutral value, using an acid or base, unless the natural pH already is suitable. In general, pH values about 3 to about 8 are preferred for tissue compatibility; the exact values chosen should also promote chemical and physical stability of the composition. In some instances, buffering agents will be included to assist with maintenance of selected pH values; typical buffers are well known in the art and include, without limitation thereto, phosphate, citrate and borate salt systems.

The compositions may contain any of a number of optional components, such as humectants, preservatives, antioxidants, chelating agents and aromatic substances. Humectants, which are hygroscopic materials such as glycerin, a polyethylene or other glycol, a polysaccharide and the like act to inhibit water loss from the composition and may add moisturizing qualities. Useful aromatic substances include camphor, menthol, eucalyptol and the like, flavors and fragrances. Preservatives are typically incorporated to establish and maintain a freedom from pathogenic organisms; representative components include benzyl alcohol, methylparaben, propylparaben, butylparaben, chlorobutanol, phenethyl alcohol (which also is a fragrance additive), phenyl mercuric acetate and benzalkonium chloride.

Pharmaceutical compositions comprising one (or more) compound(s) of the invention for use in combination with one or more other therapeutically active agent(s) are also contemplated. Non-limiting examples of such additional therapeutically active agents include, for example, beta₂ adrenoreceptor agonists, anti-histamines, anti-allergic agents, anticholinergic agents, and chemokine receptor antagonists. Additional agents are also described below. Such combinations may be administered simultaneously or sequentially (with a compound of the invention being administered either before or after the other active ingredient(s)) in separate or combined pharmaceutical formulations. For simultaneous administration, the invention thus provides, in another embodiment, pharmaceutical compositions comprising a compound of the invention (or a physiologically acceptable salt, solvate, prodrug, ester, tautomer, or isomer thereof) together with one or more other therapeutically active agent, for example, a beta₂ adrenoreceptor agonist, an antihistamine or an anti-allergic agent. The selection of the additional active agents is made on the basis of the intended use.

Compositions comprising long-acting beta₂ adrenoreceptor agonists (sometimes referred to as LABAs) are contemplated as being within the scope of the invention. Use of LABAs capable of providing a therapeutic effect over 24 hours is also contemplated. In another non-limiting embodiment, the present invention provides pharmaceutical compositions suitable for once-per-day administration comprising a compound of the invention (or a salt, solvate, ester, prodrug, tautomer, or isomer thereof) in combination with a long acting beta₂ adrenoreceptor agonist.

Non-limiting examples of beta₂-adrenoreceptor agonists include salmeterol (eg as racemate or a single enantiomer such as the R-enantiomer), salbutamol, formoterol, salmefamol, fenoterol, indacaterol, or terbutaline and salts thereof, for example the xinafoate salt of salmeterol, the sulphate salt or free base of salbutamol or the fumarate salt of formoterol. Long acting beta₂ adrenoreceptor agonists, such as salmeterol or fomoterol or indacaterol, are preferred. Preferred long acting beta₂-adrenoreceptor agonists include those described in WO 266422A.

Additional active agents include antihistamines. Non-limiting examples of anti-histamines useful in combination with the compounds of the present invention include methapyrilene, loratadine, acrivastine, astemizole, cetirizine, mizolastine, fexofenadine, azelastine, levocabastine, olopatadine, levocetirizine, and desloratadine.

Additional active agents include histamine H₁ receptor antagonists. Examples of Histamine H₁ receptor antagonists (herein also antihistamines) include, but are not limited to, Astemizole, Azatadine, Azelastine, Acrivastine, Bromphemiramine, Chlorpheniramine, Clemastine, Cyclizine, Carebastine, Cyproheptadine, Carbinoxamine, Desloratadine, Doxylamine, Diphenhydramine, Cetirizine, Dimenhydrinate, Dimethindene, Ebastine, Epinastine, Efletirizine, Fexofenadine, Hydroxyzine, Ketotifen, Loratadine, Levocabastine, Levocetirizine, Mizolastine, Mequitazine, Mianserine, Noberastine, Meclizine, Norastemizole, Picumast, Pyrilamine, Promethazine, Terfenadine, Tripelennamine, Temelastine, Trimeprazine, Triprolidine and mixtures of any two or more of the foregoing. Preferred Histamine H₁ receptors are desloratadine, loratadine, fexofenadine and ceterazine.

Desloratadine is also termed Descarboethoxyloratidine and DCL. DCL is a non-sedating antihistamine, whose technical name is 8-chloro-6,11-dihydro-11-(4-piperidylidene)-5H-benzo[5,6]cyclohepta[1,2]pyridine. This compound is described in Quercia, et al., Hosp. Formul., 28: 137-53 (1993), in U.S. Pat. No. 4,659,716, and in WO 96/20708. The use of Desloratadine for the treatment of congestion is disclosed in U.S. Pat. No. 6,432,972. DCL is an antagonist of the H₁ histamine receptor protein. The H₁ receptors are those that mediate the response antagonized by conventional antihistamines. H₁ receptors are present, for example, in the ileum, the skin, and the bronchial smooth muscle of man and other mammals. The amount of DCL which can be employed in a unit (i.e. single) dosage form of the present compositions can range from about 2.5 to about 45 mg, also from about 2.5 to about 20 mg, also from about 5 to about 10 mg. Preferred dosage amounts include 2.5 mg, 5.0 mg, 10.0 mg and 20.0 mg.

Loratadine is a non-sedating antihistamine whose technical name is 11-(4-piperidylidene)-5H-benzo-[5, 6]-cyclohepta-[1,2-b]-pyridine. The compound is described in U.S. Pat. No. 4,282,233. Loratadine is a potent tricyclic and antihistaminic drug of slow release, with a selective antagonist of peripheric H₁ receptors activity.

Fexofenadine reportedly is a non-sedating antihistamine, whose technical name is 4-[1-hydroxy-4-(4-hydroxy-diphenylmethyl)-1-piperidinyl)butyl]-α,α-dimethyl-benzene acetic acid. Preferably the pharmaceutically acceptable salt is the hydrochloride, also known as fexofenadine hydrochloride. The amount of fexofenadine which can be employed in a unit dosage form of the present composition can range from about 40 to 200 mg, also from about 60 to about 180 milligrams, also about 120 milligrams.

Cetirizine hydrochloride reportedly is an H, receptor antagonist. The chemical name is (±)-[2-[4-[(4-chlorophenyl)phenylmethyl]-1- piperazinyl]ethoxy]acetic acid, dihydrochloride. Cetirizine hydrochloride is a racemic compound with an empirical formula of C₂₁H₂₅ClN₂O₃.2HCl. Cetirizine hydrochloride is a white, crystalline powder and is water soluble. Cetirizine hydrochloride is available from Pfizer Inc., New York, N.Y., under the trade name ZYRTEC®. The amount of Cetirizine which can be employed in a unit dosage form of the present composition can range from about 0 to 40 mg, also from about 5 to about 10 milligrams. The levo isomer of Cetirizine may also be combined with Pleconaril in the formulations of the present invention. Another form of Cetirizine for use in the present invention is Cetirizine dinitrate.

Additional active agents include expectorants. Examples of expectorants suitable for use are known in the art and include, but are not limited to, ambroxol, guaiafenesin, terpin hydrate, and potassium quaicolsulfonate. Ambroxol is a bromhexine metabolite, chemically identified as trans-4(2-amino-3,5-dibromobenzil, amine) ciclohexane hydrochloride, which has been widely used during more than two decades as an expectorant agent or stimulating pulmonary surfactant factor. The compound is described in U.S. Pat. No. 3,536,712. Guaiafenesin is an expectorant, whose technical name is 3-(2-methoxyphenoxy)-1,2-propanediol. The compound is described in U.S. Pat. No. 4,390,732. Terpin hydrate is an expectorant, whose technical name is 4-hydroxy-α,α,4-trimethylcyclohexane-methanol. Potassium guaicolsulfonate is an expectorant, whose technical name is 3-Hydroxy-4-methoxybenzenesulfonic acid mix with mono-potassium 4-hydroxy-3-methoxybenzenesulfonate.

Additional active agents include decongestants. Examples of suitable decongestants for use include both oral and nasal decongestants. Examples of nasal decongestants useful in the present invention include, without being limited to, the sympathomimetic amine nasal decongestants. Those currently approved for topical use in the United States include, without limitation, levmetamfetamine (also known as 1-desoxyephedrine), ephedrine, ephedrine hydrochloride, ephedrine sulfate, naphazoline hydrochloride, oxymetazoline and pharmaceutically acceptable salts thereof, oxymetazoline hydrochloride, phenylephrine hydrochloride, and propylhexedrine. Oral decongestants for use in the present invention include, without limitation, phenylpropanolamine, phenylephrine and pseudoephedrine as well as pharmaceutically acceptable salts thereof. Pseudoephedrine and its acid additional salts, e.g., those of HCl or H₂SO₄, are recognized by those skilled in the art as a sympathomimetic therapeutic agent that is safe and effective for treating nasal congestion. They are commonly administered orally concomitantly with an antihistamine for treatment of nasal congestion associated with allergic rhinitis. When used in the present invention as a nasal decongestant it is preferred to use pseudoephedrine in amounts of equivalent to about 120 mg pseudoephedrine sulfate dosed one to 4 times daily. However, lesser amounts of pseudoephedrine sulfate may be used.

Additional active agents include histamine H₃ receptor antagonists. Examples of Histamine H₃ receptor antagonists suitable for use in the present invention include, but are not limited to, thioperamide, impromidine, Burimamide, Clobenpropit, Impentamine, Mifetidine, S-sopromidine, R-sopromidine, 3-(imidazol-4-yl)-propylguanidine (SKF-91486), 3->(4-chlorophenyl)methyl-5->2-(1H-imidazol-4yl)ethyl 1,2,3-oxadiazole (GR-175737), 4-(1-cyclohexylpentanoyl-4-piperidyl) 1H-imidazole (GT-2016), 2-{>2->4(5)-imidazolylethylthio}-5-nitropyridine (UCL-1199) Clozapine, SCH497079 and SCH539858. Additional examples are disclosed and claimed in U.S. Pat. No. 6,720,328 and United States Patent Application Publication No. 20040097483A1, both assigned to Schering Corp., and both of which are hereby incorporated by reference. Other preferred compositions may further include both H₁ and H₃ receptors antagonists as is disclosed in U.S. Pat. No. 5,869,479, also assigned to Schering Corp., which is hereby incorporated by reference. Other compounds can readily be evaluated to determine activity at H₃ receptors by known methods, including the guinea pig brain membrane assay and the guinea pig neuronal ileum contraction assay, both of which are described in U.S. Pat. No. 5,352,707. Another useful assay utilizes rat brain membranes and is described by West et al., “Identification of Two H₃-Histamine Receptor Subtypes,” Molecular Pharmacology, Vol. 38, pages 610-613 (1990).

Additional active agents include anti-cholinergic agents. Examples of anti-cholinergic agents for use in the present invention include, but are not limited to, Tiotropium, Oxitropium, Ipratropium, Methantheline, Propantheline, Dicyclomine, Scopolamine, Methscopolamine, Telenzepine, Benztropine, QNX-hemioxalate, Hexahydro-sila-difenidol hydrochloride and Pirenzepine. In one embodiment, such compositions comprising at least one compound of the invention and at least one anti-cholinergic agent (and optionally other active agents) are administered either orally or nasally in amounts that are known to, or determined by, those of skill in the art.

Additional active agents include antibiotics. Non-limiting examples include macrolides, cephalosporin, and antibacterials. Specific examples of suitable antibiotics include, but are not limited to, Tetracycline, Chlortetracycline, Bacitracin, Neomycin, Polymyxin, Gramicidin, Oxytetracycline, Chloramphenicol, Florfenicol, Gentamycin, Erythromycin, Clarithromycin, Azithromycin, Tulathromycin, Cefuroxime, Ceftibuten, Ceftiofur, Cefadroxil, Amoxicillin, Peniccilins, Amoxicillin with clavulanic acid or an other suitable beta-lactamase inhibitor, Sulfonamides, Sulfacetamide, Sulfamethizole, Sulfisoxazole; Nitrofurazone, and Sodium propionate. The therapeutic amounts of compositions which may be administered are known to one of skill in the art.

Additional active agents include P2Y₂ receptor agonists. Non-limiting examples of P2Y₂ receptor agonists for use in the present invention include, but are not limited, to diquafosol tetrasodium. Diquafosol tetrasodium is a P2Y₂ receptor agonist that activates receptors on the ocular surface and inner lining of the eyelid to stimulate the release of water, salt, mucin and lipids—the key components of natural tears. Mucin is made in specialized cells and acts to lubricate surfaces. Lipids in the eye are oily substances that form the outer-most layer of the tear film and are responsible for the prevention of excess tear fluid evaporation. In preclinical testing, diquafosol reportedly increased the secretions of natural tear components. Diquafosol is available from Inspire. P2Y₂ receptor agonists are a class of compounds that are being developed for the treatment of a variety of conditions in which mucociliary clearance (MCC) is impaired, including chronic bronchitis and cystic fibrosis (CF). Other mucolytic agents may include N-Acetylcysteine and endogenous ligand compound UTP. These compositions may be administered by routes known to those of skill in the art, including orally and nasally.

Additional active agents include Leukotriene₄ antagonists and/or inhibitors. Non-limiting examples of Leukotriene₄ antagonists and/or inhibitors suitable for use in the present invention include, but are not limited to Zileuton, Docebenone, Piripost, ICI-D2318, MK-591, MK-886, sodium 1-(((R)-(3-(2-(6,7-difluoro-2-quinolinyl)ethynyl)phenyl)-3-(2-(2-hydroxy-2-propyl)phenyl)thio)methyl)cyclopropane-acetate (also referred to herein for convenience as “compound LAcetate”); 1-(((R)-(3-(2-(2,3-dichlorothieno[3,2-b]pyridin-5-yl)-(E)-ethenyl)phenyl)-3-(2-(1-hydroxy-1-methylethyl)phenyl)propyl)thio)-methyl)cyclopropaneacetic acid (also referred to herein for convenience as “compound LAcid”), Pranlukast, Zafirlukast, and Montelukast and the compound [24[2-(4-tert-butyl-2-thiazolyl)-5-benzofuranyl]oxymethyl]phenyl]acetic acid (also referred to herein for convenience as “compound FK011” or “FR150011”). Preferred are montelukast, pranlukast, zafirlukast, compounds “FK011”, “LAcetate”, and “LAcid”. Compositions containing these constituents may be administered either orally or nasally as set forth below in amounts that are known to one of skill in the art.

Additional active agents include leukotriene D₄ antagonists. Non-limiting examples of suitable leukotriene D₄ antagonists include montelukast, which is a Leukotriene D₄ antagonist capable of antagonizing the receptors for the cysteinyl leukotrienes. The technical name of Montelukast is [R-(E)]-1-[[[1-[3-[2-(7-chloro-2-quinolinyl)ethenyl]phenyl]-3-[2-(1-hydroxy-1-methylethyl)phenyl]propyl]thio]methyl]-cyclopropaneacetic acid. This compound is described in EP 480,717. A preferred pharmaceutically acceptable salt of Montelukast is the monosodium salt, also known as Montelukast sodium. The amount of Montelukast which can be employed in a unit dosage form of the present invention can range from about one to 100 milligrams, also from about 5 to about 20 milligrams, preferably about 10 milligrams.

Additional non-limiting examples of suitable leukotriene D4 antagonists include the compound 1-(((R)-(3-(2-(6,7-difluoro-2-quinolinyl)ethenyl)phenyl)-3-(2-(2-hydroxy-2-propyl)phenyl)thio)methylcyclopropaneacetic acid, described in WO 97/28797 and U.S. Pat. No. 5,270,324. A pharmaceutically acceptable salt of this compound is the sodium salt, also known as sodium 1-(((R)-(3-(2-(6,7-difluoro-2-quinolinyl)ethenyl)phenyl)-3-(2-(2-hydroxy-2-propyl) phenyl)thio)-methylcyclopropaneacetate.

Additional non-limiting examples of suitable leukotriene D4 antagonists include the compound 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)-thio)methyl)cyclopropaneacetic acid, described in WO 97/28797 and U.S. Pat. No. 5,472,964. A pharmaceutically acceptable salt of this compound is the sodium salt, also known as sodium 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)-thio)methyl)cyclopropaneacetate.

Additional non-limiting examples of suitable leukotriene D4 antagonists include the compound pranlukast, described in WO 97/28797 and EP 173,516. The technical name for this compound is N-[4-oxo-2-(1H-tetrazol-5-yl)-4H-1-benzopyran-8-yl]-p-(4-phenylbutoxy)benzamide. The amount of Pranlukast which can be employed in a unit dosage form can range from about 100 to about 700 mg, preferably from about 112 to about 675 mg; also from about 225 mg to about 450 mg; also from about 225 to about 300 mg.

Additional non-limiting examples of suitable leukotriene D4 antagonists include the compound, described in WO 97/28797 and EP 199,543. The technical name for this compound is cyclopentyl-3,2-methoxy-4-[(o-tolylsulfonyl)carbamoyl]benzyl]-1-methylindole-5-carbamate.

Additional non-limiting examples of suitable leukotriene D4 antagonists include the compound [2-[[2-(4-tert-butyl-2-thiazolyl)-5-benzofuranyl]oxymethyl]phenyl]acetic acid, described in U.S. Pat. No. 5,296,495 and Japanese Patent JP 08325265A. An alternative name for this compound is 2-[[[2-[4-(1,1-dimethylethyl)-2-thiazolyl]-5-benzofuranyl]oxy]methyl]-benzeneacetic acid. The code number for this compound is FK011 or FR150011.

Additional active agents include pharmaceutically acceptable zinc salts, including those water soluble salts reported to have beneficial effects against the common cold. Typically such preparations comprise an aqueous or saline solution with a concentration of ionic zinc below that which causes irritation to mucus membranes. Generally the ionic zinc in such solutions is present substantially as unchelated zinc and is in the form of free ionic solution. Zinc ionic solutions for use in the present invention will typically contain substantially unchelated zinc ions in a concentration of from about 0.004 to about 0.12% (w/vol). Preferably the substantially unchelated ionic zinc compound can comprise a mineral acid salt of zinc selected from the group consisting of zinc sulfate, zinc chloride, and zinc acetate. These compositions may be administered either orally or nasally in amounts that are known to, or readily determined by, those of skill in the art.

Additional active agents include SYK kinase analogs. SYK kinase analogs are a class of molecules which work by blocking SYK kinase. Compound R112, available from Rigel Pharmaceuticals, Inc. is an example of an SYK kinase analog. A recent study reportedly showed a greater than 20% relative improvement for R112 over placebo (an absolute difference of 9% over placebo) and up to 38% improvement for R112 from baseline measurements (prior to drug initiation) of symptoms associated with chronic nasal congestion (e.g. stuffy nose) over a placebo.

Additional active agents include 5-lipoxygenase inhibitors. As used herein, the term “5-lipoxygenase inhibitor” (also referred to as a “5-LO inhibitor”) includes any agent, or compound that inhibits, restrains, retards or otherwise interacts with the enzymatic action of 5-lipoxygenase. Examples of 5-lipoxygenase inhibitors include, but not limited to, zileuton, docebenone, piripost, and the like. As used herein, the associated term “5-lipoxygenase activating protein antagonist” or “FLAP antagonist” includes any agent or compound that inhibits, retrains, retards or otherwise interacts with the action or activity of 5-lipoxygenase activating protein, examples of which include, but not limited, “FLAP antagonists” MK-591 and MK-886.

Additional active agents include those known to relieve oropharyngeal discomfort, including, for example, sore throats, cold or canker sores, and painful gums. Such active agents include topical anesthetics such as phenol, hexylresorcinol, salicyl alcohol, benzyl alcohol, dyclonine, dibucaine, benzocaine, buticaine, cetylpyridinium chloride, diperidon, clove oil, menthol, camphor, eugenol and others. Medicaments of the invention intended for application to the skin may similarly include a therapeutic agent for relieving skin discomfort including, but not limited to, lidocaine, benzocaine, tetracaine, dibucaine, pramoxine, diphenhydramine, and benzyl alcohol.

Additional active agents useful in combination with compound(s) of the invention include salicylates, such as aspirin, NSAIDs (non-steroidal anti-inflammatory agents such as indomethacin, sulindac, mefenamic, meclofenamic, tolfenamic, tolmetin, ketorolac, dicofenac, ibuprofen, naproxen, fenoprofen, ketoprofen, flurbirofin, or oxaprozin), TNF inhibitors such as etanercept or infliximab, IL-1 receptor antagonists, cytotoxic or immunosuppressive drugs such as methotrexate, leflunomide, azathiorpine, or cyclosporine, a gold compound, hydroxychloroquine or sulfasalazine, penicillamine, darbufelone, and p38 kinase inhibitors, sodium cromoglycate, nedocromil sodium, PDE₄ inhibitors, leukotriene antagonists, iNOS inhibitors, tryptase and elastase inhibitors, beta-2 integrin antagonists, adenosine 2a agonists; antiinfective agents such as antibiotics, antivirals; anticholinergic compounds, such as ipratropium (e.g., as the bromide), tiotropium (e.g., as the bromide), glycopyrronium (e.g., as the bromide), atropine, and oxitropium, or salts or other forms of any of the foregoing.

Additional active agents suitable for use in combination with one or more compounds of the invention include those useful for addressing one or more side effects associated with the use of steroids. Non-limiting examples include one or more inhibitors of osteoclast-mediated bone resportion. Suitable osteoclast-mediated bone resportion inhibitors include bisphosphonates (also called diphosphonates), such as Pamidronate (APD, Aredia®), Risedronate (Actonel®), Neridronate, Olpadronate, Alendronate (Fosamax®), Ibandronate (Boniva®), Risedronate (Actonel®), and Zoledronate (Zometa®).

Additional active agents suitable for use in combination with one or more compounds of the invention are described in WO03/035668, which are incorporated herein by reference.

Additional active agents suitable for use in combination with one or more compounds of the invention include chemokine receptor antagonists. Non-limiting examples of suitable chemokine receptor antagonists include CXCR1 and/or and CXCR2 antagonists. Non-limiting examples include SCH527123. See, e.g., Chapman, et al., “A novel, orally active CXCR1/2 receptor antagonist, SCH 527123, inhibits neutrophil recruitment, mucus production and goblet cell hyperplasia in animal models of pulmonary inflammation”, jpet.106.119040v1, May 11, 2007.

The combinations referred to herein may conveniently be presented for use in the form of a pharmaceutical formulation and thus pharmaceutical formulations comprising a combination as defined above together with a pharmaceutically acceptable diluent or carrier represent additional embodiments of the present invention. Appropriate doses of known therapeutic agents will be readily appreciated by those skilled in the art.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

1. A compound, or a pharmaceutically acceptable salt, solvate, ester, prodrug, or isomer thereof, of Formula (I): 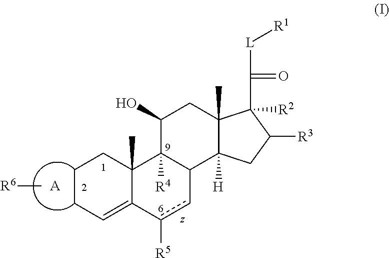
wherein:
ring A is a 5-membered heteroaryl ring containing from 1 to 2 ring heteroatoms, wherein each said ring heteroatom is independently selected from the group consisting of O, N, and S;
the dotted line at z represents an optional single or double bond;
L is a divalent moiety selected from the group consisting of 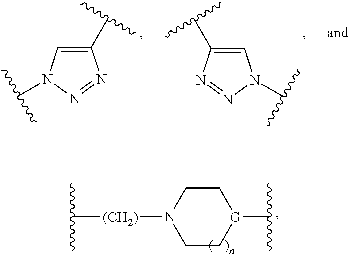
wherein G is N or CH and n is an integer from 0 to 2, with the proviso that when n is 0, G is CH,
or, alternatively, -L- is a divalent moiety selected from the group consisting of —CH₂S—, —S—, —CH₂—, —OCH₂—, —CH₂O—, —SCH₂—, —CH₂—S—CH₂—C(O)—NH—, —CH₂S(O)—, —CH₂S(O)₂—, —NR¹¹—N(R¹¹)—C(O)—, —N(R¹¹)—S(O)—, —N(R¹¹)—S(O)₂—, —NR¹¹O—, —CH₂N(R¹¹)—, —CH₂—N(R¹¹)—C(O)—, —CH₂—N(R¹¹)—C(O)—N(R¹¹)—, —CH₂—N(R¹¹)—C(O)O—, —CH₂N(R¹¹)C(═NH)NR¹¹—, —CH₂—N(R¹¹)—S(O)—, and —CH₂—N(R¹¹)—S(O)₂—,
R¹ is selected from the group consisting of —CN, alkyl, alkynyl, aryl, arylalkyl-, heteroarylfused aryl-, heteroarylfused arylalkyl-, cycloalkylfused aryl-, cycloalkylfused arylalkyl-, heteroaryl, heteroarylalkyl-, benzofused heteroaryl-, benzofused heteroarylalkyl-, heteroarylfused heteroaryl-, heteroarylfused heteroarylalkyl-, cycloalkyl, cycloalkenyl, cycloalkylalkyl-, cycloalkenylalkyl-, heterocycloalkyl, heterocycloalkenyl, heterocycloalkylalkyl-, heterocycloalkenylalkyl-, benzofused heterocycloalkyl-, benzofused heterocycloalkenyl-, benzofused heterocycloalkylalkyl-, benzofused heterocycloalkenylalkyl-, heteroarylfused heterocycloalkenyl-, and heteroarylfused heterocycloalkenylalkyl-,
wherein each said hetero ring-containing moiety of R¹ and each said heterofused containing moiety of R¹ independently contains 1, 2, or 3 ring heteroatoms independently selected from the group consisting of any combination of N, O, and S,
wherein each said R¹ group is unsubstituted or optionally substituted with from 1 to 5 substituents, which may be the same or different, each independently selected from the group consisting of halogen, hydroxy, —CN, oxo, oxide, alkyl, alkenyl, alkynyl, haloalkyl, haloalkoxy-, hydroxyalkyl-, heteroalkyl, cyanoalkyl-, alkoxy, optionally substituted aryl, optionally substituted —O-aryl, optionally substituted —O-alkyl-aryl, optionally substituted heteroaryl, optionally substituted arylalkyl-, optionally substituted arylalkoxy, optionally substituted heterocycloalkyl, optionally substituted heterocycloalkylalkyl-, optionally substituted —O-heterocycloalkyl, —N(R⁷)₂, -alkylN(R⁷)₂, —NC(O)R⁷, —C(O)R⁷, —CO₂R⁷, —SO₂R⁷, and —SO₂N(R⁷)₂, wherein said optional substituents are present from 1 to 4 times and may be the same or different, each independently selected from the group consisting of alkyl, halogen, haloalkyl, hydroxyl, —CN, and —N(R¹¹)₂;
and wherein the benzo portion of each said benzofused R¹ group is optionally further fused to another ring selected from the group consisting of heteroaryl, cycloalkyl, cycloalkenyl, heterocycloalkyl, and heterocycloalkenyl,
and wherein the alkyl- portion of said arylalkyl-, heteroarylfused arylalkyl-, cycloalkylfused arylalkyl-, heteroarylalkyl-, benzofused heteroarylalkyl-, heteroarylfused heteroarylalkyl-, cycloalkylalkyl-, cycloalkenylalkyl-, heterocycloalkylalkyl-, heterocycloalkenylalkyl-, benzofused heterocycloalkylalkyl-, benzofused heterocycloalkenylalkyl-, and heteroarylfused heterocycloalkenylalkyl-of R¹ is optionally substituted with one or more substituents independently selected from the group consisting of alkyl, haloalkyl, and spirocycloalkyl;
R² is selected from the group consisting of —OR⁸;
R³ is selected from the group consisting of H, —OH, and alkyl;
or R² and R³ are taken together to form a moiety of formula 2: 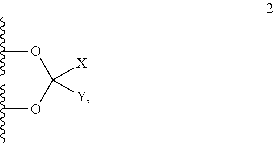
wherein X and Y are each independently selected from the group consisting of hydrogen, alkyl, haloalkyl, cycloalkyl, heterocycloalkyl, aryl, and heteroaryl,
wherein each of said alkyl, haloalkyl, cycloalkyl, heterocycloalkyl, aryl and heteroaryl of X and Y is optionally independently unsubstituted or substituted with from 1 to 4 substituents independently selected from the group consisting of alkyl, halogen, haloalkyl, hydroxy, —N(R⁷)₂, and —CN,
or X and Y of formula 2 are taken together with the carbon atom to which they are attached to form a 3 to 7-membered cycloalkyl or heterocycloalkyl ring, which ring is optionally substituted with from 1 to 4 substituents independently selected from the group consisting of alkyl, halogen, haloalkyl, hydroxy, —N(R⁷)₂ and —CN,
or R² and R³ taken together form a moiety of formula 3: 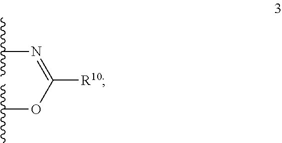
R⁴ is selected from the group consisting of Fl, halogen, and alkyl;
R⁵ is selected from the group consisting of H, halogen, and alkyl
R⁶ is selected from the group consisting of H, alkyl, -alkyl-CN, -alkyl-OH, alkoxy, heteroalkyl, —O-heteroalkyl, haloalkyl, aryl, arylalkyl-, naphthyl, naphthylalkyl-, heteroarylfused aryl, heteroarylfused arylalkyl-, cycloalkylfused aryl, cycloalkylfused arylalkyl-, heteroaryl, heteroarylalkyl-, benzofused heteroaryl, benzofused heteroarylalkyl-, heteroarylfused heteroaryl, heteroarylfused heteroarylalkyl-, cycloalkyl, cycloalkenyl, cycloalkylalkyl-, cycloalkenylalkyl-, heterocycloalkyl, heterocycloalkenyl, heterocycloalkylalkyl-, heterocycloalkenylalkyl-, benzofused heterocycloalkyl, benzofused heterocycloalkenyl, benzofused heterocycloalkylalkyl-, benzofused heterocycloalkenylalkyl-, heteroarylfused heterocycloalkenyl, and heteroarylfused heterocycloalkenylalkyl-,
wherein each said hetero ring-containing moiety of R⁶ contains 1, 2, or 3 ring heteroatoms independently selected from the group consisting of any combination of N, O, and S, and
wherein each said R⁶ (when other than H) is unsubstituted or substituted with from 1 to 4 groups independently selected from the group consisting of halogen, —CN, —OH, alkyl, haloalkyl, alkoxy, and —N(R⁷);
each R⁷ is independently selected from the group consisting of hydrogen, alkyl, haloalkyl, aryl, and heteroaryl,
or, two groups R⁷ attached to the same nitrogen atom form a 3- to 7-membered heterocycloalkyl group;
R⁸ selected from the group consisting of hydrogen, alkyl, haloalkyl, aryl, heteroaryl, arylalkyl, heteroarylalkyl, —C(O)R⁹, and —C(O)NHR⁹;
each R⁹ is independently selected from the group consisting of alkyl, haloalkyl, aryl, heteroaryl, cycloalkyl, and heterocycloalkyl, each optionally substituted with 1 to 4 substituents independently selected from the group consisting of alkyl, halogen, haloalkyl, hydroxy, —N(R⁷), and —CN;
each R¹⁰ is independently selected from the group consisting of hydrogen and alkyl; and
each R¹¹ is independently selected from the group consisting of hydrogen and alkyl.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. A compound of claim 1, or a pharmaceutically acceptable salt, solvate, ester, prodrug, or isomer thereof, said compound being selected from the group consisting of:

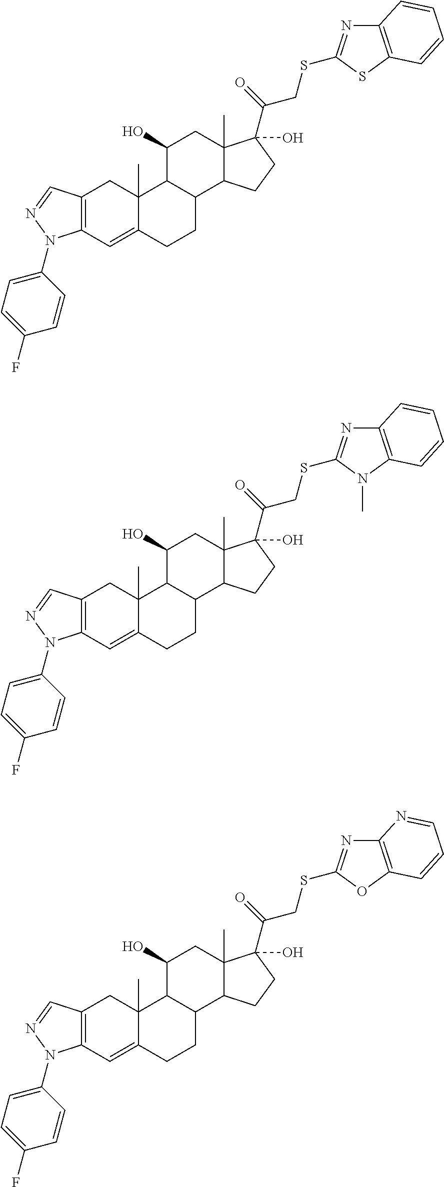

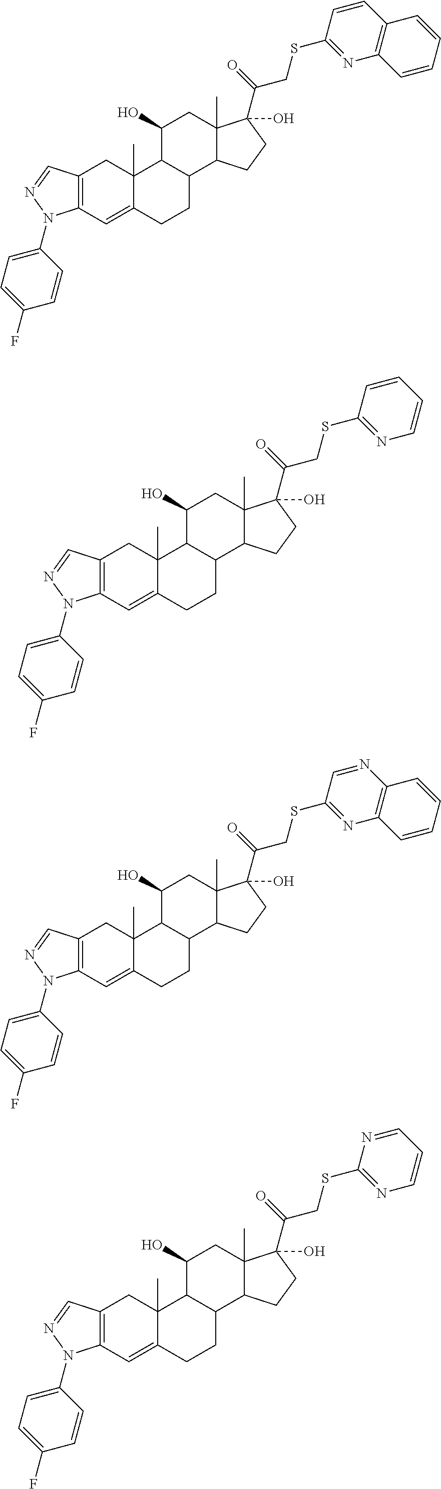

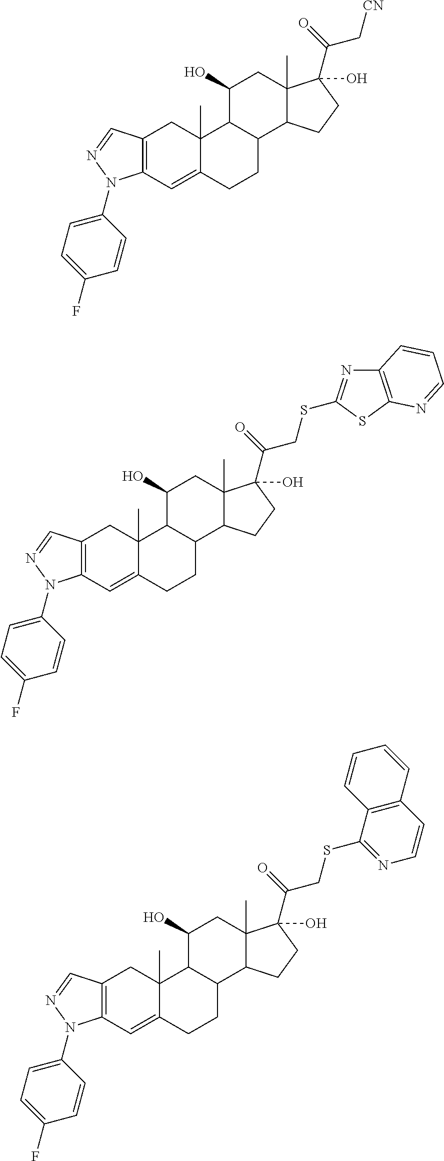

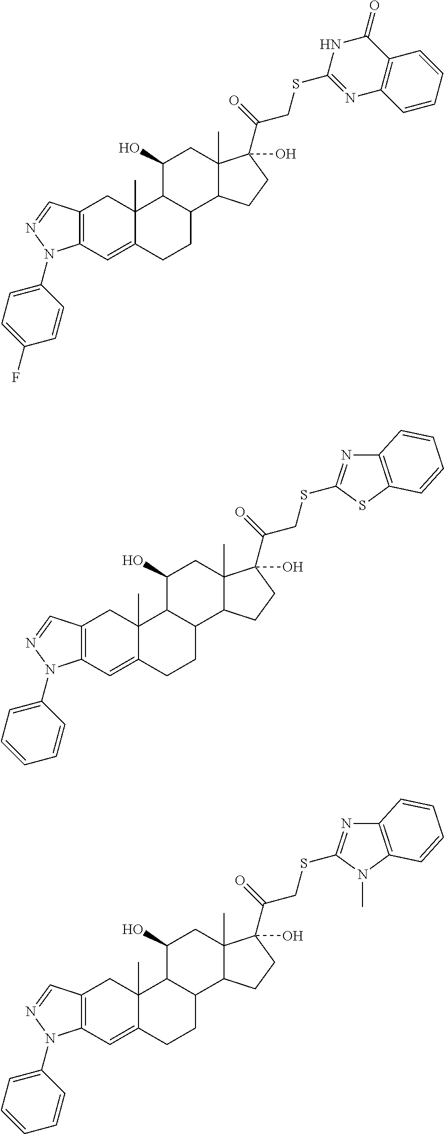

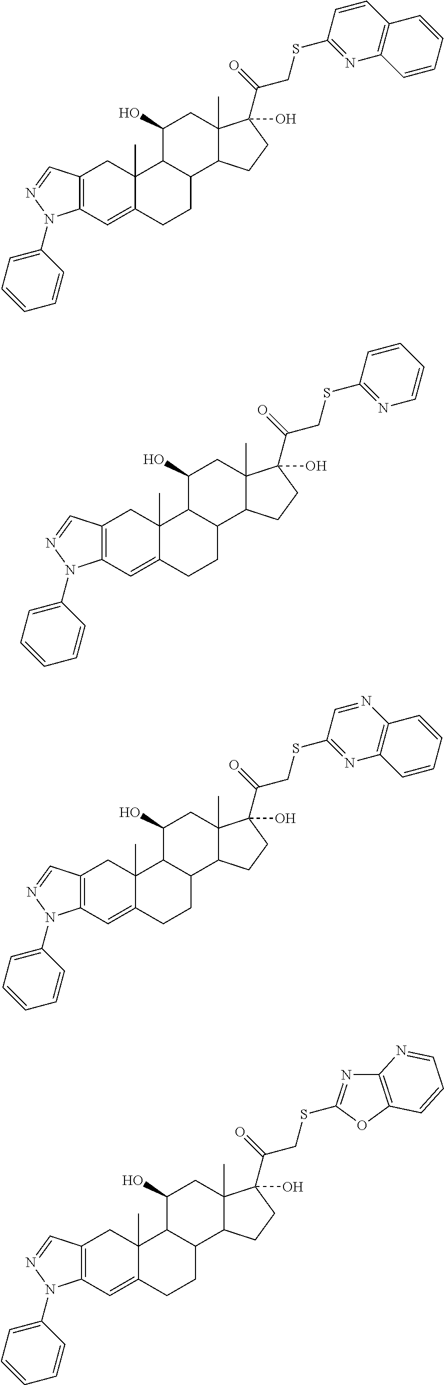

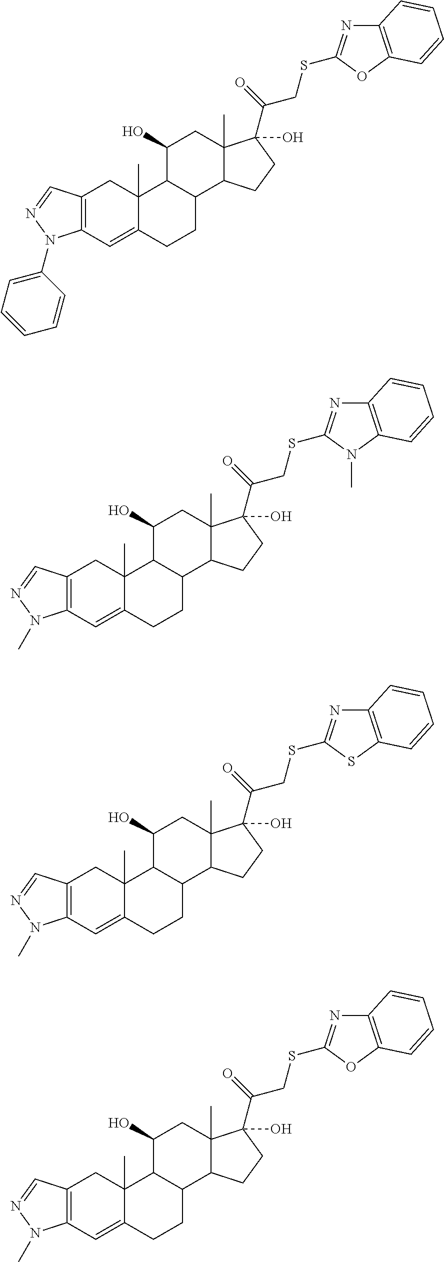

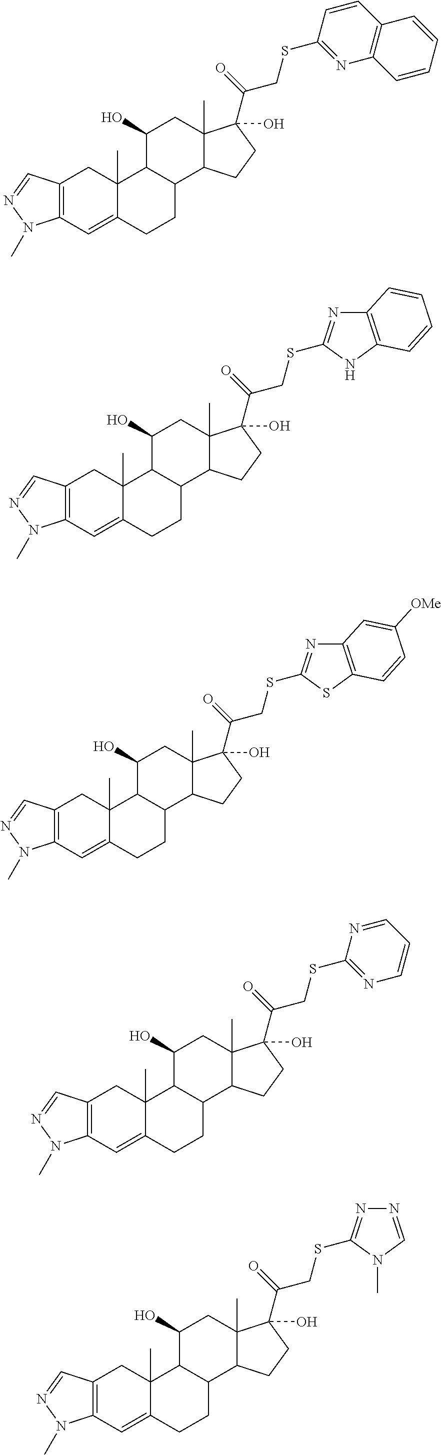

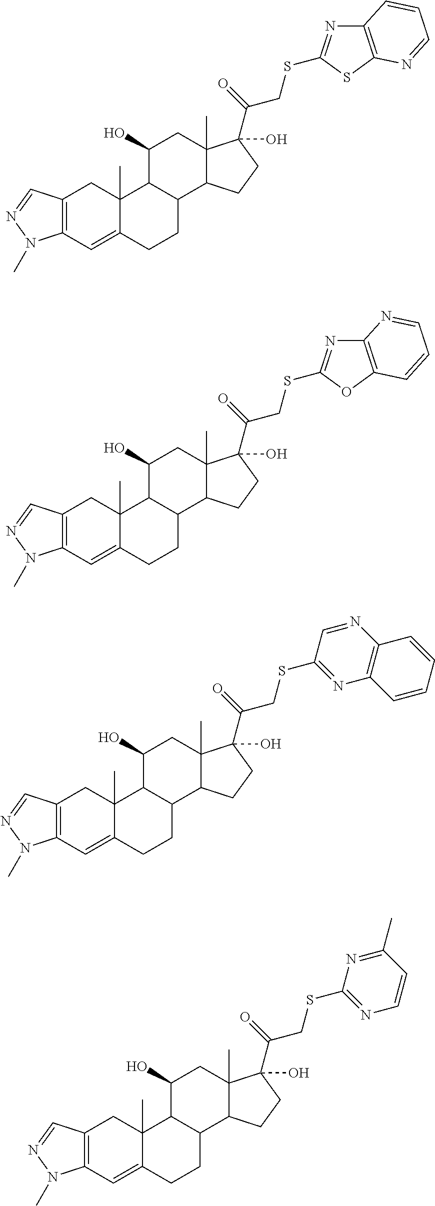

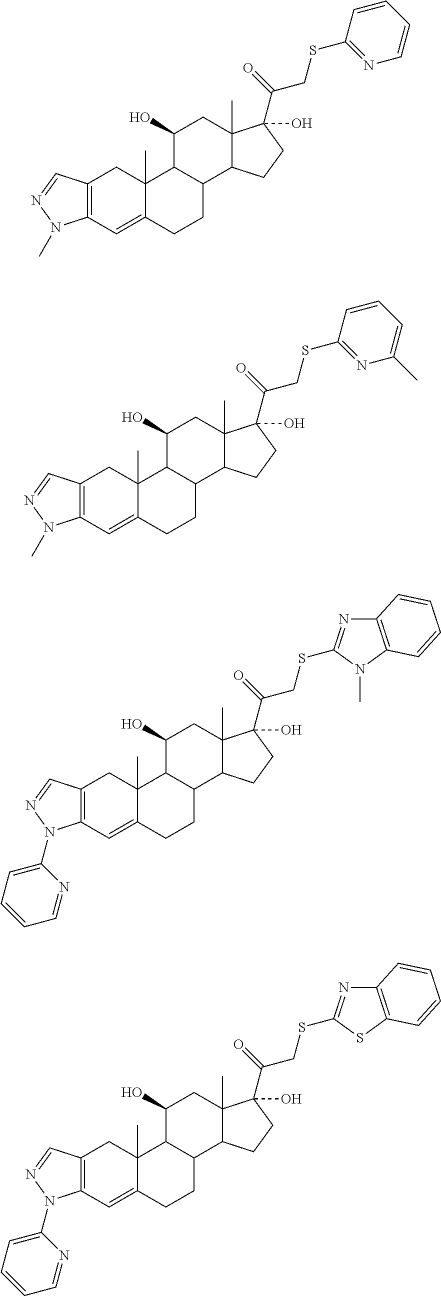

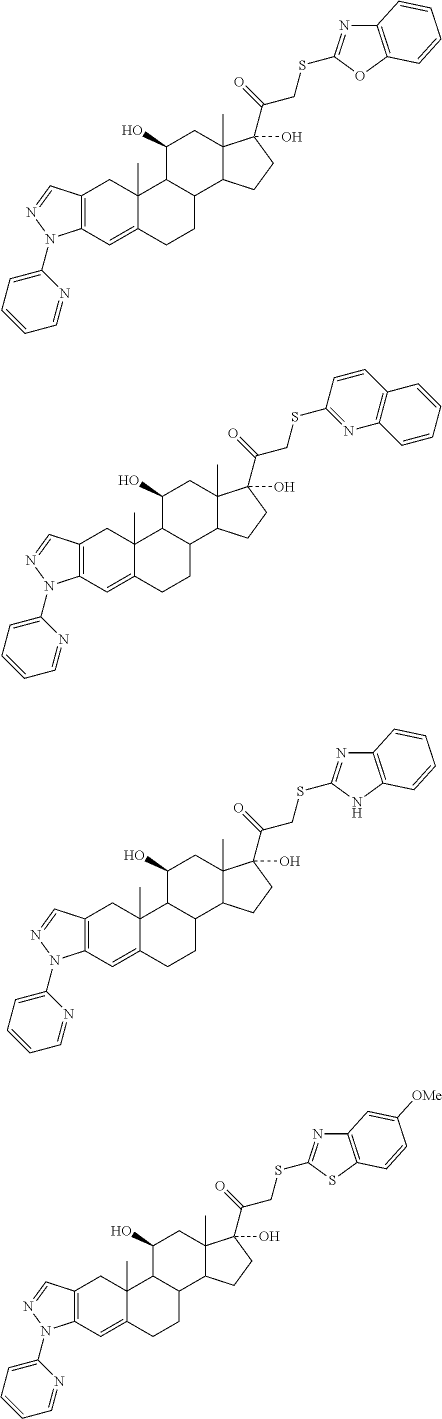

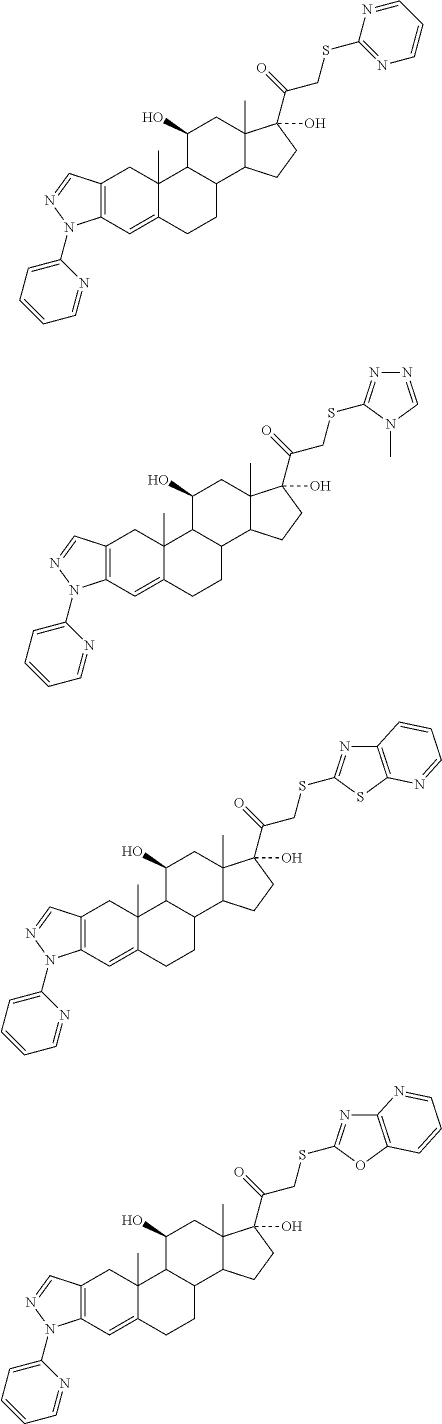

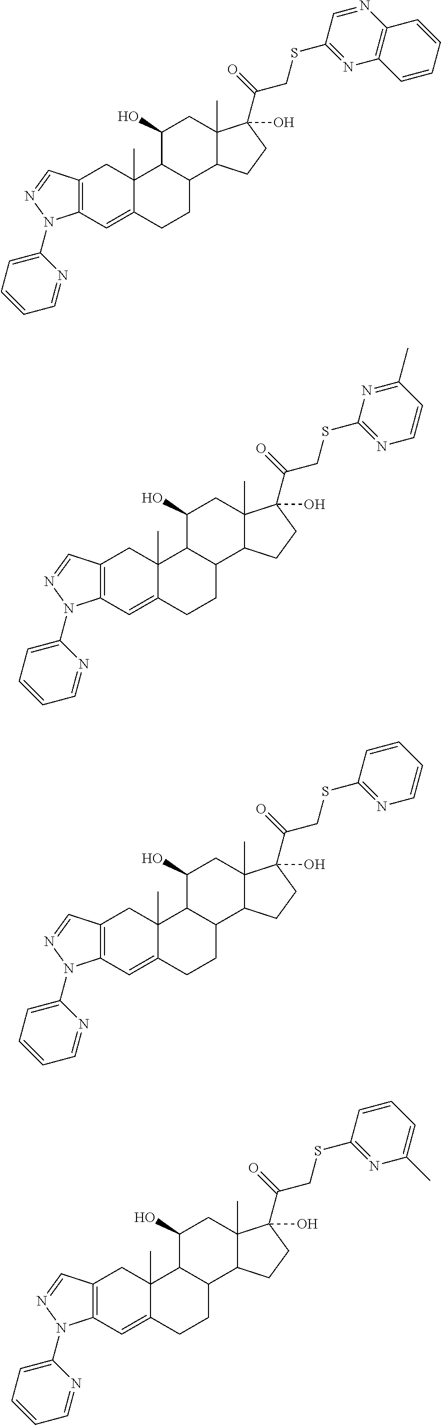

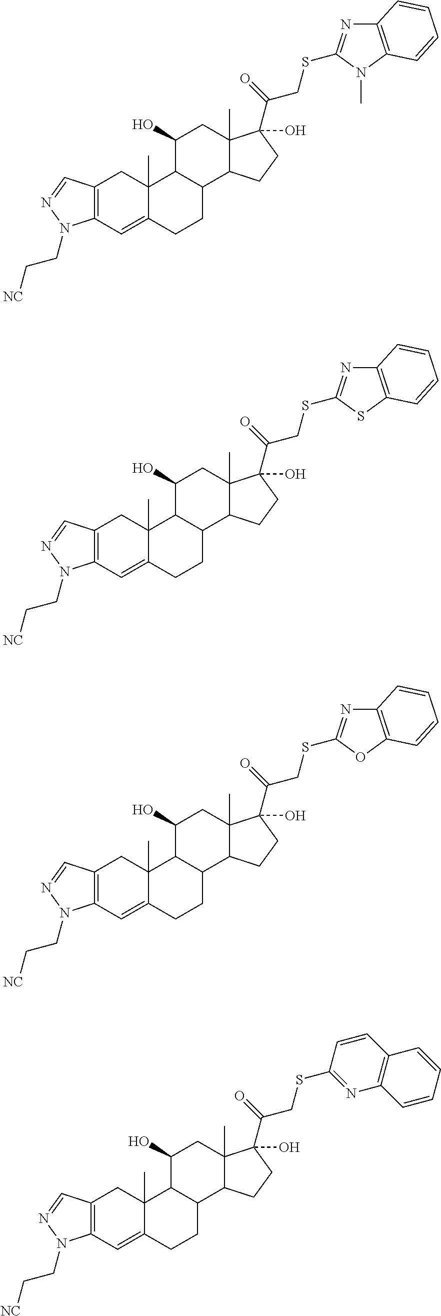

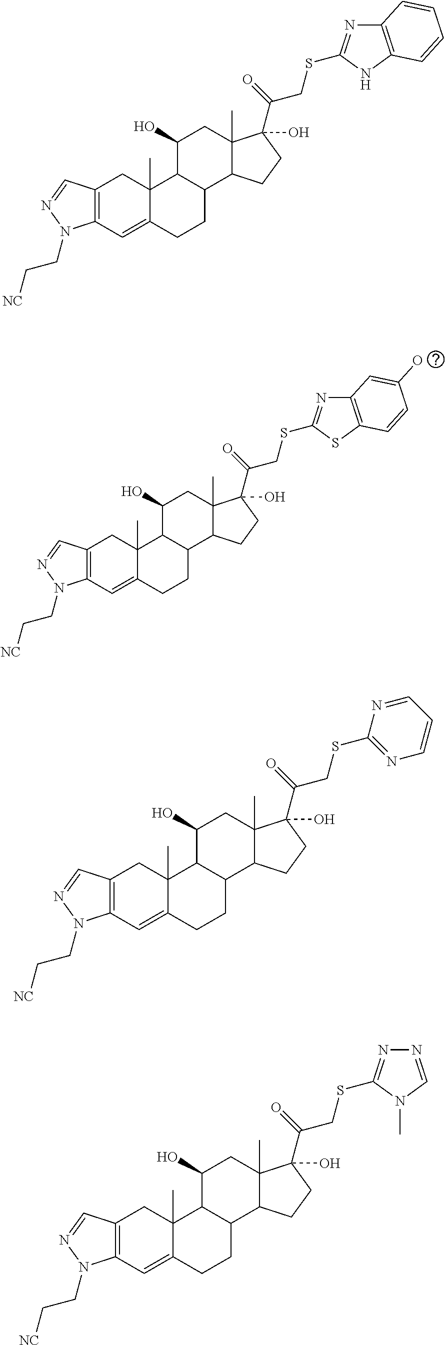

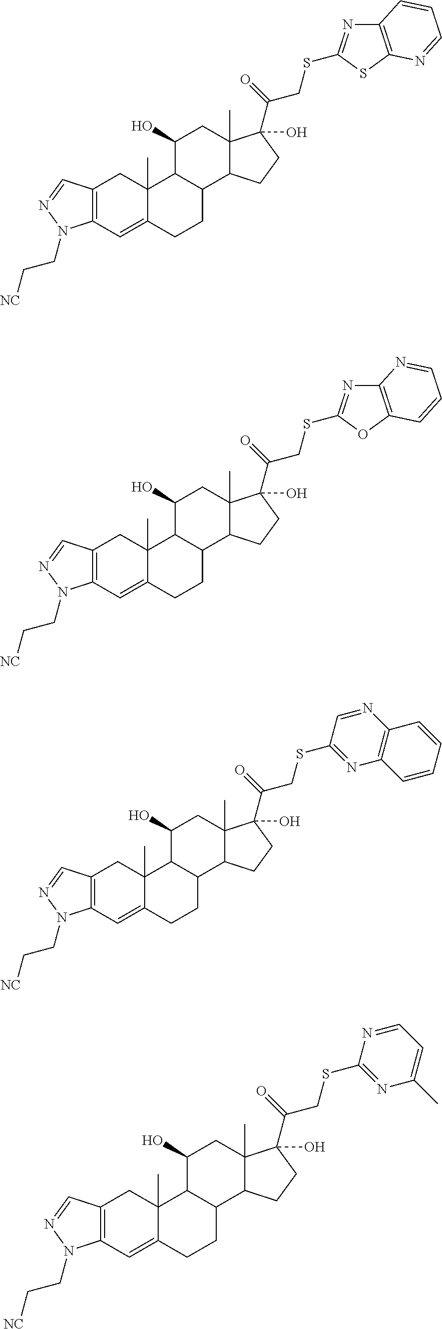

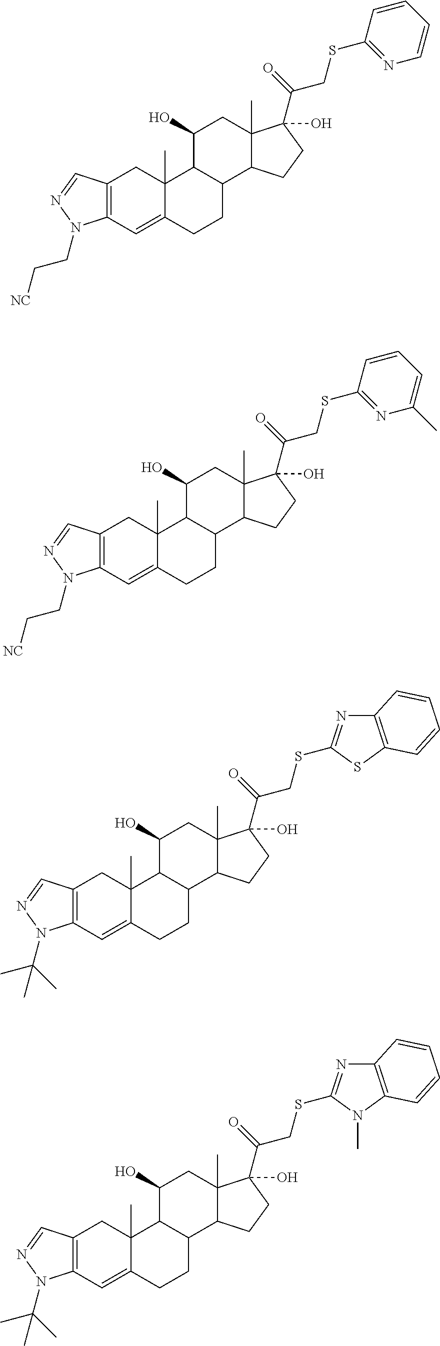

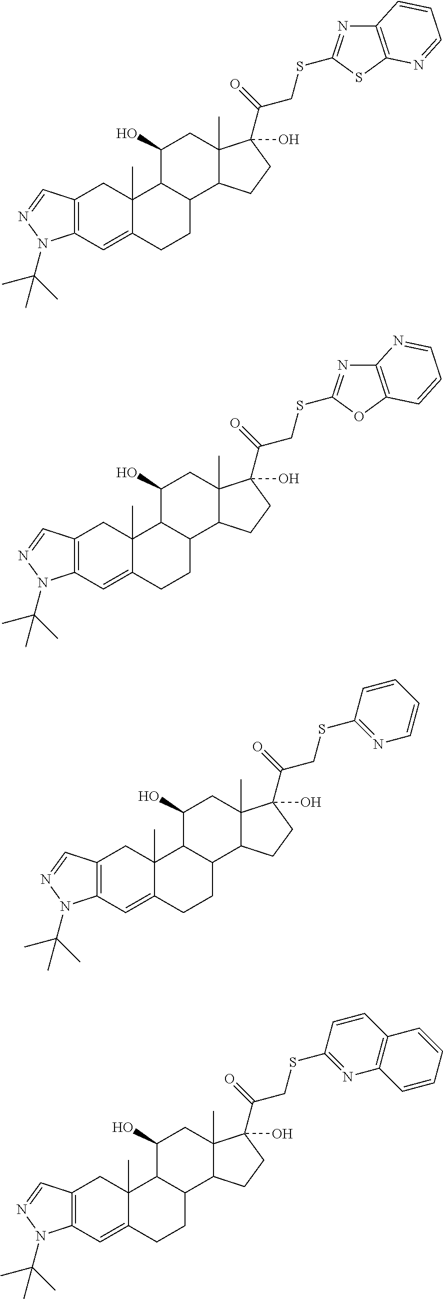

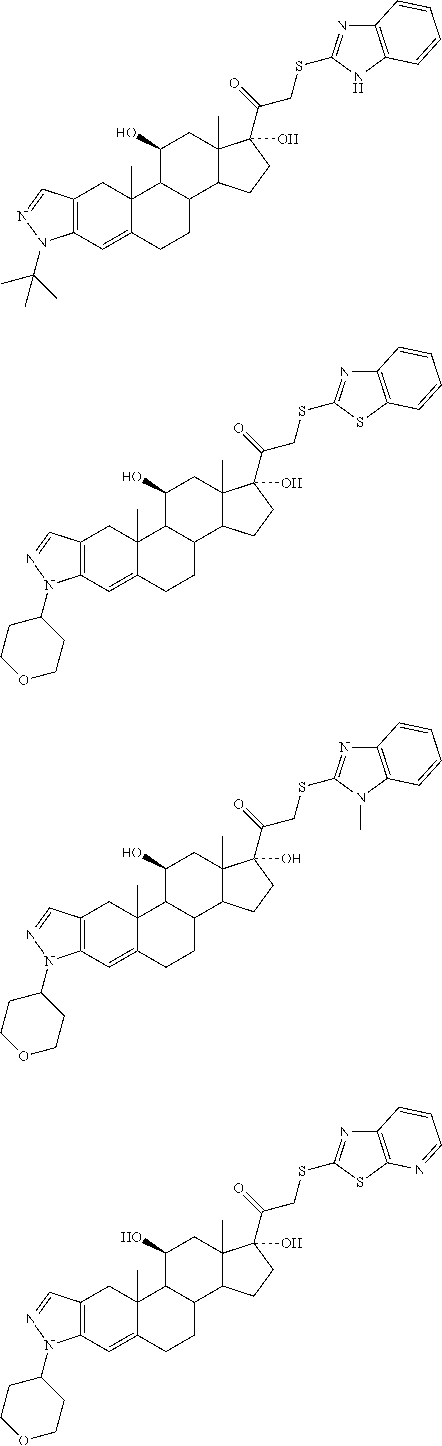

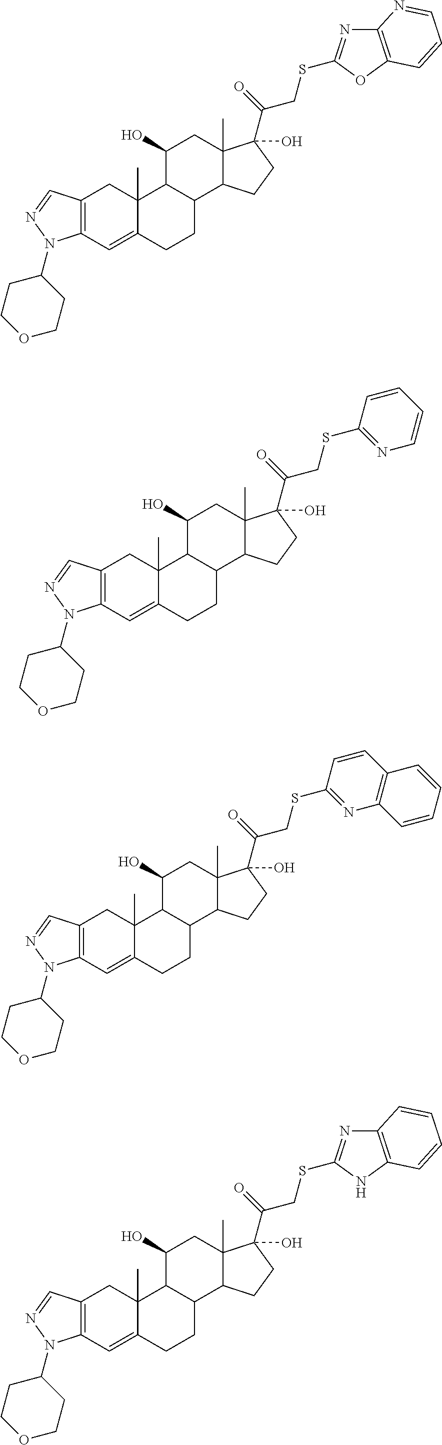

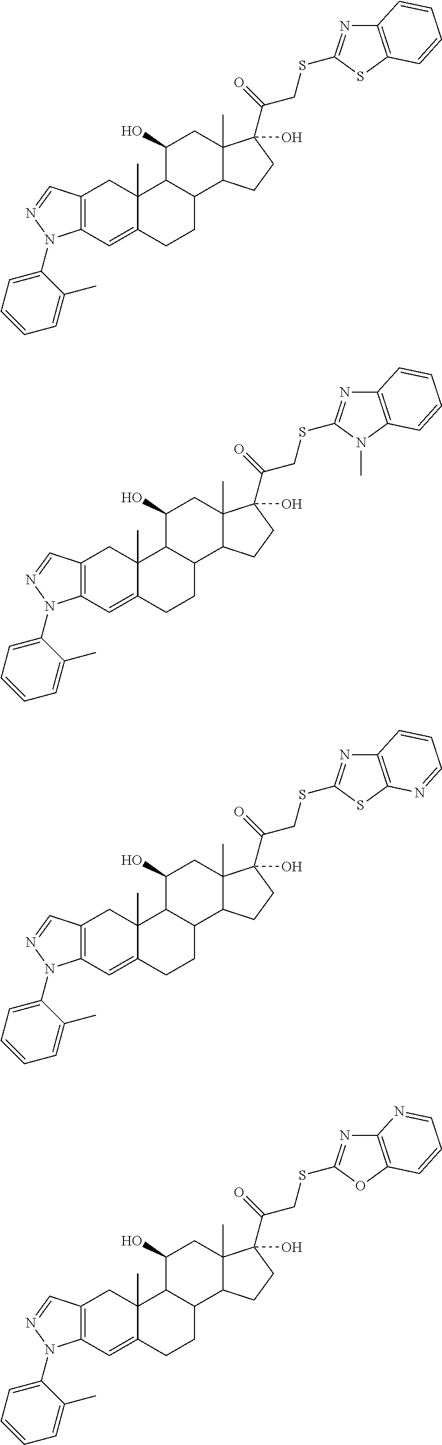

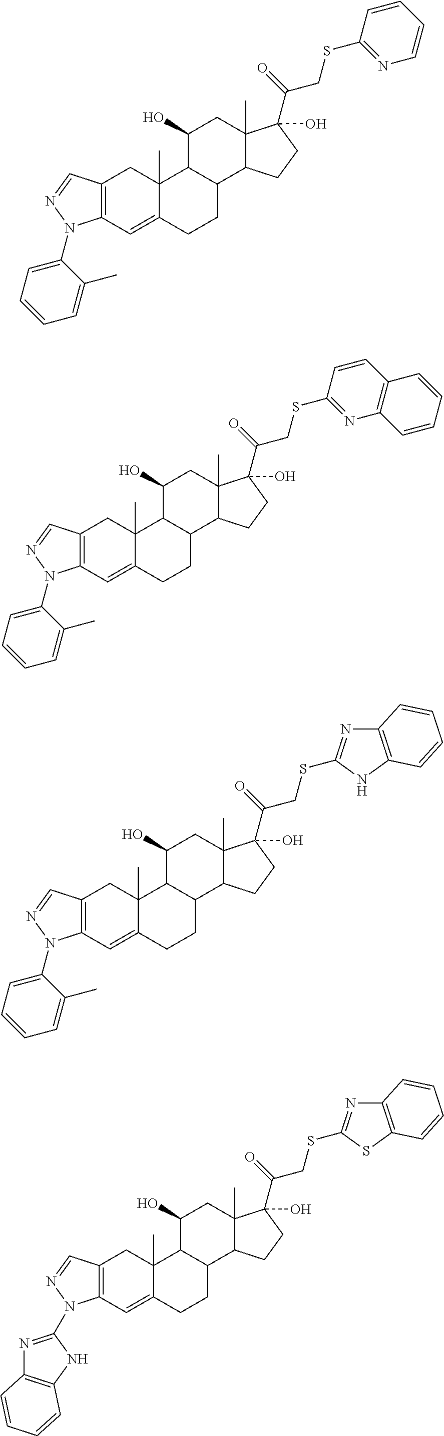

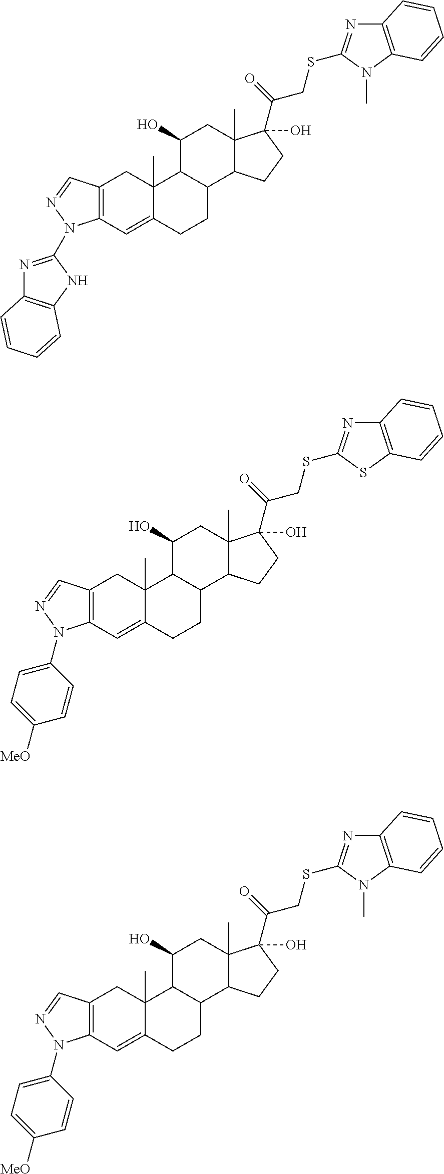

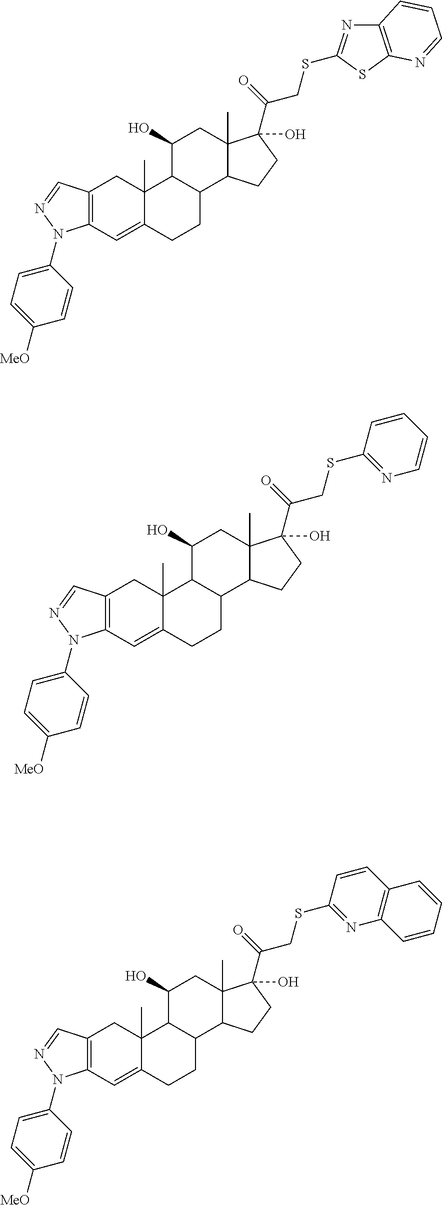

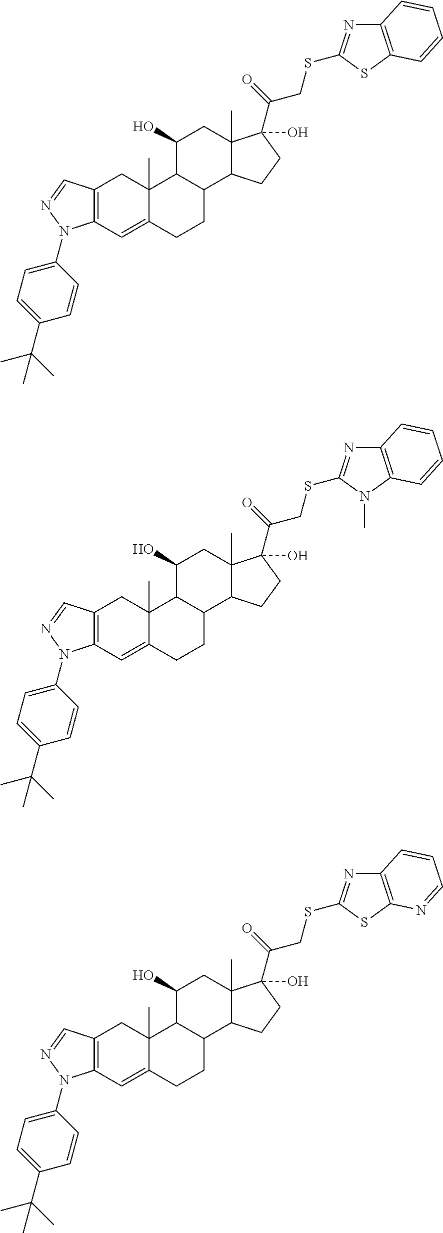

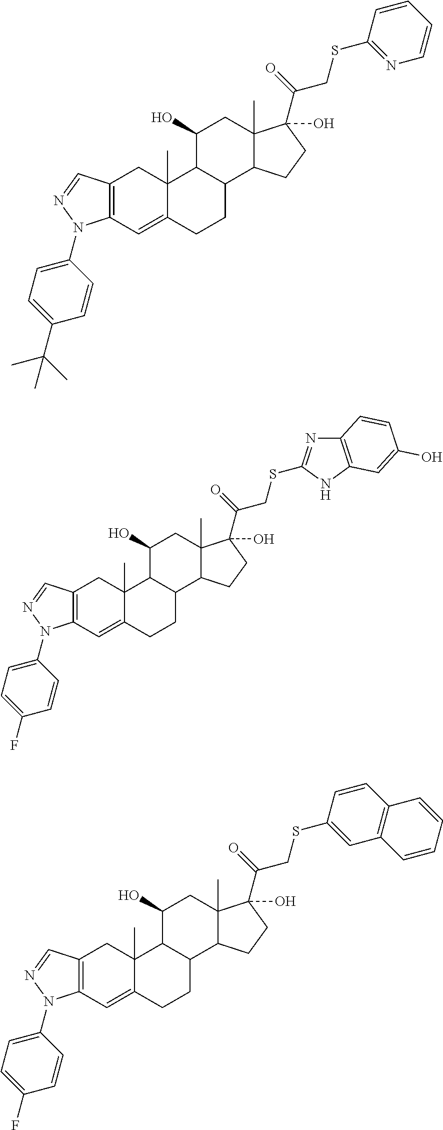

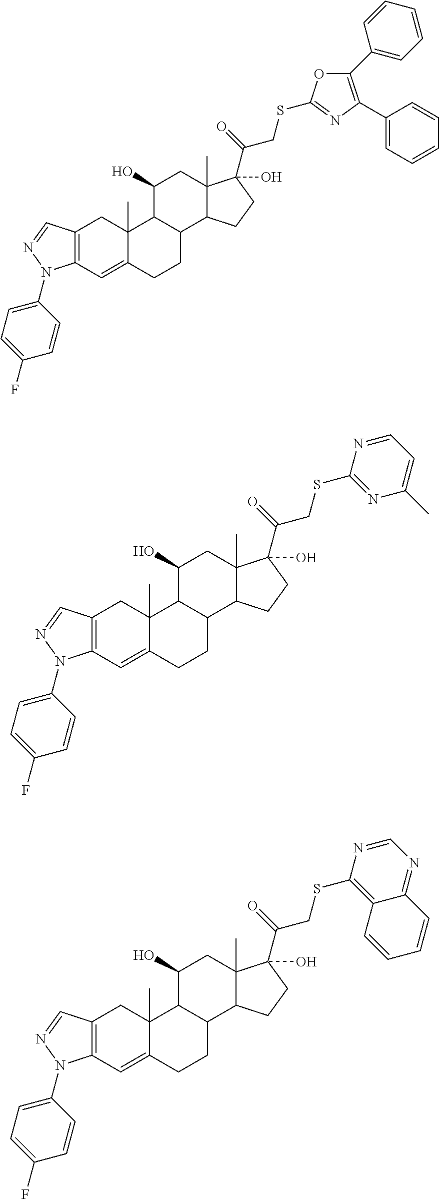

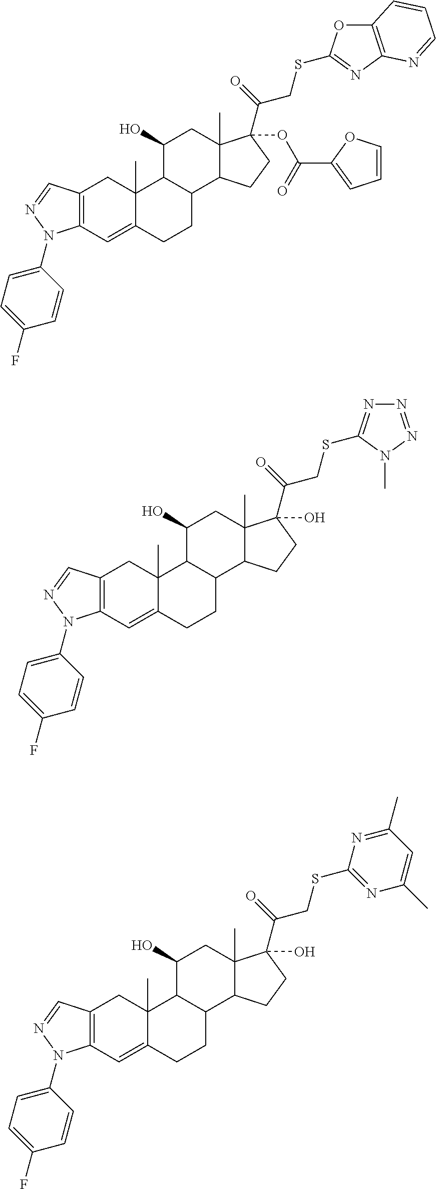

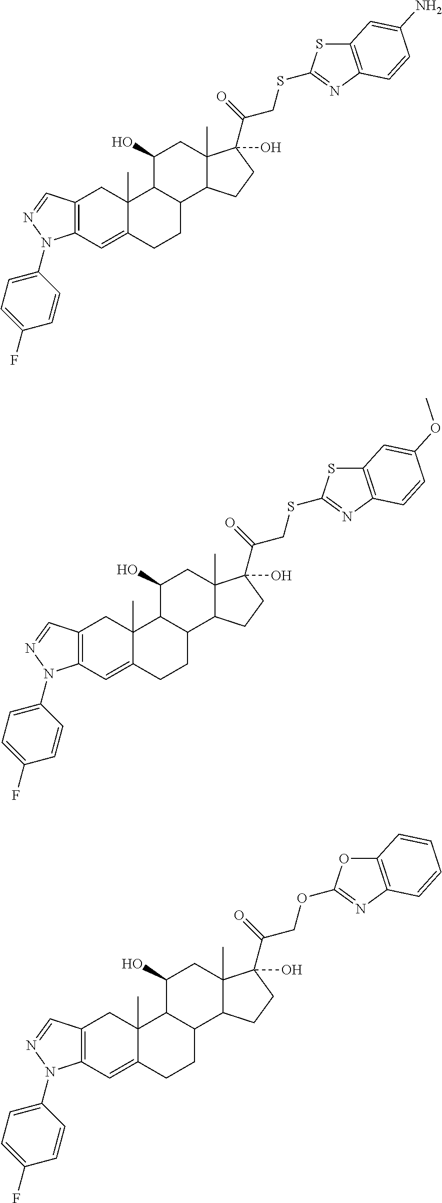

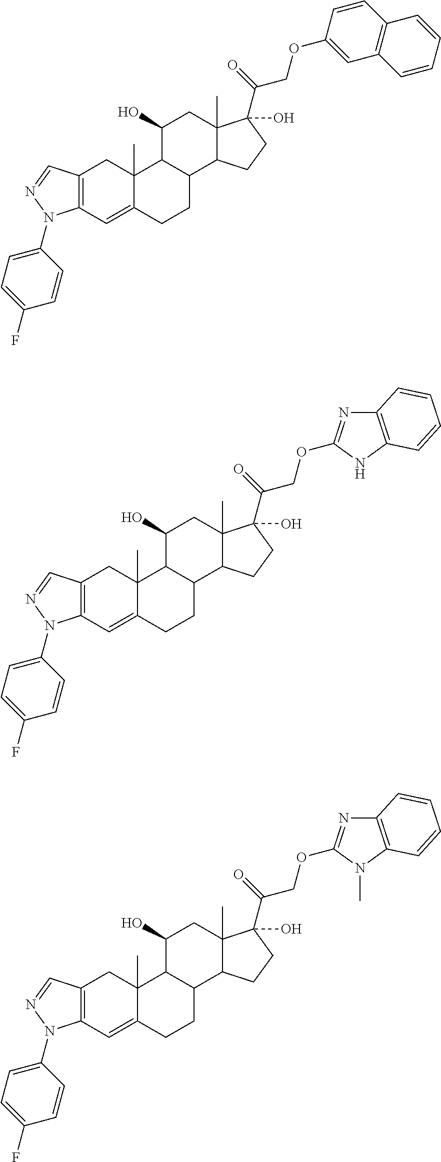

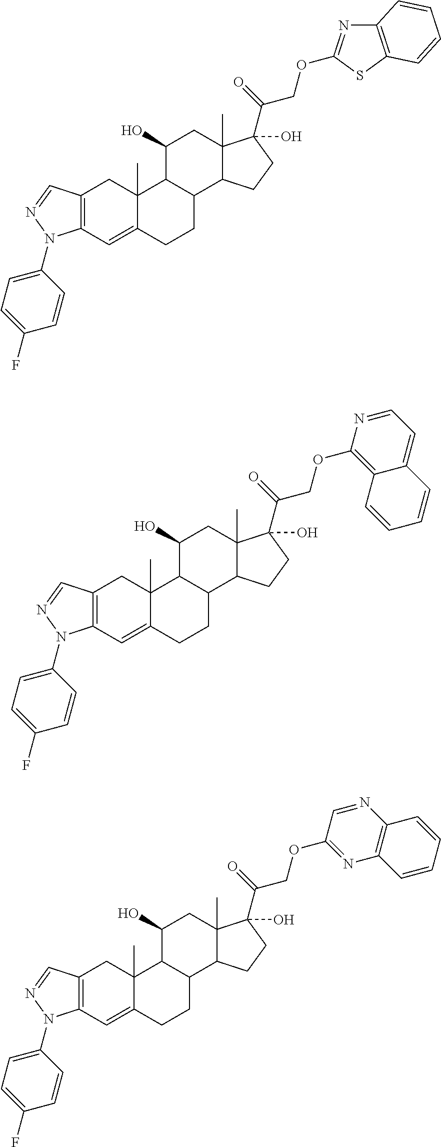

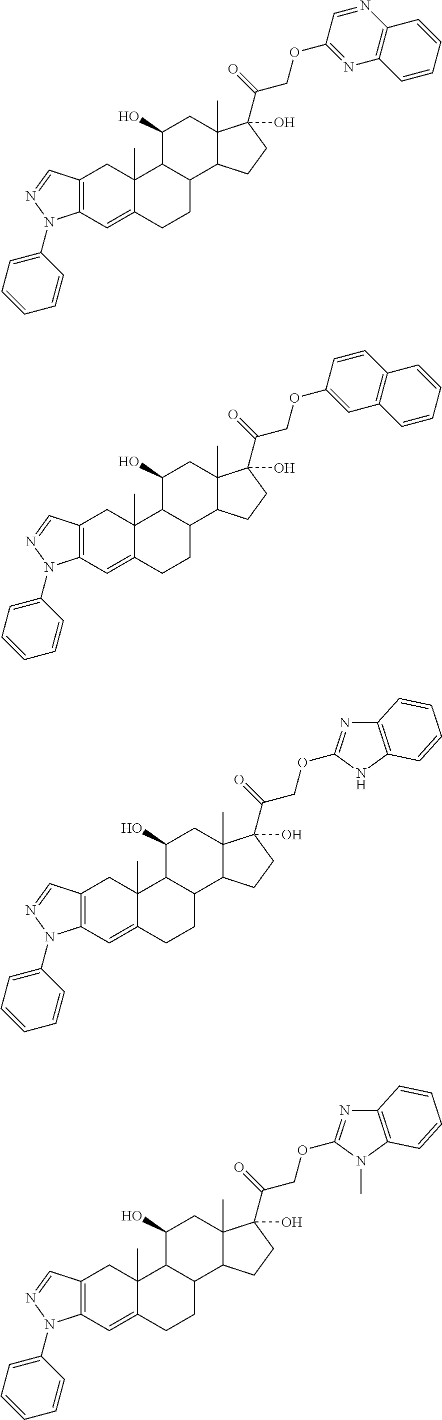

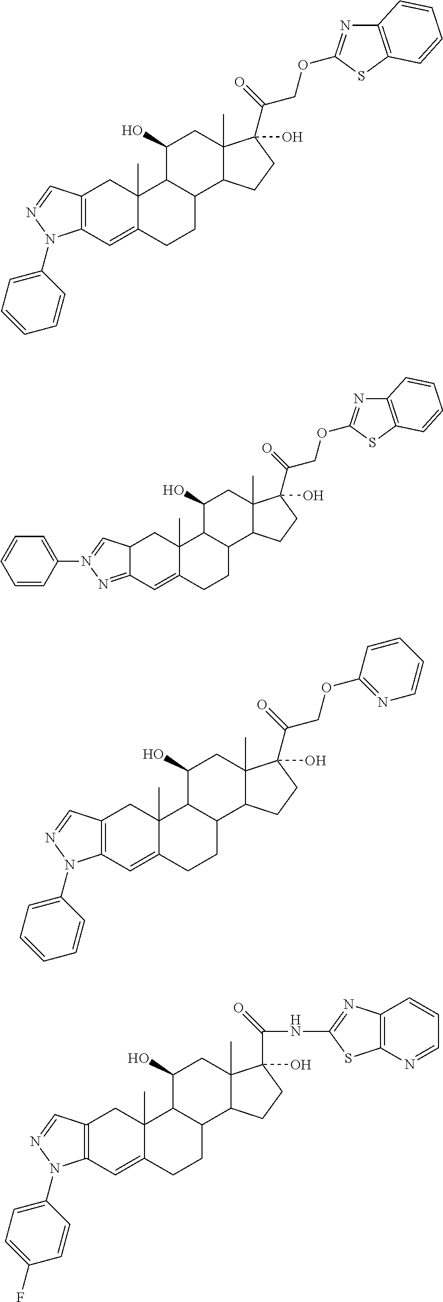

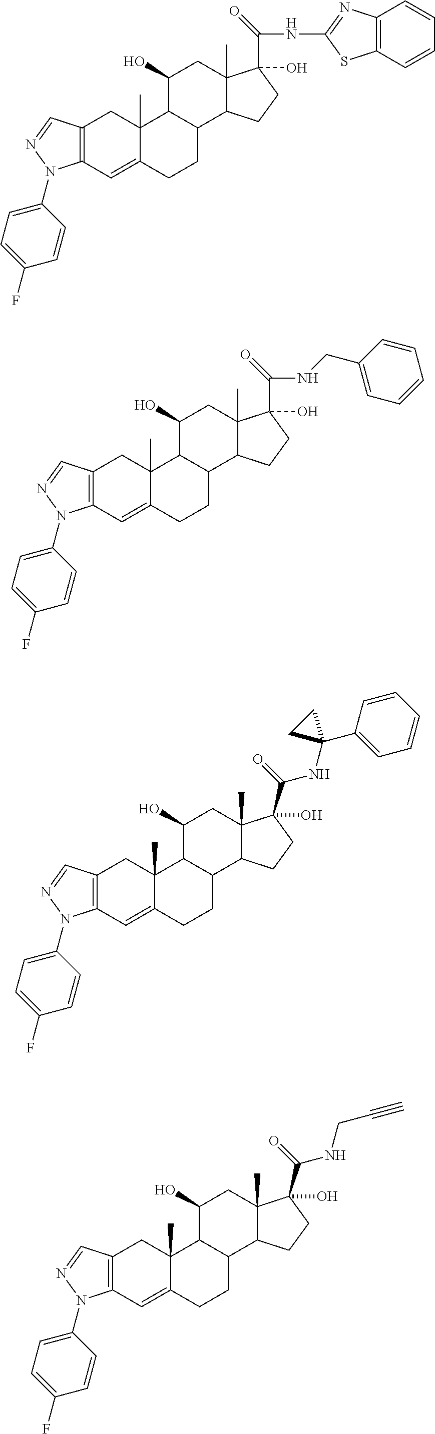

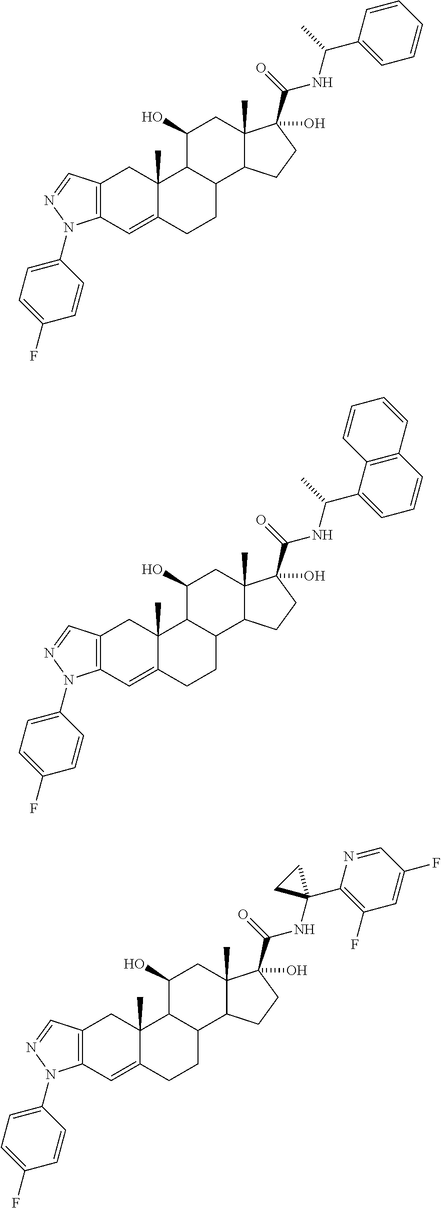

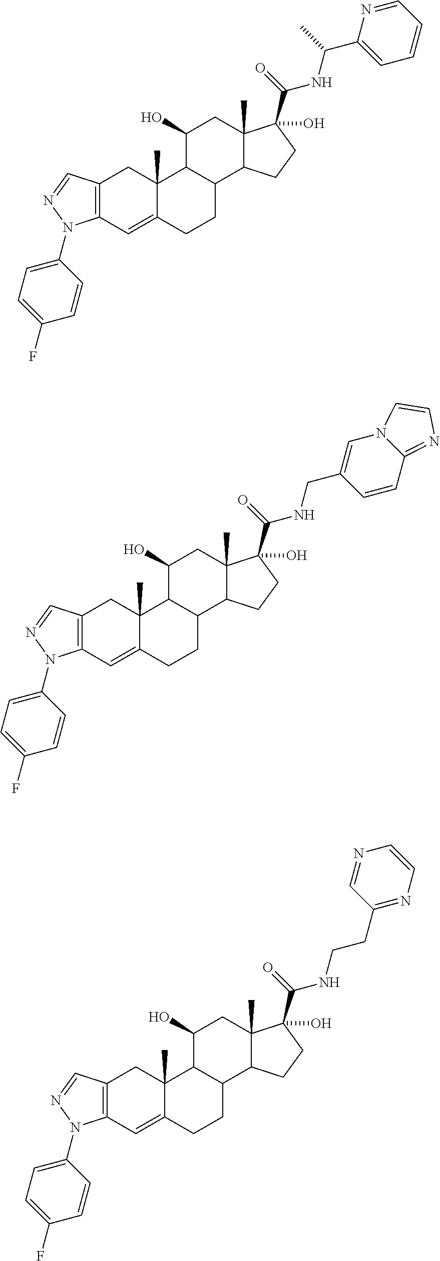

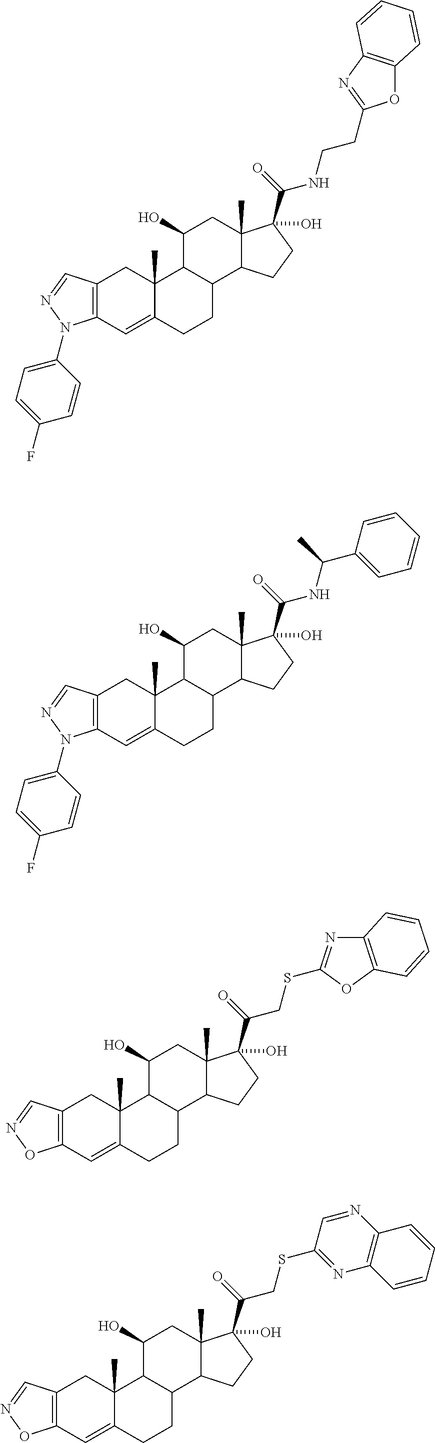

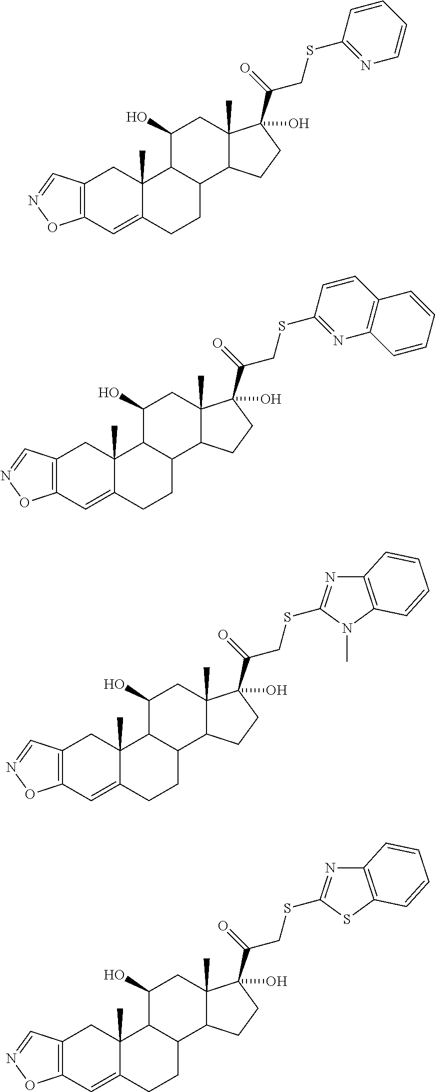

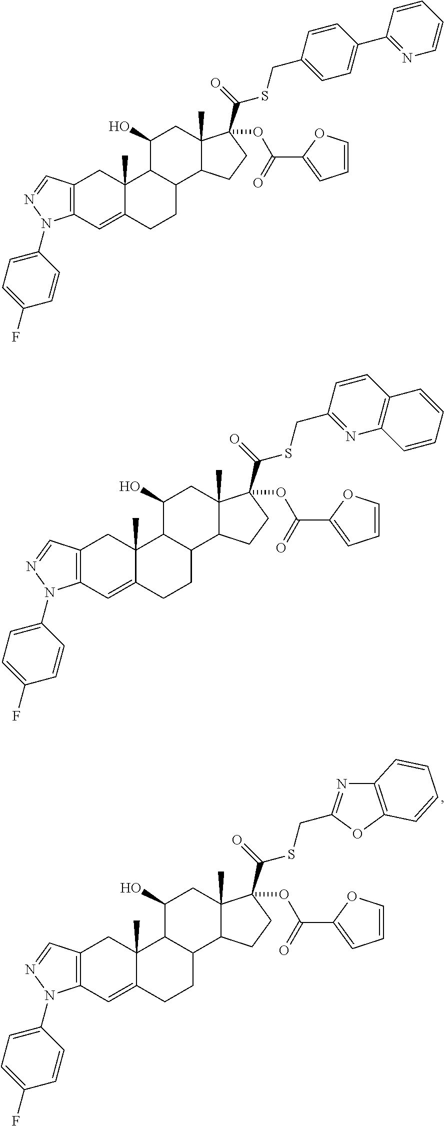

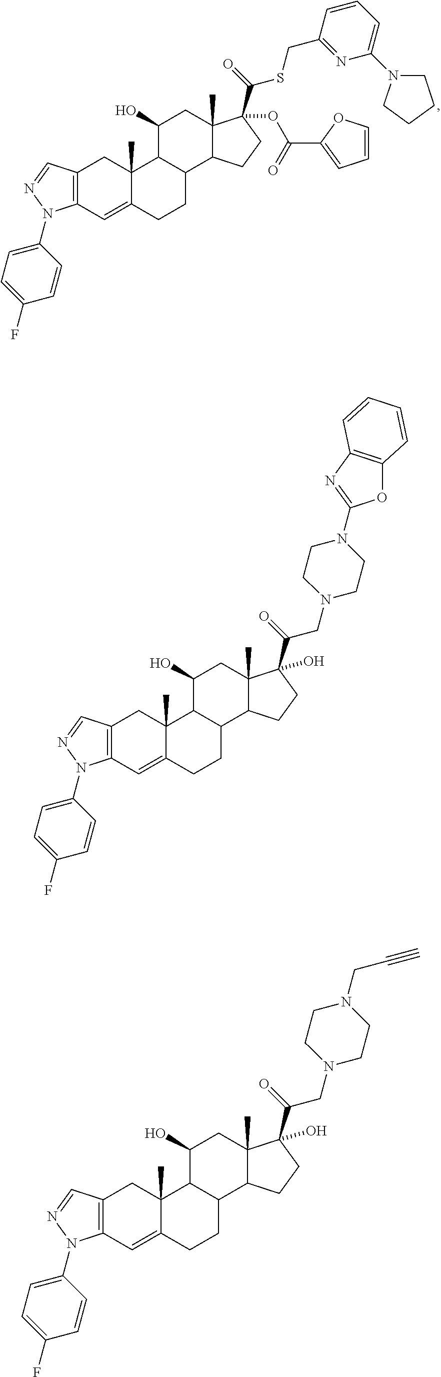

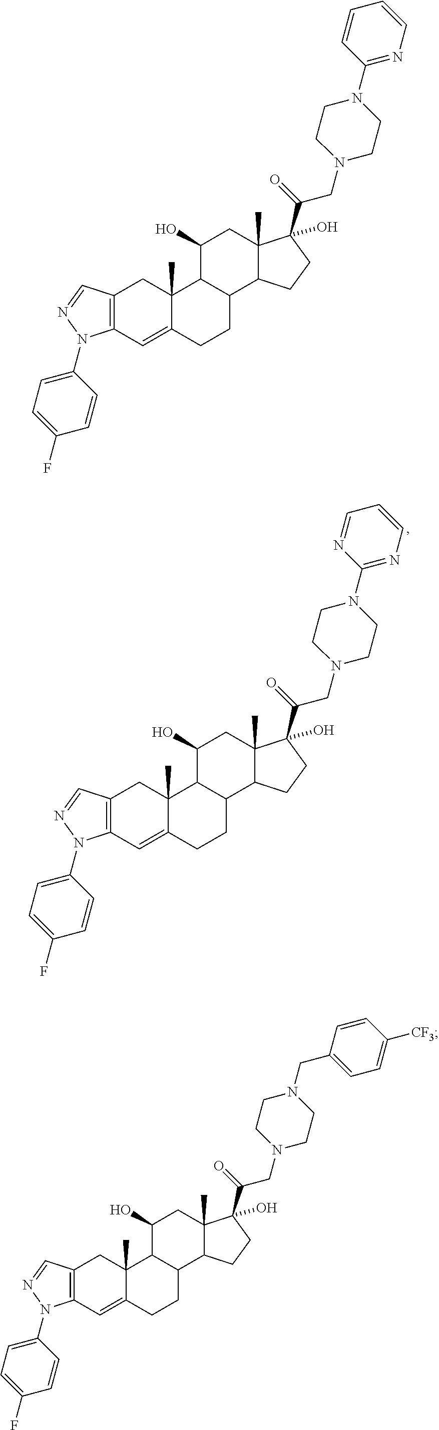

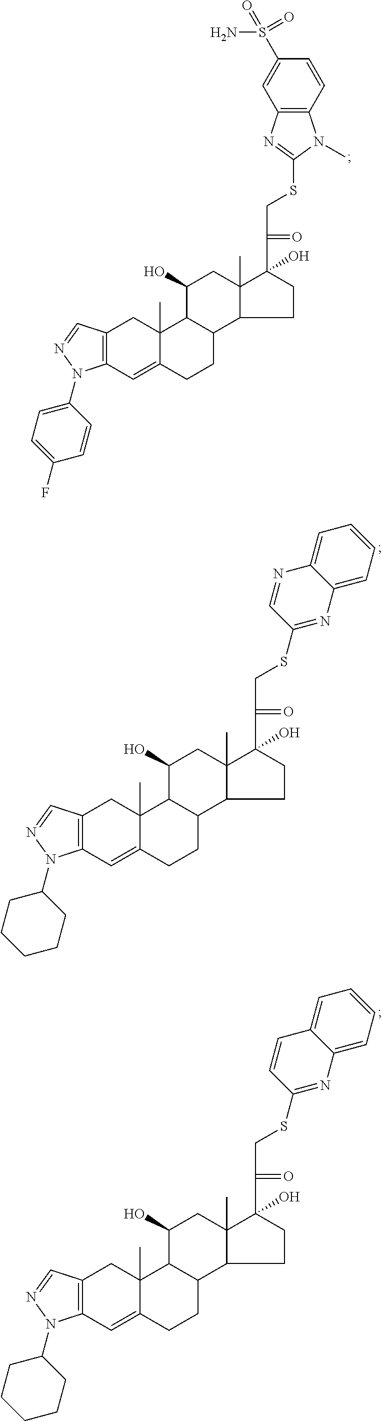

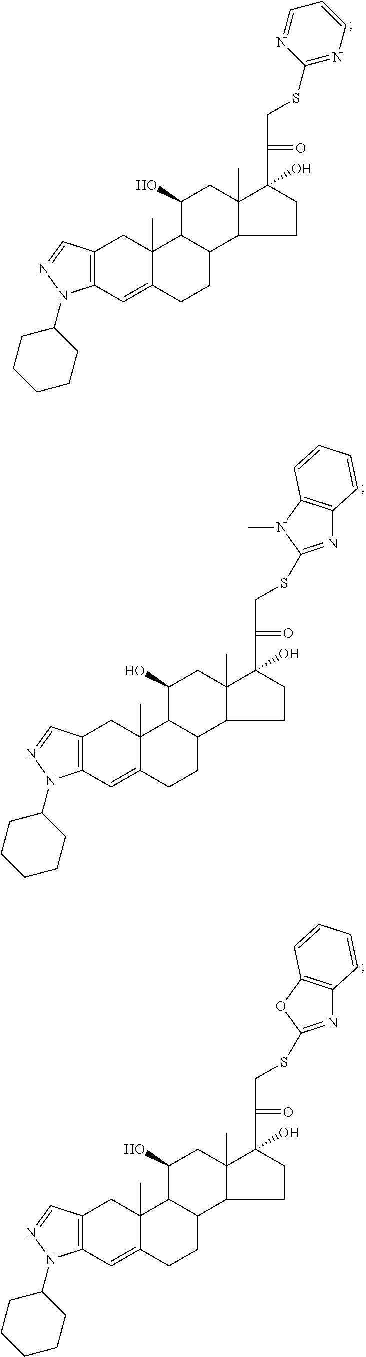

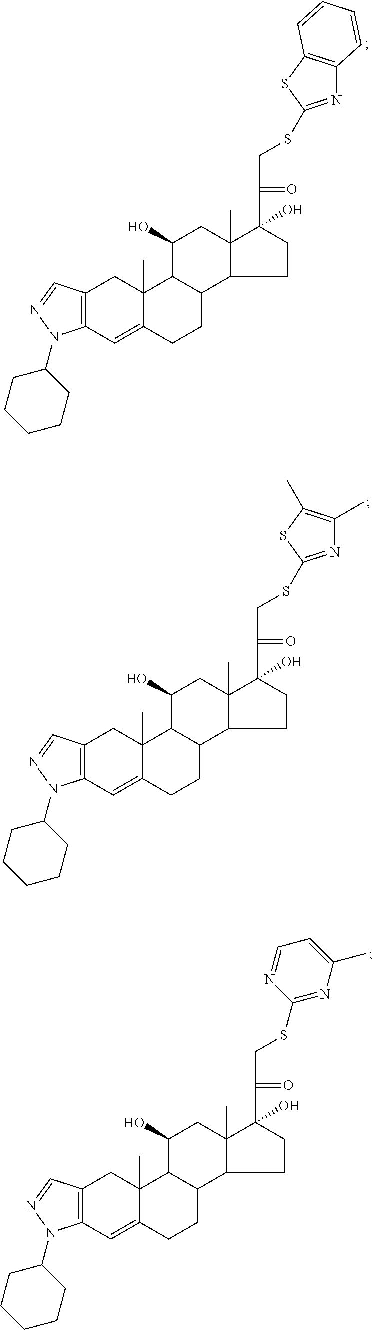

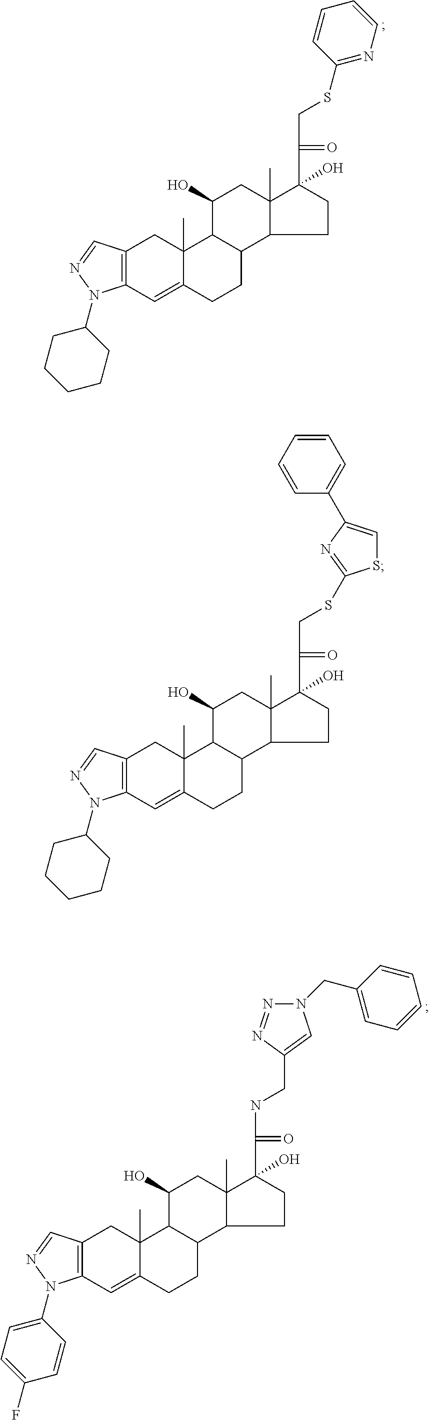

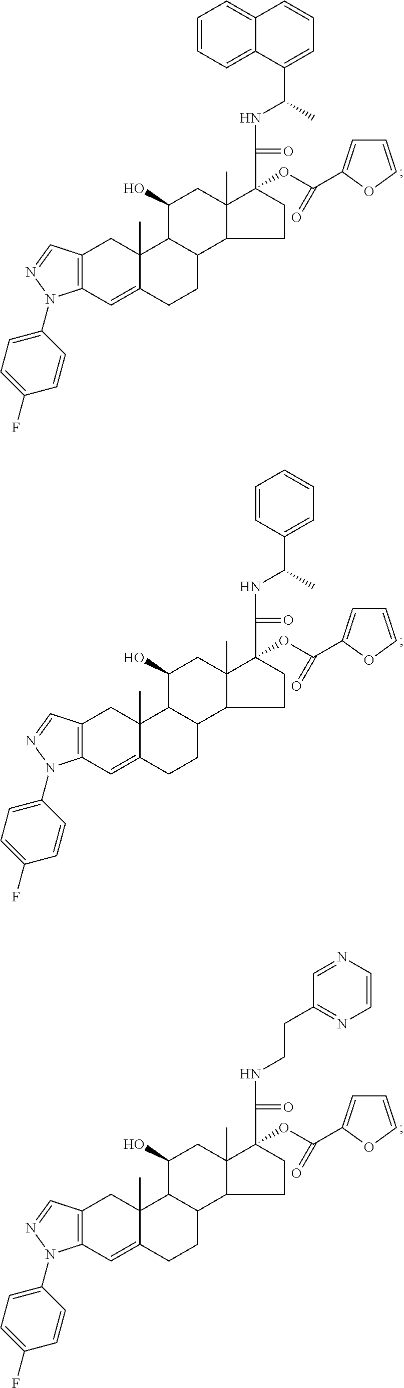

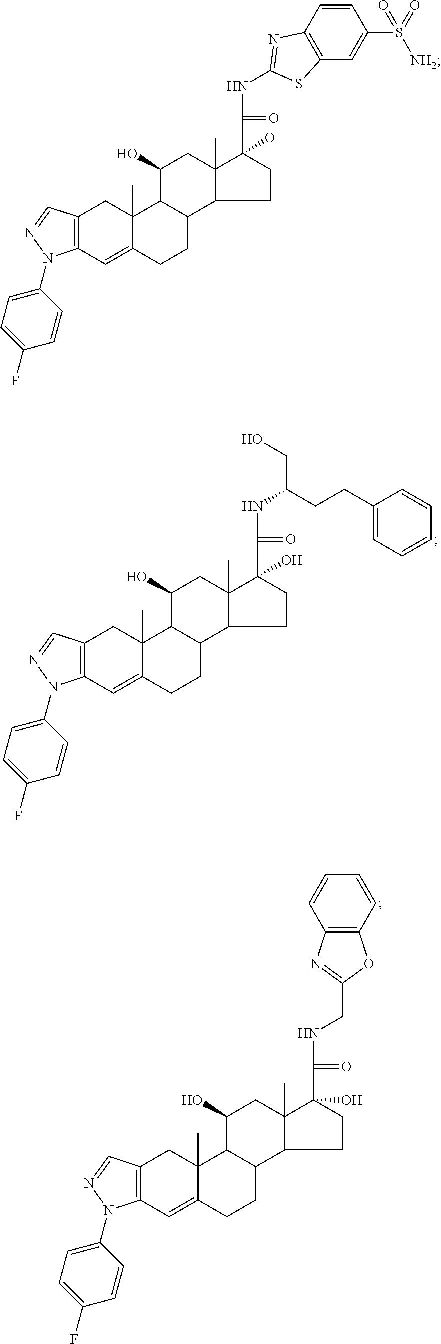

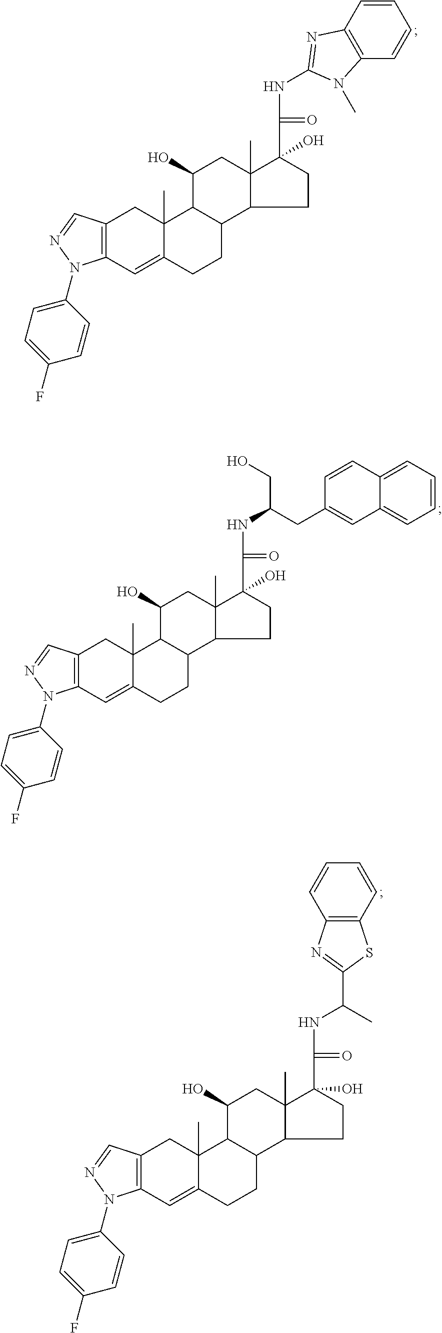

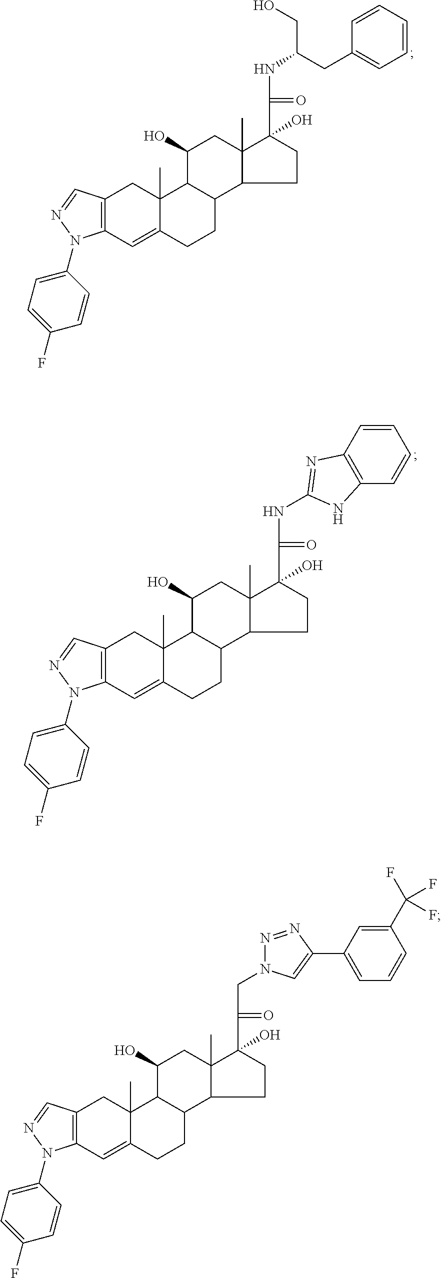

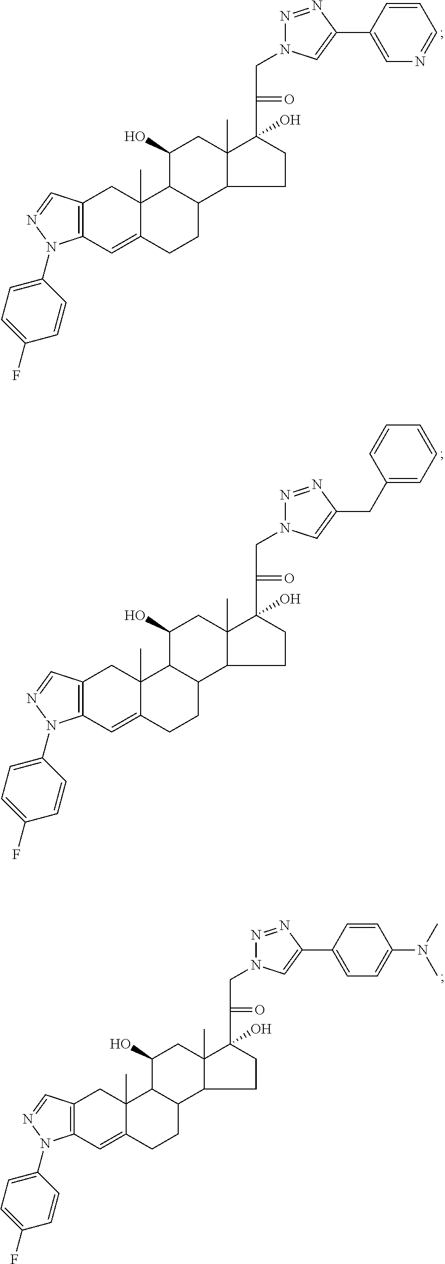

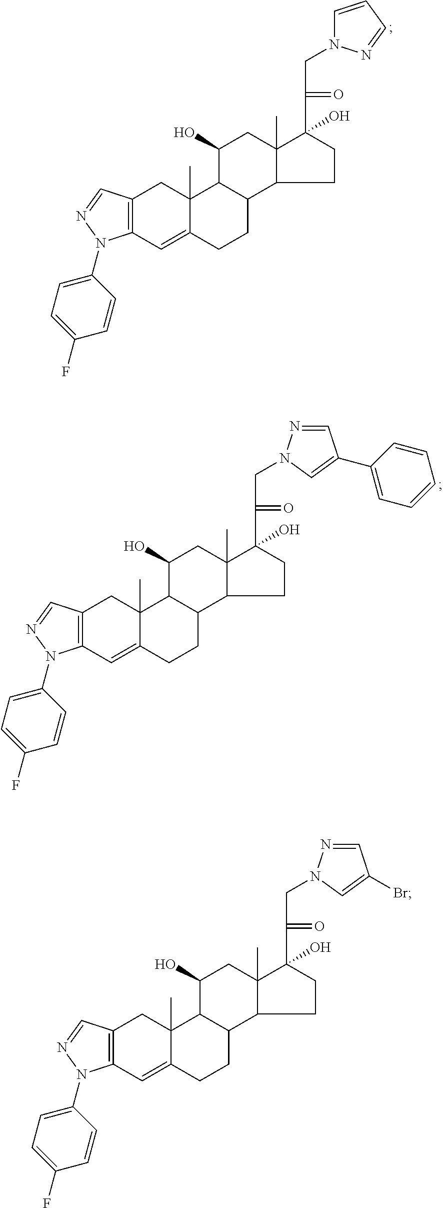

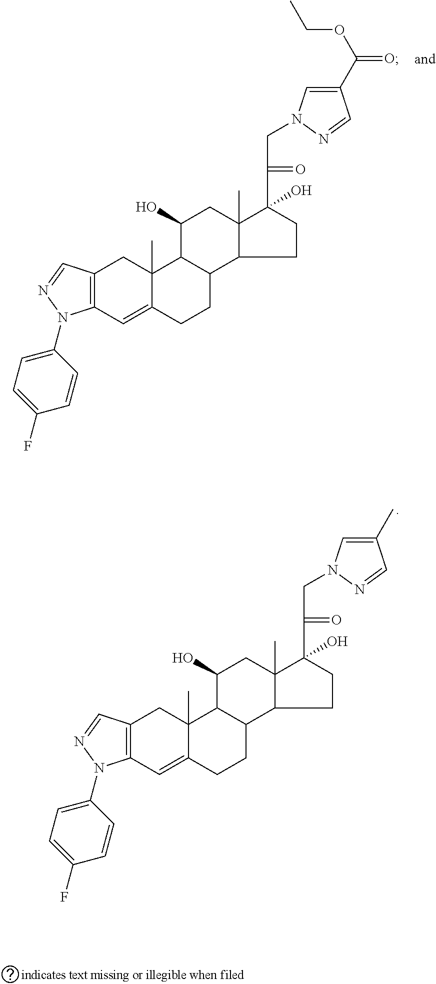

21. A pharmaceutical composition comprising a compound of claim 1, or a pharmaceutically acceptable salt thereof, optionally in admixture with one or more pharmaceutically acceptable diluents or carriers.

22. A pharmaceutical composition comprising a compound of claim 1, or a pharmaceutically acceptable salt thereof, and a propellant, optionally in combination with a surfactant or cosolvent.

23. A pharmaceutical composition comprising a compound of claim 1, or a pharmaceutically acceptable salt thereof, and a propellant, formulated for topical use.

24. A pharmaceutical composition according to claim 23, formulated for dermatological use.

25. A pharmaceutical composition comprising a compound of claim 1, pharmaceutically acceptable salt, thereof, and a propellant, formulated for inhalation.

26. A pharmaceutical composition comprising a compound of claim 1, or pharmaceutically acceptable salt thereof, and a propellant, formulated for injection.

27. A pharmaceutical composition comprising a compound claim 1, or a pharmaceutically acceptable salt thereof, and a propellant, formulated for oral use.

28. A pharmaceutical composition according to claim 27, which further comprises at least one additional therapeutically active agent.

28. A pharmaceutical composition according to claim 28, wherein said at least one additional therapeutically active agent is selected from a beta₂ adrenoreceptor agonist, an antihistamine Hᵢ receptor antagonist, an antihistamine H₂ receptor antagonist, an antihistamine H₃ receptor antagonist, an anti-allergic agent, an anticholinergic agent, an expectorant, a decongestant, an antibiotic, a P2Y₂ receptor agonist, a leukotriene₄ antagonist, leukotriene D₄ antagonist, a pharmaceutically acceptable zinc salt, an SYK kinase analog, a 5-lipoxygenase inhibitor, an oropharyngeal discomfort relieving agent, a non-steroidal anti-inflammatory, a TNF inhibitor, an IL-1 receptor antagonist, a cytotoxic or immunosuppressive drug, a p38 kinase inhibitor, a PDE₄ inhibitor, an iNOS inhibitor, a beta-2 integrin antagonist, an adenosine 2a agonist, an antiinfactive agent, an antiviral agent, a chemokine receptor antagonist, and an inhibitor of osteoclast-mediated bone resportion inhibitor.

30. A method for the treatment or prophylaxis of an immune, autoimmune, or inflammatory disease or condition in a patient in need thereof comprising administering an effective amount of a compound of claim 1.

31. A method for the treatment of a skin disease or conditions in a patient in need thereof comprising administering an effective amount of a compound of claim 1.

32. A method of claim 31, wherein said skin disease or condition is selected from eczema, posriasis, allergic dermatitis, atopic dermatitis, neurodermatitis, pruritis, and hypersensitivity reactions.

33. A method for the treatment or prophylaxis of an inflammatory condition of the nose, throat, or lungs in a patient in need thereof comprising administering an effective amount of a compound of claim 1.

34. A method of claim 33, wherein said condition is selected from asthma, allergen-induced asthmatic reactions, rhinitis, hayfever, allergic rhinitis, rhinosinusitis, sinusitis, nasal polyps, chronic bronchitis, chronic obstructive pulmonary disease, interstitial lung disease, and fibrosis.

35. A method for the treatment or prophylaxis of inflammatory bowel conditions in a patient in need thereof comprising administering an effective amount of a compound of claim 1.

36. A method of claim 35, wherein said condition is selected from ulcerative colitis and Chron's disease.

37. A method for the treatment or prophylaxis of an autoimmune disease in a patient in need thereof comprising administering an effective amount of a compound of claim 1.

38. A method of claim 37, wherein said condition is rheumatoid arthritis.

39. A method for the treatment or prophylaxis of multiple sclerosis comprising administering to a patient in need thereof an effective amount of a compound according to claim 1.

40. A method for the treatment or prophylaxis of diseases and conditions of the eye, comprising administering to a patient in need thereof an effective amount of a compound of claim 1.

41. A method of claim 40, wherein said disease or conditions are selected allergic and nonallergic conjunctivitis.
